SEQUENCE LISTING (527 sequences)

SEQ ID NO: 1 - DNA, 150 bp, Klebsiella phage KP34
  Source of Sequence: NCBI:txid674081
gcccatctaccatgcagggtggtgacgtagtaggtattgtacagcagattggtttctatttagtgaaagcaggtggagctctgatcgataacatggctatcctgtttgcaatcggtatcggtgttggtatgtccgaagacaacgacggta

SEQ ID NO: 2 - DNA, 150 bp, Klebsiella phage KP34
  Source of Sequence: NCBI:txid674081
agaatctgccttttcgcggacgtaaagaaaatcatccaccttcgccatcagcttattctgccactcctgttcctcttcggaaacagtttccttttccgtggtttcttcacttgtatcatttccggatataaccgttccatctgctgttgc

SEQ ID NO: 3 - DNA, 150 bp, Klebsiella phage KP34
  Source of Sequence: NCBI:txid674081
ttccgactctgaagggcgacgaaaagactggtgcagaaattattctgcgtgcactggaagcaccgctccgtcagattgcagcaaatgctggtctggaaggcagtgttatcatcgacaagattcggagaagccgcaaggttggttatggct

SEQ ID NO: 4 - DNA, 150 bp, Klebsiella phage KP34
  Source of Sequence: NCBI:txid674081
atgttatcgatcagagctccacctgctttcactaaatagaaaccaatctgctgtacaatacctactacgtcaccaccctgcatggtagatgggcaaagagcatatccaagacccatcaggataccacagataggaaggcaagcaacaggt

SEQ ID NO: 5 - DNA, 150 bp, Klebsiella phage KP34
  Source of Sequence: NCBI:txid674081
aagagggttcgggatgtcattttcgggaaaagtcaaagaggaacttgcaggacagcttagtctggcaaggcactgtcaggtggcagaactggcggcactgctctgtggctgtggccgtgtggaaaaaatgtcggatggaaacaggaaact

SEQ ID NO: 6 - DNA, 150 bp, Klebsiella phage KP34
  Source of Sequence: NCBI:txid674081
gtaagagaaggaggaaaaaagtaattatgatgaaattcttacagaaattaggtaaagctttgatgttacctgttgcttgccttcctatctgtggtatcctgatgggtcttggatatgctctttgcccatctaccatgcagggtggtgacg

SEQ ID NO: 7 - DNA, 150 bp, Klebsiella phage KP34
  Source of Sequence: NCBI:txid674081
atctgtggtatcctgatgggtcttggatatgctctttgcccatctaccatgcagggtggtgacgtagtaggtattgtacagcagattggtttctatttagtgaaagcaggtggagctctgatcgataacatggctatcctgtttgcaatc

SEQ ID NO: 8 - DNA, 150 bp, Klebsiella phage KP34
  Source of Sequence: NCBI:txid674081
tccagcggcaaaaagaaagtaagtgaatattgttatagtttataccagtttatcacagactgcgacattcagagaaaattgaaggaacaggagctttccttcaaagcaaaaggggaaaaagccctggaaaaggaatacgcgcagatatac

SEQ ID NO: 9 - DNA, 150 bp, Klebsiella phage KP34; identical to SEQ ID NO: 4
  Source of Sequence: NCBI:txid674081

SEQ ID NO: 10 - DNA, 150 bp, Klebsiella phage KP34
  Source of Sequence: NCBI:txid674081
cgcttatcggacttacaaacattttcaacacaatctcaaccaatatgcagttgaggagcaaggaatttgcatcgcttaaatctatcggtatgacaaagaaagaatttaaccgtatgattcgccttgaaagccttatgtacggcataaaat

SEQ ID NO: 11 - DNA, 150 bp, Klebsiella phage KP34
  Source of Sequence: NCBI:txid674081
gaacgggttcgcaattttgctgaatgcaatgtaagcagttgtaccttcttcaagagttttgataacagatacagcacctacagacagcagagttgtcagcatcagccaagaagccagagctgcaagtccacctgtaccgtcgttgtcttc

SEQ ID NO: 12 - DNA, 150 bp, Klebsiella phage KP34
  Source of Sequence: NCBI:txid674081
ggatagccatgttatcgatcagagctccacctgctttcactaaatagaaaccaatctgctgtacaatacctactacgtcaccaccctgcatggtagatgggcaaagagcatatccaagacccatcaggataccacagataggaaggcaag

SEQ ID NO: 13 - DNA, 150 bp, Klebsiella phage KP34
  Source of Sequence: NCBI:txid674081
tgacggaaaagttccgctggaatatgcagcctttttgcttacccagtataaggacatgcacacggaaacttacgatacgatgtcagaagtcctggaaaggtattatgcggaaaagaatacgatcacacgcatccgccagaaatcttccga

SEQ ID NO: 14 - DNA, 150 bp, Klebsiella phage KP34
  Source of Sequence: NCBI:txid674081
aaatttgataaatccgagcgagataaggttgaatacttcgtcagcgatatgtggaggactttttcagatatcagttcggtttggttcaaaaacgcaacgaaaattgttgataaataccactggatacggcagattatgtacgcatttgag

SEQ ID NO: 15 - DNA, 150 bp, Klebsiella phage KP34
  Source of Sequence: NCBI:txid674081
gtccacctgtaccgtcgttgtcttcggacataccaacaccgataccgattgcaaacaggatagccatgttatcgatcagagctccacctgctttcactaaatagaaaccaatctgctgtacaatacctactacgtcaccaccctgcatgg

SEQ ID NO: 16 - DNA, 150 bp, Klebsiella phage KP34
  Source of Sequence: NCBI:txid674081
ctgatgggtcttggatatgctctttgcccatctaccatgcagggtggtgacgtagtaggtattgtacagcagattggtttctatttagtgaaagcaggtggagctctgatcgataacatggctatcctgtttgcaatcggtatcggtgtt

SEQ ID NO: 17 - DNA, 150 bp, Klebsiella phage KP34
  Source of Sequence: NCBI:txid674081
aaattttgcaatctgccaaaagtcacttgcttgaatgtagccgtattgaaattctcatcccaaagtattatactgctttttataaaaaggcacacacaagcataccgtgtcttacttttcccgacgcaagcgcaaatgtttgcacatcgg

SEQ ID NO: 18 - DNA, 150 bp, Klebsiella phage KP34
  Source of Sequence: NCBI:txid674081
tctgccgcggcatggtggccatcgtggtctgctcgctggtgctgagccgcctgtttggcatgaatggtgtgtgggcagcgttcccggcatcggaactgctcaccgcagtgctgaccctgtttttgctgctgcgcggtaaaaagagatcgg

SEQ ID NO: 19 - DNA, 150 bp, Klebsiella phage KP34; identical to SEQ ID NO: 12
  Source of Sequence: NCBI:txid674081

SEQ ID NO: 20 - DNA, 150 bp, Klebsiella phage KP34
  Source of Sequence: NCBI:txid674081
gataatccaaatatgcaatcagcggttgataaactattaggcattgaggagtcaaaaataacagtaagtcagaaagttgctcgtagtggtgatgatcgtgcagttgacgctttgaaaatgacttctgaccgtgtggctaagtttgaaaac

SEQ ID NO: 21 - DNA, 150 bp, Klebsiella phage KP34
  Source of Sequence: NCBI:txid674081
taggtattgtacagcagattggtttctatttagtgaaagcaggtggagctctgatcgataacatggctatcctgtttgcaatcggtatcggtgttggtatgtccgaagacaacgacggtacaggtggacttgcagctctggcttcttggc

SEQ ID NO: 22 - DNA, 150 bp, Klebsiella phage KP34
  Source of Sequence: NCBI:txid674081
tttcatcaccccttttatttactccaaaatcggctccaccagctccggggcggctctggctgctgccgttccggctctgccggaactacgcaatcctctgattttcttcatgttctgtacctctttcgtaaattccgattcataatgtaa

SEQ ID NO: 23 - DNA, 150 bp, Klebsiella phage KP34
  Source of Sequence: NCBI:txid674081
cagctcgtagccgtcatcagtggacttgtaggtgtacagggtatcagcaattgcggttaccttgtcggtgtcggtagctgcatcgttaacctttttgccgtctaccttgcgaaccttgatttccttgttagtgccatcggtaaagtaagc

SEQ ID NO: 24 - DNA, 150 bp, Klebsiella phage KP34
  Source of Sequence: NCBI:txid674081
tatcgatcagagctccacctgctttcactaaatagaaaccaatctgctgtacaatacctactacgtcaccaccctgcatggtagatgggcaaagagcatatccaagacccatcaggataccagatcgtaagagcgtcgtgtagggaaaga

SEQ ID NO: 25 - DNA, 150 bp, Klebsiella phage KP34
  Source of Sequence: NCBI:txid674081
gagcaggctaccttcatttttgaacctctgaatgcaaaatctattgcaccaacgttctgtacctcgttgcacactcttacgcagtcaccgcagaggatacacttgttggcgtccctggtgatagatacagaagaatcatcgatacaaggc

SEQ ID NO: 26 - DNA, 150 bp, Klebsiella phage KP34
  Source of Sequence: NCBI:txid674081
acatatccgcagaatccagccagaaaaagagctggaggaggattctcattaagtaagagaaggaggaaaaaagtaattatgatgaaattcttacagaaattaggtaaagctttgatgttacctgttgcttgccttcctatctgtggtatc

SEQ ID NO: 27 - DNA, 150 bp, Klebsiella phage KP34
  Source of Sequence: NCBI:txid674081
cctacagacagcagagttgtcagcatcagccaagaagccagagctgcaagtccacctgtaccgtcgttgtcttcggacataccaacaccgataccgattgcaaacaggatagccatgttatcgatcagagctccacctgctttcactaaa

SEQ ID NO: 28 - DNA, 150 bp, Klebsiella phage KP34
  Source of Sequence: NCBI:txid674081
cagagctccacctgctttcactaaatagaaaccaatctgctgtacaatacctactacgtcaccaccctgcatggtagatgggcaaagagcatatccaagacccatcaggataccacagataggaaggcaagcaacaggtaacatcaaagc

SEQ ID NO: 29 - DNA, 150 bp, Klebsiella phage KP34
  Source of Sequence: NCBI:txid674081
cacctgcacagatcagattgtacttgcttcccattggtcctacgaatgtgtagagggaaagtgccatggtacggatttcttttctcatcaggtaaaggtttgctgcgtagtattcgttgtacacaccaacaccctttaaaatacaggatg

SEQ ID NO: 30 - DNA, 150 bp, Klebsiella phage KP34
  Source of Sequence: NCBI:txid674081
agggtggtgacgtagtaggtattgtacagcagattggtttctatttagtgaaagcaggtggagctctgatcgataacatggctatcctgtttgcaatcggtatcggtgttggtatgtccgaagacaacgacggtacaggtggacttgcag

SEQ ID NO: 31 - DNA, 150 bp, Klebsiella phage KP34
  Source of Sequence: NCBI:txid674081
tgtcagcatcagccaagaagccagagctgcaagtccacctgtaccgtcgttgtcttcggacataccaacaccgataccgattgcaaacaggatagccatgttatcgatcagagctccacctgctttcactaaatagaaaccaatctgctg

SEQ ID NO: 32 - DNA, 150 bp, Klebsiella phage KP34
  Source of Sequence: NCBI:txid674081
agagcacggccggcggcatccggctggacaccctcttcctcgacgagggcttcggctccctcgacgacgagagtctggaacaggccatccgggtcctctccggcctgaccgaaggcgaccggctcgtgggcatcatctcccacgtcgccg

SEQ ID NO: 33 - DNA, 150 bp, Klebsiella phage KP34
  Source of Sequence: NCBI:txid674081
gatatgtacgagataggctttgaatgggacgatcatgtaagacttcgggatacgctgaaaaacatcaaaggtaaatttctgctgtcctacaacgactgcgatgaaattcgtgaactgtacaacggattttcaatgtttgatttttcccga

SEQ ID NO: 34 - DNA, 150 bp, Klebsiella phage KP34; identical to SEQ ID NO: 21
  Source of Sequence: NCBI:txid674081

SEQ ID NO: 35 - DNA, 150 bp, Klebsiella phage KP34
  Source of Sequence: NCBI:txid674081
cggcacggtgctcatgcccacagcatcggcccccatgcccgcaaaggcacggatctcggcggccgtttcatagcagggacccatgaagcccagataaacgccctcttgataggaaatgcccagctcgtcggcggtctgtttggcgagatt

SEQ ID NO: 36 - DNA, 150 bp, Klebsiella phage KP34
  Source of Sequence: NCBI:txid674081
agaaattaggtaaagctttgatgttacctgttgcttgccttcctatctgtggtatcctgatgggtcttggatatgctctttgcccatctaccatgcagggtggtgacgtagtaggtattgtacagcagattggtttctatttattgaaag

SEQ ID NO: 37 - DNA, 150 bp, Klebsiella phage KP34
  Source of Sequence: NCBI:txid674081
agcaggaagaaccgatcacaccggcgatgataccgatgaacgggttcgcaattttgctgaatgcaatgtaagcagttgtaccttcttcaagagttttgataacagatacagcacctacagacagcagagttgtcagcatcagccaagaag

SEQ ID NO: 38 - DNA, 150 bp, Klebsiella phage KP34
  Source of Sequence: NCBI:txid674081
gatatgctctttgcccatctaccatgcagggtggtgacgtagtaggtattgtacagcagattggtttctatttagtgaaagcaggtggagctctgatcgataacatggctatcctgtttgcaatcggtatcggtgttggtatgtccgaag

SEQ ID NO: 39 - DNA, 150 bp, Klebsiella phage KP34
  Source of Sequence: NCBI:txid674081
ccatgttatcgatcagagctccacctgctttcactaaatagaaaccaatctgctgtacaatacctactacgtcaccaccctgcatggtagatgggcaaagagcatatccaagacccatcaggataccacagataggaaggcaagcaacag

SEQ ID NO: 40 - DNA, 150 bp, Klebsiella phage KP34
  Source of Sequence: NCBI:txid674081
gtagtaggtattgtacagcagattggtttctatttagtgaaagcaggtggagctctgatcgataacatggctatcctgtttgcaatcggtatcggtgttggtatgtccgaagacaacgacggtacaggtggacttgcagctctggcttct

SEQ ID NO: 41 - DNA, 150 bp, Klebsiella phage KP34
  Source of Sequence: NCBI:txid674081
gcaaacaggatagccatgttatcgatcagagctccacctgctttcactaaatagaaaccaatctgctgtacaatacctactacgtcaccaccctgcatggtagatgggcaaagagcatatccaagacccatcaggataccacagatagga

SEQ ID NO: 42 - DNA, 150 bp, Klebsiella phage KP34
  Source of Sequence: NCBI:txid674081
gtcaaccagagcgattcttccactggcgtttcctctgcaattgatggtacttgtgatattggtatggcatccagagaactgaaagattcggaaacctcgcagggtgttgtttccacagtcattgcagtcgatgggattgcagtcattgtc

SEQ ID NO: 43 - DNA, 150 bp, Klebsiella phage KP34
  Source of Sequence: NCBI:txid674081
gggaagaattttcggcgaaacagccgcacttatctataccgcgggaactatcgccaagtatgcaggccttacagactcaggacgtaccctttctatccatatgtattcactttcaatcgagggactttatatgaacgaagcaagcgctac

SEQ ID NO: 44 - DNA, 150 bp, Klebsiella phage KP34
  Source of Sequence: NCBI:txid674081
tggtttttccatcagggcaaacgtcccctctgaaacataggtataggtcagcatgctttttacctccatgcgctttgatatttccggacttgagtataccacgcagctatggcaaagagcaagccgcaaaaggcccgccctttgcggcag

SEQ ID NO: 45 - DNA, 150 bp, Klebsiella phage KP34
  Source of Sequence: NCBI:txid674081
gggggtcttggcggccaccacggcgttgccgtaggtctcgcccggcacccagccgaagccatccatgatgcacagaagaacaggtttctttgccataaaattgaatctcctttgaatttaacaaatcagcgcccggcacagcgctggaaa

SEQ ID NO: 46 - DNA, 150 bp, Klebsiella phage KP34
  Source of Sequence: NCBI:txid674081
gattgcaaacaggatagccatgttatcgatcagagctccacctgctttcactaaatagaaaccaatctgctgtacaatacctactacgtcaccaccctgcatggtagatgggcaaagagcatatccaagacccatcaggataccacagat

SEQ ID NO: 47 - DNA, 150 bp, Klebsiella phage KP34; identical to SEQ ID NO: 46
  Source of Sequence: NCBI:txid674081

SEQ ID NO: 48 - DNA, 150 bp, Klebsiella phage KP34
  Source of Sequence: NCBI:txid674081
cttacagaaattaggtaaagctttgatgttacctgttgcttgccttcctatctgtggtatcctgatgggtcttggatatgctctttgcccatctaccatgcagggtggtgacgtagtaggtattgtacagcagattggtttctatttagt

SEQ ID NO: 49 - DNA, 150 bp, Klebsiella phage KP34
  Source of Sequence: NCBI:txid674081
atctctgcggagaaagcggctgcggtctctgtgcgttaaagcagttatccaatgcgggaatcacacatttaaagctcgtcggacgcggaaactatgccaactttatggaacgcgacatccggaatctgcgctgggcactcgatattcttg

SEQ ID NO: 50 - DNA, 150 bp, Klebsiella phage KP34
  Source of Sequence: NCBI:txid674081
catgcataaccgagtctccatccggtcattgcatgcgatttggaaaatccgttgatcacgattgttcgctccctcattcccggaatggaggcaatggatacatgattgtccagataggtcagttccgcatagatctcgtcactcaggaca

SEQ ID NO: 51 - DNA, 150 bp, Klebsiella phage KP34
  Source of Sequence: NCBI:txid674081
ctgtaccgtcgttgtcttcggacataccaacaccgataccgattgcaaacaggatagccatgttatcgatcagagctccacctgctttcactaaatagaaaccaatctgctgtacaatacctactacgtcaccaccctgcatggtagatg

SEQ ID NO: 52 - DNA, 150 bp, Klebsiella phage KP34
  Source of Sequence: NCBI:txid674081
aattatcatgatcgactaccttcagctgatgagcggaagcggaaagagtgattcccgtcagcaggaactttctgatatttcccgttcgcttaaggcacttgcgagagaactgagcgtgccggtcagtgcgctttcgcatctgagccgtgc

SEQ ID NO: 53 - DNA, 95 bp, Klebsiella phage KP34
  Source of Sequence: NCBI:txid674081
aacacattggaagcttcctcgccaatgccatggtctgccgtaattaagtaatagtacgtgtaaagctcactggtcttttcgttgtagttgacggt

SEQ ID NO: 54 - DNA, 150 bp, Klebsiella phage KP34
  Source of Sequence: NCBI:txid674081
tctctcccttacgtcttaatctgaacacatttcagagcttatgatatctagcagaaaatctgcgatatcattatacacctcacggcggttggtttcgatcacaagctcgtgacgtgcattttcgtatatcttcagctcaacgtcacagcc

SEQ ID NO: 55 - DNA, 150 bp, Klebsiella phage KP34
  Source of Sequence: NCBI:txid674081
cagcagagttgtcagcatcagccaagaagccagagctgcaagtccacctgtaccgtcgttgtcttcggacataccaacaccgataccgattgcaaacaggatagccatgttatcgatcagagctccacctgctttcactaaatagaaacc

SEQ ID NO: 56 - DNA, 150 bp, Klebsiella phage KP34
  Source of Sequence: NCBI:txid674081
taccatgcagggtggtgacgtagtaggtattgtacagcagattggtttctatttagtgaaagcaggtggagctctgatcgataacatggctatcctgtttgcaatcggtatcggtgttggtatgtccgaagacaacgacggtacaggtgg

SEQ ID NO: 57 - DNA, 150 bp, Klebsiella phage KP34
  Source of Sequence: NCBI:txid674081
gactccacggaacaaattcgctgtctccgtgttcgcctattacatatgcgtgaatattgcgtgggtcaacctcaaaatattcgccgagaagatatctgagccgtgccgtgtcaagggtagttcccgtgccgatgatgtttgacgcattaa

SEQ ID NO: 58 - DNA, 150 bp, Klebsiella phage KP34
  Source of Sequence: NCBI:txid674081
taaagctttgatgttacctgttgcttgccttcctatctgtggtatcctgatgggtcttggatatgctctttgcccatctaccatgcagggtggtgacgtagtaggtattgtacagcagattggtttctatttagtgaaagcaggtggagc

SEQ ID NO: 59 - DNA, 150 bp, Klebsiella phage KP34; identical to SEQ ID NO: 39
  Source of Sequence: NCBI:txid674081

SEQ ID NO: 60 - DNA, 150 bp, Klebsiella phage KP34
  Source of Sequence: NCBI:txid674081
aaacctattgtaatctttgcagtatgtatgggtatagtaaatgtaatctttgcttttgacttaacaggttacgaaagaagagtgccaagtgaaaataatgttgtaagtatagatgcctttgactatatgggacatattagttacgatagt

SEQ ID NO: 61 - DNA, 150 bp, Klebsiella phage KP34
  Source of Sequence: NCBI:txid674081
tcgcaattttgctgaatgcaatgtaagcagttgtaccttcttcaagagttttgataacagatacagcacctacagacagcagagttgtcagcatcagccaagaagccagagctgcaagtccacctgtaccgtcgttgtcttcggacatac

SEQ ID NO: 62 - DNA, 120 bp, Klebsiella phage KP34
  Source of Sequence: NCBI:txid674081
taaacctctttgacctatgagacttaaaagaggatagttcgtcaattacaatcatatcgtagttaaaggacagttcgctcttatttattaaccagtctacattttccctattgagtaaat

SEQ ID NO: 63 - DNA, 150 bp, Klebsiella phage KP34
  Source of Sequence: NCBI:txid674081
tgaaatccaaggtgatgttttaacagatatatattcatatggcaatatttataattatattcctttttttacaagtgaaaataaaatttattatagtacaattaaaaatggaaaattaaaaaaacctgccttattaagtggatgggcaga

SEQ ID NO: 64 - DNA, 150 bp, Klebsiella phage KP34
  Source of Sequence: NCBI:txid674081
tgcccaaccgaaaccgccccatttttgctttggcttttcaagtttgcggtacattttaatcggacttgtctgtgccgctctgcccaccgaaaattccattatcaaacacggaattcccaaaacaataagacagatgatgtacacaagcat

SEQ ID NO: 65 - DNA, 150 bp, Klebsiella phage KP34
  Source of Sequence: NCBI:txid674081
gtcagacatggcaaggcaagtgaatattccctacagcgcactatataacagccttttcaacgaaaaacgggatcgtgatctgcgagtagatgagtttcttacggtatgcaagtatctggatgtcgatccgatgatcttttggaaaccatg

SEQ ID NO: 66 - DNA, 150 bp, Klebsiella phage KP34
  Source of Sequence: NCBI:txid674081
caacaccgataccgattgcaaacaggatagccatgttatcgatcagagctccacctgctttcactaaatagaaaccaatctgctgtacaatacctactacgtcaccaccctgcatggtagatgggcaaagagcatatccaagacccatca

SEQ ID NO: 67 - DNA, 150 bp, Klebsiella phage KP34
  Source of Sequence: NCBI:txid674081
caaagacggtactttaacatacctgcgtccggacggaaaaacacaggtaaccgtagagtacgatgagaacggcaaaccgttccgtcttgatgcggttgttctttccactcagcatgatccggaagtatcccaggagcagattcatgagga

SEQ ID NO: 68 - DNA, 150 bp, Klebsiella phage KP34
  Source of Sequence: NCBI:txid674081
gctttcactaaatagaaaccaatctgctgtacaatacctactacgtcaccaccctgcatggtagatgggcaaagagcatatccaagacccatcaggataccacagataggaaggcaagcaacaggtaacatcaaagctttacctaatttc

SEQ ID NO: 69 - DNA, 149 bp, Klebsiella phage KP34
  Source of Sequence: NCBI:txid674081
agcgcggtggatttgccgcagccggagccgcccagcagcaccaccatttcgcccggattgatttccaggttgaaatcgtccaacgccttgacggtcgaaccggggtagatcttgtctacaccaagcatctgcacaccgccgccgcgcgc

SEQ ID NO: 70 - DNA, 150 bp, Klebsiella phage KP34
  Source of Sequence: NCBI:txid674081
tcttacagaaattaggtaaagctttgatgttacctgttgcttgccttcctatctgtggtatcctgatgggtcttggatatgctctttgcccatctaccatgcagggtggtgacgtagtaggtattgtacagcagattggtttctatttag

SEQ ID NO: 71 - DNA, 150 bp, Klebsiella phage KP34
  Source of Sequence: NCBI:txid674081
gaaaagaaacctcttggatatagaaaaaatatcatatatgtactataatgaaacagtgacaaaaaatgcacataatgtgtgggaaaggtaatatcatgaaagaaaaaaatgtgtcattagagaataaagtgattttagtaaccggtgctg

SEQ ID NO: 72 - DNA, 150 bp, Klebsiella phage KP34
  Source of Sequence: NCBI:txid674081
catgcagggtggtgacgtagtaggtattgtacagcagattggtttctatttagtgaaagcaggtggagctctgatcgataacatggctatcctgtttgcaatcggtatcggtgttggtatgtccgaagacaacgacggtacaggtggact

SEQ ID NO: 73 - DNA, 150 bp, Klebsiella phage KP34
  Source of Sequence: NCBI:txid674081
gagctccacctgctttcactaaatagaaaccaatctgctgtacaatacctactacgtcaccaccctgcatggtagatgggcaaagagcatatccaagacccatcaggataccacagataggaaggcaagcaacaggtaacatcaaagctt

SEQ ID NO: 74 - DNA, 150 bp, Klebsiella phage KP34
  Source of Sequence: NCBI:txid674081
ttacagaaattaggtaaagctttgatgttacctgttgcttgccttcctatctgtggtatcctgatgggtcttggatatgctctttgcccatctaccatgcagggtggtgacgtagtaggtattgtacagcagattggtttctatttagtg

SEQ ID NO: 75 - DNA, 150 bp, Klebsiella phage KP34
  Source of Sequence: NCBI:txid674081
taccgattgcaaacaggatagccatgttatcgatcagagctccacctgctttcactaaatagaaaccaatctgctgtacaatacctactacgtcaccaccctgcatggtagatgggcaaagagcatatccaagacccatcaggataccac

SEQ ID NO: 76 - DNA, 150 bp, Klebsiella phage KP34
  Source of Sequence: NCBI:txid674081
ataaaaaccgtcctttttaaagaaaatagggcaagtcatataatatataactcgccctttgagtttgaatatatgactgcaaatgcagtttttatcttaatttaatatgaacctcacggagctgctgttctgttacctcacccggtgaac

SEQ ID NO: 77 - DNA, 150 bp, Klebsiella phage KP34
  Source of Sequence: NCBI:txid674081
ggtgacgtagtaggtattgtacagcagattggtttctatttagtgaaagcaggtggagctctgatcgataacatggctatcctgtttgcaatcggtatcggtgttggtatgtccgaagacaacgacggtacaggtggacttgcagctctg

SEQ ID NO: 78 - DNA, 150 bp, Klebsiella phage KP34
  Source of Sequence: NCBI:txid674081
tacagacagcagagttgtcagcatcagccaagaagccagagctgcaagtccacctgtaccgtcgttgtcttcggacataccaacaccgataccgattgcaaacaggatagccatgttatcgatcagagctccacctgctttcactaaata

SEQ ID NO: 79 - DNA, 150 bp, Klebsiella phage KP34
  Source of Sequence: NCBI:txid674081
gtgatataaaggtcaaacaggttcttgaagttatcaagataggttcctttgatcgcatctgtggatgtgattccgtcttctttgtactcataatagatcggaaggtttgcaaggtgtgtcttgaagcgccagtcactggaagaatcaaag

SEQ ID NO: 80 - DNA, 150 bp, Klebsiella phage KP34
  Source of Sequence: NCBI:txid674081
aaaagcagcgaatgaacttcagatacccgtaaatacgctgtacggctggatcagaaaagtaaaaatcggaagccttgatattggctgcggagaacgcagcccggaagaatctctgaatatcgccgaagaaaaccagcagctgaggaagcg

SEQ ID NO: 81 - DNA, 150 bp, Klebsiella phage KP34
  Source of Sequence: NCBI:txid674081
ggtggtgacgtagtaggtattgtacagcagattggtttctatttagtgaaagcaggtggagctctgatcgataacatggctatcctgtttgcaatcggtatcggtgttggtatgtccgaagacaacgatggtacaggtggacttgcagct

SEQ ID NO: 82 - DNA, 150 bp, Klebsiella phage KP34
  Source of Sequence: NCBI:txid674081
ccgattgcaaacaggatagccatgttatcgatcagagctccacctgctttcactaaatagaaaccaatctgctgtacaatacctactacgtcaccaccctgcatggtagatgggcaaagagcatatccaagacccatcaggataccacag

SEQ ID NO: 83 - DNA, 150 bp, Klebsiella phage KP34
  Source of Sequence: NCBI:txid674081
ccgattaccgtaaataaaaatggaagcggtaccgttaaaatcaataacgaagaggtaacggattctaaaactgttgaaaaagacagtacggtagaactggaagttaccccggcaaaagatacctacatcaagaaacttgtaatcggcgga

SEQ ID NO: 84 - DNA, 150 bp, Klebsiella phage KP34
  Source of Sequence: NCBI:txid674081
atgttacctgttgcttgccttcctatctgtggtatcctgatgggtcttggatatgctctttgcccatctaccatgcagggtggtgacgtagtaggtattgtacagcagattggtttctatttagtgaaagcaggtggagctctgatcgat

SEQ ID NO: 85 - DNA, 150 bp, Klebsiella phage KP34
  Source of Sequence: NCBI:txid674081
cggaaaagaacagccacagacagtaccggtgtatgtgaagttttccaatgagagagatcagaagctctccatttatacggaaatcagtgaagcagcagatgggcagctgactgtgaagaaagtaccgtcgcagaagaaagcggcagctca

SEQ ID NO: 86 - DNA, 150 bp, Klebsiella phage KP34
  Source of Sequence: NCBI:txid674081
tgctccacaaggcttcgctttgatcttaaagataaaaattctattgacaaagctgccttagaaagaaccgatggcatattacaggtaatggaagttgccgggcaaacacaggttgtaattggttcaaatgttcaatatgtatacgatgaa

SEQ ID NO: 87 - DNA, 150 bp, Klebsiella phage KP34
  Source of Sequence: NCBI:txid674081
gagctggaggaggattctcattaagtaagagaaggaggaaaaaagtaattatgatgaaattcttacagaaattaggtcaagctttgatgttacctgttgcttgccttcctatctgtggtatcctgatgggtcttggatatgctctttgcc

SEQ ID NO: 88 - DNA, 150 bp, Klebsiella phage KP34
  Source of Sequence: NCBI:txid674081
gaagaataaacatcttcctgtcctttaattgttgcaaatagggggtacaaatgcgtcaagttatagctagagctatttgtatcccctgttagcatggttcttcacctgtatttctatttcctatttcattcttttgccacaaccttttac

SEQ ID NO: 89 - DNA, 150 bp, Klebsiella phage KP34
  Source of Sequence: NCBI:txid674081
gggtggtgacgtagtaggtattgtacagcagattggtttctatttagtgaaagcaggtggagctctgatcgataacatggctatcctgtttgcaatcggtatcggtgttggtatgtccgaagacaacgacggtacaggtggacttgcagc

SEQ ID NO: 90 - DNA, 150 bp, Klebsiella phage KP34
  Source of Sequence: NCBI:txid674081
tgtggtatcctgatgggtcttggatatgctctttgcccatctaccatgcagggtggtgacgtagtaggtattgtacagcagattggtttctatttagtgaaagcaggtggagctctgatcgataacatggctatcctgtttgcaatcggt

SEQ ID NO: 91 - DNA, 150 bp, Klebsiella phage KP34
  Source of Sequence: NCBI:txid674081
aaataccggatatgttatacttccattatcaatatccttataaaaatcatctttgttcatatatgatttagcgcatttatatccatttttagtaagccataaatacatttcaaatttatctaaagatgtctcgcacaattcataagatga

SEQ ID NO: 92 - DNA, 149 bp, Klebsiella phage KP34
  Source of Sequence: NCBI:txid674081
atatcaaaatcgtaaattatcatattggaaaattgagaactttgtgctatattgttagagtaatgaaaaacaaatcattttttttgggaggaacttatcatgaagaaaaaagtaatcgctggtttacttggaacagcaatggtagcatc

SEQ ID NO: 93 - DNA, 150 bp, Klebsiella phage KP34
  Source of Sequence: NCBI:txid674081
cgatcagagctccacctgctttcactaaatagaaaccaatctgctgtacaatacctactacgtcaccaccctgcatggtagatgggcaaagagcatatccaagacccatcaggataccacagataggaaggcaagcaacaggtaacatca

SEQ ID NO: 94 - DNA, 150 bp, Klebsiella phage KP34
  Source of Sequence: NCBI:txid674081
gtatcctgatgggtcttggatatgctctttgcccatctaccatgcagggtggtgacgtagtaggtattgtacagcagattggtttctatttagtgaaagcaggtggagctctgatcgataacatggctatcctgtttgcaatcggtatcg

SEQ ID NO: 95 - DNA, 115 bp, Klebsiella phage KP34
  Source of Sequence: NCBI:txid674081
cgtttgagatgagacccttcccggataacaatcgcggtctctatattaaaggtttttcgcaataatgtaaagctttttcttgcaactgcttcattttctgtctggatccacagtt

SEQ ID NO: 96 - DNA, 150 bp, Klebsiella phage KP34
  Source of Sequence: NCBI:txid674081
ggtaaagctttgatgttacctgttgcttgccttcctatctgtggtatcctgatgggtcttggatatgctctttgcccatctaccatgcagggtggtgacgtagtaggtattgtacagcagattggtttctatttagtgaaagcaggtgga

SEQ ID NO: 97 - DNA, 150 bp, Klebsiella phage KP34
  Source of Sequence: NCBI:txid674081
ccacctgctttcactaaatagaaaccaatctgctgtacaatacctactacgtcaccaccctgcatggtagatgggcaaagagcatatccaagacccatcaggataccacagataggaaggcaagcaacaggtaacatcaaagctttacct

SEQ ID NO: 98 - DNA, 150 bp, Klebsiella phage KP34
  Source of Sequence: NCBI:txid674081
atacagcacctacagacagcagagttgtcagcatcagccaagaagccagagctgcaagtccacctgtaccgtcgttgtcttcggacataccaacaccgataccgattgcaaacaggatagccatgttatcgatcagagctccacctgctt

SEQ ID NO: 99 - DNA, 150 bp, Klebsiella phage KP34; identical to SEQ ID NO: 77
  Source of Sequence: NCBI:txid674081

SEQ ID NO: 100 - DNA, 150 bp, Klebsiella phage KP34
  Source of Sequence: NCBI:txid674081
tattcgctgtttccacaaaaaaccggacggaacttcggggcttcttccctcttgacaattccggatcttgagataagaagcaagccgcagcttctttccctcttgtaaaatccggtcaaaacggttatgatagaaccaaataaaaaacaa

SEQ ID NO: 101 - DNA, 150 bp, Klebsiella phage KP34
  Source of Sequence: NCBI:txid674081
tttcactaaatagaaaccaatctgctgtacaatacctactacgtcaccaccctgcatggtagatgggcaaagagcatatccaagacccatcaggataccacagataggaaggcaagcaacaggtaacatcaaagctttacctaatttctg

SEQ ID NO: 102 - DNA, 150 bp, Klebsiella phage KP34
  Source of Sequence: NCBI:txid674081
ctaccatgcagggtggtgacgtagtaggtattgtacagcagattggtttctatttagtgaaagcaggtggagctctgatcgataacatggctatcctgtttgcaatcggtatcggtgttggtatgtccgaagacaacgacggtacaggtg

SEQ ID NO: 103 - DNA, 150 bp, Klebsiella phage KP34
  Source of Sequence: NCBI:txid674081
ttcagaattctttcaaaaaattacgattttacacaattctaccatttttgcatggtaaaataaggtcgtagaaaaaacaaaggatgtggctcaatatgcaacactgtcccgcccgcgcggcacagcggcagatcggaagagcacacgtct

SEQ ID NO: 104 - DNA, 150 bp, Klebsiella phage KP34
  Source of Sequence: NCBI:txid674081
tgcttgccttcctatctgtggtatcctgatgggtcttggatatgctctttgcccatctaccatgcagggtggtgacgtagtaggtattgtacagcagattggtttctatttagtgaaagcaggtggagctctgatcgataacatggctat

SEQ ID NO: 105 - DNA, 150 bp, Klebsiella phage KP34
  Source of Sequence: NCBI:txid674081
ggtattgtacagcagattggtttctatttagtgaaagcaggtggagctctgatcgataacatggctatcctgtttgcaatcggtatcggtgttggtatgtccgaagacaacgacggtacaggtggacttgcagctctggcttcttggctg

SEQ ID NO: 106 - DNA, 150 bp, Klebsiella phage KP34
  Source of Sequence: NCBI:txid674081
ttgccttcctatctgtggtatcctgatgggtcttggatatgctctttgcccatctaccatgcagggtggtgacgtagtaggtattgtacagcagattggtttctatttagtgaaagcaggtggagctctgatcgataacatggctatcct

SEQ ID NO: 107 - DNA, 150 bp, Klebsiella phage KP34
  Source of Sequence: NCBI:txid674081
gcatcagccaagaagccagagctgcaagtccacctgtaccgtcgttgtcttcggacataccaacaccgataccgattgcaaacaggatagccatgttatcgatcagagctccacctgctttcactaaatagaaaccaatctgctgtacaa

SEQ ID NO: 108 - DNA, 150 bp, Klebsiella phage KP34
  Source of Sequence: NCBI:txid674081
gttatcgatcagagctccacctgctttcactaaatagaaaccaatctgctgtacaatacctactacgtcaccaccctgcatggtagatgggcaaagagcatatccaagacccatcaggataccacagataggaaggcaagcaacaggtaa

SEQ ID NO: 109 - DNA, 150 bp, Klebsiella phage KP34
  Source of Sequence: NCBI:txid674081
gataccgattgcaaacaggatagccatgttatcgatcagagctccacctgctttcactaaatagaaaccaatctgctgtacaatacctactacgtcaccaccctgcatggtagatgggcaaagagcatatccaagacccatcaggatacc

SEQ ID NO: 110 - DNA, 150 bp, Klebsiella phage KP34
  Source of Sequence: NCBI:txid674081
gcttaagaggtctgcatctactacggtgaacttagttctcttcgaagtatctaccactgattcgaatatatactctccagcttgtgcagttacaaactcataaccagcaccacctgcatcataagtagttactttaccaacttcccttat

SEQ ID NO: 111 - DNA, 150 bp, Klebsiella phage KP34
  Source of Sequence: NCBI:txid674081
tatcgatcagagctccacctgctttcactaaatagaaaccaatctgctgtacaatacctactacgtcaccaccctgcatggtagatgggcaaagagcatatccaagacccatcaggataccacagataggaaggcaagcaacaggtaaca

SEQ ID NO: 112 - DNA, 150 bp, Klebsiella phage KP34
  Source of Sequence: NCBI:txid674081
cgcaattttgctgaatgcaatgtaagcagttgtaccttcttcaagagttttgataacagatacagcacctacagacagcagagttgtcagcatcagccaagaagccagagctgcaagtccacctgtaccgtcgttgtcttcggacatacc

SEQ ID NO: 113 - DNA, 150 bp, Klebsiella phage KP34
  Source of Sequence: NCBI:txid674081
ccatctaccatgcagggtggtgacgtagtaggtattgtacagcagattggtttctatttagtgaaagcaggtggagctctgatcgataacatggctatcctgtttgcaatcggtatcggtgttggtatgtccgaagacaacgacggtaca

SEQ ID NO: 114 - DNA, 150 bp, Klebsiella phage KP34; identical to SEQ ID NO: 113
  Source of Sequence: NCBI:txid674081

SEQ ID NO: 115 - DNA, 150 bp, Klebsiella phage KP34
  Source of Sequence: NCBI:txid674081
cggcaagggccagatattcccctgcggcaagctctgccgcagcattggtgttggccgcaatgcctttgttttcgatctttttgtatacgatgcgttgattctttgtctgatattcctccacgatgcgcttcacatcggcatgttctgcat

SEQ ID NO: 116 - DNA, 150 bp, Klebsiella phage KP34
  Source of Sequence: NCBI:txid674081
ggtatcctgatgggtcttggatatgctctttgcccatctaccatgcagggtggtgacgtagtaggtattgtacagcagattggtttctatttagtgaaagcaggtggagctctgatcgataagatcggaagagcacacgtctgaactcca

SEQ ID NO: 117 - DNA, 150 bp, Klebsiella phage KP34; identical to SEQ ID NO: 108
  Source of Sequence: NCBI:txid674081

SEQ ID NO: 118 - DNA, 150 bp, Klebsiella phage KP34
  Source of Sequence: NCBI:txid674081
atcctgatgggtcttggatatgctctttgcccatctaccatgcagggtggtgacgtagtaggtattgtacagcagattggtttctatttagtgaaagcaggtggagctctgatcgataacatggctatcctgtttgcaatcggtatcggt

SEQ ID NO: 119 - DNA, 150 bp, Klebsiella phage KP34
  Source of Sequence: NCBI:txid674081
tgccatgtatagtcaacttcaagcatggagtttgcaaaatctctgccataaactgaataaactgcggaatatatcttgccgtcccctgctgtcaatatctcaactttattatccctgatgaacttgaatgatatctcagggtgtgaaagg

SEQ ID NO: 120 - DNA, 150 bp, Klebsiella phage KP34; identical to SEQ ID NO: 73
  Source of Sequence: NCBI:txid674081

SEQ ID NO: 121 - DNA, 150 bp, Klebsiella phage KP34
  Source of Sequence: NCBI:txid674081
ccacggcgttcgatacgacaagctcggcggcgcccttgcctttgccgtgcgaaacggcgcacaggctcagctcaatcacgctgccctcgctcgcatagcgcgtggcgttgtccagtagcaactcaaccacctgcgccaccgccgcggcat

SEQ ID NO: 122 - DNA, 150 bp, Klebsiella phage KP34
  Source of Sequence: NCBI:txid674081
cctatctgtggtatcctgatgggtcttggatatgctctttgcccatctaccatgcagggtggtgacgtagtaggtattgtacagcagattggtttctatttagtgaaagcaggtggagctctgatcgataacatggctatcctgtttgca

SEQ ID NO: 123 - DNA, 150 bp, Klebsiella phage KP34
  Source of Sequence: NCBI:txid674081
gcaagtccacctgtactgtcgttgtcttcggacataccaacaccgataccgattgcaaacaggatagccatgttatcgatcagagctccacctgctttcactaaatagaaaccaatctgctgtacaatacctactacgtcaccaccctgc

SEQ ID NO: 124 - DNA, 150 bp, Klebsiella phage KP34
  Source of Sequence: NCBI:txid674081
ctttcactaaatagaaaccaatctgctgtacaatacctactacgtcaccaccctgcatggtagatgggcaaagagcatatccaagacccatcaggataccacagataggaaggcaagcaacaggtaacatcaaagctttacctaatttct

SEQ ID NO: 125 - DNA, 150 bp, Klebsiella phage KP34
  Source of Sequence: NCBI:txid674081
atgagagaaggaatccatccgaattactatcaggcaactgtaacctgcaactgcggtaatacttttgtaacaggatctaccaagcaggacatccacgtagaaatctgctccaaatgtcatccgttctatacaggacagcagaaagctagt

SEQ ID NO: 126 - DNA, 150 bp, Klebsiella phage KP34; identical to SEQ ID NO: 122
  Source of Sequence: NCBI:txid674081

SEQ ID NO: 127 - DNA, 150 bp, Klebsiella phage KP34
  Source of Sequence: NCBI:txid674081
agacagcagagttgtcagcatcagccaagaagccagagctgcaagtccacctgtaccgtcgttgtcttcggacataccaacaccgataccgattgcaaacaggatagccatgttatcgatcagagctccacctgctttcactaaatagaa

SEQ ID NO: 128 - DNA, 150 bp, Klebsiella phage KP34
  Source of Sequence: NCBI:txid674081
agtaggtattgtacagcagattggtttctatttagtgaaagcaggtggagctctgatcgataacatggctatcctgtttgcaatcggtatcggtgttggtatgtccgaagacaacgacggtacaggtggacttgcagctctggcttcttg

SEQ ID NO: 129 - DNA, 150 bp, Klebsiella phage KP34
  Source of Sequence: NCBI:txid674081
gcaagtccacctgtaccgtcgttgtcttcggacataccaacaccgataccgattgcaaacaggatagccatgttatcgatcagagctccacctgctttcactaaatagaaaccaatctgctgtacaatacctactacgtcaccaccctgc

SEQ ID NO: 130 - DNA, 150 bp, Klebsiella phage KP34
  Source of Sequence: NCBI:txid674081
atacctgctttcttccgggaatacagcgaaacgaaagcgtgccggactgtattcctggagtttgatgaaaaatttgaaatcacataccctatcggggtatctaaaatatagcgcggatgaaaagaaatgtcaataagctgataaaatatg

SEQ ID NO: 131 - DNA, 150 bp, Klebsiella phage KP34
  Source of Sequence: NCBI:txid674081
ggatgggaatttataatcttctgatgaagactggaatgggaatctggccgtctgggataattgccctgctgattttgcttcaatacggcgtgatgactggtggaagtgtgtctactatgcgtgcagtctgtatgtttttgatatctgtag

SEQ ID NO: 132 - DNA, 150 bp, Klebsiella phage KP34
  Source of Sequence: NCBI:txid674081
ctggccgtacatatccgcagaatccagccagaaaaagagctggaggaggattctcattaagtaagagaaggaggaaaaaagtaattatgatgaaattcttacagaaattaggtaaagctttgatgttacctgttgcttgccttcctatct

SEQ ID NO: 133 - DNA, 150 bp, Klebsiella phage KP34
  Source of Sequence: NCBI:txid674081
gatgggtcttggatatgctctttgcccatctaccatgcagggtggtgacgtagtaggtattgtacagcagattggtttctatttagtgaaagcaggtggagctctgatcgataacatggctatcctgtttgcaatcggtatcggtgttgg

SEQ ID NO: 134 - DNA, 150 bp, Klebsiella phage KP34
  Source of Sequence: NCBI:txid674081
tgcagggtggtgacgtagtaggtattgtacagcagattggtttctatttagtgaaagcaggtggagctctgatcgataacatggctatcctgtttgcaatcggtatcggtgttggtatgtccgaagacaacgacggtacaggtggacttg

SEQ ID NO: 135 - DNA, 150 bp, Klebsiella phage KP34
  Source of Sequence: NCBI:txid674081
tcgatcagagctccacctgctttcactaaatagaaaccaatctgctgtacaatacctactacgtcaccaccctgcatggtagatgggcaaagagcatatccaagacccatcaggataccacagataggaaggcaagcaacaggtaacatc

SEQ ID NO: 136 - DNA, 150 bp, Klebsiella phage KP34; identical to SEQ ID NO: 72
  Source of Sequence: NCBI:txid674081

SEQ ID NO: 137 - DNA, 150 bp, Klebsiella phage KP34
  Source of Sequence: NCBI:txid674081
ctgcgaaaacggcggaaattccgttataagaacaccgtttttctttatcctgttacgcaaatcctcatttgttttaaggtaatttgttccgagaccgcagccgagcaccgcaacggttttcttgtccacgctcaatgcgccctcatgcga

SEQ ID NO: 138 - DNA, 150 bp, Klebsiella phage KP34
  Source of Sequence: NCBI:txid674081
cctgtgcctggttctcttcgcggtggggcggagcttttggttcatgttgttggtgttcctgctggcggggcttatgcgcaccatcaaggagcctgtgctggccgcctggatgaacgaccatgtggatgagaaaatgcgcgccacagtctt

SEQ ID NO: 139 - DNA, 150 bp, Klebsiella phage KP34
  Source of Sequence: NCBI:txid674081
gatgaccgcagcccttcggaaagagttcggttttcatcttccttcgatattctgcatcacttttggccgtatcaagaatatcgagtgcccagcgcagattccggatgtcgcgttccataaagttggcatagtttccgcgtccgacgagct

SEQ ID NO: 140 - DNA, 150 bp, Klebsiella phage KP34
  Source of Sequence: NCBI:txid674081
agccatgttatcgatcagagctccacctgctttcactaaatagaaaccaatctgctgtacaatacctactacgtcaccaccctgcatggtagatgggcaaagagcatatccaagacccatcaggataccacagataggaaggcaagcaac

SEQ ID NO: 141 - DNA, 150 bp, Klebsiella phage KP34
  Source of Sequence: NCBI:txid674081
gaaattaggtaaagctttgatgttacctgttgcttgccttcctatctgtggtatcctgatgggtcttggatatgctctttgcccatctaccatgcagggtggtgacgtagtaggtattgtacagcagattggtttctatttagtgaaagc

SEQ ID NO: 142 - DNA, 150 bp, Klebsiella phage KP34
  Source of Sequence: NCBI:txid674081
cttcgacgaagacgatgattcccgtatcgtcgcgtgaaacgatatcgagttcgccatatcggcagtgccagttgcggtcgagcgtctgccagccttggctttccagccatgcggccgcatactgttcgccgagttcgccgacctgtcgtg

SEQ ID NO: 143 - DNA, 150 bp, Klebsiella phage KP34
  Source of Sequence: NCBI:txid674081
gatgggtcttggatatgctctttgcccatctaccatgcaggggggtgacgtagtaggtattgtacagcagattggtttctatttagtgaaagcaggtggagctctgatcgataacatggctatcctgtttgcaatcggtatcggtgttgg

SEQ ID NO: 144 - DNA, 150 bp, Klebsiella phage KP34
  Source of Sequence: NCBI:txid674081
cgattgcaaacaggatagccatgttatcgatcagagctccacctgctttcactaaatagaaaccaatctgctgtacaatacctactacgtcaccaccctgcatggtagatgggcaaagagcatatccaagacccatcaggataccacaga

SEQ ID NO: 145 - DNA, 150 bp, Klebsiella phage KP34; identical to SEQ ID NO: 68
  Source of Sequence: NCBI:txid674081

SEQ ID NO: 146 - DNA, 150 bp, Klebsiella phage KP34
  Source of Sequence: NCBI:txid674081
tgttacctgttgcttgccttcctatctgtggtatcctgatgggtcttggatatgctctttgcccatctaccatgcagggtggtgacgtagtaggtattgtacagcagattggtttctatttagtgaaagcaggtggagctctgatcgata

SEQ ID NO: 147 - DNA, 150 bp, Klebsiella phage KP34
  Source of Sequence: NCBI:txid674081
gaatcactcgaagtactgcggcgaaagtgattgccggttattttcatacagaggcaactcatgtcggcggttgttatgatgcatattctgttcgtgacaatgacggcaggatgtggaagattatgcgtgacgcaagcgtccgttgtgaaa

SEQ ID NO: 148 - DNA, 150 bp, Klebsiella phage KP34
  Source of Sequence: NCBI:txid674081
tgtcttcggacataccaacaccgataccgattgcaaacaggatagccatgttatcgatcagagctccacctgctttcactaaatagaaaccaatctgctgtacaatacctactacgtcaccaccctgcatggtagatgggcaaagagcat

SEQ ID NO: 149 - DNA, 150 bp, Klebsiella phage KP34
  Source of Sequence: NCBI:txid674081
attgttgtaatctgcgcaggtgcaaatcaaaagcccaccgaaagcaggcttcagcttttgcagaaaaacgcagttgtattttcttcaattgtgccaaaggttgtgcaaagcggttttgagggaattttccttgttgcgacaaaccccgtt

SEQ ID NO: 150 - DNA, 150 bp, Klebsiella phage KP34
  Source of Sequence: NCBI:txid674081
acctccgcagctaccacctgccactggtccggctctactcatcagttcccggagcatcgtatcaacatcatcgacaccccgggccacgttgactttaccgtagaggtagagcgctccctgcgtgtactggacggttccgtaaccgtattc

SEQ ID NO: 151 - DNA, 150 bp, Klebsiella phage KP34
  Source of Sequence: NCBI:txid674081
ttggatatgctctttgcccatctaccatgcagggtggtgacgtagtaggtattgtacagcagattggtttctatttagtgaaagcaggtggagctctgatcgataacatggctatcctgtttgcaatcggtatcggtgttggtatgtccg

SEQ ID NO: 152 - DNA, 150 bp, Klebsiella phage KP34
  Source of Sequence: NCBI:txid674081
tgctctttgcccatctaccatgcagggtggtgacgtagtaggtattgtacagcagattggtttctatttagtgaaagcaggtggagctctgatcgataacatggctatcctgtttgcaatcggtatcggtgttggtatgtccgaagacaa

SEQ ID NO: 153 - DNA, 150 bp, Klebsiella phage KP34; identical to SEQ ID NO: 152
  Source of Sequence: NCBI:txid674081

SEQ ID NO: 154 - DNA, 150 bp, Klebsiella phage KP34
  Source of Sequence: NCBI:txid674081
atcgatcagagctccacctgctttcactaaatagaaaccaatctgctgtacaatacctactacgtcaccaccctgcatggtagatgggcaaagagcatatccaagacccatcaggataccacagataggaaggcaagcaacaggtaacat

SEQ ID NO: 155 - DNA, 150 bp, Klebsiella phage KP34; identical to SEQ ID NO: 154
  Source of Sequence: NCBI:txid674081

SEQ ID NO: 156 - DNA, 150 bp, Klebsiella phage KP34
  Source of Sequence: NCBI:txid674081
actaaatagaaaccaatctgctgtacaatacctactacgtcaccaccctgcatggtagatgggcaaagagcatatccaagacccatcaggataccacagataggaaggcaagcaacaggtaacatcaaagctttacctaatttctgtaag

SEQ ID NO: 157 - DNA, 150 bp, Klebsiella phage KP34
  Source of Sequence: NCBI:txid674081
cttctttgctctgttcctgcaacattgcagtcaattcactgtttccttttgtcataccctgaataattgttgatgtaaactttttaatctccggaatgatgcaacgagaaccaaaattaaacatcgcgtcaatctcagaaacaccgttat

SEQ ID NO: 158 - DNA, 150 bp, Klebsiella phage KP34; identical to SEQ ID NO: 7
  Source of Sequence: NCBI:txid674081

SEQ ID NO: 159 - DNA, 150 bp, Klebsiella phage KP34
  Source of Sequence: NCBI:txid674081
caggtaaaagttccgtatttcgcacatactcaggtaaaaaaagtaaaagaaattcaaaagtatatggtttccaatttgcgccagcattacactcttgaacaattgtccgagcaatttgacattcctcttacttccatgaaagcatgtttt

SEQ ID NO: 160 - DNA, 150 bp, Klebsiella phage KP34
  Source of Sequence: NCBI:txid674081
cttcggacataccaacaccgataccgattgcaaacaggatagccatgttatcgatcagagctccacctgctttcactaaatagaaaccaatctgctgtacaatacctactacgtcaccaccctgcatggtagatgggcaaagagcatatc

SEQ ID NO: 161 - DNA, 150 bp, Klebsiella phage KP34
  Source of Sequence: NCBI:txid674081
gtcttcggacataccaacaccgataccgattgcaaacaggatagccatgttatcgatcagagctccacctgctttcactaaatagaaaccaatctgctgtacaatacctactacgtcaccaccctgcatggtagatgggcaaagagcata

SEQ ID NO: 162 - DNA, 150 bp, Klebsiella phage KP34
  Source of Sequence: NCBI:txid674081
ttgcaaacaggatagccatgttatcgatcagagctccacctgctttcactaaatagaaaccaatctgctgtacaatacctactacgtcaccaccctgcatggtagatgggcaaagagcatatccaagacccatcaggataccacagatag

SEQ ID NO: 163 - DNA, 150 bp, Klebsiella phage KP34
  Source of Sequence: NCBI:txid674081
tgttcctttgaatttattgtagcaggaagaaccgatcacaccggcgatgataccgatgaacgggttcgcaattttgctgaatgcaatgtaagcagttgtaccttcttcaagagttttgataacagatacagcacctacagacagcagagt

SEQ ID NO: 164 - DNA, 150 bp, Klebsiella phage KP34
  Source of Sequence: NCBI:txid674081
acactgtccggaccttcattggtcgcaacaaactgtttttttagttcgatataggttttctgatattctgtcattggtcgcacctcccatcaatcccaccagaaataccagacggtggactgccacaggacatctgccaaggagccaatg

SEQ ID NO: 165 - DNA, 150 bp, Klebsiella phage KP34
  Source of Sequence: NCBI:txid674081
cttcaagagttttgataacagatacagcacctacagacagcagagttgtcagcatcagccaagaagccagagctgcaagtccacctgtaccgtcgttgtcttcggacataccaacaccgataccgattgcaaacaggatagccatgttat

SEQ ID NO: 166 - DNA, 150 bp, Klebsiella phage KP34
  Source of Sequence: NCBI:txid674081
gacgattctcatggccacctgcatcttctatgaattcctgcttgcgaaaaagtatccgagggacaaacggttctgccataatgcgctggtctttgcaccggataagaccgtcctccagtcgctccgtgagatcatgacctttgataaaac

SEQ ID NO: 167 - DNA, 150 bp, Klebsiella phage KP34
  Source of Sequence: NCBI:txid674081
ctttgatgttacctgttgcttgccttcctatctgtggtatcctgatgggtcttggatatgctctttgcccatctaccatgcagggtggtgacgtagtaggtattgtacagcagattggtttctatttagtgaaagcaggtggagctctga

SEQ ID NO: 168 - DNA, 150 bp, Klebsiella phage KP34
  Source of Sequence: NCBI:txid674081
cgctgaagaatgacagccaatccggcagctttgttcctttgaatttattgtagcaggaagaaccgatcacaccggcgatgataccgatgaacgggttcgcaattttgctgaatgcaatgtaagcagttgtaccttcttcaagagttttga

SEQ ID NO: 169 - DNA, 150 bp, Klebsiella phage KP34; identical to SEQ ID NO: 96
  Source of Sequence: NCBI:txid674081

SEQ ID NO: 170 - DNA, 150 bp, Klebsiella phage KP34; identical to SEQ ID NO: 96
  Source of Sequence: NCBI:txid674081

SEQ ID NO: 171 - DNA, 150 bp, Klebsiella phage KP34
  Source of Sequence: NCBI:txid674081
aaacaggatagccatgttatcgatcagagctccacctgctttcactaaatagaaaccaatctgctgtacaatacctactacgtcaccaccctgcatggtagatgggcaaagagcatatccaagacccatcaggataccacagataggaag

SEQ ID NO: 172 - DNA, 150 bp, Klebsiella phage KP34
  Source of Sequence: NCBI:txid674081
acacttgttgagcgccgcagcggttgcgtcattccagcttccatattggttgagctggccgatacccatcgactggcccaccagatcctcggcctgctgggcaataaactccgctcgcgatgcatggtcaacaagctccttgagcgccgc

SEQ ID NO: 173 - DNA, 150 bp, Klebsiella phage KP34
  Source of Sequence: NCBI:txid674081
aacaataattgcagcagtaattgcagcagtagcatcgttagtatcggcaggaattgcattgtataatgcaaaagattcaaaaaaaggagcggcacaaagagatgaagtgaataatagaacaaatagtgaaatagctatgtttgagcaaga

SEQ ID NO: 174 - DNA, 150 bp, Klebsiella phage KP34
  Source of Sequence: NCBI:txid674081
tttgcccatctaccatgcagggtggtgacgtagtaggtattgtacagcagattggtttctatttagtgaaagcaggtggagctctgatcgataacatggctatcctgtttgcaatcggtatcggtgttggtatgtccgaagacaacgacg

SEQ ID NO: 175 - DNA, 150 bp, Klebsiella phage KP34
  Source of Sequence: NCBI:txid674081
atcggcgctgtgccttctccggaaccctaccagaagcgcaccgctcacggcatgatcctgggcctgaacccccacagctttgtcaacctgcccgctgaggagcaggaaaagctgctgaaggagtacggcagccagaaggccgctgaaaag

SEQ ID NO: 176 - DNA, 150 bp, Klebsiella phage KP34
  Source of Sequence: NCBI:txid674081
gaagccagagctgcaagtccacctgtaccgtcgttgtcttcggacataccaacaccgataccgattgcaaacaggatagccatgttatcgatcagagctccacctgctttcactaaatagaaaccaatctgctgtacaatacctactacg

SEQ ID NO: 177 - DNA, 150 bp, Klebsiella phage KP34
  Source of Sequence: NCBI:txid674081
aaattaggtaaagctttgatgttacctgttgcttgccttcctatctgtggtatcctgatgggtcttggatatgctctttgcccatctaccatgcagggtggtgacgtagtaggtattgtacagcagattggtttctatttagtgaaagca

SEQ ID NO: 178 - DNA, 150 bp, Klebsiella phage KP34
  Source of Sequence: NCBI:txid674081
acaggatagccatgttatcgatcagagctccacctgctttcactaaatagaaaccaatctgctgtacaatacctactacgtcaccaccctgcatggtagatgggcaaagagcatatccaagacccatcaggataccacagataggaaggc

SEQ ID NO: 179 - DNA, 150 bp, Klebsiella phage KP34; identical to SEQ ID NO: 178
  Source of Sequence: NCBI:txid674081

SEQ ID NO: 180 - DNA, 150 bp, Klebsiella phage KP34
  Source of Sequence: NCBI:txid674081
aacattgaagaaatcaaaatgattgatttttccgatctgagcaatatgcgaaagttcggataatcttaatttaacaaatcaaataagtccccatcccactgttttaattgcaaaaaacagtgaatggggacttttttcgattattctatt

SEQ ID NO: 181 - DNA, 150 bp, Klebsiella phage KP34
  Source of Sequence: NCBI:txid674081
aaactgatgaccctctaagtccttgataacgtcggcaagatgaccgatagccgtgtgctcgggcggctgtgaggagtgtccgccctttgcgttgacgcttatttcatagtttatgcttcccttttcggcaattcccacgcctgcaagatt

SEQ ID NO: 182 - DNA, 150 bp, Klebsiella phage KP34; identical to SEQ ID NO: 144
  Source of Sequence: NCBI:txid674081

SEQ ID NO: 183 - DNA, 150 bp, Klebsiella phage KP34
  Source of Sequence: NCBI:txid674081
tagcaggaagaaccgatcacaccggcgatgataccgatgaacgggttcgcaattttgctgaatgcaatgtaagcagttgtaccttcttcaagagttttgataacagatacagcacctacagacagcagagttgtcagcatcagccaagaa

SEQ ID NO: 184 - DNA, 150 bp, Klebsiella phage KP34
  Source of Sequence: NCBI:txid674081
gaaattcttacagaaattaggtaaagctttgatgttacctgttgcttgccttcctatctgtggtatcctgatgggtcttggatatgctctttgcccatctaccatgcagggtggtgacgtagtaggtattgtacagcagattggtttcta

SEQ ID NO: 185 - DNA, 150 bp, Klebsiella phage KP34
  Source of Sequence: NCBI:txid674081
caaacaggatagccatgttatcgatcagagctccacctgctttcactaaatagaaaccaatctgctgtacaatacctactacgtcaccaccctgcatggtagatgggcaaagagcatatccaagacccatcaggataccacagataggaa

SEQ ID NO: 186 - DNA, 150 bp, Klebsiella phage KP34; identical to SEQ ID NO: 73
  Source of Sequence: NCBI:txid674081

SEQ ID NO: 187 - DNA, 150 bp, Klebsiella phage KP34; identical to SEQ ID NO: 73
  Source of Sequence: NCBI:txid674081

SEQ ID NO: 188 - DNA, 150 bp, Klebsiella phage KP34
  Source of Sequence: NCBI:txid674081
aggtaagagtaccactagctgcaatcttgccatccacttcagcctttgcttcaaccacggcaatggtaccgcgacgttttacaaacgttgccgtcataactaattgatcacctggtacaacttgcttcttgaatttaaccttgtccatac

SEQ ID NO: 189 - DNA, 150 bp, Klebsiella phage KP34
  Source of Sequence: NCBI:txid674081
gttgcttgccttcctatctgtggtatcctgatgggtcttggatatgctctttgcccatctaccatgcagggtggtgacgtagtaggtattgtacagcagattggtttctatttagtgaaagcaggtggagctctgatcgataacatggct

SEQ ID NO: 190 - DNA, 150 bp, Klebsiella phage KP34; identical to SEQ ID NO: 189
  Source of Sequence: NCBI:txid674081

SEQ ID NO: 191 - DNA, 150 bp, Klebsiella phage KP34
  Source of Sequence: NCBI:txid674081
ctggcatccggcggcacctctgtggctgcgctgttcatggccggttacattcccggcatcatctgggcgctgtgctgctgcgtggtgggcgtgctgctggcggtcaagctgggctataaaggcacccccggcaagtttgactggaaaaat

SEQ ID NO: 192 - DNA, 150 bp, Klebsiella phage KP34
  Source of Sequence: NCBI:txid674081
gattgccagccctagcggtctgaaatcaaaagtatgtgtgtcttttgtttttctcattttaaatcacctagcatgagtttatacttcatgttataaatttagaatgtgttaaaattgcatatagtatgcagtaataattcaaaagctgtt

SEQ ID NO: 193 - DNA, 150 bp, Klebsiella phage KP34
  Source of Sequence: NCBI:txid674081
cgacgcttctttttcaatggcgcacagctggctttcgtagcggttggtcaggatggcgatgtcgcctttttccacggtattttccagcatttcgggcgtagcggtgccgatgcggatgtggccggtggtgcacacgtcgtcctcgcggcc

SEQ ID NO: 194 - DNA, 150 bp, Klebsiella phage KP34
  Source of Sequence: NCBI:txid674081
attctatcattgggaagaattgtgccggaaaaagggcttcagtatttaattaaggcatttagggaatgttcaagtgataggaaacttgtaatcgctggaggatcggagtcgaacaaggattactataatcagctccttgcattagcagag

SEQ ID NO: 195 - DNA, 150 bp, Klebsiella phage KP34
  Source of Sequence: NCBI:txid674081
aacagatacagcacctacagacagcagagttgtcagcatcagccaagaagccagagctgcaagtccacctgtaccgtcgttgtcttcggacataccaacaccgataccgattgcaaacaggatagccatgttatcgatcagagctccacc

SEQ ID NO: 196 - DNA, 150 bp, Klebsiella phage KP34
  Source of Sequence: NCBI:txid674081
ttgtcttcggacataccaacaccgataccgattgcaaacaggatagccatgttatcgatcagagctccacctgctttcactaaatagaaaccaatctgctgtacaatacctactacgtcaccaccctgcatggtagatgggcaaagagca

SEQ ID NO: 197 - DNA, 150 bp, Klebsiella phage KP34; identical to SEQ ID NO: 196
  Source of Sequence: NCBI:txid674081

SEQ ID NO: 198 - DNA, 150 bp, Klebsiella phage KP34
  Source of Sequence: NCBI:txid674081
acttctatatctgtattcccaagtttactgtattccatagtccactccttattttaacctggcatactctttttctgatactgcctcgcaccactcattggaacagtctaccccgggtacctcaattgccagatgggaaaaccactcatc

SEQ ID NO: 199 - DNA, 150 bp, Klebsiella phage KP34
  Source of Sequence: NCBI:txid674081
gtacccggttgaatatgcgtgcagcgagaagattgcgcattatataaaaatgcgctatgatcacgaaattacagatgaggagacggcttatctggccgtacatatccgcagaatccagccagaaaaagagctggaggaggattctcatta

SEQ ID NO: 200 - DNA, 150 bp, Klebsiella phage KP34
  Source of Sequence: NCBI:txid674081
gatgaacgggttcgcaattttgctgaatgcaatgtaagcagttgtaccttcttcaagagttttgataacagatacagcacctacagacagcagagttgtcagcatcagccaagaagccagagctgcaagtccacctgtaccgtcgttgtc

SEQ ID NO: 201 - DNA, 150 bp, Klebsiella phage KP34
  Source of Sequence: NCBI:txid674081
ttaccttgtgaaattttgcaattcttttcattgtctcttccttttctgttcggttttatgcatcatgcagcacaatttttcctgttttcagactgtcctgcacatcgatcactttctgattcgcgctgcccttccagtgcagcttcgtat

SEQ ID NO: 202 - DNA, 150 bp, Klebsiella phage KP34
  Source of Sequence: NCBI:txid674081
cgataccgattgcaaacaggatagccatgttatcgatcagagctccacctgctttcactaaatagaaaccaatctgctgtacaatacctactacgtcaccaccctgcatggtagatgggcaaagagcatatccaagacccatcaggatac

SEQ ID NO: 203 - DNA, 150 bp, Klebsiella phage KP34; identical to SEQ ID NO: 108
  Source of Sequence: NCBI:txid674081

SEQ ID NO: 204 - DNA, 150 bp, Klebsiella phage KP34; identical to SEQ ID NO: 27
  Source of Sequence: NCBI:txid674081

SEQ ID NO: 205 - DNA, 150 bp, Klebsiella phage KP34
  Source of Sequence: NCBI:txid674081
cggacataccaacaccgataccgattgcaaacaggatagccatgttatcgatcagagctccacctgctttcactaaatagaaaccaatctgctgtacaatacctactacgtcaccaccctgcatggtagatgggcaaagagcatatccaa

SEQ ID NO: 206 - DNA, 150 bp, Klebsiella phage KP34
  Source of Sequence: NCBI:txid674081
actgtaaactgaatatatttgtctgtccagataattccataagtatgatatgcctcagcactcttagcctgtgttaaacctgttgcatagtttttaataccatgtgaccatgactggttgttgaatctttcgcaatggatagtctgtggg

SEQ ID NO: 207 - DNA, 150 bp, Klebsiella phage KP34; identical to SEQ ID NO: 105
  Source of Sequence: NCBI:txid674081

SEQ ID NO: 208 - DNA, 150 bp, Klebsiella phage KP34
  Source of Sequence: NCBI:txid674081
gccttggcgtggagagcatcatggcaggcatcatcgtcaacaccggcctttacaccatcaatctggccgtgatgggcttttcctccaccatgtcgctggtcaagaccgacactgttttctcgctggcaaagggctcactgagcttccttg

SEQ ID NO: 209 - DNA, 150 bp, Klebsiella phage KP34; identical to SEQ ID NO: 140
  Source of Sequence: NCBI:txid674081

SEQ ID NO: 210 - DNA, 150 bp, Klebsiella phage KP34
  Source of Sequence: NCBI:txid674081
accatgaagggtggtgacgtagtaggtattgtacagcagattggtttctatttagtgaaagcaggtggagctctgatcgataacatggctatcctgtttgcaatcggtatcggtgttggtatgtccgaagacaacgacggtacaggtgga

SEQ ID NO: 211 - DNA, 150 bp, Klebsiella phage KP34; identical to SEQ ID NO: 97
  Source of Sequence: NCBI:txid674081

SEQ ID NO: 212 - DNA, 150 bp, Klebsiella phage KP34; identical to SEQ ID NO: 177
  Source of Sequence: NCBI:txid674081

SEQ ID NO: 213 - DNA, 150 bp, Klebsiella phage KP34; identical to SEQ ID NO: 68
  Source of Sequence: NCBI:txid674081

SEQ ID NO: 214 - DNA, 150 bp, Klebsiella phage KP34
  Source of Sequence: NCBI:txid674081
tgcccatctaccatgcagggtggtgacgtagtaggtattgtacagcagattggtttctatttagtgaaagcaggtggagctctgatcgataacatggctatcctgtttgcaatcggtatcggtgttggtatgtccgaagacaacgacggt

SEQ ID NO: 215 - DNA, 150 bp, Klebsiella phage KP34
  Source of Sequence: NCBI:txid674081
ccgttatctgaaagaaatcaaagcgtgattttggatacattttaaaagataagtacaaaaaataagaagtgggagtcttcaccgactaagataaaaagatactgaacagttgtagaaggagataaagaaaatgggacagatcaaagttga

SEQ ID NO: 216 - DNA, 150 bp, Klebsiella phage KP34
  Source of Sequence: NCBI:txid674081
aggtattgtacagcagattggtttctatttagtgaaagcaggtggagctctgatcgataacatggctatcctgtttgcaatcggtatcggtgttggtatgtccgaagacaacgacggtacaggtggacttgcagctctggcttcttggct

SEQ ID NO: 217 - DNA, 150 bp, Klebsiella phage KP34
  Source of Sequence: NCBI:txid674081
agcggacttggcatgaacaacatggtaatcacgatgtatgcggataatgacacgatcgtgaatgggggaaaggtatgtaatatgaaggaaatacgcggtacagaagtgtaccgctattttcaggataacggattgaataagggattatat

SEQ ID NO: 218 - DNA, 150 bp, Klebsiella phage KP34
  Source of Sequence: NCBI:txid674081
attaagtaagagaaggaggaaaaaagtaattatgatgaaattcttacagaaattaggtaaagctttgatgttacctgttgcttgccttcctatctgtggtatcctgatgggtcttggatatgctctttgcccatctaccatgcagggtgg

SEQ ID NO: 219 - DNA, 150 bp, Klebsiella phage KP34
  Source of Sequence: NCBI:txid674081
cctgctttcactaaatagaaaccaatctgctgtacaatacctactacgtcaccaccctgcatggtagatgggcaaagagcatatccaagacccatcaggataccacagataggaaggcaagcaacaggtaacatcaaagctttacctaat

SEQ ID NO: 220 - DNA, 150 bp, Klebsiella phage KP34
  Source of Sequence: NCBI:txid674081
ctctttgcccatctaccatgcagggtggtgacgtagtaggtattgtacagcagattggtttctatttagtgaaagcaggtggagctctgatcgataacatggctatcctgtttgcaatcggtatcggtgttggtatgtccgaagacaacg

SEQ ID NO: 221 - DNA, 150 bp, Klebsiella phage KP34
  Source of Sequence: NCBI:txid674081
cctgatgggtcttggatatgctctttgcccatctaccatgcagggtggtgacgtagtaggtattgtacagcagattggtttctatttagtgaaagcaggtggagctctgatcgataacatggctatcctgtttgcaatcggtagatcgga

SEQ ID NO: 222 - DNA, 150 bp, Klebsiella phage KP34
  Source of Sequence: NCBI:txid674081
aatggtagtaaggttattgagtctgaggtggatacgaccagtcaatcgatgagcctatcaagtagcctttcggagagcacatcagaagtggtatcagaatccatctctctctctgaagaagcttccgcttccttatcggaagaagcctca

SEQ ID NO: 223 - DNA, 150 bp, Klebsiella phage KP34
  Source of Sequence: NCBI:txid674081
ctgatccgttttcagggaatttttgagcattcggcggtccagtccaagaccgtccaggaatatatgtttatgctgatattcattattccgggatatttacttctttatcaggcctttgatctttatacaccgatgcgaatgcaggggcgt

SEQ ID NO: 224 - DNA, 150 bp, Klebsiella phage KP34
  Source of Sequence: NCBI:txid674081
gtgacggagcaaggcccgggcggcaagatgatgcgcatcagccccaaggtggatcacaagttgggctcggcaagcgcggtttacgattcggccagcgggtgctatcgctgtgggtggcagatcgaaaacggcgaccaaatcacagtcacc

SEQ ID NO: 225 - DNA, 150 bp, Klebsiella phage KP34
  Source of Sequence: NCBI:txid674081
cagccaagaagccagagctgcaagtccacctgtaccgtcgttgtcttcggacataccaacaccgataccgattgcaaacaggatagccatgttatcgatcagagctccacctgctttcactaaatagaaaccaatctgctgtacaatacc

SEQ ID NO: 226 - DNA, 150 bp, Klebsiella phage KP34; identical to SEQ ID NO: 82
  Source of Sequence: NCBI:txid674081

SEQ ID NO: 227 - DNA, 150 bp, Klebsiella phage KP34
  Source of Sequence: NCBI:txid674081
acctcgcccttcacgccgatgatatcgcccacgtccagcttcttgaaggcggcataaggctcttcgcccagctcatcgcggcggacgtacagctgaatgtcgcccttatcgtcccgcaggtgggcaaagcttgccttgcccattacgcgc

SEQ ID NO: 228 - DNA, 150 bp, Klebsiella phage KP34
  Source of Sequence: NCBI:txid674081
tatgctctttgcccatctaccatgcagggtggtgacgtagtaggtattgtacagcagattggtttctatttagtgaaagcaggtggagctctgatcgataacatggctatcctgtttgcaatcggtatcggtgttggtatgtccgaagac

SEQ ID NO: 229 - DNA, 150 bp, Klebsiella phage KP34; identical to SEQ ID NO: 228
  Source of Sequence: NCBI:txid674081

SEQ ID NO: 230 - DNA, 150 bp, Klebsiella phage KP34; identical to SEQ ID NO: 228
  Source of Sequence: NCBI:txid674081

SEQ ID NO: 231 - DNA, 150 bp, Klebsiella phage KP34
  Source of Sequence: NCBI:txid674081
acgactccgcaaaccgcggccacttctacaaacaggttttctctgcttatgagtcgtttctcgtcattgccagctcggttttgattgtgtttgtccgtccgttttccgacttgctgattagtaccagcacttacacagaatatgcggatg

SEQ ID NO: 232 - DNA, 150 bp, Klebsiella phage KP34
  Source of Sequence: NCBI:txid674081
gttagtgcgccctgtggtccttttaataagtattatgggtgtgaaaaaggaataatgtcgccgctttacaccgtttataaatatatcgattgttctacggattataccgaatacctttcgtacttcttttcttccgcaaattggcatcgc

SEQ ID NO: 233 - DNA, 150 bp, Klebsiella phage KP34; identical to SEQ ID NO: 214
  Source of Sequence: NCBI:txid674081

SEQ ID NO: 234 - DNA, 150 bp, Klebsiella phage KP34
  Source of Sequence: NCBI:txid674081
cccgttgaagcagtccactgccagttctttgtagaaatcttgcacagtgcccacactgccattataggagccttaagtaaaacctccagtcccataacaatcagcatctgtacctgtgtaatatcatttgtagaacgtgtaattaaactt

SEQ ID NO: 235 - DNA, 150 bp, Klebsiella phage KP34
  Source of Sequence: NCBI:txid674081
tccacctgctttcactaaatagaaaccaatctgctgtacaatacctactacgtcaccaccctgcatggtagatgggcaaagagcatatccaagacccatcaggataccacagataggaaggcaagcaacaggtaacatcaaagctttacc

SEQ ID NO: 236 - DNA, 150 bp, Klebsiella phage KP34
  Source of Sequence: NCBI:txid674081
aagtccacctgtaccgtcgttgtcttcggacataccaacaccgataccgattgcaaacaggatagccatgttatcgatcagagctccacctgctttcactaaatagaaaccaatctgctgtacaatacctactacgtcaccaccctgcat

SEQ ID NO: 237 - DNA, 150 bp, Klebsiella phage KP34
  Source of Sequence: NCBI:txid674081
tgcaagtccacctgtaccgtcgttgtcttcggacataccaacaccgataccgattgcaaacaggatagccatgttatcgatcagagctccacctgctttcactaaatagaaaccaatctgctgtacaatacctactacgtcaccaccctg

SEQ ID NO: 238 - DNA, 150 bp, Klebsiella phage KP34; identical to SEQ ID NO: 237
  Source of Sequence: NCBI:txid674081

SEQ ID NO: 239 - DNA, 150 bp, Klebsiella phage KP34
  Source of Sequence: NCBI:txid674081
agcagagttgtcagcatcagccaagaagccagagctgcaagtccacctgtaccgtcgttgtcttcggacataccaacaccgataccgattgcaaacaggatagccatgttatcgatcagagctccacctgctttcactaaatagaaacca

SEQ ID NO: 240 - DNA, 150 bp, Klebsiella phage KP34
  Source of Sequence: NCBI:txid674081
gcaccgatcttcagtttccggatgatcttgtttttcttttttgatgcaccttcccccacatagagatatggataccatttcatattttttcctttctcctgccatcgaattcttgttctattttaccagacacagaaacataatgcaacg

SEQ ID NO: 241 - DNA, 150 bp, Klebsiella phage KP34; identical to SEQ ID NO: 102
  Source of Sequence: NCBI:txid674081

SEQ ID NO: 242 - DNA, 88 bp, Klebsiella phage KP34
  Source of Sequence: NCBI:txid674081
gctgtcttcgcatggttatccaccaacaagagcaggggcagaacaggcatatcaggattacatggatggaaagattgatgttccagaa

SEQ ID NO: 243 - DNA, 150 bp, Klebsiella phage KP34
  Source of Sequence: NCBI:txid674081
ttatcgatcagagctccacctgctttcactaaatagaaaccaatctgctgtacaatacctactacgtcaccaccctgcatggtagatgggcaaagagcatatccaagacccatcaggataccacagataggaaggcaagcaacaggtaac

SEQ ID NO: 244 - DNA, 150 bp, Klebsiella phage KP34
  Source of Sequence: NCBI:txid674081
ggtatcctgatgggtcttggatatgctctttgcccatctaccatgcagggtggtgacgtagtaggtattgtacagcagattggtttctatttagtgaaagcaggtggagctctgatcgataacatggctatcctgtttgcaatctgtatc

SEQ ID NO: 245 - DNA, 150 bp, Klebsiella phage KP34
  Source of Sequence: NCBI:txid674081
gtcgttgtcttcggacataccaacaccgataccgattgcaaacaggatagccatgttatcgatcagagctccacctgctttcactaaatagaaaccaatctgctgtacaatacctactacgtcaccaccctgcatggtagatgggcaaag

SEQ ID NO: 246 - DNA, 150 bp, Klebsiella phage KP34
  Source of Sequence: NCBI:txid674081
gatcagagctccacctgctttcactaaatagaaaccaatctgctgtacaatacctactacgtcaccaccctgcatggtagatgggcaaagagcatatccaagacccatcaggataccacagataggaaggcaagcaacaggtaacatcaa

SEQ ID NO: 247 - DNA, 150 bp, Klebsiella phage KP34
  Source of Sequence: NCBI:txid674081
agaagctgttaagatgatggcaagaatcgctgaacgtacagagaaggaaatcaactaccgcaaacgtttcaatgatatggctaaatacactgacccaggtatcacagacgctatctgccatgctacctgcagcacagcttacgacctcga

SEQ ID NO: 248 - DNA, 150 bp, Klebsiella phage KP34
  Source of Sequence: NCBI:txid674081
tgataccgatgaacgggttcgcaattttgctgaatgcaatgtaagcagttgtaccttcttcaagagttttgataacagatacagcacctacagacagcagagttgtcagcatcagccaagaagccagagctgcaagtccacctgtaccgt

SEQ ID NO: 249 - DNA, 150 bp, Klebsiella phage KP34
  Source of Sequence: NCBI:txid674081
cttcttcaagagttttgataacagatacagcacctacagacagcagagttgtcagcatcagccaagaagccagagctgcaagtccacctgtaccgtcgttgtcttcggacataccaacaccgataccgattgcaaacaggatagccatgt

SEQ ID NO: 250 - DNA, 150 bp, Klebsiella phage KP34
  Source of Sequence: NCBI:txid674081
cagagctccacctgctttcactaaatagaaaccaatctgctgtacaatacctactacgtcaccaccctgcatggtagatgggcaaagagcatatccaagacccatcaggataccacagataggagatcggaagagcacacgtctgaactc

SEQ ID NO: 251 - DNA, 150 bp, Klebsiella phage KP34
  Source of Sequence: NCBI:txid674081
gccaagaagccagagctgcaagtccacctgtaccgtcgttgtcttcggacataccaacaccgataccgattgcaaacaggatagccatgttatcgatcagagctccacctgctttcactaaatagaaaccaatctgctgtacaataccta

SEQ ID NO: 252 - DNA, 150 bp, Klebsiella phage KP34; identical to SEQ ID NO: 82
  Source of Sequence: NCBI:txid674081

SEQ ID NO: 253 - DNA, 150 bp, Klebsiella phage KP34
  Source of Sequence: NCBI:txid674081
cagagctccacctgctttcactaaatagaaaccaatctgctgtacaatacctactacgtcaccaccctgcatggtagatgggcaaagagcatatccaagacccatcaggataccacagataggaatgcaagcaacaggtaacatcaaagc

SEQ ID NO: 254 - DNA, 149 bp, Klebsiella phage KP34
  Source of Sequence: NCBI:txid674081
taccatgcagggtggtgacgtagtaggtattgtacagcagattggtttctatttagtgaaagcaggtggagctctgatcgataacatggctatcctgtttgcaatcggtatcggtgttggtatgtccgaagacaacgacggtacaggtg

SEQ ID NO: 255 - DNA, 150 bp, Klebsiella phage KP34
  Source of Sequence: NCBI:txid674081
gccatgttatcgatcagagctccacctgctttcactaaatagaaaccaatctgctgtacaatacctactacgtcaccaccctgcatggtagatgggcaaagagcatatccaagacccatcaggataccacagataggaaggcaagcaaca

SEQ ID NO: 256 - DNA, 150 bp, Klebsiella phage KP34
  Source of Sequence: NCBI:txid674081
aaatgcgctatgatcacgaaattacagatgaggagacggcttatctggccgtacatatccgcagaatccagccagaaaaagagctggaggaggattctcattaagtaagagaaggaggaaaaaagtaattatgatgaaattcttacagaa

SEQ ID NO: 257 - DNA, 150 bp, Klebsiella phage KP34
  Source of Sequence: NCBI:txid674081
cttccgataaacgaatatatttgattatccgccaaaaatttgttgaattctcggcggatttgcgttattattacataggtaaccgcttgaatatcaaaacggaaattttaccacggtaaggctgtgaaattgcggccgtatgctttagga

SEQ ID NO: 258 - DNA, 150 bp, Klebsiella phage KP34
  Source of Sequence: NCBI:txid674081
cgggtcatgattctggacgaggcgacctcctccgtcgatacccgaaccgaaatccagattcaaaaggcaatggatcacctcatggagggcagaaccagcttcatcattgcccatcggctttcgaccatccgaaatgcagatttgattctg

SEQ ID NO: 259 - DNA, 150 bp, Klebsiella phage KP34
  Source of Sequence: NCBI:txid674081
cagagctgcaagtccacctgtaccgtcgttgtcttcggacataccaacaccgataccgattgcaaacaggatagccatgttatcgatcagagctccacctgctttcactaaatagaaaccaatctgctgtacaatacctactacgtcacc

SEQ ID NO: 260 - DNA, 150 bp, Klebsiella phage KP34; identical to SEQ ID NO: 259
  Source of Sequence: NCBI:txid674081

SEQ ID NO: 261 - DNA, 150 bp, Klebsiella phage KP34
  Source of Sequence: NCBI:txid674081
tcagaatatggatctgtctcaatgtcctttggaatgtatgatacctattataatccctatacgtatgcatacagctacccaggaagtgcaggcgtgaaccatgcaataactctgattggctgggatgacaattattccagggagaatttt

SEQ ID NO: 262 - DNA, 150 bp, Klebsiella phage KP34
  Source of Sequence: NCBI:txid674081
ctttgcccatctaccatgcagggtggtgacgtagtaggtattgtacagcagattggtttctatttagtgaaagcaggtggagctctgatcgataacatggctatcctgtttgcaatcggtatcggtgttggtatgtccgaagacaacgac

SEQ ID NO: 263 - DNA, 150 bp, Klebsiella phage KP34; identical to SEQ ID NO: 77
  Source of Sequence: NCBI:txid674081

SEQ ID NO: 264 - DNA, 150 bp, Klebsiella phage KP34
  Source of Sequence: NCBI:txid674081
tggtgacgtagtaggtattgtacagcagattggtttctatttagtgaaagcaggtggagctctgatcgataacatggctatcctgtttgcaatcggtatcggtgttggtatgtccgaagacaacgacggtacaggtggacttgcagctct

SEQ ID NO: 265 - DNA, 150 bp, Klebsiella phage KP34
  Source of Sequence: NCBI:txid674081
attgcaaacaggatagccatgttatcgatcagagctccacctgctttcactaaatagaaaccaatctgctgtacaatacctactacgtcaccaccctgcatggtagatgggcaaagagcatatccaagacccatcaggataccacagata

SEQ ID NO: 266 - DNA, 150 bp, Klebsiella phage KP34; identical to SEQ ID NO: 104
  Source of Sequence: NCBI:txid674081

SEQ ID NO: 267 - DNA, 150 bp, Klebsiella phage KP34
  Source of Sequence: NCBI:txid674081
ttaaacccatacaaggccaccgcatacaggaatgccacaatcgtaaaaatcaccgtatgagggaatatcttccctatgaggctcaccgtaagcgaatttccacccatctgcaaccattcgcgggccgtcgagtatttaatctcggaaccg

SEQ ID NO: 268 - DNA, 117 bp, Klebsiella phage KP34
  Source of Sequence: NCBI:txid674081
ccgaaaaaagagaaaaatacaaagaaactgctttttgtgttataataccagtacattatgagcaggtaatgaactgtgaagtattttttcagttatgataaatatacagttttgttt

SEQ ID NO: 269 - DNA, 150 bp, Klebsiella phage KP34
  Source of Sequence: NCBI:txid674081
caccgataccgattgcaaacaggatagccatgttatcgatcagagctccacctgctttcactaaatagaaaccaatctgctgtacaatacctactacgtcaccaccctgcatggtagatgggcaaagagcatatccaagacccatcagga

SEQ ID NO: 270 - DNA, 150 bp, Klebsiella phage KP34; identical to SEQ ID NO: 66
  Source of Sequence: NCBI:txid674081

SEQ ID NO: 271 - DNA, 150 bp, Klebsiella phage KP34; identical to SEQ ID NO: 56
  Source of Sequence: NCBI:txid674081

SEQ ID NO: 272 - DNA, 150 bp, Klebsiella phage KP34; identical to SEQ ID NO: 134
  Source of Sequence: NCBI:txid674081

SEQ ID NO: 273 - DNA, 149 bp, Klebsiella phage KP34
  Source of Sequence: NCBI:txid674081
atagccgaaagtccagcatctcctatggtattgcctacaaaatatccatccactatcgcatatattcctgataatgcaaatgctataatagatggaataacatatttaaaaaataatgatttatgattcatttttcttacaatctcctt

SEQ ID NO: 274 - DNA, 150 bp, Klebsiella phage KP34
  Source of Sequence: NCBI:txid674081
gcgcaatctcaggaatcaggactattgtctgttttccctgggcgatcgtgctctcgatcaatttcatatatacctgggttttcccacttcctgtcacgccctcaatcaacattggacggggaaatggcttttcccactcttccagaatct

SEQ ID NO: 275 - DNA, 150 bp, Klebsiella phage KP34; identical to SEQ ID NO: 77
  Source of Sequence: NCBI:txid674081

SEQ ID NO: 276 - DNA, 150 bp, Klebsiella phage KP34
  Source of Sequence: NCBI:txid674081
cgttgagcgaggagatctgcccgcgcagcgggatgctgacgagaaagtcacacttttcccgcaccagccggctgatgccggtgccctccgacccgatgacgatgcaggccgggccggtcagatccgttttccacagctcgtttgcgccct

SEQ ID NO: 277 - DNA, 150 bp, Klebsiella phage KP34
  Source of Sequence: NCBI:txid674081
cttggatatgctctttgcccatctaccatgcagggtggtgacgtagtaggtattgtacagcagattggtttctatttagtgaaagcaggtggagctctgatcgataacatggctatcctgtttgcaatcggtatcggtgttggtatgtcc

SEQ ID NO: 278 - DNA, 150 bp, Klebsiella phage KP34
  Source of Sequence: NCBI:txid674081
gcgctcgatgcgctcttcgccgaatgcttcggatacttcaacgtgaccgacctgccatacgtgtcgatggactactacgtgaacaacgattacgtgcagtccgacaacgaatggattcacctgtacaaccaccagtggcgcaaccaggtg

SEQ ID NO: 279 - DNA, 150 bp, Klebsiella phage KP34
  Source of Sequence: NCBI:txid674081
tgatgttacctgttgcttgccttcctatctgtggtatcctgatgggtcttggatatgctctttgcccatctaccatgcagggtggtgacgtagtaggtattgtacagcagattggtttctatttagtgaaagcaggtggagctctgatcg

SEQ ID NO: 280 - DNA, 150 bp, Klebsiella phage KP34
  Source of Sequence: NCBI:txid674081
attctcagcgtctgtgatattctcgaatcctgttacctttgtctctccgtcctttggtttgatcagaacgactttgttctcaagaagatccttaatggtgtcttcctttgtcagtttttcctctgtgagggcatccatctgtgtatctgc

SEQ ID NO: 281 - DNA, 150 bp, Klebsiella phage KP34; identical to SEQ ID NO: 27
  Source of Sequence: NCBI:txid674081

SEQ ID NO: 282 - DNA, 150 bp, Klebsiella phage KP34
  Source of Sequence: NCBI:txid674081
aggaggaaaaaagtaattatgatgaaattcttacagaaattaggtaaagctttgatgttacctgttgcttgccttcctatctgtggtatcctgatgggtcttggatatgctctttgcccatctaccatgcagggtggtgacgtagtaggt

SEQ ID NO: 283 - DNA, 150 bp, Klebsiella phage KP34
  Source of Sequence: NCBI:txid674081
tatcccgcacctgctgtactactggcgctccagccccaccagcgtggcaagcaacatttcggccaagacctactgtctggaagctgcggtcaaggccctgtatgcccactacgagcgtgtgggagatcggaagagcgtcgtgtagggaaa

SEQ ID NO: 284 - DNA, 150 bp, Klebsiella phage KP34
  Source of Sequence: NCBI:txid674081
agcgagaagattgcgcattatataaaaatgcgctatgatcacgaaattacagatgaggagacggcttatctggccgtacatatccgcagaatccagccagaaaaagagctggaggaggattctcattaagtaagagaaggaggaaaaaag

SEQ ID NO: 285 - DNA, 150 bp, Klebsiella phage KP34
  Source of Sequence: NCBI:txid674081
ctacagacagcagagttgtcagcatcagccaagaagccagagctgcaagtccacctgtaccgtcgttgtcttcggacataccaacaccgataccgattgcaaacaggatagccatgttatcgatcagagctccacctgctttcactaaat

SEQ ID NO: 286 - DNA, 150 bp, Klebsiella phage KP34
  Source of Sequence: NCBI:txid674081
gcagggtggtgacgtagtaggtattgtacagcagattggtttctatttagtgaaagcaggtggagctctgatcgataacatggctatcctgtttgcaatcggtatcggtgttggtatgtccgaagacaacgacggtacaggtggacttgc

SEQ ID NO: 287 - DNA, 150 bp, Klebsiella phage KP34
  Source of Sequence: NCBI:txid674081
attaccgctatcagacagacatcatgatcataacgattgtaattctggtagtgcttgtgcagatcctgcagggacttggcatgatgctctccaggaagctggataagcgaaaagcataaaaagatatttgacagaagattgtaatcacga

SEQ ID NO: 288 - DNA, 123 bp, Klebsiella phage KP34
  Source of Sequence: NCBI:txid674081
gatcagagctccacctgctttcactaaatagaaaccaatctgctgtacaatacctactacgtcaccaccctgcatggtagatgggcaaagagcatatccaagacccatcagtataccacagat

SEQ ID NO: 289 - DNA, 150 bp, Klebsiella phage KP34; identical to SEQ ID NO: 237
  Source of Sequence: NCBI:txid674081

SEQ ID NO: 290 - DNA, 150 bp, Klebsiella phage KP34
  Source of Sequence: NCBI:txid674081
cacgatgtccgaaatcattcagtgcgtcgcagatatgcgcacgcaggatgtgaaggcaatccgcgagttcggcaagttcgtccccgcctttgccgcctttgagaagaaccgcctgagcctctatcgtgaggcggcgaaggaagccgagga

SEQ ID NO: 291 - DNA, 150 bp, Klebsiella phage KP34
  Source of Sequence: NCBI:txid674081
tcgatctccggctgcagcggcccaaagggcagatagagggctgcgtctgcctcgctgcagagcatttcgtgggggttgcgtgacacgggccgagtgtccaccgcaaactcatatcccggcagcgcggcgtgcagccgtgccatcaggatg

SEQ ID NO: 292 - DNA, 150 bp, Klebsiella phage KP34
  Source of Sequence: NCBI:txid674081
tggaaacagtatgatatcggtaaatgattttgccatgctgcagccgggagatattatccggctggatcgcaaggtggaggacgaattggatatttatgttggaaatattcggaaatttactgcacttcccggatcatcgggggataaata

SEQ ID NO: 293 - DNA, 150 bp, Klebsiella phage KP34
  Source of Sequence: NCBI:txid674081
accgattgcaaacaggatagccatgttatcgatcagagctccacctgctttcactaaatagaaaccaatctgctgtacaatacctactacgtcaccaccctgcatggtagatgggcaaagagcatatccaagacccatcaggagatcgga

SEQ ID NO: 294 - DNA, 150 bp, Klebsiella phage KP34; identical to SEQ ID NO: 262
  Source of Sequence: NCBI:txid674081

SEQ ID NO: 295 - DNA, 150 bp, Klebsiella phage KP34; identical to SEQ ID NO: 262
  Source of Sequence: NCBI:txid674081

SEQ ID NO: 296 - DNA, 150 bp, Klebsiella phage KP34
  Source of Sequence: NCBI:txid674081
ggtatcctgatgggtcttggatatgctctttgcccatctaccatgcagggtggtgacgtagtaggtattgtacagcagattggtttctatttagtgaaagcaggtggagctctgatcgataacatggctatcctgtttgcaatcggtatc

SEQ ID NO: 297 - DNA, 150 bp, Klebsiella phage KP34
  Source of Sequence: NCBI:txid674081
gatctctttgaagaaattgctcgtatctatggatatgaccgcttgccaaccagccttccaaaagacgatggtacagcaggtgaattgactgcgacacaaaaacttcgccgccaagttcgtaccattgctgaaggggcaggtttgacagaa

SEQ ID NO: 298 - DNA, 150 bp, Klebsiella phage KP34; identical to SEQ ID NO: 82
  Source of Sequence: NCBI:txid674081

SEQ ID NO: 299 - DNA, 150 bp, Klebsiella phage KP34
  Source of Sequence: NCBI:txid674081
gctctggagaagatcgtcaagggcatctgcttcgacaacgcaaagcgctacttcaacctgtaagagaagtgcctgaacctgaataaggcataacacaaagcccccggaacggtgtgaaacgttccgggggcttttgctgtaagcagataa

SEQ ID NO: 300 - DNA, 150 bp, Klebsiella phage KP34
  Source of Sequence: NCBI:txid674081
aagggcgatgtgttcgtggtgggctgctcgtccagcgagatcgtgggcgggcacatcggcaaggattccagtctggaagccgcgtaggccgtgtacgcgggcattgccccagtgctggctgaaaacggcatctggctggccgcgcagtgc

SEQ ID NO: 301 - DNA, 149 bp, Klebsiella phage KP34
  Source of Sequence: NCBI:txid674081
ctcccatggatcttcttttaagaaacgaagggatgcatcgaatgtagcaccaccccagcactcaacagcgttgtatccgactttatccattttatcaacgatcggaagcatctgttctgttgtcatacgtgttgcgatcagggactgat

SEQ ID NO: 302 - DNA, 150 bp, Klebsiella phage KP34
  Source of Sequence: NCBI:txid674081
gcagggtggtgacgtagtaggtattgtacagcagattggtttctatttagtgaaagcaggtggagctctgatcgataacatgtctatcctgtttgcaatcggtatcggtgttggtatgtccgaagacaacgacggtacaggtggacttgc

SEQ ID NO: 303 - DNA, 150 bp, Klebsiella phage KP34; identical to SEQ ID NO: 286
  Source of Sequence: NCBI:txid674081

SEQ ID NO: 304 - DNA, 150 bp, Klebsiella phage KP34
  Source of Sequence: NCBI:txid674081
agagaagataagaatcagcaccaatgccatccagaactcgggaatacagctggatatcgtacccagtttacataaaatctggtctgccagccggttttcgtaccaggcacacaggattcccagaagcaatgctcccgcgaacataatgac

SEQ ID NO: 305 - DNA, 150 bp, Klebsiella phage KP34
  Source of Sequence: NCBI:txid674081
cgtcgttgtcttcggacataccaacaccgataccgattgcaaacaggatagccatgttatcgatcagagctccacctgctttcactaaatagaaaccaatctgctgtacaatacctactacgtcaccaccctgcatggtagatgggcaaa

SEQ ID NO: 306 - DNA, 134 bp, Klebsiella phage KP34
  Source of Sequence: NCBI:txid674081
atgatcacgaaattacagatgaggagacggcttatctggccgtacatatccgcagaatccagccagaaaaagagctggaggaggattctcattaagtaagagaaggaggacaaaagtaattatgatgaaattct

SEQ ID NO: 307 - DNA, 150 bp, Klebsiella phage KP34; identical to SEQ ID NO: 84
  Source of Sequence: NCBI:txid674081

SEQ ID NO: 308 - DNA, 150 bp, Klebsiella phage KP34
  Source of Sequence: NCBI:txid674081
acttcctatcctcataatttatttgagtattttgattatatttattacgttcataaacctttacattccctttatattgaaattcataacagtagttatactcttttttatattctaaaaaacgtaaatcaggattacaataatctatta

SEQ ID NO: 309 - DNA, 150 bp, Klebsiella phage KP34
  Source of Sequence: NCBI:txid674081
attcggaggactgcgtgccgtatccgaatttgatatgaatatcgggaaaggtcagctctatggcctgatcggaccgaacggtgccggaaaaacaaccatatttaacttgctgacaggtgtttacaaaccggatgaaggtattgtaaaatt

SEQ ID NO: 310 - DNA, 150 bp, Klebsiella phage KP34; identical to SEQ ID NO: 167
  Source of Sequence: NCBI:txid674081

SEQ ID NO: 311 - DNA, 150 bp, Klebsiella phage KP34; identical to SEQ ID NO: 108
  Source of Sequence: NCBI:txid674081

SEQ ID NO: 312 - DNA, 150 bp, Klebsiella phage KP34
  Source of Sequence: NCBI:txid674081
cgatcacaccggcgatgataccgatgaacgggttcgcaattttgctgaatgcaatgtaagcagttgtaccttcttcaagagttttgataacagatacagcacctacagacagcagagttgtcagcatcagccaagaagccagagctgcaa

SEQ ID NO: 313 - DNA, 150 bp, Klebsiella phage KP34
  Source of Sequence: NCBI:txid674081
cctgatgggtcttggatatgctctttgcccatctaccatgcagggtggtgacgtagtaggtattgtacagcagattggtttctatttagtgaaagcaggtggagctctgatcgataacatggctatcctgtttgcaatcggtatcggtgt

SEQ ID NO: 314 - DNA, 150 bp, Klebsiella phage KP34
  Source of Sequence: NCBI:txid674081
tacagaaattaggtaaagctttgatgttacctgttgcttgccttcctatctgtggtatcctgatgggtcttggatatgctctttgcccatctaccatgcagggtggtgacgtagtaggtattgtacagcagattggtttctatttagtga

SEQ ID NO: 315 - DNA, 150 bp, Klebsiella phage KP34
  Source of Sequence: NCBI:txid674081
ctcggccagcagcgcccacgcagcccatgccgtgggcaccgccgccgcaagggccagccccggcagcgccagtgccgggcatgtatggaacgcactgcccggcggcagggctgccaccgcgcacaaggcctgtgcagcccaaagctccca

SEQ ID NO: 316 - DNA, 150 bp, Klebsiella phage KP34
  Source of Sequence: NCBI:txid674081
gaagcagattgcagatggtgattattctgtcagggttccagtgcatacagaagatgaaattggagaactttcagtcagttttaattatatgacagaacaattgatagaaaagttaatgaaattggatcaattgttaaaaaaccaggaaga

SEQ ID NO: 317 - DNA, 150 bp, Klebsiella phage KP34
  Source of Sequence: NCBI:txid674081
gttcaatgcttgttaccttctctacaaccggttccggctttggctgtgcattgacaggcttcttacgcattgtgtttctattgttattgttattgttgttcttattattgttcttgccgtttctggatgtatttcttccttcaaaatctc

SEQ ID NO: 318 - DNA, 150 bp, Klebsiella phage KP34
  Source of Sequence: NCBI:txid674081
tgacgtagtaggtattgtacagcagattggtttctatttagtgaaagcaggtggagctctgatcgataacatggctatcctgtttgcaatcggtatcggtgttggtatgtccgaagacaacgacggtacaggtggacttgcagctctggc

SEQ ID NO: 319 - DNA, 150 bp, Klebsiella phage KP34; identical to SEQ ID NO: 262
  Source of Sequence: NCBI:txid674081

SEQ ID NO: 320 - DNA, 150 bp, Klebsiella phage KP34; identical to SEQ ID NO: 84
  Source of Sequence: NCBI:txid674081

SEQ ID NO: 321 - DNA, 150 bp, Klebsiella phage KP34
  Source of Sequence: NCBI:txid674081
cctatctgtggtatcctgatgggtcttggatatgctctttgcccatctaccatgcagggtggtgacgtagtaggtattgtacagcagattggtttctatttagtgaaagcaggtggagctctgagatcggaagagcgtcgtggagggaca

SEQ ID NO: 322 - DNA, 150 bp, Klebsiella phage KP34; identical to SEQ ID NO: 84
  Source of Sequence: NCBI:txid674081

SEQ ID NO: 323 - DNA, 150 bp, Klebsiella phage KP34
  Source of Sequence: NCBI:txid674081
caggcgaagaatgtgccctgctggttgcagcccgcgtaaaatacctcgcttttctgcccgatgccaacgctgtgcagctggccttccagccagcttacgctgcggccggaggctttcaggttgtcttccatcagctgcccgtccagaatc

SEQ ID NO: 324 - DNA, 149 bp, Klebsiella phage KP34
  Source of Sequence: NCBI:txid674081
aggaattctgacggtaacggtcgttccgacatcaagctcactttcgattttaatgccgtaacgctcgccgaagtagattttcaaacggtcgtcaacattcttgacaccgataccctttgagtcgctcctgcccttttcaagcacctgcc

SEQ ID NO: 325 - DNA, 150 bp, Klebsiella phage KP27
  Source of Sequence: NCBI:txid1129147
tttgtggcggggtgcaggggcggcagcgcactgctggggtcaaggggcaacgcccattggcaggttaagggcggcagccattatcgggtcaaggacgaagtgccttggcaggttgagagatcggaagagcgtcgtgtagggaaagagtgt

SEQ ID NO: 326 - DNA, 150 bp, Klebsiella phage KP27
  Source of Sequence: NCBI:txid1129147
cctttgtggcggggtgcaggggcggcagcgcactgctggggtcaaggggcaacgcccattggcaggttaagggcggcagccattatcgggtcaaggacgaagtgccttggcaggttgagagatcggaagagcgtcgtgtagggaaagagt

SEQ ID NO: 327 - DNA, 150 bp, Klebsiella phage KP27
  Source of Sequence: NCBI:txid1129147
gggtgtagttggggccgtccggctcaaaggtgcgggtgcgcagggcgtcggcaaaactcttgccctgactcatcaggtcgtagatggtatcggtctggtcgccgttggtcacaatgtgggtcttgccaagggtcagcaccggattataaa

SEQ ID NO: 328 - DNA, 150 bp, Klebsiella phage KP27
  Source of Sequence: NCBI:txid1129147
ctcaacctgccaaggcacttcgtccttgacccgataatggctgccgcccttaacctgccaatgggcgttgccccttgaccccagcagtgcgctgccgcccctgcaccccgccacaaaggagatcggaagagcacacgtctgaactccagt

SEQ ID NO: 329 - DNA, 150 bp, Klebsiella phage KP27
  Source of Sequence: NCBI:txid1129147
aagtacaccaccaactattccaataccgcttcctgcaatcccaccttttatagcaccacccacagtatctacgaaagtttctttccaatttacctttctatccaaaatcacctgtgttactattgacgaagtaaaagaaacaacaccacc

SEQ ID NO: 330 - DNA, 150 bp, Klebsiella phage KP27
  Source of Sequence: NCBI:txid1129147
ggaaatttccaacaataattgcaggggcaatcagtggtggtgttgtttcttttacttcgtcaatagtaacacaggtgattttggatagaaaggtaaattggaaagaaactttcgtagatactgtgggtggtgctataaaaggtgggattg

SEQ ID NO: 331 - DNA, 150 bp, Klebsiella phage KP27
  Source of Sequence: NCBI:txid1129147
ttactgagtatccaaaaacacttaacagatataattattgttggaataattccttaatatatgtggattatgatgggaaatttccaacaataattgcaggggcaatcagtggtggtgttgtttcttttacttcgtcaatagtaacacagg

SEQ ID NO: 332 - DNA, 150 bp, Klebsiella phage KP27
  Source of Sequence: NCBI:txid1129147
gcccctgtagcacctgtagcaagtacaccaccaactattccaataccgcttcctgcaatcccaccttttatagcaccacccacagtatctacgaaagtttctttccaatttacctttctatccaaaatcacctgtgttactattgacgaa

SEQ ID NO: 333 - DNA, 150 bp, Klebsiella phage vB_KpnP_KpV289
  Source of Sequence: NCBI:txid1671396
gactttatctttaccaccttctgctgattcaagagttttagcattttgatcagctttcaggtcaattgttttctttttgctttcaccaagagctttcatttcgataatcatcttagcttttgtatctttaatttcaattgtcattgtcaa

SEQ ID NO: 334 - DNA, 150 bp, Klebsiella phage vB_KpnP_KpV289
  Source of Sequence: NCBI:txid1671396
agagttttagcattttgatcagctttcaggtcaattgttttctttttgctttcaccaagagctttcatttcgataatcatcttagcttttgtatctttaatttcaattgtcattgtcaaatcaaattgatcaatcacctgctcaagttgt

SEQ ID NO: 335 - DNA, 150 bp, Klebsiella phage KLPN1
  Source of Sequence: NCBI:txid1647408
aaaggttacctcgctgccagcttttgcatatggattgttcaagattgcactaaggtctactttacctttgtgcggctcatcaactgcatctgtagcaaccaggagatccatacgtcctaccatctctttcaggcttctcacacccagttt

SEQ ID NO: 336 - DNA, 150 bp, Escherichia virus 186
  Source of Sequence: NCBI:txid29252
agcagcgcatcctgagagcggcacgggtgccagcgcttgccatccttccccataatccagccatgaccgcagtgcattgcaggactttgcttaacgagcagtgatgcaaaagatggttctttagtcagcataaccacctcagatcagacc

SEQ ID NO: 337 - DNA, 150 bp, Escherichia virus 186
  Source of Sequence: NCBI:txid29252
gcgtccacatgaaattcaaatttatccttgaaacgaatattctggatggccagatagctggaagcatgttccagttctttttccagagtgatcatatcttttccctgacttaaggagatccggaaaaatttagccagaagagagaccatc

SEQ ID NO: 338 - DNA, 131 bp, Escherichia virus 186
  Source of Sequence: NCBI:txid29252
gtcatcgtcgcgatcgatgcaagtgagaacattttattcttccagatattcttcagtccctgcattatcgtgtagaaaaatgtactaatcctcatcgctataatcaccctcgctgtcctcaacgacaacgc

SEQ ID NO: 339 - DNA, 150 bp, Escherichia virus 186
  Source of Sequence: NCBI:txid29252
tagaaagcatcctgcaccacgcggcgcttgacgctctccgtccagctttcgtcccagcggtcaacggaaaacgcccacgccagatatggcagaaagctgaccggacacgttgccgggttccacaagtcgcgtagcggcacctgcagatcg

SEQ ID NO: 340 - DNA, 150 bp, Escherichia virus 186
  Source of Sequence: NCBI:txid29252
gatccaaacatcaatggggacaatggtgtcgctgccgccccagacgctgtagagatcgtggaaaataccgccgccgacaccgcacgcgccgtaggaaacacagattttatgatcgggggcagactcatacgcccgaagtgcaggcatacg

SEQ ID NO: 341 - DNA, 150 bp, Escherichia virus 186
  Source of Sequence: NCBI:txid29252
tcttctttccacccatattgtgtcggatgtggaacagattgcagataaaattctgatgatgaaagaggggcagcttatttttgaagggaccagacaggaaacaggaagtgatctggaaaagctttatcttgaaaaatttggggaggtgaa

SEQ ID NO: 342 - DNA, 150 bp, Escherichia virus 186
  Source of Sequence: NCBI:txid29252
tcgaagaaaccagccaggcattttccgccctgaaaaacaccctcgacaaaaccgaaagtttcagccagccgcgacgtacaaaagccagtggcggtggtggcgacgagctgctgaccgactgctgataaaccgcagaccgaaaccgggcgg

SEQ ID NO: 343 - DNA, 150 bp, Escherichia virus 186
  Source of Sequence: NCBI:txid29252
tccctgatgtggatccgaagcgggtgcgcgagctgatgcgccttgagcagacggtttccgatgcgcgcctgcgcaacgccatcaagaccggcatggcggaaaccaatgcagagctttacgactaccggctgcgccagattgccgccgggt

SEQ ID NO: 344 - DNA, 150 bp, Escherichia virus 186
  Source of Sequence: NCBI:txid29252
atcaggcaggccaccgacagaccattcgacggtgagcgcgtcatcgtcgaggccgaggtcaatcgccgccgcgccattcatgccgccgccgcgatagttttcgagcttgcgggtcagcttcggcagcgtcacggattcaacaacgcccat

SEQ ID NO: 345 - DNA, 150 bp, Escherichia virus 186
  Source of Sequence: NCBI:txid29252
ccaccgcaacagcctacgccaccgcgcgcgcactcggcctgcgtgcctatatcgaccagaccgtcggctggcacaaaaccctgtctaacgtcggcgtgcagggtgttaccggcatcagtgcgtcagtgttttgggatttgcaggcatccg

SEQ ID NO: 346 - DNA, 150 bp, Escherichia virus 186
  Source of Sequence: NCBI:txid29252
cgaactgagaagcgataaagctaaaaaagcacgctttcatgccgaggccatcgacccggaaaagggcagcgctaccggctatgtcgctaaatacatttccaaaaatattgacggttatgcacttgatggcgagctcgacgacgaaagcgg

SEQ ID NO: 347 - DNA, 98 bp, Escherichia virus 186
  Source of Sequence: NCBI:txid29252
tggtcagcaccattgccaagcgcaataaactgacggccagtgtcgcggattcactgaaaaaaaaaccggtaccgcatatagacccgtcgcaggagacc

SEQ ID NO: 348 - DNA, 150 bp, Escherichia virus 186
  Source of Sequence: NCBI:txid29252
tggtcagcaccattgcaaagcgcaataaactgacggccagtgtcgcggattcactgaaaaaaataccggtaccgcatatcgaccagtcgcaggagtccgacgccgtatttctgacccggctcgctgaccgcaacggggcggcggtgtcag

SEQ ID NO: 349 - DNA, 148 bp, Escherichia virus 186
  Source of Sequence: NCBI:txid29252
tgatgtcgggcgtgctgctgctgcccgtgcggaccggaacgggggctttctaccgcaagcggatcggccgcgtcctcctggcgctggtcttctggtcgctgacgctgcccgtgctctactacctgtatatgcgctacgtcgggacctc

SEQ ID NO: 350 - DNA, 150 bp, Escherichia virus 186
  Source of Sequence: NCBI:txid29252
ttctacgctcccggcggaaaaccggacggcatcaaaatcgtgccactgagtgaggtcgccacaaaggatgactttttcaacatcaagaaagccagcgccgccgacctgatggacgcgcaccgcgtaccgttccagctcatgggcggcaag

SEQ ID NO: 351 - DNA, 150 bp, Escherichia virus 186
  Source of Sequence: NCBI:txid29252
cagcgacgatgcgcgcctgaatgatgtgcatgaggcggtgaccaccgtcagcgaacatgtgcagaccaacctgaccaagcaagacgagcgcctttccgctatggaaaccgcgtttgccaccttcaaacaggagctgaccggcaaggtcga

SEQ ID NO: 352 - DNA, 150 bp, Escherichia virus 186
  Source of Sequence: NCBI:txid29252
tgcatcgactgataaggcagggtgcgacgttcaaacacaaacattccaagagcaagcatcatcatcagcctccttaatcgtgcatcatgctagcgcgggctttggctcgcttgtcgcgttcatatttttctaacgcatcctgcaactggt

SEQ ID NO: 353 - DNA, 150 bp, Escherichia virus 186
  Source of Sequence: NCBI:txid29252
agaaaggtgctttctctccagcctgagtgcgtgcgttttgatgtttatcgcactgctgcggtactggagcaaaatcagggcagtcaacgagccaatgccttttttatcagcttttgcaaaaatgcattgccccgtcttgaactggtcgcc

SEQ ID NO: 354 - DNA, 150 bp, Escherichia virus 186
  Source of Sequence: NCBI:txid29252
cgaaagcggtatcaagctcaccaccgattgccagcagcagaacctgctcaccgaccgagggagcccaccacgtccgcgaacgaccggcgcgggtggtcagccagttcagccatgttgtctggatcccgccgctttgtacgcggcacagcc

SEQ ID NO: 355 - DNA, 150 bp, Escherichia virus 186
  Source of Sequence: NCBI:txid29252
gcccgacttttccagcttgctggtgataggttccagatgcttggggatgacatttttgatgagcacgttgccgcctgtgatcgtggcggcgaccatgtagctgccggcctcgatctgatccgggatgatggaataggtgcagccgtgcat

SEQ ID NO: 356 - DNA, 150 bp, Escherichia virus 186
  Source of Sequence: NCBI:txid29252
cttctaaaaatttcaaaattccctgataaatctctccggaatcagaatatttctgctctgccacttcctctgacagcttggatgtatcgatccaattcttcgaaatggcataatttaaatactggttcagattgatcgtttcctcttttg

SEQ ID NO: 357 - DNA, 150 bp, Escherichia virus 186; identical to SEQ ID NO: 354
  Source of Sequence: NCBI:txid29252

SEQ ID NO: 358 - DNA, 150 bp, Escherichia virus 186
  Source of Sequence: NCBI:txid29252
ggtgcttcatatggagcatggcggtggaaataattctacgtttactacccgtgtagtaacttctgcaggatccgacacctattcagttattgctgcggcactttcttctctgaaaggtccgaaacatggcggtgccaatatcaaagtagt

SEQ ID NO: 359 - DNA, 150 bp, Escherichia virus 186
  Source of Sequence: NCBI:txid29252
aaaaccaaccaacaaactcaacgtatatccacagtacggagccagataaagtccgaaagggaaataaataatagctccggcaatagattgggtgatataattagtcagactcatcctaccataataacgaagatttgatacaaaattctt

SEQ ID NO: 360 - DNA, 150 bp, Escherichia virus 186
  Source of Sequence: NCBI:txid29252
ggtacgcatcccggtaatatcgacatacatttcgccagcgtcgccggtctcaaagctgatggcggtaagccggatgcgcggctcccacttctggatagccgaatagcacgccaccataatttgcagcctgagcgccgggttttgcggcat

SEQ ID NO: 361 - DNA, 150 bp, Escherichia virus 186
  Source of Sequence: NCBI:txid29252
atacgcaggcggtttgccgacccggtcattagacgcccagcggtaattcttcgagtgctgcatcgactgataaggcagggtgcgacgttcaaacacaaacattccaagagcaagcatcatcatcagcctccttaatcgtgcatcatgcta

SEQ ID NO: 362 - DNA, 150 bp, Escherichia virus 186
  Source of Sequence: NCBI:txid29252
aaaacaggatgagaatctgaaagatcctaacgcagatccagaagttatggaattattcaacgaagtttataccaaatggatgggaagtacattagtcaagaatctatattccaaagtgcttattacactgggaatggagtatctggacac

SEQ ID NO: 363 - DNA, 150 bp, Escherichia virus 186
  Source of Sequence: NCBI:txid29252
gttctgctacgcggatcccgaaaatacgaatctcttcacggtgcagcttcgcgcggatacgcgcccataagccagtaagatagctttgtgtatctgccgggctggctccgttccatttgctgttacggtagcccgctttggtggtggcgt

SEQ ID NO: 364 - DNA, 150 bp, Escherichia virus 186
  Source of Sequence: NCBI:txid29252
cgttcagtttggcgagctgggtcagataggcattgaacttaaaacgggtttcctgacgcatagtatttcctgtttgaattaatcggttagtcacagcatcgggcggggttgccgcccggtttcggtctgcggtttatcagcagtcggtca

SEQ ID NO: 365 - DNA, 150 bp, Escherichia virus 186; identical to SEQ ID NO: 364
  Source of Sequence: NCBI:txid29252

SEQ ID NO: 366 - DNA, 150 bp, Escherichia virus 186
  Source of Sequence: NCBI:txid29252
atcgccgtcacggcgctcaggtagtggaaaacacgcacattctcgccgtcgatttcctcggcgtcaggcacgtcggccagtgtcttaaacccggcggcaatctggcgcagccggtagtcgtaaagctctgcattggtttccgccatgccg

SEQ ID NO: 367 - DNA, 150 bp, Escherichia virus 186
  Source of Sequence: NCBI:txid29252
tcgactacgactatacgccggttccaccactggaaagcctgaccctgcgccagcgtatcaccgataaatatctggtgaatctggccgaatcggtcaacagctaaggagcctgaaacaacatggcactaccccgcaaactcaaatatatga

SEQ ID NO: 368 - DNA, 150 bp, Escherichia virus 186
  Source of Sequence: NCBI:txid29252
taataccgtcgacaatgtcacggataagcgatgcggtgatgggcttgtcgaccgcccacatgtgcgcctcggccattgtgtcggccagcacctgagcggtgcgggtgtagttctcaaacaggaaaagcgggtcatcagaacaggtgcggt

SEQ ID NO: 369 - DNA, 150 bp, Escherichia virus 186
  Source of Sequence: NCBI:txid29252
tagaccgttaccggcgcgccaccgataaactgaaactgacgaatatgccagcgcgcggcccaggcagaaacggcaggtgcggtctctttcaacagttcgccgctttcgtcgtcgagctcgccatcaagtgcataaccgtcaatatttttg

SEQ ID NO: 370 - DNA, 150 bp, Escherichia virus 186
  Source of Sequence: NCBI:txid29252
gtgccgtgaaaaccgaattgtgaaaaagtgtccagtgctgcgctaagaatcgcttttttcttcgcgcttactgcgcgcgcacgtttacccgttgttttcactgcgccttgcgtcatgcgctctcccctcttgtagattggcaacaggata

SEQ ID NO: 371 - DNA, 150 bp, Escherichia virus 186
  Source of Sequence: NCBI:txid29252
ctctggaaatgcttcaaattttctgggataacggaaagagctttaatgaagctgattctgtgagatatctgttcagaaacgggaaaattcagacggagtttgaacttccggagaataccacaatgcttcgccttgatccgggcgaaatgt

SEQ ID NO: 372 - DNA, 150 bp, Escherichia virus 186
  Source of Sequence: NCBI:txid29252
ctgatcagcggcatgatcagataaagtcctatcagcatatagagataccagagcggcgtggtgtcgtagcagaagttgaagacgaaggtccccatcttgtgcagcgtcgcttcgccggtgaacagcgcggggtcgatcgacggactggag

SEQ ID NO: 373 - DNA, 150 bp, Escherichia virus 186
  Source of Sequence: NCBI:txid29252
agtcgtttacctccctgaacctgtcctgcaggggtgacagctcgttacgcacaaatacttttgccaccttctcaacgtcaccgagcgaaccgatattctcgggcttgccgcccatgagctggaacggtacgcggtgcgcgtccatcaggt

SEQ ID NO: 374 - DNA, 150 bp, Escherichia virus 186
  Source of Sequence: NCBI:txid29252
ccattccaacaaggaaaggagcccaatatggaatttatcagagaagattgtatttacgcaagacagtcagtagaccgcaaggacagtatcagcattgaaagtcagatcgacttttgcaagtatgaattgaaaggtgggagctgccgggta

SEQ ID NO: 375 - DNA, 149 bp, Escherichia virus 186
  Source of Sequence: NCBI:txid29252
agctcgcttggtgggtatcaggcttatcagcctgtcacggctcccgccggtcgtacctatattgaccagagcagcccgacctatcaaatcaacctgccgggtggtggcgcgccgggtggtcaattgggtaaccagttgcaggatgcgtt

SEQ ID NO: 376 - DNA, 150 bp, Escherichia virus 186
  Source of Sequence: NCBI:txid29252
cacgctggaagttgacgatccggcgatggtagcaaagcagatggaactggtgctggaaggctgtgtaagccgaatgcgggtgaatcgtcgccaggcggatgtcgacactgcacatcggctggcggaagatatcctgcgcttcgcccgctg

SEQ ID NO: 377 - DNA, 150 bp, Escherichia virus 186
  Source of Sequence: NCBI:txid29252
caagaagtaaaaagatgaaaagaaaaatattagttactgttcacataccactatcccgcgaggtgttttggcatgaatatgccaaaatcccgagacatacaagccaaaattccattgccgcaggcaatctggaatgaattttggcaaagt

SEQ ID NO: 378 - DNA, 150 bp, Escherichia virus 186
  Source of Sequence: NCBI:txid29252
caggagctcgcacgcgcgattcgcaacctcatccgctcaggtgtggtgactgaggttgataccgtgcaggggctgtgccgcgtacaaagcggcgggatccagacaacatggctgaactggctgaccacccgcgccggtcgttcgcggacg

SEQ ID NO: 379 - DNA, 150 bp, Escherichia virus 186
  Source of Sequence: NCBI:txid29252
taaaccatgacgagccccgcacagcgtcacatgatgcgggtctcggcctctcaagccgcgcagcgggaacaagccccgctgcgccatgcaaccgcctacgagcagatgctggtaaagctggccgatgaccgtcgcacgttaaaaaccatc

SEQ ID NO: 380 - DNA, 150 bp, Escherichia virus 186
  Source of Sequence: NCBI:txid29252
gtcagagcatgcgcgacatactgctgacgccggtcggctcgcgggtgatgcgccgtgaatatggctcgcttctgtcggcgctgattgatatgccgcaaaacccggcgctcaggctgcaaattatggtggcgtgctattcggctatccaga

SEQ ID NO: 381 - DNA, 150 bp, Escherichia virus 186
  Source of Sequence: NCBI:txid29252
gagttgattatggctagtgacagtgaactcattcgccagactgccggaactgcttttgatatgtggcaaaagttcgcttttacatttgtactggttgcttcttttgtattgctttatcaacgggataggttcaagaattttgtatcaaat

SEQ ID NO: 382 - DNA, 150 bp, Escherichia virus 186
  Source of Sequence: NCBI:txid29252
atcttctgcaacgcacttcaggcataaaattgcgctatatcacatctactggagatactttgccggtagaatggataaaagagacagagaaaatgcttactggaaccatagtggtgccgatgtacggaattaccgaatgtaagagagtgg

SEQ ID NO: 383 - DNA, 150 bp, Escherichia virus 186
  Source of Sequence: NCBI:txid29252
gctaaacggtcatgaatttatcgccgggcgacaggcttgcccggtcagacaacccgatttaaccaaacaggaaagactatgcgtcaggaaacccgttttaaattcaatgcctacatgtcccgcgttgctgagttgaacggcatagacccg

SEQ ID NO: 384 - DNA, 150 bp, Escherichia virus 186
  Source of Sequence: NCBI:txid29252
gagaatgcgcggcttaacgccggtgaccgcttcggcagtcaacagcgccttgataccggtgtatttaccgttctcatccgtgccgccgatgatattggaaatggtctgcgcttccgcgtcttctccggtaccttcggcaacacgcacgac

SEQ ID NO: 385 - DNA, 150 bp, Klebsiella_phage_vB_Kpn_IME260
gagggaaggagacacagtggttataacggaactgacccggctagggcgttctgtaaaggaactgttcaccatcatcgagagggtgcatgatgccggagcctcgataaagtcgctccgtgagacttggcttgacaccaccacaccgcaggg

SEQ ID NO: 386 - DNA, 150 bp, Klebsiella_phage_vB_Kpn_IME260
gccgttgagaaactgaacgagggcggcatattggctttcgtcacatcaagaggtatcgctgacacacaaggcaaccaattcgtgcgtgactatcttgtacaccgttgcaatctgattacggcattacgtttgtctgatagcctgttcatg

SEQ ID NO: 387 - DNA, 150 bp, Klebsiella_phage_vB_Kpn_IME260
ggttcgcaaatcacgttcaatatcggaccgtatgccgctcggaatttggagcttggcaacgatttcgttaaatttgacgcaacgtttaacggtgtcgctgaaagtattcaggttcccgtcggcaacatagaggcggtgtttgtcagagag

SEQ ID NO: 388 - DNA, 150 bp, Klebsiella_phage_vB_Kpn_IME260
gcttctgcaatggctgtttctgagctttcagcacgtatttgcaatgtttggggcaacgatcctggtgcctaccctgacgggcttacccgtttcggtggcactttttgcaagcggtgtgggaacgctgatttatacgtttgtgacaaagcg

SEQ ID NO: 389 - DNA, 144 bp, Klebsiella_phage_vB_Kpn_IME260
aatcaagtctcctgatgaatatcaaaaaaaggcggctgaaattaaagcaacacagctacttggcatagaattactttccagcgtttatatgcttgctattttgaatatgattcttatgggtgatggcagttcaaatattttgaa

SEQ ID NO: 390 - DNA, 150 bp, Klebsiella_phage_vB_Kpn_IME260
atgactcatatctgacaccgccgttaaaccgcgtacggcagtctctaccgtgcgggcaaagttgggggaatccatggtgtgggcaatattcaacgaacctaaattgcaggaaatatcatgacctgtacgggcatagtcgagattctcgtc

SEQ ID NO: 391 - DNA, 150 bp, Klebsiella_phage_vB_Kpn_IME260
accgtatttacgactcgctgtgacacattatttggtgcgacgtattgtgtaatggctccagaacatccttatgtggacgagattacaacttcagaacagaaggaagctgttgatgcctataaagccatctgcgcaacgaaatccgatctg

SEQ ID NO: 392 - DNA, 150 bp, Klebsiella_phage_vB_Kpn_IME260
cggcctcttaattggtgtaactgcgaaaggccgaagcgttctgcgttttcaataagcattaccgaggcgttcggcacattcactccaacctcgatgacagttgtggctaccatgatgtgtgcctgtccggatgcaaataattgcatctga

SEQ ID NO: 393 - DNA, 150 bp, Klebsiella_phage_vB_Kpn_IME260
aatcccgatgtcaagtcgatatttgatttccggtatgaagacttctcacttgaagattatgatcctcatccacacattaaagccattgtagccgtatgattgcagaacgacctcaagtaatatcgataattgtggcctgcgccggtgcag

SEQ ID NO: 394 - DNA, 150 bp, Klebsiella_phage_vB_Kpn_IME260
ggtataatacaccttccagttcgtgtgctgatccaaaagtgccttcagctccagtacaatagccaggtccaaatccttttcctgaacccccagcttaattgcccccggctgtccgcccccatggccggcatcaatcaccagtaccttttc

SEQ ID NO: 395 - DNA, 150 bp, Klebsiella_phage_vB_Kpn_IME260
actccatcaggatcatccagggtatcgccccttactacgagaagtaccactttgtctccatctcccccgagatgtgccgcctggccgtgaccatgagcgagcggtacatcaccgaccgcttcctgcccgacaaggccatcgacctcattg

SEQ ID NO: 396 - DNA, 150 bp, Klebsiella_phage_vB_Kpn_IME260
accagctctgaaagccgcattagtaattttctcttgtgggagcttgcctatagtgagctctactttacctccgttttatggccggattttggtaaaagagaatttaaagaagcattattgtggtacgcatcaagagaaaggcgctttggt

SEQ ID NO: 397 - DNA, 150 bp, Klebsiella_phage_vB_Kpn_IME260
catggtttttaccccagaatagagagagaatctatcttgtcggacgtcttgcaaaaaaatgtaccggagacgtatttcctttcccggcgccgggtggaatgcatggttcgttcgggaaagatgcgactgctgcctctgtatgtggaacaa

SEQ ID NO: 398 - DNA, 150 bp, Klebsiella_phage_vB_Kpn_IME260
ctttttgcaagaaatcttcaaatcggagactcaggagacgtagacctttttatccgaaccagctctgaaagccgcattagtaattttctcttgtgggagcttgcctatagtgagctctactttacctccgttttatggccggattttggt

SEQ ID NO: 399 - DNA, 150 bp, Klebsiella_phage_vB_Kpn_IME260
aaaatcgattgcttgcaagatctggtacgtaaattcagtgtaagagatccctacttctaaacggctagcaacaacttctttattcaacatcgtattaacgttgaacaatttaccataatcgcgcaagaaatctaataaactgatctcttt

SEQ ID NO: 400 - DNA, 150 bp, Klebsiella_phage_vB_Kpn_IME260
tttacctacccccggcggccctaccaggcagaggatcggtcccttgattttgttgacacggctttgaaccgcaagatactcaaggatacgatctttcacgcgctccagaccataatggtcggtatcaaggatttcctgcgcctgacgcag

SEQ ID NO: 401 - DNA, 150 bp, Klebsiella_phage_vB_Kpn_IME260
taaaaaggccgttacattgccacttggttcttctagaaatttcaacagactgttagcggcactgatcgtcatcttttcagcattttgaatgataaaaagcttccgattgctctcgaccccacttttgacgaattcagctttcaaatatct

SEQ ID NO: 402 - DNA, 150 bp, Klebsiella_phage_vB_Kpn_IME260
ggaccgtatgccgctcggaatttggagcttggcaacgatttcgttaaatttgacgcaacgtttaacggtgtcgctgaaagtattcaggttcccgtcggcaacatagaggcggtgtttgtcagagagaccggctttggcatcccgttcgag

SEQ ID NO: 403 - DNA, 150 bp, Klebsiella_phage_vB_Kpn_IME260
cagcgtccgatatttgatcggccactttgtcggggtgtccttcagacaccgattcggatgtgaataaatatcccattctaaattgaaaattaagaattaaaaattaaaagttaattcgttaacaaccttgggaagttatcagagtagata

SEQ ID NO: 404 - DNA, 150 bp, Klebsiella_phage_vB_Kpn_IME260
agcttggcaacgatttcgttaaatttgacgcaacgtttaacggtgtcgctgaaagtattcaggttcccgtcggcaacatagaggcggtgtttgtcagagagactggctttggcatcccgttcaaggtcaaacttaaacctgaccaagaac

SEQ ID NO: 405 - DNA, 150 bp, Klebsiella_phage_vB_Kpn_IME260
aaggcccgcgaggctgcccgcaaagcccgcgagatgacccgccgcaagaacgtgcttgactcgtttagtatgcccggcaagctggccgactgcgccagcaaggatgcctcccagtccgaaatcttcattgtagagggcgactcggcaggt

SEQ ID NO: 406 - DNA, 150 bp, Klebsiella_phage_vB_Kpn_IME260
ggggaatatcggaggccatgaccggatcaaagcggtggtggatacggcaaaagagcggggaattccgatccgtgtgggcgtaaacagcggctctctggaaaaagagctggtggaaaaataccatggagttacagcggaaggaatcgtaga

SEQ ID NO: 407 - DNA, 150 bp, Klebsiella phage_vB_KpnM_KB57
agcacgtgctctatgcagcatgctgctggcgcatcaagcctgtcgaaaaacagccctgagtgcttgtcgaagaatgatacaaagcgatggcccattttgtttatggtgagcacctcctgctcatcctccggcatccgctcgcttaccgga

SEQ ID NO: 408 - DNA, 150 bp, Klebsiella phage_vB_KpnM_KB57
cgactattcaaaatcgcacagcgcatcaaagggccaaccttcgataaatactctcattcgccgtcgcaggctatcgacgtgctggaagttgcaatattcggcgaggatgaatcttgccgcgccgccatgctcgcagccgccccgcagcta

SEQ ID NO: 409 - DNA, 150 bp, Klebsiella phage_vB_KpnM_KB57
aaactggcaaaagtcattcgggagttcaacgaagaagacgaaaggccattagcaaaaatggcgcggatcattcgtgataatccacacccgacaaacgaatgcgacatgccgaaagcgcagccagtgccggatgactaccagcacctacga

SEQ ID NO: 410 - DNA, 150 bp, Klebsiella phage_vB_KpnM_KB57
atcctccggcatccgctcgcttaccggaatccatttacccggcgcggtggcgggttcactgccgggtagctgcggggcggctgcgagcatggcggcgcggcaagattcatcctcgccgaatattgcaacttccagcacgtcgatagcctg

SEQ ID NO: 411 - DNA, 150 bp, Klebsiella phage_vB_KpnM_KB57
ctcctgcggtgcggccggcagcggcatccagtgagttaccagcacgtgctctatgcagcatgctgctggcgcatcaagcctgtcgaaaaacagccctgagtgcttgtcgaagaatgatacaaagcgatggcccattttgtttatggtgag

SEQ ID NO: 412 - DNA, 150 bp, Klebsiella phage_vB_KpnM_KB57
acggcaaaaacgggaaatacaatgtcacggagcgtgtacgaggatggacagatgaagacgaaattgggttattcgtccaggtcggcgcgattctgcgaggtgaatcagaaatcacctggggggagccactttatctctctggagtcgtca

SEQ ID NO: 413 - DNA, 150 bp, Klebsiella phage_vB_KpnM_KB57
gagtgcttgtcgaagaatgatacaaagcgatggcccattttgtttatggtgagcacctcctgctcatcctccggcatccgctcgcttaccggaatccatttacccggcgcggtggcgggttcactgccgggtagctgcggggcggctgcg

SEQ ID NO: 414 - DNA, 150 bp, Klebsiella phage_vB_KpnM_KB57
tgagacgacaaatccctggttgataattgtggttagcttggaaaggtcgtccgcacttgtgccgtactgccttgtcgctctttccagtctcgcatacagcgacgctgtggcatctaaacttcctctcgtttgctgtgtaatgttgaatac

SEQ ID NO: 415 - DNA, 150 bp, Klebsiella phage_vB_KpnM_KB57
gccacgcgcagcagccggtactgcagccagtgatgtttattgatggtgacatttcatccgaggacgccgataaactggcaaaagtcattcgggagttcaacgaagaagacgaaaggccattagcaaaaatggcgcggatcattcgtgata

SEQ ID NO: 416 - DNA, 150 bp, Klebsiella phage_vB_KpnM_KB57
agacgaaaggccattagcaaaaatggcgcggatcattcgtgataatccacacccgacaaacgaatgcgacatgccgaaagcgcagccagtgccggatgactaccagcacctacgagagctctaccacgcgcaggaaaagcgactattcaa

SEQ ID NO: 417 - DNA, 150 bp, Klebsiella phage_vB_KpnM_KB57
gaatgcgacatgccgaaagcgcagccagtgccggatgactaccagcacctacgagagctctaccacgcgcaggaaaagcgactattcaaaatcgcacagcgcatcaaagggccaaccttcgataaatactctcattcgccgtcgcaggct

SEQ ID NO: 418 - DNA, 150 bp, Klebsiella phage_vB_KpnM_KB57
cagccagtgatgtttattgatggtgacatttcatccgaggacgccgataaactggcaaaagtcattcgggagttcaacgaagaagacgaaaggccattagcaaaaatggcgcggatcattcgtgataatccacacccgacaaacgaatgc

SEQ ID NO: 419 - DNA, 150 bp, Klebsiella phage_vB_KpnM_KB57
ggcatccagtgagttaccagcacgtgctctatgcagcatgctgctggcgcatcaagcctgtcgaaaaacagccctgagtgcttgtcgaagaatgatacaaagcgatggcccattttgtttatggtgagcacctcctgctcatcctccggc

SEQ ID NO: 420 - DNA, 150 bp, Klebsiella phage_vB_KpnM_KB57
aatagagatcgcccgtgcgtaaaccagcctcaacgtgaccaacaggaatacgctgataaaacgccgccaggctggttgccagcgtcgttgttgtgtcgccgtgaaccagcacgacgtctggtttgaactcggcaagaataggttttagcc

SEQ ID NO: 421 - DNA, 150 bp, Klebsiella phage_vB_KpnM_KB57
cgcggatcattcgtgataatccacacccgacaaacgaatgcgacatgccgaaagcgcagccagtgccggatgactaccagcacctacgagagctctaccacgcgcaggaaaagcgactattcaaaatcgcacagcgcatcaaagggccaa

SEQ ID NO: 422 - DNA, 150 bp, Klebsiella phage_vB_KpnM_KB57
cttctgctgcagcaacccttcgccggcgggctgcatgctttcgacgataatctggtagtcgccgcgcggctcatacagcgtgatgttggcgcgaaccagcacctgctgcccgtgctgcgggcggaacgtgacccggcggttgctgttgcg

SEQ ID NO: 423 - DNA, 150 bp, Klebsiella phage_vB_KpnM_KB57
agaccgattatctcgatctctatcaggtacactggccgcagcgcccaaccaactgtttcggcaaactgggctacacctgggccgattccgcgccggtcgtcactctcctggatacgctggatgctctggctgaattccagcgtgccggca

SEQ ID NO: 424 - DNA, 150 bp, Klebsiella phage_vB_KpnM_KB57
ccctcatcaacatcataaaaacgcgtaatcaggctggcgatggtagatttacctgaccctgagcgccctaccagcgccacggttttcccttccggaatattgaggtcaatattgcgcaacgcagcgacttcacgccccggatatctgaag

SEQ ID NO: 425 - DNA, 150 bp, Klebsiella phage_vB_KpnM_KB57
catcgttactcttatcaaacgacagcgtgttaatgcccatcagttctcccagcgagttcaccagcgcgccgccagagttaccgtggttaatggaagcatcggtttgtaggaagttttgccgcccggtcgggttcagaccgattcgccccg

SEQ ID NO: 426 - DNA, 150 bp, Klebsiella phage_vB_KpnM_KB57
attttcgataaagatggtttcgcccggttgaaccagtgacgcggcaaactcggcgagttcacgcttcagcgtatagttgctcatcatacgggtttcgacgtcatcactatcaagcgaaactgcaaagccatgtgcacggcggaggtaact

SEQ ID NO: 427 - DNA, 150 bp, Klebsiella phage_vB_KpnM_KB57
cagctcaccgatgaaggaaaaaaaatctattccagtattgagctgcatccgcagtttgcccttaacggtaaggcgtatgtggtcggcctggcgatgacggacaccccggcaagtctggggactgagcgccttaaatttgccgcgcagcag

SEQ ID NO: 428 - DNA, 150 bp, Klebsiella phage_vB_KpnM_KB57
ttccggcgcacagacgtattccgctctatcgccacgcgcagcagccggtactgcagccagtgatgtttattgatggtgacatttcatccgaggacgccgataaactggcaaaagtcattcgggagttcaacgaagaagacgaaaggccat

SEQ ID NO: 429 - DNA, 150 bp, Klebsiella phage_vB_KpnM_KB57
gcatcaaagggccaaccttcgataaatactctcattcgccgtcgcaggctatcgacgtgctggaagttgcaatattcggcgaggatgaatcttgccgcgccgccatgctcgcagccgccccgcagctacccggcagtgaacccgccaccg

SEQ ID NO: 430 - DNA, 150 bp, Klebsiella phage_vB_KpnM_KB57
gaaagcgtgaactcaatttttttcgccttaccatagcgatcaaactcctgatgcgtttcctgcaagcccgtgatgacatacattccgtaaatggatccgacgccatctatgagcggccaggccagcccggtgtaggccattgtcgagacg

SEQ ID NO: 431 - DNA, 150 bp, Klebsiella phage_vB_KpnM_KB57
cagcaggttagcttgaacggtaagttgatcggcaccaggtctgggtggcagattttctttctgtcgggcttcattacgggacatcagcccgttctgagtcatcgttgaataaaacgcagcacgagcggcactgtccgcacgacgcagccc

SEQ ID NO: 432 - DNA, 150 bp, Klebsiella phage_vB_KpnM_KB57
cctgtcgaaaaacagccctgagtgcttgtcgaagaatgatacaaagcgatggcccattttgtttatggtgagcacctcctgctcatcctccggcatccgctcgcttaccggaatccatttacccggcgcggtggcgggttcactgccggg

SEQ ID NO: 433 - DNA, 150 bp, Klebsiella phage_vB_KpnM_KB57
tggagtaactccggctggagcacgctggcagcgcagaacccgaagtccggcaaatacatcattcaggccacgaagacatccagcggctcaacggcctgtgaccaggtggtgaagctgacgggtggccagacgtaccgcgtgggtgcctgg

SEQ ID NO: 434 - DNA, 150 bp, Klebsiella phage_vB_KpnM_KB57
aagatagttttggtttaactccggtttctccgctgaaacggacctcgatggtaactggtcgcgcgaaacagcggcgtgcctatacctcgacaccaacccaaacagatatggcatggatttttaatgatgcgcaagctcaggcctttgaag

SEQ ID NO: 435 - DNA, 150 bp, Klebsiella phage_vB_KpnM_KB57
atagggggtcaatttgttgagaatatccagtatggccacgaacacctccacggcgatgatcccgccgctgtcacggtcgaccagctccagacggtcgcctgcgtaatcgacatacatcttgtcgccggccttgtgttccagatgcgctat

SEQ ID NO: 436 - DNA, 150 bp, Klebsiella phage_vB_KpnM_KB57
taccagcacctacgagagctctaccacgcgcaggaaaagcgactattcaaaatcgcacagcgcatcaaagggccaaccttcgataaatactctcattcgccgtcgcaggctatcgacgtgctggaagttgcaatattcggcgaggatgaa

SEQ ID NO: 437 - DNA, 150 bp, Klebsiella phage_vB_KpnM_KB57
cggacagcggaacggggatttcccgggaagagcggcagaagctgtttcagcggttctaccggggcggcaatgcggcggaggatagcgtgggcatcggcctggcactggccaaagaggtggtggaacaacaacatgggcatattcaggtgg

SEQ ID NO: 438 - DNA, 150 bp, Klebsiella phage_vB_KpnM_KB57
ggcggaatattctttggaagaagtttcacagccgattggtattacagggtatgaattaagcaatgcgctcagcaaagaatatcaacatagtcttcctacaatagaaacgctggagtctgaagtcgcaaaaatgatggatacaaaatagtc

SEQ ID NO: 439 - DNA, 150 bp, Klebsiella phage_vB_KpnM_KB57
ggtcagcagcatgacgcagcggtcgatgccgatgcccatgccgcccgtggggggcaggccgtattccagggcggtgaggaagtcctcgtccatcatgcccgcctcgtcgtcccccttggcccgcagctccacctgcttctggaaccgctg

SEQ ID NO: 440 - DNA, 150 bp, Klebsiella phage_vB_KpnM_KB57
ggcgtaatagaccagaccactgcaatcaaatccggtgcgcggagaggtcccgccccagcgataaggtttgccaatctgatccatcaactttgacatcgcggtgctttgcgctttttgtacccgcgctttatgcgcatcggcgatagtcag

SEQ ID NO: 441 - DNA, 150 bp, Klebsiella phage_vB_KpnM_KB57
tatggtgagcacctcctgctcatcctccggcatccgctcgcttaccggaatccatttacccggcgcggtggcgggttcactgccgggtagctgcggggcggctgcgagcatggcggcgcggcaagattcatcctcgccgaatattgcaac

SEQ ID NO: 442 - DNA, 150 bp, Klebsiella phage_vB_KpnM_KB57
tcggtttagcgtgtcggtgaacattttttcaacattctcatcccttatatcgttccaggctgagagtttttcaagtgttgagtattcactagtcccgcgatagttctctaatagctgccccgtggtcaggcccggctgcgcaacacagag

SEQ ID NO: 443 - DNA, 150 bp, Klebsiella phage_vB_KpnM_KB57
aggcgaaaacgtagccccgcgactgcaacagctcgcgcagcggctcatggttctcgataattccgttatgcacgaccacaatatggtcagaaacatgcggatgcgcgttgccttcagagggttcgccgtgcgtcgcccagcgggtatggg

SEQ ID NO: 444 - DNA, 150 bp, Klebsiella phage_vB_KpnM_KB57
  misc_feature (65)..(65): n is a, c, g, or t
ccagtaactggtgggtctgtgcttcatgcagatacatttccagctttggaaaggtctggtgcagnatagggataatatgcggtagcaggtacggtccaactgtgggaatcaaaccaatgtgcagcggcccggacatcgtctcgccctgct

SEQ ID NO: 445 - DNA, 150 bp, Klebsiella phage_vB_KpnM_KB57
gggcggctttattatgtgcggcgtgctgttctgcctggtttcttttatcgtgaagaaagcggggaccggctggctggacgtgctgtttccacctgcggcaatgggcgcaatcgttgccgtcatcggtctggagctggcgggcgtagctgc

SEQ ID NO: 446 - DNA, 150 bp, Klebsiella phage_vB_KpnM_KB57
ctgtcggagagaatgctgtggtcggtgccggtagcgtggttacccgcgatgttccgcccatgaccattgtagctggtaatccggcaaagataatcagaaaaatagacgaaaaataaacctatagtgatgattgagaacaaggaaataagc

SEQ ID NO: 447 - DNA, 150 bp, Klebsiella phage_vB_KpnM_KB57
aggatgaatcttgccgcgccgccatgctcgcagccgccccgcagctacccggcagtgaacccgccaccgcgccgggtaaatggattccggtaagcgagcggatgccggaggatgagcaggaggtgctcaccataaacaaaatgggccatc

SEQ ID NO: 448 - DNA, 150 bp, Klebsiella phage_vB_KpnM_KB57
aatggacatttcaccatacttacaatggtactccccaaggaggtatcatcagtcctatactggcgaatatctaccttgataaactggataagtatattgtggaatatatctcgaagttcaacaagggtaaggcgcggaaacgcaatcccg

SEQ ID NO: 449 - DNA, 150 bp, Klebsiella phage_vB_KpnM_KB57
cgaaggcttacaccggaacagcgtcttctatcgtggtcgttctggatccagggcacggcggaaaggataatggcgcatcagcgaatggtctgatagaaaaaaatatcaacctgaagattgcgcaggcatgtaaggctgaattagagaagc

SEQ ID NO: 450 - DNA, 150 bp, Klebsiella phage_vB_KpnM_KB57
ttcacatttaaataagtatcttgttgtcctgcaaaactcgcatcaggtagatcttcagctgttgcagagctgcgcactgccacatcagcttctttcataccatattctccactcaaaatttcatatgcttgaaaaatctctttgcgtaaa

SEQ ID NO: 451 - DNA, 150 bp, Klebsiella phage_vB_KpnM_KB57
aaacttttaataaagcagaaaaaagctttaaattattctgataaatggcaacttgatggcagtttagcaaaaggcgcagccatgaataacgataacatgaagttataccttagagcgtttaacagtgaatgtgatgtattgatttcaaaa

SEQ ID NO: 452 - DNA, 150 bp, Klebsiella phage_vB_KpnM_KB57
cgcgcgaacggacggtttccggacgcgcctggacgataaacagcttaccggtatggccatctttcgcccattcgatatccatcgggcggttgtagtgcttctcgatttgcactgcctgctgggccagctcctgcacttccgcgtcggtca

SEQ ID NO: 453 - DNA, 150 bp, Klebsiella phage_vB_KpnM_KB57
ttcgataaatactctcattcgccgtcgcaggctatcgacgtgctggaagttgcaatattcggcgaggatgaatcttgccgcgccgccatgctcgcagccgccccgcagctacccggcagtgaacccgccaccgcgccgggtaaatggatt

SEQ ID NO: 454 - DNA, 150 bp, Klebsiella phage_vB_KpnM_KB57
ctggaatctaacggcgatctgtataactgcgatcactttgtttatccggagcataagctgggtaatattcatcagcagagcatccgcgagctgaataatagcgagcaggccatcgcctttggtcagacacaacaagagacgctaacggcg

SEQ ID NO: 455 - DNA, 150 bp, Klebsiella phage_vB_KpnM_KB57
tcccctctgccgtggtcacgcgcgtggtcaggctactgatggccccggcgttggcggcaatattcacttcatccgtgatatccacaaaatagaagtcgtcaaaatactgagagccagacttcagggaagagtagatagccacatcaacca

SEQ ID NO: 456 - DNA, 150 bp, Klebsiella phage_vB_KpnM_KB57
actggaagagccttaatcttaacggcccaattgtcgataaacactctaccggcggcgtaggcgatgtgacttccctgatgctggggccgatggtcgctgcctgcggcggctatgtaccgatgatctccgggcgcggactcggtcataccg

SEQ ID NO: 457 - DNA, 150 bp, Klebsiella phage vB_KpnM_KpV52
gtcgaagcgaaagacgcgactccggcacaaaaagagatcaaagaaatcaatgccggcatgtactgttttaaaaataaggatcttttcgaaggtctgaaaaaattaaccaatgacaatgctcaaaaggagtattacttaacggatctagcg

SEQ ID NO: 458 - DNA, 150 bp, Klebsiella phage vB_KpnM_KpV52
gggtcgcggatcgcgccgccaaggcaggttgcggccccgccaaacggctctatctccgtaggatggttgtgcgtctcattcttgaaattcaccagccattcctccgtctgtccatccacttcaaccggaacgacaatcgagcaggcgttg

SEQ ID NO: 459 - DNA, 150 bp, Klebsiella virus 0507KN21
gatgaagatgtactatgtgggaccgcaatgtcgccatgaacgcccgcaaaagggcagaatgcgtatcttcaatcagtttggtgttgaggtcatcggtgccaaaagtccgttgctggatgtggaaacgattgcacttggatggtcgtttat

SEQ ID NO: 460 - DNA, 150 bp, Klebsiella virus 0507KN21
agcccattggcggaactggactccacgcgggttacggatgcggaatcctactgcaagaatcatttcgagcgagtagaatttcacggagtattgtttgccatcggaggcagttgtaaagaaatcctttacaactgaattgtataacaattc

SEQ ID NO: 461 - DNA, 150 bp, Klebsiella phage K5
aaacataacaccctgctgatcagtgccggttctacgttctacggctgcgcggcactggtgaacattccgccgtcgctgtttgcatcgtgcccgcttatcaccgcattcggcgcaacgttccagaataccggcgtggtggaaataccggaa

SEQ ID NO: 462 - DNA, 150 bp, Klebsiella phage K5
ggtccagctccagttctcccccaagaagccgcagcaaggtggtcttccctgccccgttgatccccaccaccccaatcttttctctgccttttatctcaaaatcaatgtgggacagcacctgaatccctcccagggagatcggaagagcac

SEQ ID NO: 463 - DNA, 150 bp, Klebsiella phage K5
cagttctgccgaccagcgataatcaccttgcttgttcgcttcttgcgccaggttgatgacgcgggcagaaccgcccagcgcctgcacgtaacgtttacccatctccacctgaccatacggatgcaggttagccgggttaccgtcgtaata

SEQ ID NO: 464 - DNA, 150 bp, Klebsiella phage K5
atatacatcaaacgatatacccaactcctcaaacgagtccttgattatcttgtggtagcggtcaacgatatcctgtggagtcacaccctcgcggcgtgcctttatagtgataggcacaccgtgctcgtcgcttccgcctaccatcacaac

SEQ ID NO: 465 - DNA, 150 bp, Klebsiella phage K5
cgcatgattgacgaacttgccaaggagagcaggaacagaagtatgaacgacaagaagcggaaggatgaatagtaaactaacacatggctctctgttcagcggcattggtggtccagaaatagctgctgaaatgatgggctggaaaaacgt

SEQ ID NO: 466 - DNA, 150 bp, Klebsiella phage K5
agtatgaagagataagatgcaagccagatttaatcgagccttttcaccgccggagaacttatgataggagccacagtcaatgccgtcgcgcatcacctgaactgaaatcttatctctcagcttaccagtcttggttacagtgaatccttc

SEQ ID NO: 467 - DNA, 150 bp, Klebsiella phage K5
caagccccaaacaggtccgccatgctgcccacaaagatgcgctggggcttcttcatctgggcagggtcccccaggcggtagcggtgcaaggtgggcttgaagccgaaggggaagggggcgaccacctccttgccattcttcctgaggaca

SEQ ID NO: 468 - DNA, 150 bp, Klebsiella phage K5
ccatccgtaagcggttcttcatggtgatgcgctgcgatgggaagccgattgtatggccggaagccactcatggagatccgaaatcaccggcggtgctagctggcaaactggcaccatggcgcacagctgcggaatgcatcgactggtcca

SEQ ID NO: 469 - DNA, 150 bp, Klebsiella phage K5
cgcctggccgatacgatccagacgaccaatacgctgctccagtagatccgggttgaatggcaggtcaaacatcaccatgtggctggcgaactggaagttacgtccttcagaaccgatttctgagcacagcagtacctgtgcgccggtgtc

SEQ ID NO: 470 - DNA, 150 bp, Klebsiella phage K5
ctccttcgatataaatattctgcgtctcatcccagtttttagacttttctttatttggttttaaagtcgcagtggttggttgttgagctaacttcattgcttgttttttaccgttccacgtaaagttatatcgctcgggagaatcatcta

SEQ ID NO: 471 - DNA, 150 bp, Klebsiella phage K5
agccccatctctttcccgagcttacggtggtcgcgcttcttagcttcctcaagaagagtgaggtattcatcgagcatctttttctttgggaaagtgatgccgtatactctgacgagctgtcctctcttttcgtcaccgcgccaataagcg

SEQ ID NO: 472 - DNA, 150 bp, Klebsiella phage K5
acccactcgcctccccagccgacgaccgaggagatgccccgctcgttgagctggttcgcggccacctggtcgaacccgcggctcttggagccctcggcgaagcgctggcgcacgacggggacggcgcggttgctccacccctcgaagggc

SEQ ID NO: 473 - DNA, 150 bp, Klebsiella phage K5
cacgcctttgcctcggcctcggtcttaccgtaacagtgcagggtgcgtttttctccgtccggatcgccaatctccaccttgattttctttttgttgtcgggttgtaggctaaccactttcagcttaattttcacgtcctcggctttctgt

SEQ ID NO: 474 - DNA, 150 bp, Klebsiella phage KP34
atagccactatgattgtagtagggctatatttattaccgatattgttttgaacaattaaaactggtctaactcctccttgttcagaacctaccgtagtttccagatcagcataatagatttgacctcttagtatatgattgtacatatcg

SEQ ID NO: 475 - DNA, 150 bp, Klebsiella phage Matisse
gtctcaaaacgttcgccgttgttattgtttacaatctgcactttctcaccggcaatcaaatttgcagcatccatcagatcttcatcgatagtaatgctacccatataattcaaattagcttccgtcacacgtacacagtgcaactttgac

SEQ ID NO: 476 - DNA, 150 bp, Klebsiella phage PKP126
cgaccagacagcctaccagcagcgacaggcgcgcgccgtacatcaggcgcgacaggatatcgcggccaacgtcatcggtgccgagcaggtgggacatgctgccgccctcctgccagaacggcggagccagcagcgcatcgcggaactgat

SEQ ID NO: 477 - DNA, 150 bp, Klebsiella_phage F19
acaccttcccaggcaaggcttgagtggcctcaaactccaacagtcatccccacccagcacccattgagacgctggggtgccttgccacatggctacgtggagatgtattcagggaaacactgaatgagccaggatggaagcagagtccac

SEQ ID NO: 478 - DNA, 150 bp, Klebsiella_phageF19
tgtcctgtggacagacagatatgcacctcccaccgcaagcggcgggccccgaccggagccactttagttacaacactcaaatacaaccaccagaaaaaccccggtcccgcgcagaactgaaaccacaaagcccctcctcataactgaaaa

SEQ ID NO: 479 - DNA, 150 bp, Klebsiella_phage F19
ataataatgaccactctgttttcagggtggttttttatttaagccgatcaagctcaatttttaggaaaagggccttagatatggttaaataagagtaaaaaggagatttgcgatgaaagatgaggtaaagtggcgccagaaaaatttatt

SEQ ID NO: 480 - DNA, 150 bp, Klebsiella_phageF19
tgcccctgcttagctgtgcggcgacggcattgtagtcaagcgaggcgaagccgaggagagacgtggtcatttccctgtcgagcgggaacgagtttccgtaccctgcggccgagccgagaggattctggtccgttacattatatgccccgt

SEQ ID NO: 481 - DNA, 150 bp, Klebsiella_phageF19
ttcttttttctcatactgtttatcctttcgacaattgtcaatttatattttcactttacatttccaaatagacaatgtaacttatactcattgccccccacaatcataataccgtcaatataattgtgtagttttaagcaaccgattgtg

SEQ ID NO: 482 - DNA, 150 bp, Klebsiella_phageF19
agataggatactaatcattgctggaataacaactttcctttgttcctctaaacatctgaatttcttatttttctcctgttcaaatgataagcatacaaaatataatttatctaaacataaaaaataaaaagcaaaaattcccttaggttt

SEQ ID NO: 483 - DNA, 150 bp, Klebsiella phage Sugarland
cattggcttgaggcaactgcgcccattgatcggggctggaataccctgctgaatctgcgttcatgggcaatggtcggcagcggcctcgtggcaatatggtcagttcgtcatccgcgctttttgatgcgctgggcaaaacgcagcctcggt

SEQ ID NO: 484 - DNA, 150 bp, Klebsiella phage Sugarland
actattctgcaaattaagggttcattatttgttggtcagttgttgctttccactgcaaggctttaaaaaataaataaaggtatcttcagaattccaacatttggaatttgaagatacctttttagtttccttataaattagggggtgtaa

SEQ ID NO: 485 - DNA, 150 bp, Klebsiella phage Sugarland
ggcagcggcggaccacagtttcgcggcgacgtggcggtgaagggcgatcgaattgtggatatggccccggcgctgaacctggcggcggagcaggtgattgacgggcaggggcgcgtgctggctcccggatttatcgatgtgcatacccat

SEQ ID NO: 486 - DNA, 150 bp, Klebsiella phage Sugarland
ggaaggcgtggcgcggcctgggcttggtcgcatggatgacgacggggtcggggtggtccggcgatggaggcggggcagaggacgggcggggcaggggagcgccggcgagctgcggtagaagatggccgggtccagcgagagctcctgtag

SEQ ID NO: 487 - DNA, 150 bp, Klebsiella phage Sugarland
acccgggatcaaacctacaaccatatcccacatggtagcgggagtaccgttcacattaatcagttcgcttgcacccggagcggctgtagcagcctgtcccaacatcgtagcgccggtaaaaccgtccactacctgtccgccaccgatacc

SEQ ID NO: 488 - DNA, 150 bp, Klebsiella phage Sugarland
atctcatctcttaatagttcattaatcaatgtcatttcattcataggcatgatcagtgaaaaaagactggaaatcataagagataatgacattacaataaattcttttttttcatgaaatgatagtttttgagctcgttcaccttgcaac

SEQ ID NO: 489 - DNA, 150 bp, Klebsiella phage Sugarland
atgcactcttttttgcgcaaaaagggctacgcgtggaatttatcgaccttgataccaccacagttacgactgataagaagtggttgatctgtattttagagcaattgatcttcaatgcgatcaaatataccaacactggaacgatcacgt

SEQ ID NO: 490 - DNA, 150 bp, Klebsiella phage Sugarland; identical to SEQ ID NO: 483

SEQ ID NO: 491 - DNA, 150 bp, Klebsiella_phage_vB_Kpn_IME260
atgccgctcggaatttggagcttggcaacgatttcgttaaatttgacgcaacgtttaacggtgtcgctgaaagtattcaggttcccgtcggcaacatagaggcggtgtttgtcagagagaccggctttggcatcccgttcgaggtcaaac

SEQ ID NO: 492 - DNA, 150 bp, Klebsiella_phage_vB_Kpn_IME260
cttcggcagtacaatatcaggatttcccggcagcttccttacctgaacgcggaaacgcaaacctcgtgagaaaagatacttccgcacaatcagttccggtttggtatcctttcccttgatagccgccatacagcggctacgctgctccgg

SEQ ID NO: 493 - DNA, 150 bp, Klebsiella_phage_vB_Kpn_IME260
tacaggcactgtttttggccgcggttcccgatcaattcctttgcaagcttcagccgttgtccttcaccgccggataaggtagtcaaaggctggccaagctgcagatagccaagtcccacatcctgcaatagagccaagattcccgtgatc

SEQ ID NO: 494 - DNA, 149 bp, Klebsiella_phage_vB_Kpn_IME260
tcttcaaatcggagactcaggagacgtagacctttttatccgaaccagctctgaaagccgcattagtaattttctcttgtgggagcttgcctatagtgagctctactttacctccgttttatggccggattttggtaaaagagaattta

SEQ ID NO: 495 - DNA, 150 bp, Klebsiella_phage_vB_Kpn_IME260
accatcaactaagagataacggtagctaaaatcttgcgccatccggaccatcgattcatagatcgccgagtccccatgcgggtggtacttacccataacatccccaacgatccgggctgatttcttatatggtttatccggagtcacacc

SEQ ID NO: 496 - DNA, 150 bp, Klebsiella_phage_vB_Kpn_IME260
ttcaatatcggaccgtatgccgctcggaatttggagcttggcaacgatttcgttaaatttgacgcaacgtttaacggtgtcgctgaaagtattcaggttcccgtcggcaacatagaggcggtgtttgtcagagagaccggctttggcatc

SEQ ID NO: 497 - DNA, 150 bp, Klebsiella_phage_vB_Kpn_IME260
ctacgcggtacatgggcacactgccgccgaattgatcgtagaccgggccgacgcccagaaagagcacatgggcttgacaacgtgggagaacgctccagatggtaaaatcctgaaaccggatgtaagcatcgccaagaactatctcaaaga

SEQ ID NO: 498 - DNA, 150 bp, Klebsiella_phage_vB_Kpn_IME260
tacgcttcctttatcgctacgagcaatcatacggatttgctgggcgatccttccggcagccgccgtttcatttgtatcgaggtgaaagggatgatagacaatgcccagcctatcgattatctccagctctatgcgcaagcggttgccgcc

SEQ ID NO: 499 - DNA, 150 bp, Klebsiella_phage_vB_Kpn_IME260
agaacgggcgaacaaagccaaaagtgaattcttatccaatatgagtcatgatatccgtacgcctatgaatgccattgtcggtatgacatccattgcttcggccaatttggagaatacaaaacaggtgcaaaattgtctgaagaagattac

SEQ ID NO: 500 - DNA, 150 bp, Klebsiella_phage_vB_Kpn_IME260
agcgtacgcaaacggttcgcaaatcacgttcaatatcggaccgtatgccgctcggaatttggagcttggcaacgatttcgttaaatttgacgcaacgtttaacggtgtcgctgaaagtattcaggttcccgtcggcaacatagaggcggt

SEQ ID NO: 501 - DNA, 150 bp, Klebsiella_phage_vB_Kpn_IME260
aacgatttcgttaaatttgacgcaacgtttaacggtgtcgctgaaagtattcaggttcccgtcggcaacatagaggcggtgtttgtcagagagaccggctttggcatcccgttcgaggtcaaacttaaacctgaccaagaacccgcgccc

SEQ ID NO: 502 - DNA, 150 bp, Klebsiella_phage_vB_Kpn_IME260
gctctgaaagccgcattagtaattttctcttgtgggagcttgcctatagtgagctctactttacctccgttttatggccggattttggtaaaagagaatttaaagaagcattattgtggtacgcatcaagagaaaggcgctttggtaaga

SEQ ID NO: 503 - DNA, 150 bp, Klebsiella_phage_vB_Kpn_IME260
tgactatatcatcgacgtactccttgcacaacgcaaatgtattatcgcccggttcttttaccgatatgccgtcggccacggtcgacacactcggcaggcactctatcttaccgtgcttcagcgactctaccatgctcggcgctccggcgg

SEQ ID NO: 504 - DNA, 150 bp, Klebsiella_phage_K64-1
cgacgttataaacaatgtgttctgcacactcttctaaagtgaaaaaatcatagaaatactgccgaaattcttctaaccattcctctgtgaaatattctggatcaagttcaacttttactttgtcacgtcttactaattcaatttcaaatt

SEQ ID NO: 505 - DNA, 150 bp, Klebsiella_phage_K64-1
catacatcagtcgactgcagccaagatatatcatgtgaatctgccggatgtcaccgaggagcagcgtcgccgtgccaagaccgccaatttcggtataatttacggtatatcggcattcggtctcgctcagagactgcgcataccgcgata

SEQ ID NO: 506 - DNA, 150 bp, Klebsiella_phage_K64-1
cgattttcgcccgtagctcggcaatctctttgcgcagccgctcctcctccagcagctgcgccggttcgcttttgctaatactctccagatcttcttccagagcatgcagctcccggcgcttaccggcggtcatcgccgtgcgccgcgcca

SEQ ID NO: 507 - DNA, 150 bp, Klebsiella_phage_K64-1
aacttcttccccttgcgtttaaattcagggaagatagctccggtcatcgtaccggatgaatcaaactccgtcgggtaacaattaccggcccgtacgatatctacagccaccttctccccggtaaggtatggcttaaagcaaataccacca

SEQ ID NO: 508 - DNA, 150 bp, Klebsiella_phage_K64-1
gcccccggattcggacagcgcggcatcgaaggcaaattctcagcgctcaaatggcttcgtgagcacgacatacccacgctcggaatatgcctcggtatgcagtgtatggtcatcgaatacgcccgcaacgtactcggacttcagcaggcc

SEQ ID NO: 509 - DNA, 150 bp, Klebsiella_virus_0507KN21
gcagggatcgtgctgcaagggactgagatcaaatcgatccgcaacagtcggatcaatctaaaagacggctttgttcgtatccgtaatggagaagactatttgtataatgttcatataagtccttatgaacagggaaacatttttaatcat

SEQ ID NO: 510 - DNA, 150 bp, Klebsiella_virus_0507KN21
aatatgggaatggtcagcgggatcctgcccgttgtcggcgtgccccttccctttatgagttacggcggtactgcgcttctgattttaggcatctgctgcggactgctgatgaaaatttccgccggacgccgcataaaagaatctttgtac

SEQ ID NO: 511 - DNA, 150 bp, Klebsiella_virus_0507KN21
ctatctgcgcaccttccgcttcacgggcgacctgtgggctgtgccggagggcactcccatcttcccccgggagcccttcctcaccgtccgcgcccccgccattgaggcccagttcatcgagaccttcctgctgctcaccctcaaccacca

SEQ ID NO: 512 - DNA, 150 bp, Klebsiella_virus_0507KN21
gtatattctttatatgtcttgaaatggtcgacttattgcgttgaaacagctccgtcatctggtcggcagtaagccacaccgattcattggcaagctgaacctcaatcttcgtctcgccaccctgcgtctgaaaaagtattatatttccgc

SEQ ID NO: 513 - DNA, 150 bp, Klebsiella_virus_0507KN21
attcatcgcatgcagaatacggcgatgaacaggcttcattccatcacgaacatcaggcagtgcacgatctacgattacagacattgaatagtccagaaaggaggttttcatttcttttgaaatatctacctgaatcagtttttccttatt

SEQ ID NO: 514 - DNA, 150 bp, Klebsiella_phage_KpV71
agatggggctggaggacaaggaaaatgcctacccctgccagctttcagggggccagtgccagcgtgtggccattgcaagggcattggccctgaatccccagatcctgttctttgacgagcctacctcggcgctggatccggaactgacag

SEQ ID NO: 515 - DNA, 150 bp, Klebsiella_phage_Matisse; identical to SEQ ID NO: 491

SEQ ID NO: 516 - DNA, 150 bp, Klebsiella_phage_Matisse; identical to SEQ ID NO: 492

SEQ ID NO: 517 - DNA, 150 bp, Klebsiella_phage_Matisse; identical to SEQ ID NO: 493

SEQ ID NO: 518 - DNA, 149 bp, Klebsiella_phage_Matisse; identical to SEQ ID NO: 494

SEQ ID NO: 519 - DNA, 150 bp, Klebsiella_phage_Matisse; identical to SEQ ID NO: 495

SEQ ID NO: 520 - DNA, 150 bp, Klebsiella_phage_Matisse; identical to SEQ ID NO: 496

SEQ ID NO: 521 - DNA, 150 bp, Klebsiella_phage_Matisse; identical to SEQ ID NO: 497

SEQ ID NO: 522 - DNA, 150 bp, Klebsiella_phage_Matisse; identical to SEQ ID NO: 498

SEQ ID NO: 523 - DNA, 150 bp, Klebsiella_phage_Matisse; identical to SEQ ID NO: 499

SEQ ID NO: 524 - DNA, 150 bp, Klebsiella_phage_Matisse; identical to SEQ ID NO: 500

SEQ ID NO: 525 - DNA, 150 bp, Klebsiella_phage_Matisse; identical to SEQ ID NO: 501

SEQ ID NO: 526 - DNA, 150 bp, Klebsiella_phage_Matisse; identical to SEQ ID NO: 502

SEQ ID NO: 527 - DNA, 150 bp, Klebsiella_phage_Matisse; identical to SEQ ID NO: 503

(19) United States
(12) Patent Application Publication
(10) Pub. No.: US 2023/0227789 A1
(43) Pub. Date: Jul. 20, 2023

(54) METHODS FOR TREATING BACTERIAL INFECTIONS

(71) Applicant: MICROBIOTA I - CENTER (MAGIC) LIMITED, Shatin, New Territories (CN)

(72) Inventors: Siew Chien NG, Hong Kong (CN); Ka Leung Francis CHAN, Tai Po, New (CN); Qin LIU, Shenyang (CN)

(21) Appl. No.: 17/783,120

(22) PCT Filed: Dec. 11, 2020
(86) PCT No.: PCT/CN2020/135614

§ 371 Date: Jun. 7, 2022

Related U.S. Application Data
(60) Provisional application No. 62/946,781, filed on Dec. 11, 2019.

(51) Int. Cl. C12N 7/00; C12Q 1/689; A61K 35/76

(52) U.S. Cl. CPC C12N 7/00; A61K 35/76; C12Q 1/689; C12N 2795/00021

(57) ABSTRACT

Methods and compositions for treating and/or preventing a bacterial infection in a subject are provided, in which the subject is administered a fecal sample obtained from a donor subject via fecal microbiota transplantation (FMT). The fecal sample contains bacteriophages that target the bacteria causing the infection. In some embodiments, the fecal sample containing the bacteriophages can be obtained from a donor subject who previously had the same infection but is now cured.

### RELATED APPLICATIONS

This application is a 371 U.S. National Phase Application of PCT/CN2020/135614, international filing date Dec. 11, 2020, which claims priority to U.S. Provisional Pat. Application No. 62/946,781, filed Dec. 11, 2019, the contents of which are hereby incorporated by reference in the entirety for all purposes.

### REFERENCE TO SUBMISSION OF A SEQUENCE LISTING AS A TEXT FILE

The present application is being filed along with a Sequence Listing in electronic format. The Sequence Listing file, entitled 080015-1329341-028710US_ST25.txt, was created on Jan. 6, 2023 and is 187,901 bytes in size. The information in electronic format of the Sequence Listing is incorporated herein by reference in its entirety.

### BACKGROUND OF THE INVENTION

Highly drug-resistant enteric bacteria, including carbapenem-resistant Enterobacteriaceae (CRE) and vancomycin-resistant Enterococci (VRE), are emerging worldwide¹. In the United States, CRE infections have been confirmed in 48 states and are estimated at 2.93 per 100,000 persons with almost 10,000 infections per year². Enterobacteriaceae causes 30% of healthcare-associated infections. Although they are susceptible to carbapenems^{3,4,5}, the emergence of bacteria with new β-lactamase enzymes with direct carbapenem-hydrolyzing activity has contributed to an increased prevalence of CRE in the past decade⁶. Some CRE bacteria have become resistant to most available antibiotics and patients carrying these bacteria are at a high risk of severe infections and a high mortality rate⁵. Currently, decolonization strategies are lacking, and targeted selective digestive decontamination resulted in short-term benefits and increased risks of resistance to the antibiotics used⁷.

Fecal microbiota transplantation (FMT) is highly effective in the treatment of recurrent Clostridioides difficile infections (CDI)⁸, and has recently emerged as a promising therapy for decolonization of intestinal multi-drug resistant microorganisms⁹. In four case series with varying study protocols, FMT resulted in 33-50% of decolonization in CRE infections¹⁰⁻¹³. However, the fate of native and introduced microbes and which species are enriched or cleared after FMT in CRE recipients remain unclear¹¹. Apart from the bacterial community, accumulating evidence showed that gut fungal (fungome) and viral microbiome (virome) which consists of eukaryotic RNA and DNA viruses and bacteriophage are also associated with FMT treatment outcome in CDI¹⁴. To date, there are limited data on how FMT affects CRE carriage in relation to the gut microbiome after FMT.

FMT can restore the gut microbial ecology, and has proven to be a breakthrough for the treatment of recurrent CDI. Furthermore, clinical trials are being conducted to evaluate its use for other conditions including treating multi-drug resistant microorganisms. There is accumulating evidence showing that the gut microbiota plays an important role in the control of intestinal colonization and infection by pathogenic bacteria. In addition, as bacteriophages propagate via exclusively lytic or lysogenic infection of bacteria, bacteriophage has the potential for eradicating multi-drug resistant microorganisms.

### BRIEF SUMMARY OF THE INVENTION

In one aspect, the disclosure features a method for identifying a donor subject for fecal microbiota trans-plantation (FMT), comprising: (a) analyzing a fecal sample obtained from a candidate subject to detect the presence of one or more predetermined species of bacteriophages in the fecal sample; and (b) determining the candidate subject as a donor subject when the presence of the one or more predetermined species of bacteriophages is detected in the fecal sample. In some embodiments, the method further comprises step (c) administering a fecal material obtained from the donor subject to a subject in need of FMT.

In some embodiments of this aspect, the subject in need of FMT has a bacterial infection, for example, a recurring or an antibiotic-resistant bacterial infection.

In another aspect, the disclosure features a method for treating or preventing a bacterial infection in a subject in need of FMT, comprising: (a) analyzing a fecal sample obtained from an individual as a proposed donor to detect the presence of one or more predetermined species of bacteriophages in the fecal sample, upon confirmation of the presence, especially at a desirable level (e.g., above an average level), of the one or more predetermined species of bacteriophages in the fecal sample the individual is chosen as FMT donor; and (b) administering to the subject in need of FMT a processed fecal sample from the donor containing the predetermined species of bacteriophages in an effective amount.

In some embodiments, the bacterial infection is an antibiotic resistant bacterial infection. In some embodiments, the bacterial infection is caused by bacteria in the family Enterobacteriaceae. In some embodiments, the bacterial infec-tion is caused by bacteria in the genus Enterococcus, Klebsiella, or Escherichia. In some embodiments, the bacterial infection is caused by carbapenem-resistant Enterobacteriaceae (CRE). In some embodiments, the bacterial infection is caused by vancomycin-resistant Enterococci (VRE). In some embodiments, the bacteria infection is caused by Klebsiella pneumonia, Klebsiella variicola, or Escherichia coli.

In some embodiments, the bacteriophage is selected from the group consisting of Klebsiella phage KP34 (NCBI:txid674081), genus KP32virus (NCBI: txid1985720), genus Kp36virus (NCBI: txid1920860), Klebsiella virus Kp15 (NCBI:txid1985328), and Klebsiella phage KP27 (NCBI:txid1129147), or from the group consisting of Klebsiella phage vB_Kpn_IME260 (NCBI: taxid 1912318), Klebsiella phage vB_KpnM_KB57 (NCBI: taxid 1719140), Klebsiella phage vB_KpnM_KpV52 (NCBI: taxid 1912321), Klebsiella virus 0507KN21 (NCBI: taxid 2169687), Klebsiella phage F19 (NCBI: taxid 1416011), Klebsiella phage K5 (NCBI: taxid 1647374), Klebsiella phage Matisse (NCBI: taxid 1675607), Klebsiella phage Sugarland (NCBI: taxid 2053603), Klebsiella phage PKP126 (NCBI: taxid 1654927), Klebsiella phage K64-1 (NCBI: taxid 1439894), Klebsiella phage KpV71 (NCBI: taxid 1796998), and Klebsiella phage Matisse (NCBI: taxid 1912318).

In some embodiments, the bacteriophage in the genus KP32virus (NCBI: txid1985720) is selected from the group consisting of Klebsiella phage K5 (NCBI:txid1647374), Klebsiella phage K11 (NCBI:txid532077), Klebsiella phage vB Kp1 (NCBI:txid1701804), Klebsiella phage KP32 (NCBI:txid674082), and Klebsiella phage vB KpnP_KpV289(NCBI:txid1671396).

In some embodiments, the bacterial infection is caused by carbapenem-resistant Klebsiella pneumonia, and the bacteriophage is selected from the group consisting of Klebsiella phage KP34 (NCBI:txid674081), genus KP32virus (NCBI: txid1985720), and genus Kp36virus (NCBI:txid1920860).

In some embodiments, the bacteriophage comprises a genome comprising a nucleic acid sequence of any one of SEQ ID NOS: 1-324 and 333-335, or any one in List 6 or 7.

In some embodiments, the bacterial infection is caused by carbapenem-resistant Klebsiella variicola, and the bacteriophage is selected from the group consisting of Klebsiella virus Kp15 (NCBI:txid1985328) and Klebsiella phage KP27 (NCBI:txid1129147).

In some embodiments, the bacteriophage comprises a genome comprising a nucleic acid sequence of any one of SEQ ID NOS:325-332.

In some embodiments, the bacterial infection is caused by carbapenem-resistant Escherichia coli, and the bacteriophage comprises a genome comprising a nucleic acid sequence of any one of SEQ ID NOS:336-384.

In some embodiments, the methods further comprise, prior to step (a), the step of obtaining the fecal sample from a candidate subject. In some embodiments, the candidate subject previously had the same bacterial infection as the subject in need of FMT and is now cured. In some embodiments, the donor subject is cured by fecal microbiota transplantation (FMT). In some embodiments, the fecal sample comprises a bacteriophage selected from the group consisting of Klebsiella phage KP34 (NCBI:txid674081), genus KP32virus (NCBI: txid1985720), and genus Kp36virus (NCBI: txid1920860). In some embodiments, the fecal sample comprises a bacteriophage comprising a sequence of any one of SEQ ID NOS: 1-324 and 333-335.

In some embodiments, the fecal sample comprises a bacteriophage selected from the group consisting of Klebsiella virus Kp15 (NCBI:txid1985328) and Klebsiella phage KP27 (NCBI:txid1129147). In some embodiments, the fecal sample comprises a bacteriophage comprising a sequence of any one of SEQ ID NOS:325-332.

In some embodiments of the methods described herein, the fecal sample is obtained from a stool bank.

In some embodiments of the methods described herein, the methods further comprise identifying the bacteria causing the bacterial infection in the subject in need of FMT.

In some embodiments of the methods described herein, the fecal material or the processed fecal sample is administered to the small intestine, the ileum, and/or the large intestine of the subject in need of FMT. In other embodiments, the fecal material or the processed fecal sample is administered via direct transfer to the GI track. In other embodiments, the fecal material or the processed fecal sample is formulated for oral administration. In other embodiments, the fecal material or the processed fecal sample is administered before food intake or together with food intake. In further embodiments, the subject in need of FMT is further administered an antibiotic.

### BRIEF DESCRIPTION OF THE DRAWINGS

FIG. 1: A flow chart depicting a method of selection of a composition to treat a bacterial infection. A method selecting bacteriophages for treating subject 1 comprises: identifying a second subject (or a combination of subjects) from a previous cohort who also has the same bacterial infection as subject 1 and was cured by receiving FMT from a third subject; characterizing the microbiome composition of the second and third subjects; virus-like particles enrichment; metagenomics sequencing/PCR to identify bacterial and viral compositions; perform bioinformatics analysis; identify phages that is specific for the bacteria; and administration of a composition comprises one or more of the said phages identified.

FIG. 2: A timeline of sample collections for donor and recipient showing sample collection times and results for CRE based on rectal swab from the recipients.

FIGS. 3A-3C: Microbiome composition in CRE-infected subjects and healthy controls. (3A) Diversity of bacteria; (3B) Diversity of fungi; and (3C) Relative abundance of Klebsiella pneumonia.

FIGS. 4A-4F: Analysis of virus compositions of donors and recipients, and the correlation between bacteria and virus. (4A) Alpha diversity (Shannon’s diversity) for the virome of donors, recipients pre-FMT, and recipient post-FMT at different times; (4B) Relative abundance of virome at order level; (4C) Alteration in the Klebsiella species after FMT; and (4D-4F) Alterations in the relative abundance of Klebsiella phages in three recipients after FMT.

FIGS. 5A-5D: Alterations of Klebsiella phages. Klebsiella phages under the genus (5A) Przondovirus (NCBI:txid1985720), (5B) Drulisvirus (NCBI:txid1920774), (5C) Webervirus (NCBI:txid1920860) (in recipient 1 and 2), and (5D) Slopekvirus (NCBI:txid1985328) (in recipient 3) increased after receiving FMT.

FIGS. 6A-6C: Relationships between Klebsiella species and Klebsiella phages. Black lines represent regressions with linear functions.

FIGS. 7A-7D: Alterations of Klebsiella phage KP34 (NCBI:txid674081) and Klebsiella phage KP27 (NCBI:txid1129147) bacteriophages. Results derived from bulk DNA metagenome sequences.

FIGS. 8A and 8B: Relative abundance of Klesbiella phages in donor and recipients pre- and post- FMT. (8A) Results derived from VLPs DNA metagenome sequences; (8B) Results derived from bulk DNA metagenome sequences.

FIG. 9: Alterations of Escherichia phages in three recipients. Results derived from VLP DNA metagenome sequences.

FIGS. 10A and 10B: Relative abundance of 10 Escherichia phages in donor and recipients pre- and post- FMT which showed the most significant increase in recipients after FMT. (10A) Results derived from VLPs metagenome sequence; (10B) Results derived from bulk DNA metagenome sequences.

FIGS. 11A-11F: FMT decolonize carbapenem-resistant Klebsiella pneumoniae and reconstitute the microbiota in mice. (A) Experimental scheme for Klebsiella pneumoniae challenge and FMT/VMT treatment. (B) Relative abundance of Klebsiella pneumoniae as compared to day 0. (C)(D)(E) Fecal microbiota composition at genus level of treated mice (n=5 mice per group). (F) Bacterial diversity was assessed using bulk metagenomic sequencing data

FIG. 12: Relative abundance levels Klebsiella virus in feces from mice treated by PBS and FMT.

FIG. 13: Relationships between Klebsiella pneumoniae and Klebsiella phages from stool of mice treated by PBS and VMT. Lines represent regressions with linear functions.

FIG. 14: Relative abundance levels Klebsiella virus in feces from mice treated by PBS and VMT.

FIG. 15: Relationships between Klebsiella pneumoniae and Klebsiella phages from stool of mice treated by PBS and VMT. Lines represent regressions with linear functions.

### DETAILED DESCRIPTION OF THE INVENTION

### I. Introduction

The invention provides methods for treating or preventing a bacterial infection in a subject in need of FMT by administering to the subject a processed fecal sample that contains the bacteriophages that inhibit the bacteria causing the bacterial infection in the subject. The processed fecal sample can first be obtained from a donor subject, analyzed for its bacteriophage content, and then processed to be ready for administration. During their studies, the present inventors performed a longitudinally and in-depth metagenomics analysis of the gut bacteriome, fungome, and virome in CRE-positive patients who successfully decolonized CRE after FMT. As described herein, the bacteria-bacteriophage correlation before and after FMT and its association with treatment outcome were explored. The inventors discovered that the bacteriophage used in the treatment showed a negative correlations between the bacteriophage and bacteria that caused infection. The determination and analysis of the species of bacteriophages in a potential donor’s fecal sample thus can be used to guide donor selection.

### II. Definitions

As used herein, the term “fecal microbiota transplantation (FMT)” refers to a medical procedure during which fecal matter containing live fecal microorganisms (bacteria, fungi, and the like) obtained from a healthy individual is transferred into the gastrointestinal tract of a recipient to restore healthy gut microflora that has been disrupted or destroyed by a variety of medical conditions. Typically, the fecal matter from a healthy donor is first processed into an appropriate form for the transplantation, which can be made through direct deposit into the lower gastrointestinal tract such as by colonoscopy, or by nasal intubation, or through oral ingestion of an encapsulated material containing dried and frozen fecal matter. Clostridium difficile infection (CDI) is the condition most commonly treated by FMT, although a number of other diseases and disorders including in the digestive system and in the nervous system have been reported to be successfully treated by FMT.

As used herein, the term “antibacterial” refers to a molecule or agent that is destructive to or inhibits the growth of bacteria.

As used herein, the term “bacteriophage” or “phage” refers to a bacteriophage isolate in which members of the isolate has substantially the same genetic makeup, such as sharing at least about any of 90%, 95%, 99%, 99.9% or more sequence identity in the genome. “Bacteriophage” or “phage” refers to the parent bacteriophage as well as the progeny or derivatives (such as genetically engineered versions) thereof. The bacteriophage can be a naturally occurring phage isolate, or a synthetic or engineered phage, including vectors, or nucleic acids that encode at least all essential genes, or the full genome of a phage to carry out the life cycle of the phage inside a host bacterium.

As used herein, a bacteriophage “targeting” or “targets” a bacterium means that the bacteriophage can infect the bacterium, and inhibit the growth of the bacterium. The bacteriophage can be either a lysogenic bacteriophage of the bacterium, or a lytic bacteriophage of the bacterium.

As used herein, the term “inhibiting” or “inhibition” refers to any detectable negative effect on a target biological process, such as RNA/protein expression of a target gene, the biological activity of a target protein, cellular signal transduction, cell proliferation, and the like. Typically, an inhibition is reflected in a decrease of at least 10%, 20%, 30%, 40%, 50%, 60%, 70%, 80%, 90% or greater in the target process (e.g., growth or proliferation of bacterial cells), or any one of the downstream parameters mentioned above, when compared to a control. “Inhibition” further includes a 100% reduction, i.e., a complete elimination, prevention, or abolition of a target biological process or signal. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least 10%, 20%, 30%, 40%, 50%, 60%, 70%, 80%, 90%, 100%, 200%, or greater such as 3, 5, 8, 10, 20-fold increase compared to a control level) in a target process or signal.

As used herein, the term “treatment” or “treating” refers to an approach for obtaining beneficial or desired results including clinical results. The beneficial or desired clinical results can include, but are not limited to, alleviating one or more symptoms resulting from the disease, diminishing the extent of the disease, stabilizing the disease (e.g., preventing or delaying the worsening of the disease), preventing or delaying the spread of the disease, delaying or slowing the progression of the disease, ameliorating the disease state, and decreasing the dose of one or more other medications required to treat the disease.

As used herein, the term “prevent” or “preventing” includes providing prophylaxis with respect to the occurrence or recurrence of a disease in a subject that may be predisposed to the disease but has not yet been diagnosed with the disease.

The term “effective amount,” as used herein, refers to an amount that is sufficient to produces an intended effect for which a substance is administered. The effect may include a desirable change in a biological process as well as the prevention, correction, or inhibition of progression of the symptoms of a disease/condition and related complications (e.g., suppressed or prevented bacterial infection) to any detectable extent. The exact amount “effective” for achieving a desired effect will depend on the nature of the therapeutic agent, the manner of administration, and the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the term “subject” refers to an animal, including, but not limited to, a cow, a goat, a sheep, a buffalo, a camel, a donkey, a llama, a horse, a pig, a human, a primate, an avian, a fish, a mule, a cat and a dog. In some embodiments, the subject is a human.

As used herein, the term “about” denotes a range of value that is +/- 10% of a specified value. For instance, “about 10” denotes the value range of 10 +/- 10 x 10%, i.e., 9 to 11.

### III. Fecal Matter Containing Bacteriophages

A fecal matter containing bacteriophages can be administered to a subject having a bacterial infection or at risk of having a bacterial infection. The fecal matters can be obtained from a donor subject or from a stool bank. The fecal matter can be processed into appropriate forms for the intended means of delivery in an FMT procedure. An FMT donor can be a healthy individual without any known diseases or disorders especially in the digestive tract, although some preference is often given to the members of the same household as the recipient. In some embodiments, a fecal matter can comprise one type of bacteriophages or can comprise two or more (e.g., three, four, five, six, seven, eight, nine, or ten) different types of bacteriophages.

### Bacteriophages

Examples of bacteriophages include, but are not limited to, Przondovirus (NCBI:txid1985720), Webervirus (NCBI:txid1920860), Slopekvirus (NCBI:txid1985328), Klebsiella phage KP27 (NCBI:txid1129147), Klebsiella phage K11 (NCBI:txid532077), Klebsiella phage K5 (NCBI:txid1647374), Klebsiella phage vB Kp1 (NCBI:txid1701804), Klebsiella phage KP32 (NCBI:txid674082), Klebsiella phage vB KpnP KpV289 (NCBI:txid1671396), Klebsiella phage F19 (NCBI:txid1416011), Klebsiella phage NTUH-K2044-K1-1 (NCBI:txid1194091), Klebsiella phage Kp2 (NCBI:txid1701805), Klebsiella phage KP34 (NCBI:txid674081), Klebsiella phage KpV41 (NCBI:txid1747282), Klebsiella phage KpV475 (NCBI:txid1852657), Klebsiella phage KpV71 (NCBI:txid1796998), Klebsiella phage vB KpnP SU503 (NCBI:txid1610834), Klebsiella phage vB_KpnP_SU552A (NCBI:txid1610835), Klebsiella phage KLPN1 (NCBI:txid1647408), Klebsiella phage Kp36 (NCBI:txid1129191), Escherichia virus 186 (NCBI:txid29252), Escherichia virus HK97 (NCBI:txid37554), Escherichia phage HK633 (NCBI:txid1147147), Escherichia virus P1 (NCBI:txid10678), Escherichia phage mEpX2 (NCBI:txid1147154), Escherichia phage TL-2011b (NCBI:txid1124654), Escherichia phage HK75 (NCBI:txid906668), Escherichia phage K30 (NCBI:txid1041524), Escherichia phage HK446 (NCBI:txid1147145), Escherichia virus HK022 (NCBI:txid10742), Escherichia phage HK629 (NCBI:txid1147148), Enterobacteria phage HK106 (NCBI:txid432198), Escherichia phage phiV10 (NCBI:txid343516), Escherichia virus P2 (NCBI:txid10679), Escherichia phage mEp234 (NCBI:txid1147157), Escherichia phage HK544 (NCBI:txid432201), Escherichia phage pro483 (NCBI:txid1649240), Escherichia phage HK542 (NCBI:txid432200), Escherichia phage Pollock (NCBI:txid1540097), Escherichia virus Lambda (NCBI:txid10710), Escherichia phage pro147 (NCBI:txid1649239), Escherichia phage Av-05 (NCBI:txid1527519), Escherichia virus Wphi (NCBI:txid103216), Escherichia phage HK639 (NCBI:txid906669), Escherichia virus Mu (NCBI:txid10677), Escherichia phage mEpX1 (NCBI:txid1147153), Escherichia phage 64795 ec1 (NCBI:txid1837842), Enterobacteria phage If1 (NCBI:txid10868), Enterobacteria phage Bp7 (NCBI:txid1052121), Enterobacteria phage RB69 (NCBI:txid12353), Escherichia virus N4 (NCBI:txid10752), Escherichia virus N15 (NCBI:txid40631), Escherichia phage FFH2 (NCBI:txid1446490), Escherichia phage phAPEC8 (NCBI:txid1229753), Escherichia phage ECBP5 (NCBI:txid1498172), Escherichia phage SUSP2 (NCBI:txid1718669), Escherichia phage 121Q (NCBI:txid1555202), Escherichia phage wV8 (NCBI:txid576791), Escherichia phage QL01 (NCBI:txid1673871), Escherichia phage V5 (NCBI:txid399183), Escherichia Stxl converting phage (NCBI:txid194948), Escherichia phage AR1 (NCBI:txid66711), Escherichia phage JSE (NCBI:txid576789), Enterobacter phage CC31 (NCBI:txid709484), Escherichia virus VR20 (NCBI:txid1913684), Escherichia virus VR26 (NCBI:txid1913686), Escherichia virus SU10 (NCBI:txid1987942), Escherichia virus K1H (NCBI:txid1911010), Escherichia virus ECB2 (NCBI:txid1987939) and Escherichia phage WG01 (NCBI:txid1837931).

In some embodiments, the bacteriophages target bacteria in the genus Klebsiella, such as Klebsiella pneumonia (e.g., carbapenem-resistant Klebsiella pneumonia). Examples of such bacteriophages include, but are not limited to, Webervirus (NCBI:txid1920860), Drulisvirus (NCBI:txid1920774), Przondovirus (NCBI:txid1985720), Klebsiella phage KLPN1 (NCBI:txid1647408), Klebsiella phage KpV71 (NCBI:txid1796998), Klebsiella phage vB_KpnP_{_}SU552A (NCBI:txid1610835), Klebsiella phage NTUH-K2044-K1-1 (NCBI:txid1194091), Klebsiella phage KpV41 (NCBI:txid1747282), Klebsiella phage KP34 (NCBI:txid674081), Klebsiella phage F19 (NCBI:txid1416011), Klebsiella phage Kp2 (NCBI:txid1701805), Klebsiella phage KpV475 (NCBI:txid1852657) and Klebsiella phage vB_Kp1 (NCBI:txid1701804). The bacteriophages that target bacteria in the genus Klebsiella, such as Klebsiella pneumonia (e.g., carbapenem-resistant Klebsiella pneumonia) can comprise a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identity to a sequence of any one of SEQ ID NOS:1-324 and 333-335. In other embodiments, the bacteriophages that target bacteria in the genus Klebsiella, such as Klebsiella pneumonia can be any of the species listed in Table 1 below.

TABLE 1

| Phage Species | Target bacteria | Taxid |
|---|---|---|
| Klebsiella phage KLPN1 | Klebsiella pneumoniae | 1647408 |
| Klebsiella phage vB Kp1 | Klebsiella pneumoniae | 1701804 |
| Klebsiella phage K11 | Klebsiella pneumoniae | 532077 |
| Klebsiella phage KP32 | Klebsiella pneumoniae | 674082 |
| Klebsiella phage vB KpnP KpV289 | Klebsiella pneumoniae | 1671396 |
| Klebsiella phage K5 | Klebsiella pneumoniae | 1647374 |
| Klebsiella phage KpV71 | Klebsiella pneumoniae | 1796998 |
| Klebsiella phage vB KpnP SU552A | Klebsiella pneumoniae | 1610835 |
| Klebsiella phage NTUH-K2044-K1-1 | Klebsiella pneumoniae | 1194091 |
| Klebsiella phage KpV41 | Klebsiella pneumoniae | 1747282 |
| Klebsiella phage KP34 | Klebsiella pneumoniae | 674081 |
| Klebsiella phage F19 | Klebsiella pneumoniae | 1416011 |
| Klebsiella phage Kp2 | Klebsiella pneumoniae | 1701805 |
| Klebsiella phage KpV475 | Klebsiella pneumoniae | 1852657 |
| Klebsiella phage vB KpnP SU503 | Klebsiella pneumoniae | 1610834 |

In other embodiments, the bacteriophages target bacteria in the genus Klebsiella, such as Klebsiella variicola (e.g., carbapenem-resistant Klebsiella variicola). Examples of such bacteriophage include, but are not limited to, Slopekvirus (NCBI:txid1985328), Klebsiella phage KP27 (NCBI:txid1129147). The bacteriophages that target bacteria in the genus Klebsiella, such as Klebsiella variicola (e.g., carbapenem-resistant Klebsiella variicola) can comprise a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identity to a sequence of any one of SEQ ID NOS:325-332. In other embodiments, the bacteriophages that target bacteria in the genus Klebsiella, such as Klebsiella variicola can be species Klebsiella phage KP27 (NCBI:txid1129147).

In yet other embodiments, the bacteriophages target bacteria in the genus Escherichia, such as Escherichia coli (e.g., carbapenem-resistant Escherichia coli). Examples of such bacteriophage include, but are not limited to, Escherichia virus 186 (NCBI:txid29252), Escherichia virus HK97 (NCBI:txid37554), Escherichia phage HK633 (NCBI:txid1147147), Escherichia virus P1 (NCBI:txid10678), Escherichia phage mEpX2 (NCBI:txid1147154), Escherichia phage TL-2011b (NCBI:txid1124654), Escherichia phage HK75 (NCBI:txid906668), Escherichia phage K30 (NCBI:txid1041524), Escherichia phage HK446 (NCBI:txid1147145), Escherichia virus HK022 (NCBI:txid10742), Escherichia phage HK629 (NCBI:txid1147148), Enterobacteria phage HK106 (NCBI:txid432198), Escherichia phage phiV10 (NCBI:txid343516), Escherichia virus P2 (NCBI:txid10679), Escherichia phage mEp234 (NCBI:txid1147157), Escherichia phage HK544 (NCBI:txid432201), Escherichia phage pro483 (NCBI:txid1649240), Escherichia phage HK542 (NCBI:txid432200), Escherichia phage Pollock (NCBI:txid1540097), Escherichia virus Lambda (NCBI:txid10710), Escherichia phage pro147 (NCBI:txid1649239), Escherichia phage Av-05 (NCBI:txid1527519), Escherichia virus Wphi (NCBI:txid103216), Escherichia phage HK639 (NCBI:txid906669), Escherichia virus Mu (NCBI:txid10677), Escherichia phage mEpX1 (NCBI:txid1147153), Escherichia phage 64795_ ecl (NCBI:txid1837842), Enterobacteria phage If1 (NCBI:txid10868), Enterobacteria phage Bp7 (NCBI:txid1052121), Enterobacteria phage RB69 (NCBI:txid12353), Escherichia virus N4 (NCBI:txid10752), Escherichia virus N15 (NCBI:txid40631), Escherichia phage FFH2 (NCBI:txid1446490), Escherichia phage phAPEC8 (NCBI:txid1229753), Escherichia phage ECBP5 (NCBI:txid1498172), Escherichia phage SUSP2 (NCBI:txid1718669), Escherichia phage 121Q (NCBI:txid1555202), Escherichia phage wV8 (NCBI:txid576791), Escherichia phage QL01 (NCBI:txidI673871), Escherichia phage V5 (NCBI:txid399183), Escherichia Stxl converting phage (NCBI:txid194948), Escherichia phage AR1 (NCBI:txid66711), Escherichia phage JSE (NCBI:txid576789), Enterobacter phage CC31 (NCBI:txid709484), Escherichia virus VR20 (NCBI:txid1913684), Escherichia virus VR26 (NCBI:txid1913686), Escherichia virus SU10 (NCBI:txid1987942), Escherichia virus K1H (NCBI:txid1911010), Escherichia virus ECB2 (NCBI:txid1987939), and Escherichia phage WG01 (NCBI:txid1837931). The bacteriophages that target bacteria in the genus Escherichia, such as Escherichia coli (e.g., carbapenem-resistant Escherichia coli) can comprise a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identity to a sequence of any one of SEQ ID NOS:336-384. In other embodiments, the bacteriophages that target bacteria in the genus Escherichia, such as Escherichia coli can be any of the species listed in Table 2 below.

TABLE 2

| Phage Species | Target bacteria | Taxid |
|---|---|---|
| Escherichia virus 186 | Escherichia coli | 29252 |
| Escherichia virus HK97 | Escherichia coli | 37554 |
| Escherichia phage HK633 | Escherichia coli | 1147147 |
| Escherichia virus P1 | Escherichia coli | 10678 |
| Escherichia phage mEpX2 | Escherichia coli | 1147154 |
| Escherichia phage TL-2011b | Escherichia coli | 1124654 |
| Escherichia phage HK75 | Escherichia coli | 906668 |
| Escherichia phage K30 | Escherichia coli | 1041524 |
| Escherichia phage HK446 | Escherichia coli | 1147145 |
| Escherichia virus HK022 | Escherichia coli | 10742 |
| Escherichia phage HK629 | Escherichia coli | 1147148 |
| Enterobacteria phage HK106 | Escherichia coli | 432198 |
| Escherichia phage phiV10 | Escherichia coli | 343516 |
| Escherichia virus P2 | Escherichia coli | 10679 |
| Escherichia phage mEp234 | Escherichia coli | 1147157 |
| Escherichia phage HK544 | Escherichia coli | 432201 |
| Escherichia phage pro483 | Escherichia coli | 1649240 |
| Escherichia phage HK542 | Escherichia coli | 432200 |
| Escherichia phage Pollock | Escherichia coli | 1540097 |
| Escherichia virus Lambda | Escherichia coli | 10710 |
| Escherichia phage pro147 | Escherichia coli | 1649239 |
| Escherichia phage Av-05 | Escherichia coli | 1527519 |
| Escherichia virus Wphi | Escherichia coli | 103216 |
| Escherichia phage HK639 | Escherichia coli | 906669 |
| Escherichia virus Mu | Escherichia coli | 10677 |
| Escherichia phage mEpX1 | Escherichia coli | 1147153 |
| Escherichia phage 64795 ec1 | Escherichia coli | 1837842 |
| Enterobacteria phage If1 | Escherichia coli | 10868 |
| Enterobacteria phage Bp7 | Escherichia coli | 1052121 |
| Enterobacteria phage RB69 | Escherichia coli | 12353 |
| Escherichia virus N4 | Escherichia coli | 10752 |
| Escherichia virus N15 | Escherichia coli | 40631 |
| Escherichia phage FFH2 | Escherichia coli | 1446490 |
| Escherichia phage phAPEC8 | Escherichia coli | 1229753 |
| Escherichia phage ECBP5 | Escherichia coli | 1498172 |
| Escherichia phage SUSP2 | Escherichia coli | 1718669 |
| Escherichia phage 121Q | Escherichia coli | 1555202 |
| Escherichia phage wV8 | Escherichia coli | 576791 |
| Escherichia phage QL01 | Escherichia coli | 1673871 |
| Escherichia phage V5 | Escherichia coli | 399183 |
| Escherichia Stx1 converting phage | Escherichia coli | 194948 |
| Escherichia phage AR1 | Escherichia coli | 66711 |
| Escherichia phage JSE | Escherichia coli | 576789 |
| Enterobacter phage CC31 | Escherichia coli | 709484 |
| Escherichia virus VR20 | Escherichia coli | 1913684 |
| Escherichia virus VR26 | Escherichia coli | 1913686 |
| Escherichia virus SU10 | Escherichia coli | 1987942 |
| Escherichia virus K1H | Escherichia coli | 1911010 |
| Escherichia virus ECB2 | Escherichia coli | 1987939 |
| Escherichia phage WG01 | Escherichia coli | 1837931 |

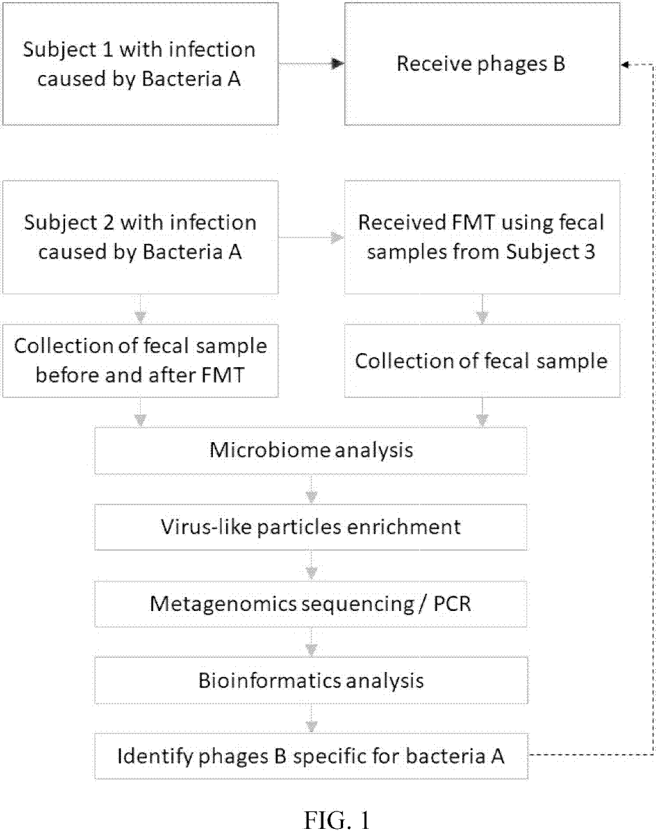

As described in the Examples section, the bacteriophage used in the treatment should show a negative correlations (r value less than 0) between the bacteriophage and bacteria that caused infection (see, e.g., FIG. 1).

The bacteriophages described herein can be lytic or lysogenic. A lytic phage has the ability to lyse out of the bacterial host cell following phage replication, and the phage progeny is able to infect new bacterial host cells. A lysogenic phage, in contrast, integrates its viral genome with the host DNA, replicating along with the host’s DNA. The lysogenic phage then undergoes replication resulting in lysis of the host cell releasing phage.

A fecal sample obtained from a donor subject can be processed to obtain a processed fecal sample that is in an appropriate form for the intended means of delivery in an FMT procedure. For example, a fecal sample for treating a Klepsiella infection can be processed as described below. Fecal samples are incubated overnight at 37° C. in LB Broth and then centrifuged at 5,500 g. The supernatant is filtered through 0.22 µm. The solution is then mixed with 2.5 mL of the host bacteria, Klebsiella (at exponential phage) and added to 10 mL LB broth for overnight incubation at 37° C. The mixture is then screened for the presence of phage by the Double-Layer Agar (DLA) method. Supernatants with positive phages are purified by picking a single plaque with a sterile pasteur pipette tip, resuspending the plaque in 1 mL LB broth, incubating for 1 h at 37° C., tittering, and plating by the DLA method.

In other examples, a composition to administer to a recipient can contain synthetic bacteriophages. Synthetic bacteriophages can be made, for example by creating functional phage particles from phage genomes modified in vitro, with transformation as the means of getting phage genomic DNA back into the host bacterium, where phage particles are produced from the genomic DNA. Recombinant DNA (rDNA) technology refers to the process of joining DNA molecules from two different sources and inserting them into a host organism, to generate products for human use. Recombinant DNA (or rDNA) is made by combining DNA from two or more sources. In practice, the process often involves combining the DNA of different organisms (e.g., bacteria and phages). Though molecular cloning technologies, a promoter can also be operatively linked to the nucleic acid of a bacteriophage. Further, other components that can promote the expression and/or activity of the bacteriophage can also be linked to the nucleic acid of the bacteriophage, e.g., a nucleic acid encoding an antimicrobial polypeptide.

For example, the following steps can be followed when generating artificial bacteriophages: isolation of genetic material via restriction enzyme digestion; amplification using PCR; ligation of DNA molecules to create recombinant DNA that is within a plasmid vector; and insertion of recombinant DNA into host cell by transformation of competent bacterial cells. The plasmid vector is now able to replicate because plasmids normally have a replication origin. However, now that the DNA insert is part of the vector’s length, the DNA is automatically replicated along with the vector. Each recombinant plasmid that enters a cell will form multiple copies of itself in that cell.

### Bacteriophage Concentration

In some embodiments, the amount of the beneficial bacteriophages in the processed fecal sample to be administered to the subject in need is expressed as a percentage over the total level of all bacteriophage species in the sample. In some embodiments, the amount of the beneficial bacteriophages is determined as greater than 10% (e.g., greater than 15%, 20%, 25%, 30%, 35%, 40%, 45%, or 50%) of total bacteriophages in the processed fecal sample. In some cases, the potential recipient is then immediately given FMT, without any further treatment or preparation such as administration of an antibiotic in the effective amount. In some embodiments, when the amount of beneficial bacteriophage is no greater than 5% (e.g., 4%, 3%, 2%, or 1%) of total bacteriophages in the processed sample, FMT is assessed as unlikely to be effective for the potential recipient. In some embodiments, amount of bacteriophages is determined by quantitative polymerase chain reaction (PCR). In some embodiments, the levels of all bacteriophage species present in the sample is determined by the Internal transcribed spacer 2 (ITS2) sequencing.

In some embodiments, the processed fecal sample comprises at least about any one of 10⁴, 10⁵, 10⁶, 10⁷, 10⁸, 10⁹, 10¹⁰, 10¹¹, or 10¹² PFU/mL of each bacteriophage. The concentration of bacteriophage can be determined using known phage titration protocols. In some embodiments, the processed fecal sample comprises an effective amount of the bacteriophages. The concentration of bacteriophage varies depending upon the carrier and method of administration. For a processed fecal sample that comprises two or more different types of bacteriophages, the relative ratio by PFU between different bacteriophages in the processed fecal sample can be chosen to optimize the efficacy of the processed fecal sample or to enhance synergy among the different bacteriophages. In some embodiments, each bacteriophage is present at about equal PFU in the processed fecal sample. In some embodiments, one bacteriophage is present at about any one of 1.5, 2, 3, 4, 5, 10 or more PFU than another bacteriophage in the processed fecal sample.

### Antibiotics

One or more antibiotics can be added to the processed fecal sample. Examples of antibiotics include, but are not limited to, amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, amoxicillin clavulanate, ampicillin/sulbactam, piperacillin/tazobactam, ticarcillin/clavulanate, bacitracin, colistin, polymyxin b, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (tmp-smx), sulfonamidochrysoidine, demeclocycline, doxycycline, minocycline, oxytetracycline, tetracycline, clofazimine, dapsone, capreomycin, cycloserine, ethambutol(bs), ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim.

### Delivery

The fecal sample obtained from the donor subject can be processed, formulated, and packaged to be in an appropriate form in accordance with the delivery means in the FMT procedure, which may be by direct deposit in the recipient’s lower gastrointestinal track (e.g., wet or semi-wet form) or by oral ingestion (e.g., frozen dried encapsulated). In some embodiments, the processed fecal sample can be formulated for FMT by direct transfer to the GI tract (e.g., via colonoscopy or via nasal intubation). In some embodiments, the processed fecal sample can be formulated for FMT by rectal deposit.

In some embodiments, the processed fecal sample comprising bacteriophages can be stored as an aqueous solution or lyophilized powder preparation. A delivery vehicle is suitable for the route of delivery or administration. In some embodiments, the delivery vehicle is suitable for oral administration. In some embodiments, the delivery vehicle is suitable for direct transfer to the GI track. In some embodiments, the delivery vehicle further stabilizes the bacteriophages, and/or enhances the efficacy of the bacteriophages on inhibiting bacterial infection.

In some embodiments, the delivery vehicle is a buffer, such as phosphate buffered saline (PBS), Luria-Bertani Broth, phage buffer (100 mM NaCl, 100 mM Tris-HCl, 0.01% (w/v) Gelatin), or Tryptic Soy broth (TSB). In some embodiments, the delivery vehicle comprises food grade oils, and inorganic salts useful for adjusting the viscosity of the bacteriophage composition. Examples of pharmaceutically acceptable carriers are well known, and one skilled in the pharmaceutical art can easily select carriers suitable for particular routes of administration (Remington’s Pharmaceutical Sciences, Mack Publishing Co., Easton, Pa., 1985). Suitable pharmaceutical carriers include, but are not limited to, sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. The final form may be sterile and may also be able to pass readily through an injection device such as a hollow needle. The proper viscosity may be achieved and maintained by the proper choice of solvents or excipients.

In some embodiments, the delivery vehicle comprises other agents, excipients, or stabilizers to improve properties of the composition, which do not reduce the effectiveness of the bacteriophage. Examples of suitable excipients and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate and mineral oil. The formulations can additionally include lubricating agents, wetting agents, emulsifying and suspending agents, preserving agents, sweetening agents or flavoring agents. Examples of emulsifying agents include tocopherol esters such as tocopheryl polyethylene glycol succinate and the like, PLURONIC®, emulsifiers based on polyoxy ethylene compounds, Span 80 and related compounds and other emulsifiers known in the art and approved for use in animals or human dosage forms. The compositions (such as pharmaceutical compositions) can be formulated so as to provide rapid, sustained or delayed release of the active ingredient after administration to an individual by employing procedures well known in the art.

In some embodiments, the processed fecal sample comprises a delivery vehicle suitable for oral administration. In some embodiments, the delivery vehicle is an aqueous medium, such as deionized water, mineral water, 5% sucrose solution, glycerol, dextran, polyethylene glycol, sorbitol, or such other formulations that maintain phage viability, and are non-toxic to animals, including lactating mammals and humans. In some embodiments, the composition is prepared by resuspending purified phage preparation in the aqueous medium.

### IV. Methods

The disclosure provides methods of treating or preventing a bacterial infection in a subject in need of FMT, comprising: (a) analyzing the fecal sample obtained from a potential donor to determine the presence and/or relative quantity of one or more species of the pertinent bacteriophages in the fecal sample, thereby determining whether potential donor can properly serve as a donor to provide fecal material advantageous in FMT; (b) processing the fecal sample that has been deemed suitable for FMT into a processed fecal sample; and (c) administering to the subject in need of FMT the processed fecal sample. A fecal sample from the subject in need of FMT can be analyzed to find the species of bacteria causing the infection, which can help to determine the species of bacteriophage needed in a fecal sample obtained from a donor subject. A fecal sample from a donor subject can be analyzed to find if the sample contains the predetermined species of bacteriophage.

As described herein, the bacteriophages in the processed fecal sample should target the bacteria that caused the bacterial infection in the subject in need. One or more methods available in the art can be used to analyze and determine the species of bacteriophage present in the fecal sample. For example, as described herein, megagenomics sequencing using PCR can be applied to determine the species of bacteriophage present in the fecal sample. The methods described herein can further comprise the step of determining the bacteria that caused the infection in the subject. For example, before the subject undergoes FMT, a stool sample can be obtained from the subject and analyzed for the bacteria that caused the infection. Once the bacteria is determined, the appropriate bacteriophage that targets the bacteria can be chosen, and a fecal sample from a donor subject containing the appropriate bacteriophage can be selected. In some embodiments, the processed fecal sample can be administered via direct transfer to the GI track. In other embodiments, the processed fecal sample can be administered orally, i.e., before food intake or together with food intake.

In one example, the bacteriophages that target bacteria in the genus Klebsiella, such as Klebsiella pneumonia (e.g., carbapenem-resistant Klebsiella pneumonia) can comprise a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identity to a sequence of any one of SEQ ID NOS: 1-324 and 333-335. In another example, the bacteriophages that target bacteria in the genus Klebsiella, such as Klebsiella variicola (e.g., carbapenem-resistant Klebsiella variicola) can comprise a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identity to a sequence of any one of SEQ ID NOS:325-332. In yet another example, the bacteriophages that target bacteria in the genus Escherichia, such as Escherichia coli (e.g., carbapenem-resistant Escherichia coli) can comprise a nucleic acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identity to a sequence of any one of SEQ ID NOS:336-384. In other embodiments, the bacteriophage that targets bacteria is selected from the group consisting of Klebsiella phage vB_Kpn_IME260 (NCBI: taxid 1912318), Klebsiella phage vB_KpnM_KB57 (NCBI: taxid 1719140), Klebsiella phage vB_KpnM_KpV52 (NCBI: taxid 1912321), Klebsiella virus 0507KN21 (NCBI: taxid 2169687), Klebsiella phage F19 (NCBI: taxid 1416011), Klebsiella phage K5 (NCBI: taxid 1647374), Klebsiella phage Matisse (NCBI: taxid 1675607), Klebsiella phage Sugarland (NCBI: taxid 2053603), Klebsiella phage PKP126 (NCBI: taxid 1654927), Klebsiella phage K64-1 (NCBI: taxid 1439894), Klebsiella phage KpV71 (NCBI: taxid 1796998), and Klebsiella phage Matisse (NCBI: taxid 1912318) and can comprise a polynucleotide sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identity to any one of the sequences set forth in List 6 or 7.

In the methods described herein, the fecal sample can be obtained from a donor subject. For example, the donor subject can be someone who previously had the same bacterial infection (i.e., caused by the same bacteria) and who is now cured. For example, the donor subject can be cured by FMT using a fecal sample that was obtained from another donor subject. Thus, the donor subject is likely to have the appropriate bacteriophage that targets the infection-causing bacteria in the subject in need of the bacteriophage. In other embodiments, a donor subject can simply be a healthy individual without any known diseases or disorders especially in the digestive tract. In another example, the fecal sample used in the methods can be obtained from a stool bank. A stool bank can have a variety of fecal samples obtained from donor subjects who previously had a bacterial infection and is now cured.

The methods described herein can be used to treat or prevent bacterial infections that are antibiotic resistant, for example, carbapenem-resistant Enterobacteriaceae (CRE) infections and vancomycin-resistant Enterococci (VRE) infections. A bacterial infection can be caused by bacteria in the family Enterobacteriaceae, such as bacteria in the genus Enterococcus, Klebsiella (e.g., Klebsiella pneumonia, Klebsiella variicola), or Escherichia (e.g., Escherichia coli). A bacteriophage used in methods described herein can comprise a sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identity to the sequence of any one of SEQ ID NOS: 1-384. A bacteriophage used in methods described herein can be any of the species listed in Tables 1 and 2 and species Klebsiella phage KP27 (NCBI:txid1129147) or any of those indicated in List 6 or 7. The bacteriophage can be administered to the small intestine, the ileum, and/or the large intestine of the subject in need of FMT. In some embodiments, the bacteriophage can be administered in combination with an antibiotic.

### V. Administration

A fecal sample containing bacteriophages obtained from a donor subject can be processed and administered to a subject in need to prevent or treat a bacterial infection in the subject. In some embodiments, the fecal sample containing the bacteriophages can be processed and formulated for oral administration. For example, the subject can ingest the processed fecal sample before food intake or together with food intake. In other examples, the processed fecal sample containing the bacteriophages can be administered by direct transfer to the GI track. For example, the subject can undergo FMT where the processed fecal sample is delivered to the small intestine, the ileum, and/or the large intestine of the subject. In other embodiments, the processed fecal sample containing the bacteriophages can also be formulated for rectal administration.

The donor subject can be someone who previously had the same bacterial infection as the subject and is now cured. For example, frozen or fresh stool can be freshly prepared on the day of administration using stool from a single donor subject or using stools from a mixture of multiple donor subjects. Fecal samples can be diluted with sterile saline (0.9%). This solution can then be blended and strained with filter. The resulting supernatant can then be used directly as fresh FMT solution or stored as frozen FMT solution to be used on another day.

The processed fecal sample containing the bacteriophages can be formulated for oral delivery. The following is an example of capsulized, freeze-dried fecal microbiota. Processing is carried out under aerobic conditions. A fecal suspension is generated in normal saline without preservatives using a commercial blender. The slurry is centrifuged at 200 g for 10 minutes to remove debris. The separate fraction was centrifuged at 6,000 × g for 15 min and re-suspended to one-half (0.5 mL) the original volume in trehalose (at 5% and 10% concentrations) in saline. The supernatant is lyophilized and stored at -80° C. Commercially available acid-resistant hypromellose capsules (DRCaps, Capsugel) are used. Double-encapsulated capsules are prepared by using a filled size 0 capsule packaged inside a size 00 capsule. Capsules are manually filled using a 24-hole filler (Capsugel) to a final concentration of about 10¹¹ cells/capsule. The capsules are stored at -80° C. in 50 mL conical tubes until needed. Once removed from the freezer, a 1 g silica gel canister (Dry Pak Industries, Encino, CA) is added to the container. Another example is a capsulized preparation of bacteriophages. The isolated phage is grown in host to make high-titer stocks by standard procedures. The high-titer phage preparations are filtered through a 0.22 µm filter. These filterates are stored at 4° C. until use. Double-encapsulated capsules are prepared by using a filled size 0 capsule packaged inside a size 00 capsule. Capsules are manually filled using a 24-hole filler (Capsugel) to a final concentration of about 3×10¹¹ PFU/capsule.

### EXAMPLES

### Example 1. Matching of Subjects Having the Same Bacterial Infection

As outlined in FIG. 1, subject 1 and subject 2 should have an infection caused by the same bacterial species, regardless the bacterial gene that causes antibiotic resistance (if the species is an antibiotics resistance organism). The bacteria causing the infection can undergo PCR, metagenomics, 16S sequencing, and/ or culturing. The species can be identified by MALDI biotyper identification. Fecal samples were cultured in ChromID Carba Smart selective chromogenic media bi-plate (bioMerieux, Marcy 1′Etoile, France). The plates were incubated at 37° C. and growth was observed after 24 hours. In case of growth, identification tests of carbapenemase-positive colonies were carried out by MALDI Biotyper systems (Bruker Daltonik, Germany).

### Example 2. Microbiome Analysis

Microbiome analysis was performed on the stool samples from three subjects having a CRE infection pre-FMT and after FMT. Microbiome analysis was also performed on samples collected from the FMT donor. In addition, stool samples from four healthy subjects, and three controls with spontaneous clearance of their CRE infection status were included. Approximately 100 mg fecal sample was prewashed with 1 mL ddH₂O and pelleted by centrifugation at 13,000×g for 1 min. The fecal pellet was resuspended in 800 µL TE buffer (pH 7.5), supplemented with 1.6 µL 2-mercaptoethanol and 500 U lyticase (Sigma), and incubated at 37° C. for 60 min. The sample was then centrifuged at 13,000×g for 2 min and the supernatant was discarded. After pretreatment, fecal DNA was subsequently extracted from the pellet using Maxwell® RSC PureFood GMO and Authentication Kit (Promega) following manufacturer’s instructions. Briefly, 1 mL of CTAB buffer was added to the fecal pellet and vortexed for 30 seconds. Then the sample was heated at 95° C. for 5 minutes. After that, the samples were vortexed thoroughly with beads at maximum speed for 15 min. Then 40 µL of proteinase K and 20 µL of RNase A was added into the sample and the mixture was incubated at 70° C. for 10 minutes. The supernatant was then obtained by centrifuging at 13,000×g for 5 min and was added in Maxwell® RSC machine for DNA extraction. The extracted fecal DNA was subject to metagenomics sequencing.

### Example 3. Virus-Like Particles Enrichment

Virus-like particles (VLPs) were enriched using protocol described in previous study¹⁴. 200 mg of stool sample was added in 400 µL saline-magnesium buffer (0.1 M NaCl, 0.002% gelatin, 0.008 M MgSO₄H₂O, 0.05 M Tris pH 7.5) and vortexed for 10 min. The sample then was centrifuged at 2,000xg and suspension was obtained. To remove the bacterial cells and residual host, the suspension was further filtered by one 0.45 mm and two 0.22 mm filters. The cleared suspension was incubated with lysozyme (1 mg/mL at 37° C. for 30 min) and chloroform (0.2x volume at RT for 10 min) in turn to degrade any remaining bacterial and host cell membranes. A DNase cocktail including 1 U Baseline zero DNase (Epicenter) and 10 U TubroDNaseI (Ambion) was added into the sample and the mixture was incubated at 65° C. for 10 min to eliminate non-virus protected DNA. VLPs were lysed (4% SDS plus 38 mg/mL Proteinase K at 56° C. for 20 min), treated with CTAB (2.5% CTAB plus 0.5 M NaCl at 65° C. for 10 min), and the nucleic acid was extracted with phenol:chloroform pH 8.0 (Invitrogen). The aqueous fraction was washed once with an equal volume of chloroform, purified, and concentrated on a column (DNA Clean & Concentrator™ 89-5, Zymo Research). VLP DNA was amplified for 2 h using Phi29 polymerase (GenomiPhi V2 kit, GE Healthcare) prior to sequencing. Four independent reactions were performed for each sample and pooled together to reduce amplification bias.

### Example 4. Megagenomics Sequencing and Analysis

Qualified fecal DNA and VLP DNA was cut into fragments, the sequencing libraries were prepared through the processes of end repairing, adding A to tails, purification, and PCR amplification. The fecal DNA libraries were sequenced on an Illumina Novaseq 6000 with PE150 sequencing strategy by Novogene and yielded an average of 48 ± 5.3 million reads (12G data) per sample. The VLPs libraries were sequenced by Illumina Novaseq 6000 with PE150 sequencing strategy by Novogene and an average of 25 ± 3.3 million reads (6G data) per sample were obtained.

Raw sequence reads were filtered and quality-trimmed using Trimmomatic v0.36¹⁵ as follows: 1) trimming with a quality sliding window of 4:8; 2) cropping sequences to remove 20 bases from the start and bases beyond 220 from the end; 3) removing sequences less than 150 bp long. Then the human host contaminate reads were filtering out by Kneaddata (web site: bitbucket.org/biobakery/kneaddata/wiki/Home, reference database: GRCh38 p12) with default argument to generate clean reads.

Taxonomic profile of fungi and viruses were determined from the fecal DNA metagenomic dataset and VLP DNA metagenomic dataset respectively, using Kraken2 v2.0.7-beta. The full NCBI fungal and viral RefSeq database¹⁶ was built from NCBI using Jellyfish by counting distinct 31-mers in the reference libraries, with each k-mer in a read mapped to the lowest common ancestor of all reference genomes with exact k-mer matches. Each query was thereafter classified to a taxon with the highest total hits of k-mer matched by pruning the general taxonomic trees affiliated with mapped genomes.

Other methods as follows for taxonomy assignment that compare a sequence in the sample to a database of known sequences are also applicable. First, sequence alignment can be conducted by BLASTN similarity searches against customized databases (with cut-off e-value <0.0001). Secondly, Bowtie2 will step deep sequence obtained quickly mapped into the database. Reads per sample are mapped against this dataset using Bowtie2 v.2.2.8 with the following parameters:-local-maxins 800 -k = 3. Genome coverage per base was calculated considering only reads with a mapping quality above 20 using view and depth Samtools commands.

### Example 5. Criteria for Bacteriophage Selection

The bacteriophage should show a negative correlations (r value less than 0) between the bacteriophage and bacteria that caused infection in any one of the fecal samples collected from subject 2 or subject 3 in FIG. 1.

### Bacterial Isolation and Whole Genome Sequencing

To isolate CRE bacteria, colonies were picked from dilution cultures (stool samples from recipient 1) by specific selective media (chromID CARBA SMART by bioMerieux, France) and streaked onto fresh agar to ensure purity. Isolated clones were resuspended in PBS plus glycerol (20%) and stored at -80° C. For animal experiments, the bacterial inoculum administered to mice was normalized to total 10⁹ CFU. Bacterial were administered to mice by oral gavage in 100 µL daily for 2 days. Genomic DNA of the strains were extracted using QIAamp DNA Mini Kit (Qiagen, Germany) and were sent to BGI Genomics (Shenzhen, China) immediately on dry ice for WGS. Sequencing was carried out using the Illumina Hiseq Xten PE150 sequencer (Illumina, United States) with a high-throughput 2 × 100 bp pair end sequencing strategy. Reads were filtered as described previously and the resulting clean reads were assembled using SPAdes software (Bankevich et al., 2012). The assemblies were further examined for characteristics.

### Animal Experiment

C57BL/6J male mice were used at 6-8 weeks of age and were randomly assigned to experimental and control groups. In all experiments, age- and gender-matched mice were used. All mice were kept at a strict 24 hr light-dark cycle, with lights on from 6am to 6pm. For antibiotic treatment, mice were given a combination vancomycin (0.125 g)-neomycin (0.25 g)-metronidazole (0.25 g)-ampicillin (0.25 g, combined in 250 ml water) in their drinking water for two weeks as previously described ¹⁸ . On the day of FMT, a fresh FMT was prepared by harvesting the stool from normal healthy mice. The stool pellets were then suspended in 100 µL sterile PBS and mice were subsequently orally gavaged with 100 µL suspension.

Viral microbial fraction transplantation (VMT) was obtained by VLP preparation. A stool pellet from the untreated healthy mice were suspended in 300 µL sterile PBS and centrifuged at 2500 g for 10 min. Then bacteria were removed in the VLP-containing supernatant using a 0.45 µm filter, which follow by a 0.22 µm filter. Using a 100 kDa centrifugal filter at 3220 g for 5 min to capture VLPs in the fecal filtrated and then washed 3 times with PBS under the same conditions. Afterwards, VLPs on the filter were suspended in 100 µL PBS and mice were orally gavaged with 100 µL suspension. All experimental procedures were approved by the Animal Ethics Committee of the Chinese University of Hong Kong.

### Example 6. Statistics

The abundance data of bacteria, viruses, and fungi were imported into R 3.3.5. Richness, diversity, and rarefaction calculations were performed using the phyloseq package. Data visualization was done in R (package ggplot2). Pearson’s correlation test was applied to determine correlations between the bacteriophage and bacteria that caused infection. For Pearson correlations, we used the cor.test function in R to conduct significance test and obtain the P values (two-sided). An r value less than 0 indicates a negative association.

### Example 7. Human Clinical Trial Design

Patients who were 18 years old or older, had two or more stool or rectal swab positive for CRE at least one week apart, and did not receive antimicrobial therapy for at least 48 hours prior to infusion of FMT were recruited to a clinical trial (NCT03479710). Patients who had an active infection of CRE or VRE requiring antimicrobial therapy, pregnancy, active gastrointestinal tract infections, or inflammatory disorders, had recent intro-abdominal surgery, had short gut syndrome, or use of medications which alter gastrointestinal motility were excluded. CRE infection was defined as the presence of any Enterobacteriaceae with resistance to any of the carbapenems. In this study, patients received 2 FMTs using frozen donor stool samples. 100 mL of FMT solution (raw stool 50 g) in 0.9% sterile saline were infused over 2-3 minutes into the distal duodenum or jejunum via oesophago-gastro-duodenoscopy (OGD). Stool samples were collected from patients before and after FMT prospectively. Recipients received FMT from the same donor for the 2 FMTs. Stools for FMT infusions were obtained from donors recruited to Stool Biobank for the Faculty of Medicine, The Chinese University of Hong Kong. Donors were volunteers from the general population, including spouses or partners, first-degree relatives, other relatives, friends, and others who were known or unknown to the potential patients. Donors need to fulfil a set of eligibility criteria and passed screening laboratory tests for infectious diseases, including CRE and VRE infections.

### Example 8. FMT for Eradication of Intestinal Colonization of CRE

Three CRE-positive patients, detected on two consecutive rectal swabs, with CRE isolates clinically identified as Klebsiella pneumonia, Klepsiella variicola, and Escherichia coli (Table 3), were successfully cleared from CRE after receiving two FMTs each (FIG. 2). Recipient 1 (female, 90 years old) had two FMTs five days apart. CRE was tested negative on day 11 after the first FMT and remained negative up to week 5 after the first FMT. She then developed foot ulcer infection and received antibiotic therapy at week 6 to week 19 after the first FMT. CRE of recipient 1 was tested positive at week 14 and 19. At week 22, it was tested negative again after completion of four courses of Augmentin. Recipient 2 (male, 70 years old) and recipient 3 (male, 74 years old) each received two FMTs on consecutive days and were tested negative for CRE at week one (recipient 2), and week five (recipient 3) after FMT.

TABLE 3

CRE species isolated from recipients

| Recipient | Isolated CRE species | CRE type |
|---|---|---|
| 1 | Klebsiella pneumoniae | oxa-181 |
| 2 | Klebsiella pneumoniae | NDM |
| | Escherichia coli | NDM |
| 3 | Klebsiella variicola | NDM |

oxa-181: blaOXA-181 gene in the isolate; NDM: New Delhi metallo-beta-lactamase gene in the isolate

### Example 9. Fecal Microbiome Profile in Donor and Recipient Pre- and Post-FMT for Selecting FMT Donor in Treating CRE Infections

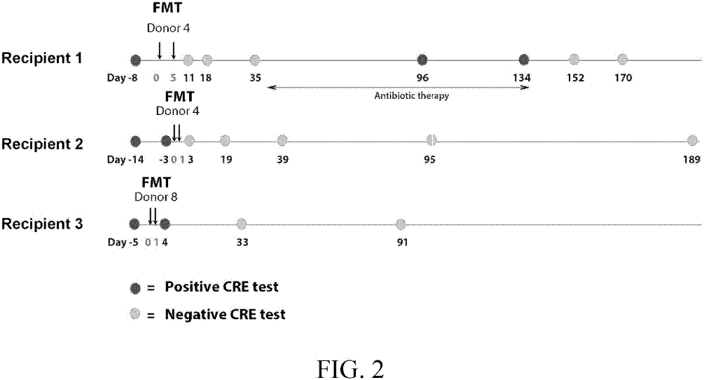

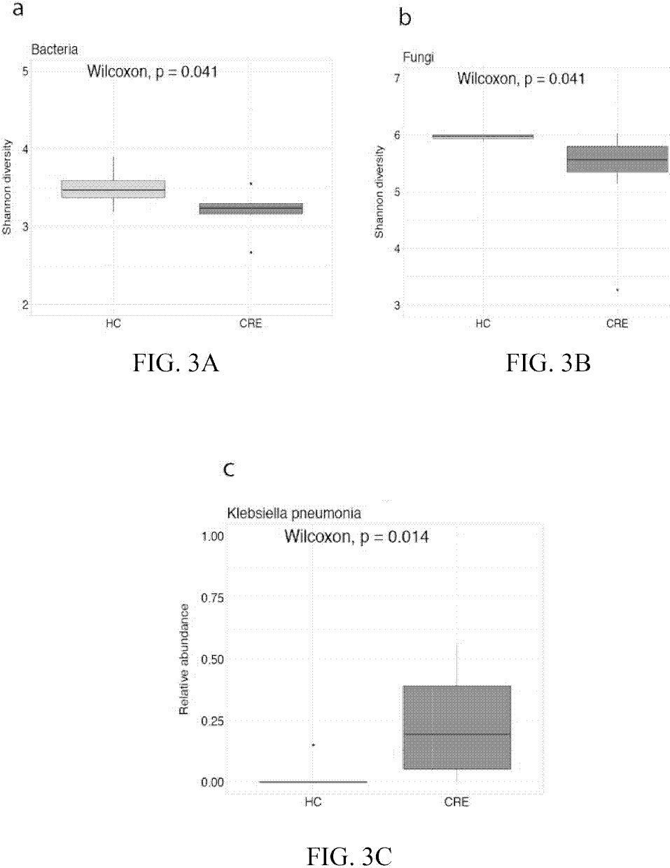

The bacteria profile of patients with CRE infections was significantly different from healthy controls. We first determined the differences in fecal microbiome between patients with CRE infections and healthy controls via shotgun metagenomic profiling. Stool of patients with CRE infections was characterized by a lower bacterial and fungal α-diversity (Shannon index P < 0.05; FIGS. 3A and 3B) and a higher level of Klebsiella pneumoniae compared with controls (average abundance 0.24% vs 0.02%; Mann-Whitney P < 0.05; FIG. 3C).

### Example 10. Fecal Viral Profile in Donor and Recipient Pre- and Post-FMT

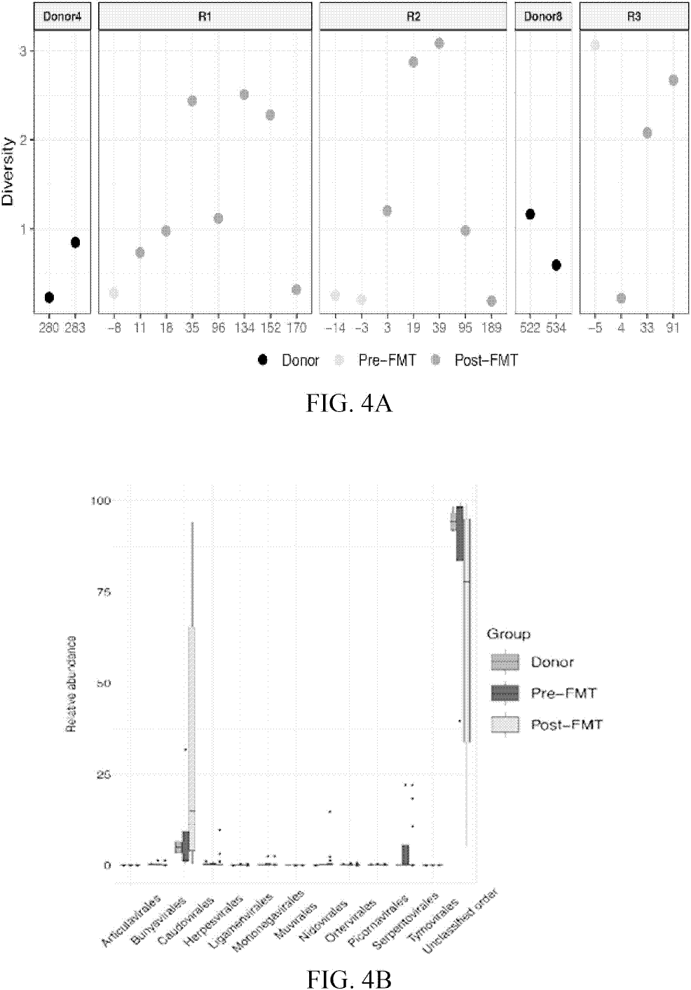

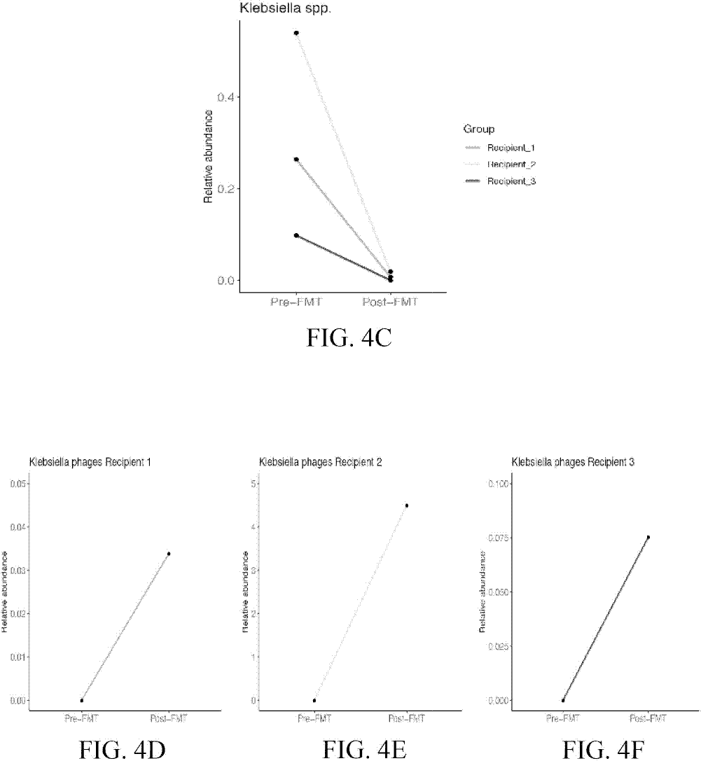

To explore alterations in the patient’s gut virome after multiple FMTs, we enriched virus like particles (VLPs) in stools and subsequently sequenced DNA extracted from VLP-enriched stool preparations. On average, we obtained 13,886,857 ± 4,552,632 (mean ± s.d.) clean paired-end reads per sample. The alpha diversity of virome demonstrated substantial variations among individuals (FIG. 4A). After FMT, the virome diversity increased in recipients 1 and 2, who had low baseline diversity indexes. However, the virome diversity of recipient 3, who had a higher baseline diversity, declined following FMT treatment. In agreement with previous observations¹⁷⁻¹⁹, the results highlighted the enormous sequence variation present in bacteriophages among donors and recipients. At the order level, Caudovirales were the predominant bacterial viruses (phages) detected in CRE-infected subjects before FMT (FIG. 4B).

### Example 11. Interactions Between Gut Virome and Bacteriome in Relation to FMT

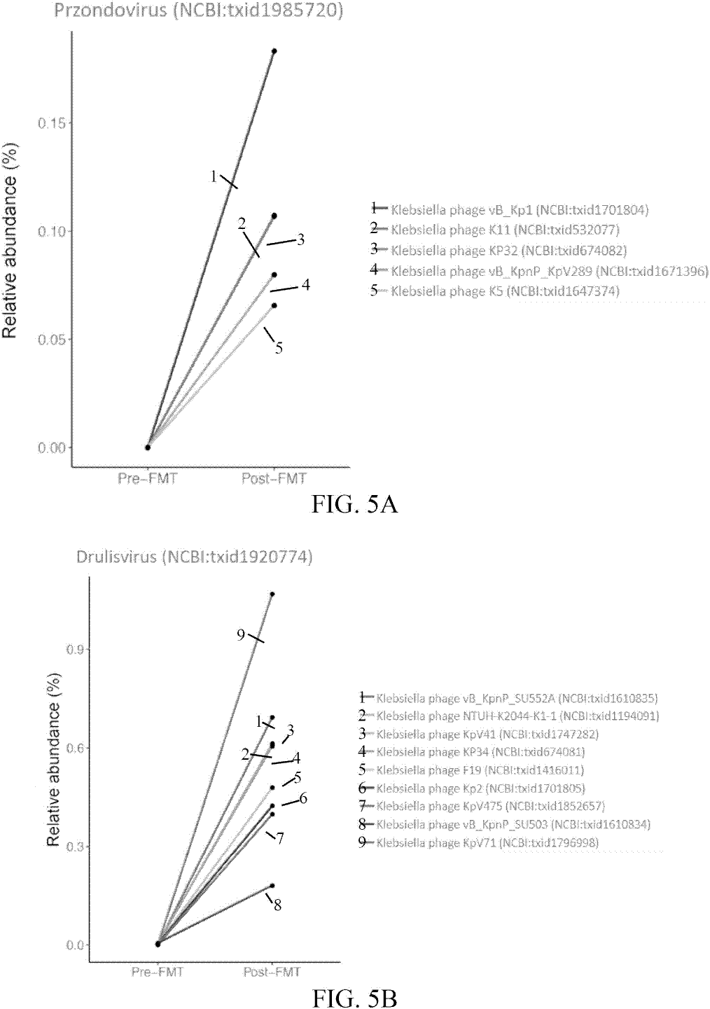

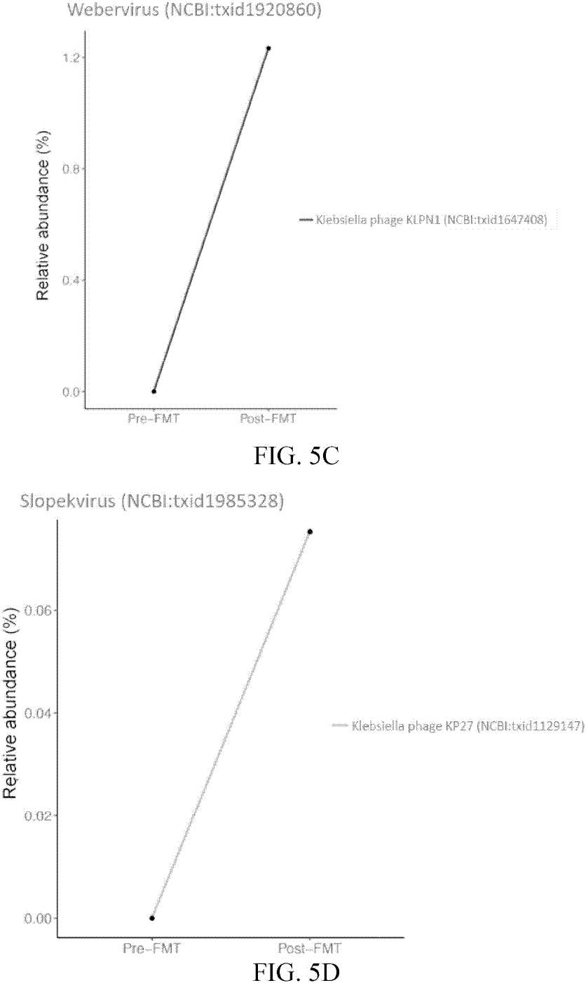

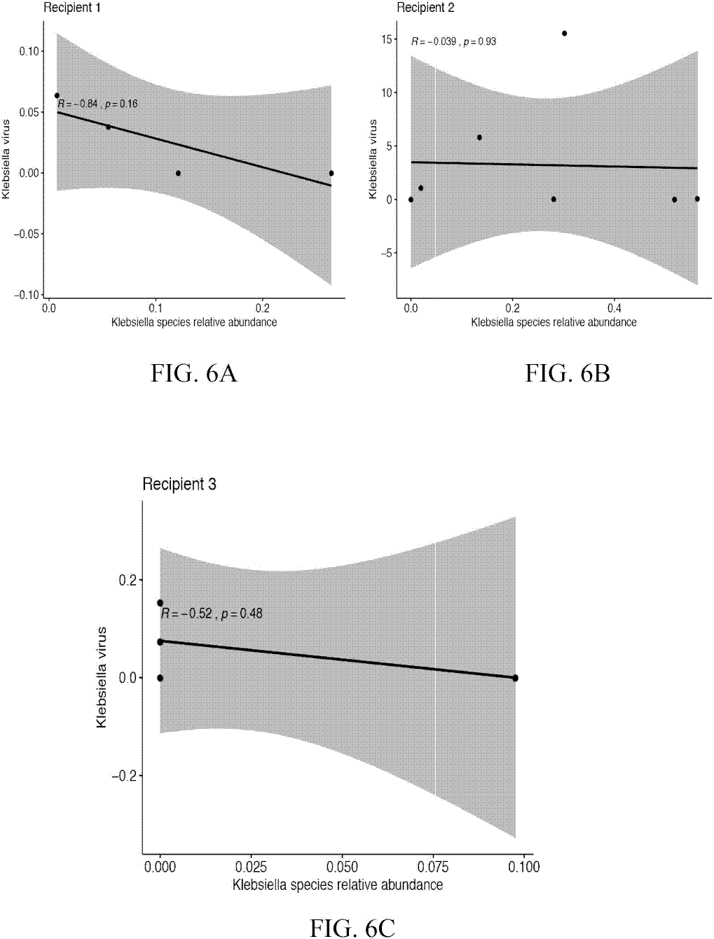

Bacteriophages are known natural predators that control the bacterial population and have a large impact on bacteria ecosystems. Strikingly, following an FMT, a substantial decrease of Klebsiella spp. abundance (clinical identified CRE species prior to FMT) was seen with a concomitant marked increase of Klebsiella phages (FIGS. 4C-4F), and most were belonged to Drulisvirus (NCBI:txid1920774), Przondovirus (NCBI:txid1985720), Webervirus (NCBI:txid1920860), and Slopekvirus (NCBI:txid1985328) (FIGS. 5A-5D). Using a linear mixed model, we found negative correlations between Klebsiella phages and Klebsiella spp. after FMT (FIGS. 6A-6C). These findings offered a potential mechanism through which FMT could confer the direct knockdown of Klebsiella spp. by lytic bacteriophages. The Klebsiella phages predation and bacteria-bacteriophage coevolution could be ascribed to the effective decolonization of CRE Klebsiella species in recipients by FMT procedure.

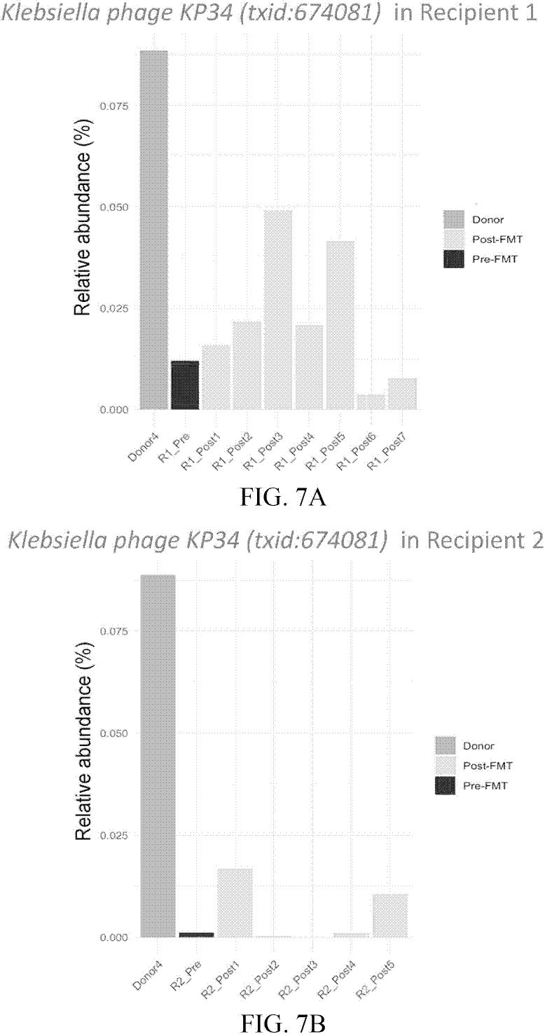

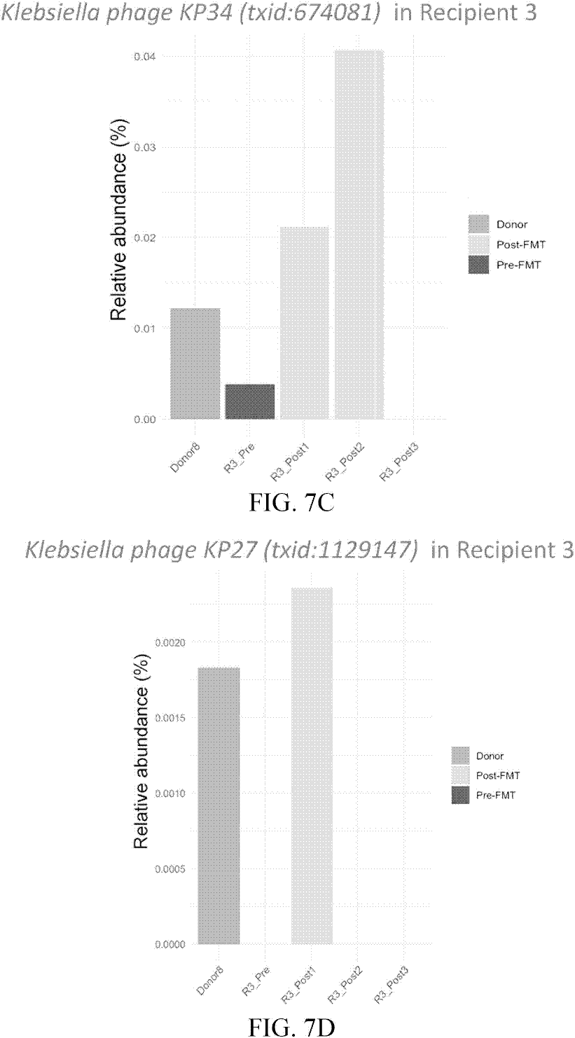

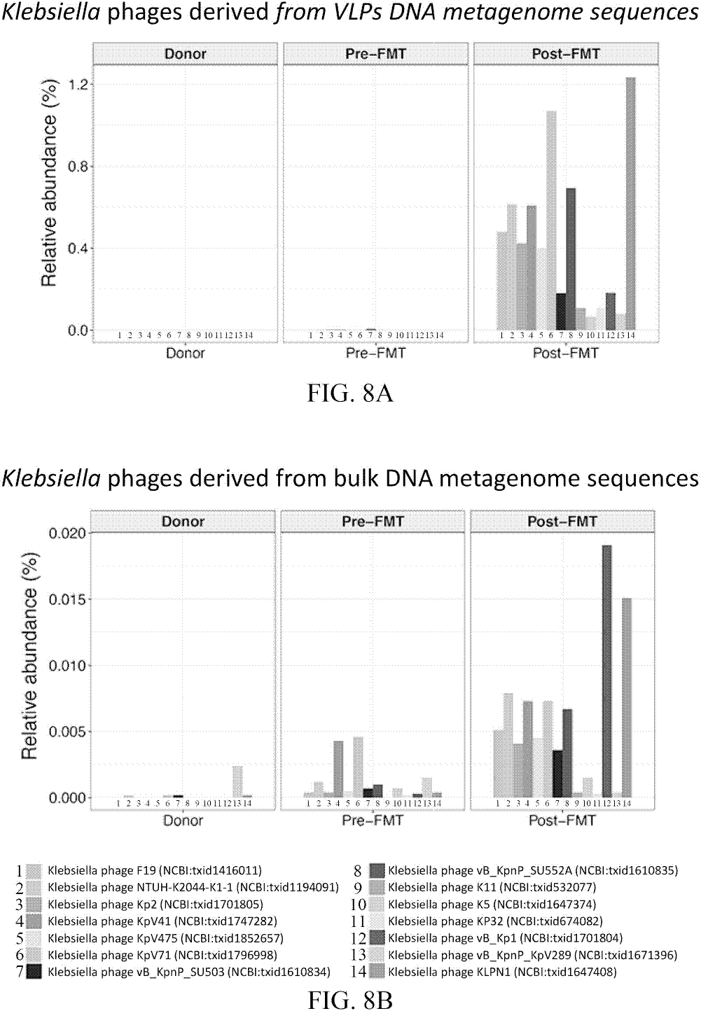

Since approach based on metagenomic analysis of VLPs focuses primarily on the free phages at the time of sampling, to test bacteriophage in full metagenome, we then further identified bacteriophages by comparing the whole metagenome sequences to viral database using the Kraken classification and alignment programs. In FMT recipients, we observed a marked increased level of Klebsiella bacteriophage (Drulisvirus (NCBI:txid1920774) and Slopekvirus (NCBI:txid1985328)) from full metagenomes and VLP in post-FMT samples (FIGS. 7A-7D; FIGS. 8A and 8B), which was likely the result of engrafted temperate phage from donor, or newly induced prophage that was triggered by FMT. Example sequences are, e.g., SEQ ID NOS: 1-332. As such, predation by phages and bacteria-bacteriophage coevolution could contribute to the effective decolonization of CRE Klebsiella species in recipients by FMT.

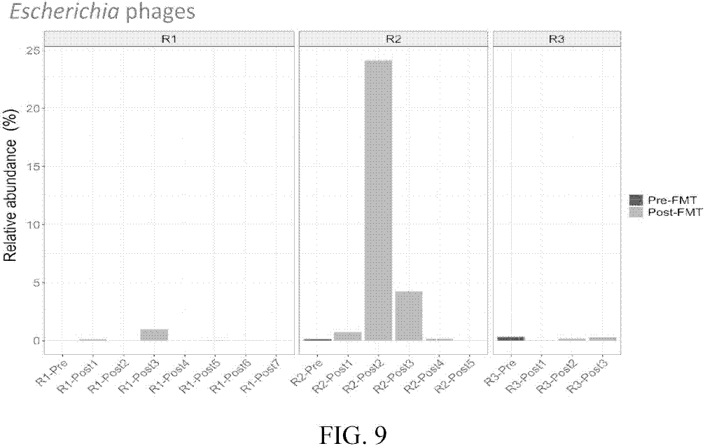

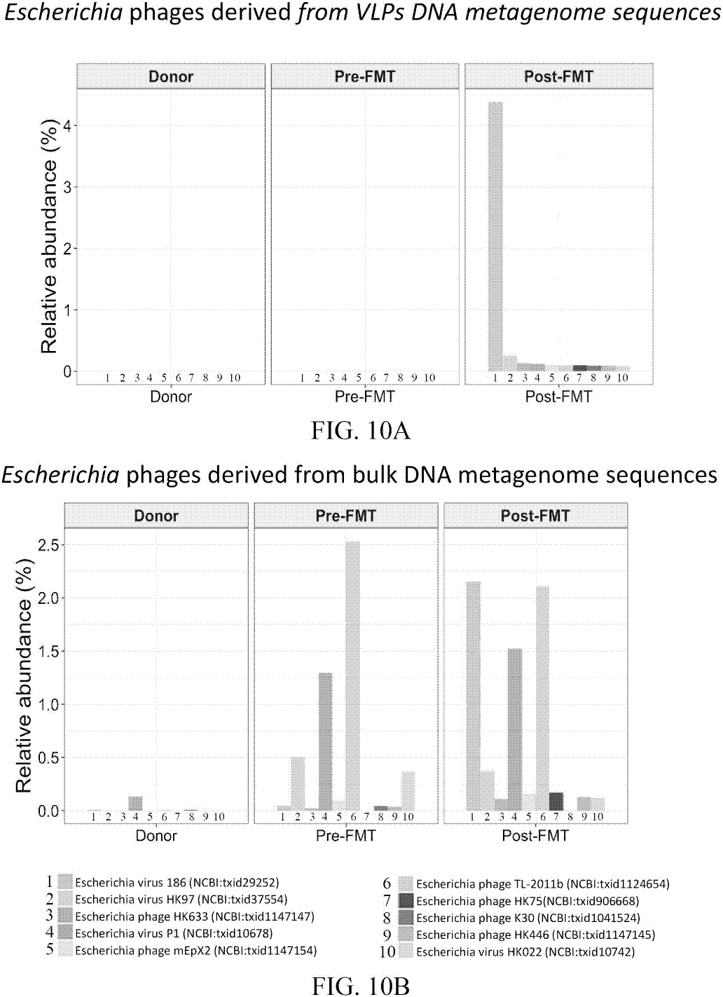

A striking increase in the relative abundance of Escherichia virus was only found in recipient 2, the only patient who carry CRE Escherichia coli (FIG. 9; FIGS. 10A and 10B). The clearance of CRE Escherichia coli after FMT indicated the inverse correlation (predator-prey relationship) between bacteriophages (Escherichia virus) and bacteria (Escherichia coli).

### Example 12. FMT Decolonize Carbapenem-Resistant Klebsiella Pneumoniae and Reconstitute the Microbiota in Mice

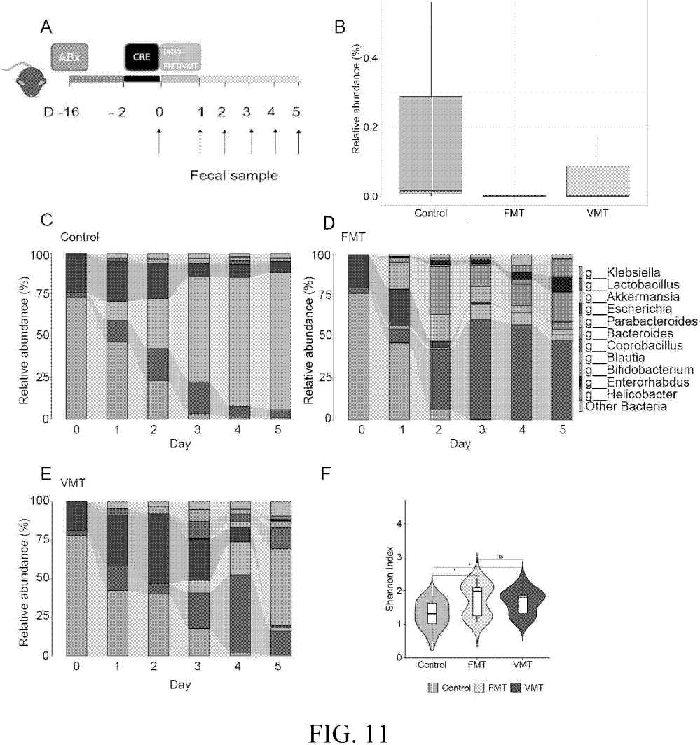

We validated the results in a parallel in vivo study of mice specifically gavaged with carbapenem-resistant Klebsiella pneumoniae. Carbapenem-resistant K. pneumoniae was introduced to antibiotics-treated mice and treated with healthy fecal microbiota/virome fraction twice by oral gavage (FIG. 11A). All mice were densely colonized with CRE prior to the treatment. While PBS-, VMT-treated animals remained colonized with CRE at day 5, mice treated with FMT showed clearance of CRE at day 4. (FIG. 11C). FMT resulted in progressive reduction in carbapenem-resistant Klebsiella pneumoniae levels and achieved clearance on day 5 and afterwards (FIG. 11B). Using metagenomic sequencing, we first characterized the fecal microbiota taxonomic composition. Taxonomic analysis demonstrated that Klebsiella genus and Escherichia genus were highly represented on day 0, approximately 75% and 20%, respectively after FMT treatment (FIG. 11C). While the microbiota of control mice were Akkermansia-dominated, FMT/VMT enabled a highly diverse population on the day 5 (FIGS. 11C-E). Despite these differences, FMT and VMT fecal microbial communities were similarly diverse, as quantified by the Shannon index (FIG. 11F).

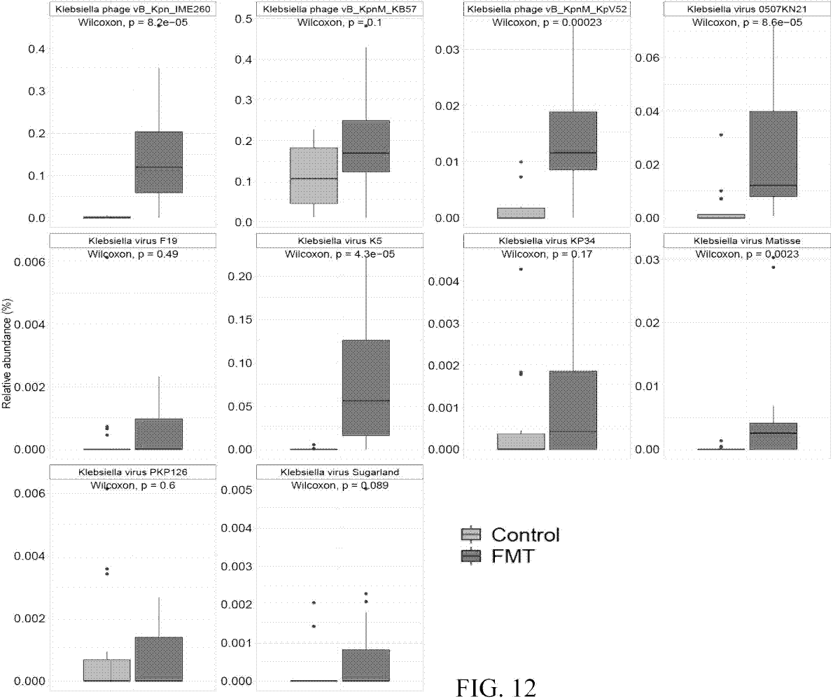

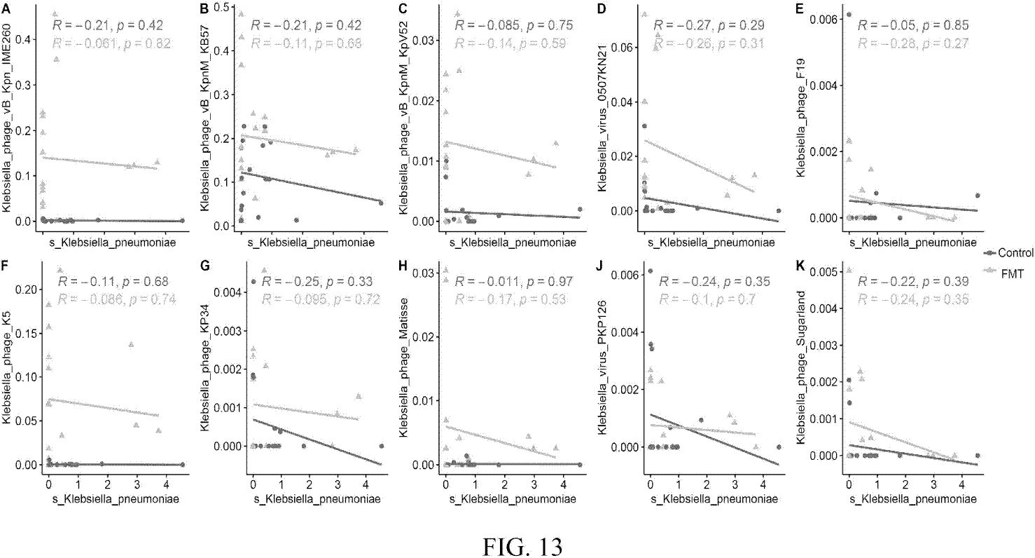

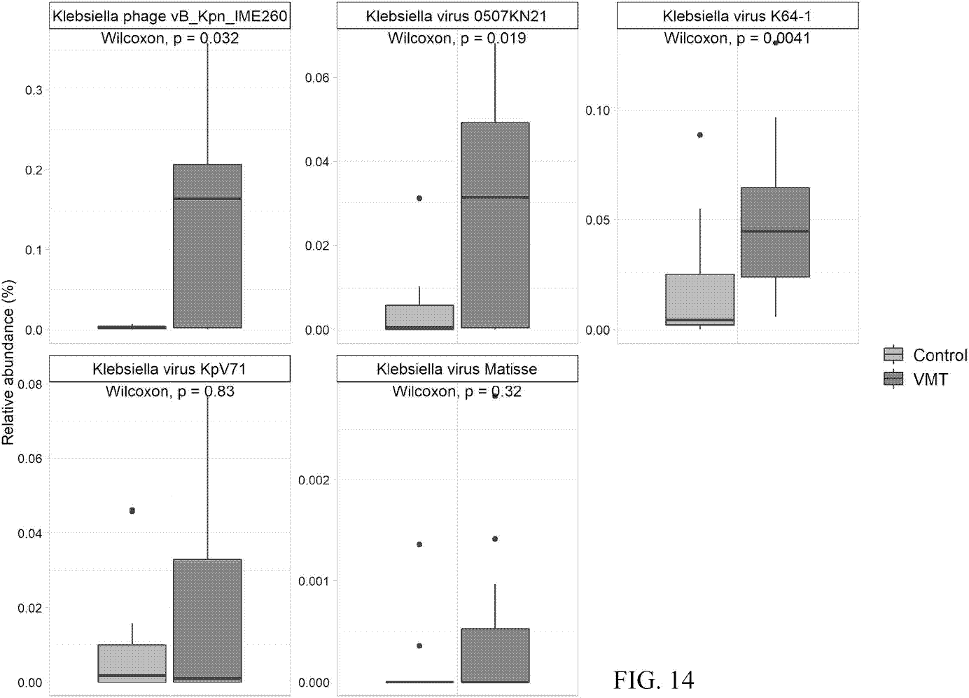

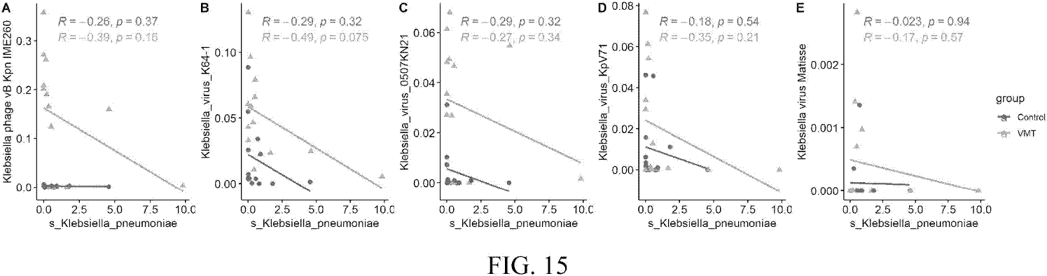

From human clinical trial, longitudinal metagenomic analysis of stool samples from CRE patients have demonstrated the bloom of Klebsiella phages after FMT. Herein, we found ten Klebsiella virus (List 6) exhibited higher relative abundance level in FMT treated mice than control animals (FIG. 12) and five Klebsiella virus (List 7) enriched in VMT group (FIG. 14). Further reflecting the dynamics of bacteriophage-bacteria interactions after FMT, correlations between ten Klebsiella phages and K. pneumoniae, calculated using a linear mixed model, were dominated by negative correlations (FIG. 13), consistent with findings in human samples (FIG. 6). Among these ten Klebsiella virus, Klebsiella phage F19, Klebsiella phage KP34, and Klebsiella phage PKP126 also shown negative correlation with K. pneumonia in human clinical trial. These findings coincide with our previous findings that FMT could confer the direct knockdown of Klebsiella spp. by bacteriophages.

All patents, patent applications, and other publications, including GenBank Accession Numbers or equivalents, cited in this application are incorporated by reference in the entirety for all purposes.

### INFORMAL SEQUENCE LISTING

List 1: Sequences that mapped to Klebsiella phage KP34 (NCBI:txid674081) which targets bacteria Klebsiella pneumoniae in bulk DNA metagenome sequences in donor/pre-FMT samples

| SEQ ID NO | Sequence | Sequence mapped to |
|---|---|---|
| 1 | GCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTA | Klebsiella phage KP34 (NCBI:txid674081) |
| 2 | AGAATCTGCCTTTTCGCGGACGTAAAGAAAATCATCCACCTTCGCCATCAGCTTATTCTGCCACTCCTGTTCCTCTTCGGAAACAGTTTCCTTTTCCGTGGTTTCTTCACTTGTATCATTTCCGGATATAACCGTTCCATCTGCTGTTGC | Klebsiella phage KP34 (NCBI:txid674081) |
| 3 | TTCCGACTCTGAAGGGCGACGAAAAGACTGGTGCAGAAATTATTCTGCGTGCACTGGAAGCACCGCTCCGTCAGATTGCAGCAAATGCTGGTCTGGAAGGCAGTGTTATCATCGACAAGATTCGGAGAAGCCGCAAGGTTGGTTATGGCT | Klebsiella phage KP34 (NCBI:txid674081) |
| 4 | ATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGT | Klebsiella phage KP34 (NCBI:txid674081) |
| 5 | AAGAGGGTTCGGGATGTCATTTTCGGGAAAAGTCAAAGAGGAACTTGCAGGACAGCTTAGTCTGGCAAGGCACTGTCAGGTGGCAGAACTGGCGGCACTGCTCTGTGGCTGTGGCCGTGTGGAAAAAATGTCGGATGGAAACAGGAAACT | Klebsiella phage KP34 (NCBI:txid674081) |
| 6 | GTAAGAGAAGGAGGAAAAAAGTAATTATGATGAAATTCTTACAGAAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACG | Klebsiella phage KP34 (NCBI:txid674081) |
| 7 | ATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATC | Klebsiella phage KP34 (NCBI:txid674081) |
| 8 | TCCAGCGGCAAAAAGAAAGTAAGTGAATATTGTTATAGTTTATACCAGTTTATCACAGACTGCGACATTCAGAGAAAATTGAAGGAACAGGAGCTTTCCTTCAAAGCAAAAGGGGAAAAAGCCCTGGAAAAGGAATACGCGCAGATATAC | Klebsiella phage KP34 (NCBI:txid674081) |
| 9 | ATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGT | Klebsiella phage KP34 (NCBI:txid674081) |
| 10 | CGCTTATCGGACTTACAAACATTTTCAACACAATCTCAACCAATATGCAGTTGAGGAGCAAGGAATTTGCATCGCTTAAATCTATCGGTATGACAAAGAAAGAATTTAACCGTATGATTCGCCTTGAAAGCCTTATGTACGGCATAAAAT | Klebsiella phage KP34 (NCBI:txid674081) |
| 11 | GAACGGGTTCGCAATTTTGCTGAATGCAATGTAAGCAGTTGTACCTTCTTCAAGAGTTTTGATAACAGATACAGCACCTACAGACAGCAGAGTTGTCAGCATCAGCCAAGAAGCCAGAGCTGCAAGTCCACCTGTACCGTCGTTGTCTTC | Klebsiella phage KP34 (NCBI:txid674081) |
| 12 | GGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAG | Klebsiella phage KP34 (NCBI:txid674081) |
| 13 | TGACGGAAAAGTTCCGCTGGAATATGCAGCCTTTTTGCTTACCCAGTATAAGGACATGCACACGGAAACTTACGATACGATGTCAGAAGTCCTGGAAAGGTATTATGCGGAAAAGAATACGATCACACGCATCCGCCAGAAATCTTCCGA | Klebsiella phage KP34 (NCBI:txid674081) |
| 14 | AAATTTGATAAATCCGAGCGAGATAAGGTTGAATACTTCGTCAGCGATATGTGGAGGACTTTTTCAGATATCAGTTCGGTTTGGTTCAAAAACGCAACGAAAATTGTTGATAAATACCACTGGATACGGCAGATTATGTACGCATTTGAG | Klebsiella phage KP34 (NCBI:txid674081) |
| 15 | GTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGG | Klebsiella phage KP34 (NCBI:txid674081) |
| 16 | CTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTT | Klebsiella phage KP34 (NCBI:txid674081) |
| 17 | AAATTTTGCAATCTGCCAAAAGTCACTTGCTTGAATGTAGCCGTATTGAAATTCTCATCCCAAAGTATTATACTGCTTTTTATAAAAAGGCACACACAAGCATACCGTGTCTTACTTTTCCCGACGCAAGCGCAAATGTTTGCACATCGG | Klebsiella phage KP34 (NCBI:txid674081) |
| 18 | TCTGCCGCGGCATGGTGGCCATCGTGGTCTGCTCGCTGGTGCTGAGCCGCCTGTTTGGCATGAATGGTGTGTGGGCAGCGTTCCCGGCATCGGAACTGCTCACCGCAGTGCTGACCCTGTTTTTGCTGCTGCGCGGTAAAAAGAGATCGG | Klebsiella phage KP34 (NCBI:txid674081) |
| 19 | GGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAG | Klebsiella phage KP34 (NCBI:txid674081) |
| 20 | GATAATCCAAATATGCAATCAGCGGTTGATAAACTATTAGGCATTGAGGAGTCAAAAATAACAGTAAGTCAGAAAGTTGCTCGTAGTGGTGATGATCGTGCAGTTGACGCTTTGAAAATGACTTCTGACCGTGTGGCTAAGTTTGAAAAC | Klebsiella phage KP34 (NCBI:txid674081) |
| 21 | TAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTGGCTTCTTGGC | Klebsiella phage KP34 (NCBI:txid674081) |
| 22 | TTTCATCACCCCTTTTATTTACTCCAAAATCGGCTCCACCAGCTCCGGGGCGGCTCTGGCTGCTGCCGTTCCGGCTCTGCCGGAACTACGCAATCCTCTGATTTTCTTCATGTTCTGTACCTCTTTCGTAAATTCCGATTCATAATGTAA | Klebsiella phage KP34 (NCBI:txid674081) |
| 23 | CAGCTCGTAGCCGTCATCAGTGGACTTGTAGGTGTACAGGGTATCAGCAATTGCGGTTACCTTGTCGGTGTCGGTAGCTGCATCGTTAACCTTTTTGCCGTCTACCTTGCGAACCTTGATTTCCTTGTTAGTGCCATCGGTAAAGTAAGC | Klebsiella phage KP34 (NCBI:txid674081) |
| 24 | TATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCAGATCGTAAGAGCGTCGTGTAGGGAAAGA | Klebsiella phage KP34 (NCBI:txid674081) |
| 25 | GAGCAGGCTACCTTCATTTTTGAACCTCTGAATGCAAAATCTATTGCACCAACGTTCTGTACCTCGTTGCACACTCTTACGCAGTCACCGCAGAGGATACACTTGTTGGCGTCCCTGGTGATAGATACAGAAGAATCATCGATACAAGGC | Klebsiella phage KP34 (NCBI:txid674081) |
| 26 | ACATATCCGCAGAATCCAGCCAGAAAAAGAGCTGGAGGAGGATTCTCATTAAGTAAGAGAAGGAGGAAAAAAGTAATTATGATGAAATTCTTACAGAAATTAGGTAAAGCTTTGATGTTACCTGTTGCTTGCCTTCCTATCTGTGGTATC | Klebsiella phage KP34 (NCBI:txid674081) |
| 27 | CCTACAGACAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAA | Klebsiella phage KP34 (NCBI:txid674081) |
| 28 | CAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGC | Klebsiella phage KP34 (NCBI:txid674081) |
| 29 | CACCTGCACAGATCAGATTGTACTTGCTTCCCATTGGTCCTACGAATGTGTAGAGGGAAAGTGCCATGGTACGGATTTCTTTTCTCATCAGGTAAAGGTTTGCTGCGTAGTATTCGTTGTACACACCAACACCCTTTAAAATACAGGATG | Klebsiella phage KP34 (NCBI:txid674081) |
| 30 | AGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAG | Klebsiella phage KP34 (NCBI:txid674081) |
| 31 | TGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTG | Klebsiella phage KP34 (NCBI:txid674081) |
| 32 | AGAGCACGGCCGGCGGCATCCGGCTGGACACCCTCTTCCTCGACGAGGGCTTCGGCTCCCTCGACGACGAGAGTCTGGAACAGGCCATCCGGGTCCTCTCCGGCCTGACCGAAGGCGACCGGCTCGTGGGCATCATCTCCCACGTCGCCG | Klebsiella phage KP34 (NCBI:txid674081) |
| 33 | GATATGTACGAGATAGGCTTTGAATGGGACGATCATGTAAGACTTCGGGATACGCTGAAAAACATCAAAGGTAAATTTCTGCTGTCCTACAACGACTGCGATGAAATTCGTGAACTGTACAACGGATTTTCAATGTTTGATTTTTCCCGA | Klebsiella phage KP34 (NCBI:txid674081) |
| 34 | TAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTGGCTTCTTGGC | Klebsiella phage KP34 (NCBI:txid674081) |
| 35 | CGGCACGGTGCTCATGCCCACAGCATCGGCCCCCATGCCCGCAAAGGCACGGATCTCGGCGGCCGTTTCATAGCAGGGACCCATGAAGCCCAGATAAACGCCCTCTTGATAGGAAATGCCCAGCTCGTCGGCGGTCTGTTTGGCGAGATT | Klebsiella phage KP34 (NCBI:txid674081) |
| 36 | AGAAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTATTGAAAG | Klebsiella phage KP34 (NCBI:txid674081) |
| 37 | AGCAGGAAGAACCGATCACACCGGCGATGATACCGATGAACGGGTTCGCAATTTTGCTGAATGCAATGTAAGCAGTTGTACCTTCTTCAAGAGTTTTGATAACAGATACAGCACCTACAGACAGCAGAGTTGTCAGCATCAGCCAAGAAG | Klebsiella phage KP34 (NCBI:txid674081) |
| 38 | GATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAG | Klebsiella phage KP34 (NCBI:txid674081) |
| 39 | CCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAG | Klebsiella phage KP34 (NCBI:txid674081) |
| 40 | GTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTGGCTTCT | Klebsiella phage KP34 (NCBI:txid674081) |
| 41 | GCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGA | Klebsiella phage KP34 (NCBI:txid674081) |
| 42 | GTCAACCAGAGCGATTCTTCCACTGGCGTTTCCTCTGCAATTGATGGTACTTGTGATATTGGTATGGCATCCAGAGAACTGAAAGATTCGGAAACCTCGCAGGGTGTTGTTTCCACAGTCATTGCAGTCGATGGGATTGCAGTCATTGTC | Klebsiella phage KP34 (NCBI:txid674081) |
| 43 | GGGAAGAATTTTCGGCGAAACAGCCGCACTTATCTATACCGCGGGAACTATCGCCAAGTATGCAGGCCTTACAGACTCAGGACGTACCCTTTCTATCCATATGTATTCACTTTCAATCGAGGGACTTTATATGAACGAAGCAAGCGCTAC | Klebsiella phage KP34 (NCBI:txid674081) |
| 44 | TGGTTTTTCCATCAGGGCAAACGTCCCCTCTGAAACATAGGTATAGGTCAGCATGCTTTTTACCTCCATGCGCTTTGATATTTCCGGACTTGAGTATACCACGCAGCTATGGCAAAGAGCAAGCCGCAAAAGGCCCGCCCTTTGCGGCAG | Klebsiella phage KP34 (NCBI:txid674081) |
| 45 | GGGGGTCTTGGCGGCCACCACGGCGTTGCCGTAGGTCTCGCCCGGCACCCAGCCGAAGCCATCCATGATGCACAGAAGAACAGGTTTCTTTGCCATAAAATTGAATCTCCTTTGAATTTAACAAATCAGCGCCCGGCACAGCGCTGGAAA | Klebsiella phage KP34 (NCBI:txid674081) |
| 46 | GATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGAT | Klebsiella phage KP34 (NCBI:txid674081) |
| 47 | GATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGAT | Klebsiella phage KP34 (NCBI:txid674081) |
| 48 | CTTACAGAAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGT | Klebsiella phage KP34 (NCBI:txid674081) |
| 49 | ATCTCTGCGGAGAAAGCGGCTGCGGTCTCTGTGCGTTAAAGCAGTTATCCAATGCGGGAATCACACATTTAAAGCTCGTCGGACGCGGAAACTATGCCAACTTTATGGAACGCGACATCCGGAATCTGCGCTGGGCACTCGATATTCTTG | Klebsiella phage KP34 (NCBI:txid674081) |
| 50 | CATGCATAACCGAGTCTCCATCCGGTCATTGCATGCGATTTGGAAAATCCGTTGATCACGATTGTTCGCTCCCTCATTCCCGGAATGGAGGCAATGGATACATGATTGTCCAGATAGGTCAGTTCCGCATAGATCTCGTCACTCAGGACA | Klebsiella phage KP34 (NCBI:txid674081) |
| 51 | CTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATG | Klebsiella phage KP34 (NCBI:txid674081) |
| 52 | AATTATCATGATCGACTACCTTCAGCTGATGAGCGGAAGCGGAAAGAGTGATTCCCGTCAGCAGGAACTTTCTGATATTTCCCGTTCGCTTAAGGCACTTGCGAGAGAACTGAGCGTGCCGGTCAGTGCGCTTTCGCATCTGAGCCGTGC | Klebsiella phage KP34 (NCBI:txid674081) |
| 53 | AACACATTGGAAGCTTCCTCGCCAATGCCATGGTCTGCCGTAATTAAGTAATAGTACGTGTAAAGCTCACTGGTCTTTTCGTTGTAGTTGACGGT | Klebsiella phage KP34 (NCBI:txid674081) |
| 54 | TCTCTCCCTTACGTCTTAATCTGAACACATTTCAGAGCTTATGATATCTAGCAGAAAATCTGCGATATCATTATACACCTCACGGCGGTTGGTTTCGATCACAAGCTCGTGACGTGCATTTTCGTATATCTTCAGCTCAACGTCACAGCC | Klebsiella phage KP34 (NCBI:txid674081) |
| 55 | CAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACC | Klebsiella phage KP34 (NCBI:txid674081) |
| 56 | TACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGG | Klebsiella phage KP34 (NCBI:txid674081) |
| 57 | GACTCCACGGAACAAATTCGCTGTCTCCGTGTTCGCCTATTACATATGCGTGAATATTGCGTGGGTCAACCTCAAAATATTCGCCGAGAAGATATCTGAGCCGTGCCGTGTCAAGGGTAGTTCCCGTGCCGATGATGTTTGACGCATTAA | Klebsiella phage KP34 (NCBI:txid674081) |
| 58 | TAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGC | Klebsiella phage KP34 (NCBI:txid674081) |
| 59 | CCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAG | Klebsiella phage KP34 (NCBI:txid674081) |
| 60 | AAACCTATTGTAATCTTTGCAGTATGTATGGGTATAGTAAATGTAATCTTTGCTTTTGACTTAACAGGTTACGAAAGAAGAGTGCCAAGTGAAAATAATGTTGTAAGTATAGATGCCTTTGACTATATGGGACATATTAGTTACGATAGT | Klebsiella phage KP34 (NCBI:txid674081) |
| 61 | TCGCAATTTTGCTGAATGCAATGTAAGCAGTTGTACCTTCTTCAAGAGTTTTGATAACAGATACAGCACCTACAGACAGCAGAGTTGTCAGCATCAGCCAAGAAGCCAGAGCTGCAAGTCCACCTGTACCGTCGTTGTCTTCGGACATAC | Klebsiella phage KP34 (NCBI:txid674081) |
| 62 | TAAACCTCTTTGACCTATGAGACTTAAAAGAGGATAGTTCGTCAATTACAATCATATCGTAGTTAAAGGACAGTTCGCTCTTATTTATTAACCAGTCTACATTTTCCCTATTGAGTAAAT | Klebsiella phage KP34 (NCBI:txid674081) |
| 63 | TGAAATCCAAGGTGATGTTTTAACAGATATATATTCATATGGCAATATTTATAATTATATTCCTTTTTTTACAAGTGAAAATAAAATTTATTATAGTACAATTAAAAATGGAAAATTAAAAAAACCTGCCTTATTAAGTGGATGGGCAGA | Klebsiella phage KP34 (NCBI:txid674081) |
| 64 | TGCCCAACCGAAACCGCCCCATTTTTGCTTTGGCTTTTCAAGTTTGCGGTACATTTTAATCGGACTTGTCTGTGCCGCTCTGCCCACCGAAAATTCCATTATCAAACACGGAATTCCCAAAACAATAAGACAGATGATGTACACAAGCAT | Klebsiella phage KP34 (NCBI:txid674081) |
| 65 | GTCAGACATGGCAAGGCAAGTGAATATTCCCTACAGCGCACTATATAACAGCCTTTTCAACGAAAAACGGGATCGTGATCTGCGAGTAGATGAGTTTCTTACGGTATGCAAGTATCTGGATGTCGATCCGATGATCTTTTGGAAACCATG | Klebsiella phage KP34 (NCBI:txid674081) |
| 66 | CAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCA | Klebsiella phage KP34 (NCBI:txid674081) |
| 67 | CAAAGACGGTACTTTAACATACCTGCGTCCGGACGGAAAAACACAGGTAACCGTAGAGTACGATGAGAACGGCAAACCGTTCCGTCTTGATGCGGTTGTTCTTTCCACTCAGCATGATCCGGAAGTATCCCAGGAGCAGATTCATGAGGA | Klebsiella phage KP34 (NCBI:txid674081) |
| 68 | GCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTTTACCTAATTTC | Klebsiella phage KP34 (NCBI:txid674081) |
| 69 | AGCGCGGTGGATTTGCCGCAGCCGGAGCCGCCCAGCAGCACCACCATTTCGCCCGGATTGATTTCCAGGTTGAAATCGTCCAACGCCTTGACGGTCGAACCGGGGTAGATCTTGTCTACACCAAGCATCTGCACACCGCCGCCGCGCGC | Klebsiella phage KP34 (NCBI:txid674081) |
| 70 | TCTTACAGAAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAG | Klebsiella phage KP34 (NCBI:txid674081) |
| 71 | GAAAAGAAACCTCTTGGATATAGAAAAAATATCATATATGTACTATAATGAAACAGTGACAAAAAATGCACATAATGTGTGGGAAAGGTAATATCATGAAAGAAAAAAATGTGTCATTAGAGAATAAAGTGATTTTAGTAACCGGTGCTG | Klebsiella phage KP34 (NCBI:txid674081) |
| 72 | CATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACT | Klebsiella phage KP34 (NCBI:txid674081) |
| 73 | GAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTT | Klebsiella phage KP34 (NCBI:txid674081) |
| 74 | TTACAGAAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTG | Klebsiella phage KP34 (NCBI:txid674081) |
| 75 | TACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCAC | Klebsiella phage KP34 (NCBI:txid674081) |
| 76 | ATAAAAACCGTCCTTTTTAAAGAAAATAGGGCAAGTCATATAATATATAACTCGCCCTTTGAGTTTGAATATATGACTGCAAATGCAGTTTTTATCTTAATTTAATATGAACCTCACGGAGCTGCTGTTCTGTTACCTCACCCGGTGAAC | Klebsiella phage KP34 (NCBI:txid674081) |
| 77 | GGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTG | Klebsiella phage KP34 (NCBI:txid674081) |
| 78 | TACAGACAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATA | Klebsiella phage KP34 (NCBI:txid674081) |
| 79 | GTGATATAAAGGTCAAACAGGTTCTTGAAGTTATCAAGATAGGTTCCTTTGATCGCATCTGTGGATGTGATTCCGTCTTCTTTGTACTCATAATAGATCGGAAGGTTTGCAAGGTGTGTCTTGAAGCGCCAGTCACTGGAAGAATCAAAG | Klebsiella phage KP34 (NCBI:txid674081) |
| 80 | AAAAGCAGCGAATGAACTTCAGATACCCGTAAATACGCTGTACGGCTGGATCAGAAAAGTAAAAATCGGAAGCCTTGATATTGGCTGCGGAGAACGCAGCCCGGAAGAATCTCTGAATATCGCCGAAGAAAACCAGCAGCTGAGGAAGCG | Klebsiella phage KP34 (NCBI:txid674081) |
| 81 | GGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGATGGTACAGGTGGACTTGCAGCT | Klebsiella phage KP34 (NCBI:txid674081) |
| 82 | CCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAG | Klebsiella phage KP34 (NCBI:txid674081) |
| 83 | CCGATTACCGTAAATAAAAATGGAAGCGGTACCGTTAAAATCAATAACGAAGAGGTAACGGATTCTAAAACTGTTGAAAAAGACAGTACGGTAGAACTGGAAGTTACCCCGGCAAAAGATACCTACATCAAGAAACTTGTAATCGGCGGA | Klebsiella phage KP34 (NCBI:txid674081) |
| 84 | ATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGAT | Klebsiella phage KP34 (NCBI:txid674081) |
| 85 | CGGAAAAGAACAGCCACAGACAGTACCGGTGTATGTGAAGTTTTCCAATGAGAGAGATCAGAAGCTCTCCATTTATACGGAAATCAGTGAAGCAGCAGATGGGCAGCTGACTGTGAAGAAAGTACCGTCGCAGAAGAAAGCGGCAGCTCA | Klebsiella phage KP34 (NCBI:txid674081) |
| 86 | TGCTCCACAAGGCTTCGCTTTGATCTTAAAGATAAAAATTCTATTGACAAAGCTGCCTTAGAAAGAACCGATGGCATATTACAGGTAATGGAAGTTGCCGGGCAAACACAGGTTGTAATTGGTTCAAATGTTCAATATGTATACGATGAA | Klebsiella phage KP34 (NCBI:txid674081) |
| 87 | GAGCTGGAGGAGGATTCTCATTAAGTAAGAGAAGGAGGAAAAAAGTAATTATGATGAAATTCTTACAGAAATTAGGTCAAGCTTTGATGTTACCTGTTGCTTGCCTTCCTATCTGTGGTATCCTGATGGGTCTTGGATATGCTCTTTGCC | Klebsiella phage KP34 (NCBI:txid674081) |
| 88 | GAAGAATAAACATCTTCCTGTCCTTTAATTGTTGCAAATAGGGGGTACAAATGCGTCAAGTTATAGCTAGAGCTATTTGTATCCCCTGTTAGCATGGTTCTTCACCTGTATTTCTATTTCCTATTTCATTCTTTTGCCACAACCTTTTAC | Klebsiella phage KP34 (NCBI:txid674081) |
| 89 | GGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGC | Klebsiella phage KP34 (NCBI:txid674081) |
| 90 | TGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGT | Klebsiella phage KP34 (NCBI:txid674081) |
| 91 | AAATACCGGATATGTTATACTTCCATTATCAATATCCTTATAAAAATCATCTTTGTTCATATATGATTTAGCGCATTTATATCCATTTTTAGTAAGCCATAAATACATTTCAAATTTATCTAAAGATGTCTCGCACAATTCATAAGATGA | Klebsiella phage KP34 (NCBI:txid674081) |
| 92 | ATATCAAAATCGTAAATTATCATATTGGAAAATTGAGAACTTTGTGCTATATTGTTAGAGTAATGAAAAACAAATCATTTTTTTTGGGAGGAACTTATCATGAAGAAAAAAGTAATCGCTGGTTTACTTGGAACAGCAATGGTAGCATC | Klebsiella phage KP34 (NCBI:txid674081) |
| 93 | CGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCA | Klebsiella phage KP34 (NCBI:txid674081) |
| 94 | GTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCG | Klebsiella phage KP34 (NCBI:txid674081) |
| 95 | CGTTTGAGATGAGACCCTTCCCGGATAACAATCGCGGTCTCTATATTAAAGGTTTTTCGCAATAATGTAAAGCTTTTTCTTGCAACTGCTTCATTTTCTGTCTGGATCCACAGTT | Klebsiella phage KP34 (NCBI:txid674081) |
| 96 | GGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGA | Klebsiella phage KP34 (NCBI:txid674081) |
| 97 | CCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTTTACCT | Klebsiella phage KP34 (NCBI:txid674081) |
| 98 | ATACAGCACCTACAGACAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTT | Klebsiella phage KP34 (NCBI:txid674081) |
| 99 | GGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTG | Klebsiella phage KP34 (NCBI:txid674081) |
| 100 | TATTCGCTGTTTCCACAAAAAACCGGACGGAACTTCGGGGCTTCTTCCCTCTTGACAATTCCGGATCTTGAGATAAGAAGCAAGCCGCAGCTTCTTTCCCTCTTGTAAAATCCGGTCAAAACGGTTATGATAGAACCAAATAAAAAACAA | Klebsiella phage KP34 (NCBI:txid674081) |
| 101 | TTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTTTACCTAATTTCTG | Klebsiella phage KP34 (NCBI:txid674081) |
| 102 | CTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTG | Klebsiella phage KP34 (NCBI:txid674081) |
| 103 | TTCAGAATTCTTTCAAAAAATTACGATTTTACACAATTCTACCATTTTTGCATGGTAAAATAAGGTCGTAGAAAAAACAAAGGATGTGGCTCAATATGCAACACTGTCCCGCCCGCGCGGCACAGCGGCAGATCGGAAGAGCACACGTCT | Klebsiella phage KP34 (NCBI:txid674081) |
| 104 | TGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTAT | Klebsiella phage KP34 (NCBI:txid674081) |
| 105 | GGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTGGCTTCTTGGCTG | Klebsiella phage KP34 (NCBI:txid674081) |
| 106 | TTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCT | Klebsiella phage KP34 (NCBI:txid674081) |
| 107 | GCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAA | Klebsiella phage KP34 (NCBI:txid674081) |
| 108 | GTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAA | Klebsiella phage KP34 (NCBI:txid674081) |
| 109 | GATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACC | Klebsiella phage KP34 (NCBI:txid674081) |
| 110 | GCTTAAGAGGTCTGCATCTACTACGGTGAACTTAGTTCTCTTCGAAGTATCTACCACTGATTCGAATATATACTCTCCAGCTTGTGCAGTTACAAACTCATAACCAGCACCACCTGCATCATAAGTAGTTACTTTACCAACTTCCCTTAT | Klebsiella phage KP34 (NCBI:txid674081) |
| 111 | TATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACA | Klebsiella phage KP34 (NCBI:txid674081) |
| 112 | CGCAATTTTGCTGAATGCAATGTAAGCAGTTGTACCTTCTTCAAGAGTTTTGATAACAGATACAGCACCTACAGACAGCAGAGTTGTCAGCATCAGCCAAGAAGCCAGAGCTGCAAGTCCACCTGTACCGTCGTTGTCTTCGGACATACC | Klebsiella phage KP34 (NCBI:txid674081) |
| 113 | CCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACA | Klebsiella phage KP34 (NCBI:txid674081) |
| 114 | CCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACA | Klebsiella phage KP34 (NCBI:txid674081) |
| 115 | CGGCAAGGGCCAGATATTCCCCTGCGGCAAGCTCTGCCGCAGCATTGGTGTTGGCCGCAATGCCTTTGTTTTCGATCTTTTTGTATACGATGCGTTGATTCTTTGTCTGATATTCCTCCACGATGCGCTTCACATCGGCATGTTCTGCAT | Klebsiella phage KP34 (NCBI:txid674081) |
| 116 | GGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAAGATCGGAAGAGCACACGTCTGAACTCCA | Klebsiella phage KP34 (NCBI:txid674081) |
| 117 | GTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAA | Klebsiella phage KP34 (NCBI:txid674081) |
| 118 | ATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGT | Klebsiella phage KP34 (NCBI:txid674081) |
| 119 | TGCCATGTATAGTCAACTTCAAGCATGGAGTTTGCAAAATCTCTGCCATAAACTGAATAAACTGCGGAATATATCTTGCCGTCCCCTGCTGTCAATATCTCAACTTTATTATCCCTGATGAACTTGAATGATATCTCAGGGTGTGAAAGG | Klebsiella phage KP34 (NCBI:txid674081) |
| 120 | GAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTT | Klebsiella phage KP34 (NCBI:txid674081) |
| 121 | CCACGGCGTTCGATACGACAAGCTCGGCGGCGCCCTTGCCTTTGCCGTGCGAAACGGCGCACAGGCTCAGCTCAATCACGCTGCCCTCGCTCGCATAGCGCGTGGCGTTGTCCAGTAGCAACTCAACCACCTGCGCCACCGCCGCGGCAT | Klebsiella phage KP34 (NCBI:txid674081) |
| 122 | CCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCA | Klebsiella phage KP34 (NCBI:txid674081) |
| 123 | GCAAGTCCACCTGTACTGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGC | Klebsiella phage KP34 (NCBI:txid674081) |
| 124 | CTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTTTACCTAATTTCT | Klebsiella phage KP34 (NCBI:txid674081) |
| 125 | ATGAGAGAAGGAATCCATCCGAATTACTATCAGGCAACTGTAACCTGCAACTGCGGTAATACTTTTGTAACAGGATCTACCAAGCAGGACATCCACGTAGAAATCTGCTCCAAATGTCATCCGTTCTATACAGGACAGCAGAAAGCTAGT | Klebsiella phage KP34 (NCBI:txid674081) |
| 126 | CCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCA | Klebsiella phage KP34 (NCBI:txid674081) |
| 127 | AGACAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAA | Klebsiella phage KP34 (NCBI:txid674081) |
| 128 | AGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTGGCTTCTTG | Klebsiella phage KP34 (NCBI:txid674081) |
| 129 | GCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGC | Klebsiella phage KP34 (NCBI:txid674081) |
| 130 | ATACCTGCTTTCTTCCGGGAATACAGCGAAACGAAAGCGTGCCGGACTGTATTCCTGGAGTTTGATGAAAAATTTGAAATCACATACCCTATCGGGGTATCTAAAATATAGCGCGGATGAAAAGAAATGTCAATAAGCTGATAAAATATG | Klebsiella phage KP34 (NCBI:txid674081) |
| 131 | GGATGGGAATTTATAATCTTCTGATGAAGACTGGAATGGGAATCTGGCCGTCTGGGATAATTGCCCTGCTGATTTTGCTTCAATACGGCGTGATGACTGGTGGAAGTGTGTCTACTATGCGTGCAGTCTGTATGTTTTTGATATCTGTAG | Klebsiella phage KP34 (NCBI:txid674081) |
| 132 | CTGGCCGTACATATCCGCAGAATCCAGCCAGAAAAAGAGCTGGAGGAGGATTCTCATTAAGTAAGAGAAGGAGGAAAAAAGTAATTATGATGAAATTCTTACAGAAATTAGGTAAAGCTTTGATGTTACCTGTTGCTTGCCTTCCTATCT | Klebsiella phage KP34 (NCBI:txid674081) |
| 133 | GATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGG | Klebsiella phage KP34 (NCBI:txid674081) |
| 134 | TGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTG | Klebsiella phage KP34 (NCBI:txid674081) |
| 135 | TCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATC | Klebsiella phage KP34 (NCBI:txid674081) |
| 136 | CATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACT | Klebsiella phage KP34 (NCBI:txid674081) |
| 137 | CTGCGAAAACGGCGGAAATTCCGTTATAAGAACACCGTTTTTCTTTATCCTGTTACGCAAATCCTCATTTGTTTTAAGGTAATTTGTTCCGAGACCGCAGCCGAGCACCGCAACGGTTTTCTTGTCCACGCTCAATGCGCCCTCATGCGA | Klebsiella phage KP34 (NCBI:txid674081) |
| 138 | CCTGTGCCTGGTTCTCTTCGCGGTGGGGCGGAGCTTTTGGTTCATGTTGTTGGTGTTCCTGCTGGCGGGGCTTATGCGCACCATCAAGGAGCCTGTGCTGGCCGCCTGGATGAACGACCATGTGGATGAGAAAATGCGCGCCACAGTCTT | Klebsiella phage KP34 (NCBI:txid674081) |
| 139 | GATGACCGCAGCCCTTCGGAAAGAGTTCGGTTTTCATCTTCCTTCGATATTCTGCATCACTTTTGGCCGTATCAAGAATATCGAGTGCCCAGCGCAGATTCCGGATGTCGCGTTCCATAAAGTTGGCATAGTTTCCGCGTCCGACGAGCT | Klebsiella phage KP34 (NCBI:txid674081) |
| 140 | AGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAAC | Klebsiella phage KP34 (NCBI:txid674081) |
| 141 | GAAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGC | Klebsiella phage KP34 (NCBI:txid674081) |
| 142 | CTTCGACGAAGACGATGATTCCCGTATCGTCGCGTGAAACGATATCGAGTTCGCCATATCGGCAGTGCCAGTTGCGGTCGAGCGTCTGCCAGCCTTGGCTTTCCAGCCATGCGGCCGCATACTGTTCGCCGAGTTCGCCGACCTGTCGTG | Klebsiella phage KP34 (NCBI:txid674081) |
| 143 | GATGGGTCTTGGATATGCTCTTTGCCCATCTACCATGCAGGGGGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGG | Klebsiella phage KP34 (NCBI:txid674081) |
| 144 | CGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGA | Klebsiella phage KP34 (NCBI:txid674081) |
| 145 | GCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTTTACCTAATTTC | Klebsiella phage KP34 (NCBI:txid674081) |
| 146 | TGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATA | Klebsiella phage KP34 (NCBI:txid674081) |
| 147 | GAATCACTCGAAGTACTGCGGCGAAAGTGATTGCCGGTTATTTTCATACAGAGGCAACTCATGTCGGCGGTTGTTATGATGCATATTCTGTTCGTGACAATGACGGCAGGATGTGGAAGATTATGCGTGACGCAAGCGTCCGTTGTGAAA | Klebsiella phage KP34 (NCBI:txid674081) |
| 148 | TGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCAT | Klebsiella phage KP34 (NCBI:txid674081) |
| 149 | ATTGTTGTAATCTGCGCAGGTGCAAATCAAAAGCCCACCGAAAGCAGGCTTCAGCTTTTGCAGAAAAACGCAGTTGTATTTTCTTCAATTGTGCCAAAGGTTGTGCAAAGCGGTTTTGAGGGAATTTTCCTTGTTGCGACAAACCCCGTT | Klebsiella phage KP34 (NCBI:txid674081) |
| 150 | ACCTCCGCAGCTACCACCTGCCACTGGTCCGGCTCTACTCATCAGTTCCCGGAGCATCGTATCAACATCATCGACACCCCGGGCCACGTTGACTTTACCGTAGAGGTAGAGCGCTCCCTGCGTGTACTGGACGGTTCCGTAACCGTATTC | Klebsiella phage KP34 (NCBI:txid674081) |
| 151 | TTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCG | Klebsiella phage KP34 (NCBI:txid674081) |
| 152 | TGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAA | Klebsiella phage KP34 (NCBI:txid674081) |
| 153 | TGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAA | Klebsiella phage KP34 (NCBI:txid674081) |
| 154 | ATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACAT | Klebsiella phage KP34 (NCBI:txid674081) |
| 155 | ATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACAT | Klebsiella phage KP34 (NCBI:txid674081) |
| 156 | ACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTTTACCTAATTTCTGTAAG | Klebsiella phage KP34 (NCBI:txid674081) |
| 157 | CTTCTTTGCTCTGTTCCTGCAACATTGCAGTCAATTCACTGTTTCCTTTTGTCATACCCTGAATAATTGTTGATGTAAACTTTTTAATCTCCGGAATGATGCAACGAGAACCAAAATTAAACATCGCGTCAATCTCAGAAACACCGTTAT | Klebsiella phage KP34 (NCBI:txid674081) |
| 158 | ATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATC | Klebsiella phage KP34 (NCBI:txid674081) |
| 159 | CAGGTAAAAGTTCCGTATTTCGCACATACTCAGGTAAAAAAAGTAAAAGAAATTCAAAAGTATATGGTTTCCAATTTGCGCCAGCATTACACTCTTGAACAATTGTCCGAGCAATTTGACATTCCTCTTACTTCCATGAAAGCATGTTTT | Klebsiella phage KP34 (NCBI:txid674081) |
| 160 | CTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATC | Klebsiella phage KP34 (NCBI:txid674081) |
| 161 | GTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATA | Klebsiella phage KP34 (NCBI:txid674081) |
| 162 | TTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAG | Klebsiella phage KP34 (NCBI:txid674081) |
| 163 | TGTTCCTTTGAATTTATTGTAGCAGGAAGAACCGATCACACCGGCGATGATACCGATGAACGGGTTCGCAATTTTGCTGAATGCAATGTAAGCAGTTGTACCTTCTTCAAGAGTTTTGATAACAGATACAGCACCTACAGACAGCAGAGT | Klebsiella phage KP34 (NCBI:txid674081) |
| 164 | ACACTGTCCGGACCTTCATTGGTCGCAACAAACTGTTTTTTAGTTCGATATAGGTTTTCTGATATTCTGTCATTGGTCGCACCTCCCATCAATCCCACCAGAAATACCAGACGGTGGACTGCCACAGGACATCTGCCAAGGAGCCAATG | Klebsiella phage KP34 (NCBI:txid674081) |
| 165 | CTTCAAGAGTTTTGATAACAGATACAGCACCTACAGACAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTAT | Klebsiella phage KP34 (NCBI:txid674081) |
| 166 | GACGATTCTCATGGCCACCTGCATCTTCTATGAATTCCTGCTTGCGAAAAAGTATCCGAGGGACAAACGGTTCTGCCATAATGCGCTGGTCTTTGCACCGGATAAGACCGTCCTCCAGTCGCTCCGTGAGATCATGACCTTTGATAAAAC | Klebsiella phage KP34 (NCBI:txid674081) |
| 167 | CTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGA | Klebsiella phage KP34 (NCBI:txid674081) |
| 168 | CGCTGAAGAATGACAGCCAATCCGGCAGCTTTGTTCCTTTGAATTTATTGTAGCAGGAAGAACCGATCACACCGGCGATGATACCGATGAACGGGTTCGCAATTTTGCTGAATGCAATGTAAGCAGTTGTACCTTCTTCAAGAGTTTTGA | Klebsiella phage KP34 (NCBI:txid674081) |
| 169 | GGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGA | Klebsiella phage KP34 (NCBI:txid674081) |
| 170 | GGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGA | Klebsiella phage KP34 (NCBI:txid674081) |
| 171 | AAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAG | Klebsiella phage KP34 (NCBI:txid674081) |
| 172 | ACACTTGTTGAGCGCCGCAGCGGTTGCGTCATTCCAGCTTCCATATTGGTTGAGCTGGCCGATACCCATCGACTGGCCCACCAGATCCTCGGCCTGCTGGGCAATAAACTCCGCTCGCGATGCATGGTCAACAAGCTCCTTGAGCGCCGC | Klebsiella phage KP34 (NCBI:txid674081) |
| 173 | AACAATAATTGCAGCAGTAATTGCAGCAGTAGCATCGTTAGTATCGGCAGGAATTGCATTGTATAATGCAAAAGATTCAAAAAAAGGAGCGGCACAAAGAGATGAAGTGAATAATAGAACAAATAGTGAAATAGCTATGTTTGAGCAAGA | Klebsiella phage KP34 (NCBI:txid674081) |
| 174 | TTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACG | Klebsiella phage KP34 (NCBI:txid674081) |
| 175 | ATCGGCGCTGTGCCTTCTCCGGAACCCTACCAGAAGCGCACCGCTCACGGCATGATCCTGGGCCTGAACCCCCACAGCTTTGTCAACCTGCCCGCTGAGGAGCAGGAAAAGCTGCTGAAGGAGTACGGCAGCCAGAAGGCCGCTGAAAAG | Klebsiella phage KP34 (NCBI:txid674081) |
| 176 | GAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACG | Klebsiella phage KP34 (NCBI:txid674081) |
| 177 | AAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCA | Klebsiella phage KP34 (NCBI:txid674081) |
| 178 | ACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGC | Klebsiella phage KP34 (NCBI:txid674081) |
| 179 | ACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGC | Klebsiella phage KP34 (NCBI:txid674081) |
| 180 | AACATTGAAGAAATCAAAATGATTGATTTTTCCGATCTGAGCAATATGCGAAAGTTCGGATAATCTTAATTTAACAAATCAAATAAGTCCCCATCCCACTGTTTTAATTGCAAAAAACAGTGAATGGGGACTTTTTTCGATTATTCTATT | Klebsiella phage KP34 (NCBI:txid674081) |
| 181 | AAACTGATGACCCTCTAAGTCCTTGATAACGTCGGCAAGATGACCGATAGCCGTGTGCTCGGGCGGCTGTGAGGAGTGTCCGCCCTTTGCGTTGACGCTTATTTCATAGTTTATGCTTCCCTTTTCGGCAATTCCCACGCCTGCAAGATT | Klebsiella phage KP34 (NCBI:txid674081) |
| 182 | CGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGA | Klebsiella phage KP34 (NCBI:txid674081) |
| 183 | TAGCAGGAAGAACCGATCACACCGGCGATGATACCGATGAACGGGTTCGCAATTTTGCTGAATGCAATGTAAGCAGTTGTACCTTCTTCAAGAGTTTTGATAACAGATACAGCACCTACAGACAGCAGAGTTGTCAGCATCAGCCAAGAA | Klebsiella phage KP34 (NCBI:txid674081) |
| 184 | GAAATTCTTACAGAAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTA | Klebsiella phage KP34 (NCBI:txid674081) |
| 185 | CAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAA | Klebsiella phage KP34 (NCBI:txid674081) |
| 186 | GAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTT | Klebsiella phage KP34 (NCBI:txid674081) |
| 187 | GAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTT | Klebsiella phage KP34 (NCBI:txid674081) |
| 188 | AGGTAAGAGTACCACTAGCTGCAATCTTGCCATCCACTTCAGCCTTTGCTTCAACCACGGCAATGGTACCGCGACGTTTTACAAACGTTGCCGTCATAACTAATTGATCACCTGGTACAACTTGCTTCTTGAATTTAACCTTGTCCATAC | Klebsiella phage KP34 (NCBI:txid674081) |
| 189 | GTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCT | Klebsiella phage KP34 (NCBI:txid674081) |
| 190 | GTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCT | Klebsiella phage KP34 (NCBI:txid674081) |
| 191 | CTGGCATCCGGCGGCACCTCTGTGGCTGCGCTGTTCATGGCCGGTTACATTCCCGGCATCATCTGGGCGCTGTGCTGCTGCGTGGTGGGCGTGCTGCTGGCGGTCAAGCTGGGCTATAAAGGCACCCCCGGCAAGTTTGACTGGAAAAAT | Klebsiella phage KP34 (NCBI:txid674081) |
| 192 | GATTGCCAGCCCTAGCGGTCTGAAATCAAAAGTATGTGTGTCTTTTGTTTTTCTCATTTTAAATCACCTAGCATGAGTTTATACTTCATGTTATAAATTTAGAATGTGTTAAAATTGCATATAGTATGCAGTAATAATTCAAAAGCTGTT | Klebsiella phage KP34 (NCBI:txid674081) |
| 193 | CGACGCTTCTTTTTCAATGGCGCACAGCTGGCTTTCGTAGCGGTTGGTCAGGATGGCGATGTCGCCTTTTTCCACGGTATTTTCCAGCATTTCGGGCGTAGCGGTGCCGATGCGGATGTGGCCGGTGGTGCACACGTCGTCCTCGCGGCC | Klebsiella phage KP34 (NCBI:txid674081) |
| 194 | ATTCTATCATTGGGAAGAATTGTGCCGGAAAAAGGGCTTCAGTATTTAATTAAGGCATTTAGGGAATGTTCAAGTGATAGGAAACTTGTAATCGCTGGAGGATCGGAGTCGAACAAGGATTACTATAATCAGCTCCTTGCATTAGCAGAG | Klebsiella phage KP34 (NCBI:txid674081) |
| 195 | AACAGATACAGCACCTACAGACAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACC | Klebsiella phage KP34 (NCBI:txid674081) |
| 196 | TTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCA | Klebsiella phage KP34 (NCBI:txid674081) |
| 197 | TTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCA | Klebsiella phage KP34 (NCBI:txid674081) |
| 198 | ACTTCTATATCTGTATTCCCAAGTTTACTGTATTCCATAGTCCACTCCTTATTTTAACCTGGCATACTCTTTTTCTGATACTGCCTCGCACCACTCATTGGAACAGTCTACCCCGGGTACCTCAATTGCCAGATGGGAAAACCACTCATC | Klebsiella phage KP34 (NCBI:txid674081) |
| 199 | GTACCCGGTTGAATATGCGTGCAGCGAGAAGATTGCGCATTATATAAAAATGCGCTATGATCACGAAATTACAGATGAGGAGACGGCTTATCTGGCCGTACATATCCGCAGAATCCAGCCAGAAAAAGAGCTGGAGGAGGATTCTCATTA | Klebsiella phage KP34 (NCBI:txid674081) |
| 200 | GATGAACGGGTTCGCAATTTTGCTGAATGCAATGTAAGCAGTTGTACCTTCTTCAAGAGTTTTGATAACAGATACAGCACCTACAGACAGCAGAGTTGTCAGCATCAGCCAAGAAGCCAGAGCTGCAAGTCCACCTGTACCGTCGTTGTC | Klebsiella phage KP34 (NCBI:txid674081) |
| 201 | TTACCTTGTGAAATTTTGCAATTCTTTTCATTGTCTCTTCCTTTTCTGTTCGGTTTTATGCATCATGCAGCACAATTTTTCCTGTTTTCAGACTGTCCTGCACATCGATCACTTTCTGATTCGCGCTGCCCTTCCAGTGCAGCTTCGTAT | Klebsiella phage KP34 (NCBI:txid674081) |
| 202 | CGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATAC | Klebsiella phage KP34 (NCBI:txid674081) |
| 203 | GTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAA | Klebsiella phage KP34 (NCBI:txid674081) |
| 204 | CCTACAGACAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAA | Klebsiella phage KP34 (NCBI:txid674081) |
| 205 | CGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAA | Klebsiella phage KP34 (NCBI:txid674081) |
| 206 | ACTGTAAACTGAATATATTTGTCTGTCCAGATAATTCCATAAGTATGATATGCCTCAGCACTCTTAGCCTGTGTTAAACCTGTTGCATAGTTTTTAATACCATGTGACCATGACTGGTTGTTGAATCTTTCGCAATGGATAGTCTGTGGG | Klebsiella phage KP34 (NCBI:txid674081) |
| 207 | GGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTGGCTTCTTGGCTG | Klebsiella phage KP34 (NCBI:txid674081) |
| 208 | GCCTTGGCGTGGAGAGCATCATGGCAGGCATCATCGTCAACACCGGCCTTTACACCATCAATCTGGCCGTGATGGGCTTTTCCTCCACCATGTCGCTGGTCAAGACCGACACTGTTTTCTCGCTGGCAAAGGGCTCACTGAGCTTCCTTG | Klebsiella phage KP34 (NCBI:txid674081) |
| 209 | AGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAAC | Klebsiella phage KP34 (NCBI:txid674081) |
| 210 | ACCATGAAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGA | Klebsiella phage KP34 (NCBI:txid674081) |
| 211 | CCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTTTACCT | Klebsiella phage KP34 (NCBI:txid674081) |
| 212 | AAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCA | Klebsiella phage KP34 (NCBI:txid674081) |
| 213 | GCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTTTACCTAATTTC | Klebsiella phage KP34 (NCBI:txid674081) |
| 214 | TGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGT | Klebsiella phage KP34 (NCBI:txid674081) |
| 215 | CCGTTATCTGAAAGAAATCAAAGCGTGATTTTGGATACATTTTAAAAGATAAGTACAAAAAATAAGAAGTGGGAGTCTTCACCGACTAAGATAAAAAGATACTGAACAGTTGTAGAAGGAGATAAAGAAAATGGGACAGATCAAAGTTGA | Klebsiella phage KP34 (NCBI:txid674081) |
| 216 | AGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTGGCTTCTTGGCT | Klebsiella phage KP34 (NCBI:txid674081) |
| 217 | AGCGGACTTGGCATGAACAACATGGTAATCACGATGTATGCGGATAATGACACGATCGTGAATGGGGGAAAGGTATGTAATATGAAGGAAATACGCGGTACAGAAGTGTACCGCTATTTTCAGGATAACGGATTGAATAAGGGATTATAT | Klebsiella phage KP34 (NCBI:txid674081) |
| 218 | ATTAAGTAAGAGAAGGAGGAAAAAAGTAATTATGATGAAATTCTTACAGAAATTAGGTAAAGCTTTGATGTTACCTGTTGCTTGCCTTCCTATCTGTGGTATCCTGATGGGTCTTGGATATGCTCTTTGCCCATCTACCATGCAGGGTGG | Klebsiella phage KP34 (NCBI:txid674081) |
| 219 | CCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTTTACCTAAT | Klebsiella phage KP34 (NCBI:txid674081) |
| 220 | CTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACG | Klebsiella phage KP34 (NCBI:txid674081) |
| 221 | CCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTAGATCGGA | Klebsiella phage KP34 (NCBI:txid674081) |
| 222 | AATGGTAGTAAGGTTATTGAGTCTGAGGTGGATACGACCAGTCAATCGATGAGCCTATCAAGTAGCCTTTCGGAGAGCACATCAGAAGTGGTATCAGAATCCATCTCTCTCTCTGAAGAAGCTTCCGCTTCCTTATCGGAAGAAGCCTCA | Klebsiella phage KP34 (NCBI:txid674081) |
| 223 | CTGATCCGTTTTCAGGGAATTTTTGAGCATTCGGCGGTCCAGTCCAAGACCGTCCAGGAATATATGTTTATGCTGATATTCATTATTCCGGGATATTTACTTCTTTATCAGGCCTTTGATCTTTATACACCGATGCGAATGCAGGGGCGT | Klebsiella phage KP34 (NCBI:txid674081) |
| 224 | GTGACGGAGCAAGGCCCGGGCGGCAAGATGATGCGCATCAGCCCCAAGGTGGATCACAAGTTGGGCTCGGCAAGCGCGGTTTACGATTCGGCCAGCGGGTGCTATCGCTGTGGGTGGCAGATCGAAAACGGCGACCAAATCACAGTCACC | Klebsiella phage KP34 (NCBI:txid674081) |
| 225 | CAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACC | Klebsiella phage KP34 (NCBI:txid674081) |
| 226 | CCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAG | Klebsiella phage KP34 (NCBI:txid674081) |
| 227 | ACCTCGCCCTTCACGCCGATGATATCGCCCACGTCCAGCTTCTTGAAGGCGGCATAAGGCTCTTCGCCCAGCTCATCGCGGCGGACGTACAGCTGAATGTCGCCCTTATCGTCCCGCAGGTGGGCAAAGCTTGCCTTGCCCATTACGCGC | Klebsiella phage KP34 (NCBI:txid674081) |
| 228 | TATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGAC | Klebsiella phage KP34 (NCBI:txid674081) |
| 229 | TATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGAC | Klebsiella phage KP34 (NCBI:txid674081) |
| 230 | TATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGAC | Klebsiella phage KP34 (NCBI:txid674081) |
| 231 | ACGACTCCGCAAACCGCGGCCACTTCTACAACAGGTTTTCTCTGCTTATGAGTCGTTTCTCGTCATTGCCAGCTCGGTTTTGATTGTGTTTGTCCGTCCGTTTTCCGACTTGCTGATTAGTACCAGCACTTACACAGAATATGCGGATG | Klebsiella phage KP34 (NCBI:txid674081) |
| 232 | GTTAGTGCGCCCTGTGGTCCTTTTAATAAGTATTATGGGTGTGAAAAAGGAATAATGTCGCCGCTTTACACCGTTTATAAATATATCGATTGTTCTACGGATTATACCGAATACCTTTCGTACTTCTTTTCTTCCGCAAATTGGCATCGC | Klebsiella phage KP34 (NCBI:txid674081) |
| 233 | TGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGT | Klebsiella phage KP34 (NCBI:txid674081) |
| 234 | CCCGTTGAAGCAGTCCACTGCCAGTTCTTTGTAGAAATCTTGCACAGTGCCCACACTGCCATTATAGGAGCCTTAAGTAAAACCTCCAGTCCCATAACAATCAGCATCTGTACCTGTGTAATATCATTTGTAGAACGTGTAATTAAACTT | Klebsiella phage KP34 (NCBI:txid674081) |
| 235 | TCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAAAGCTTTACC | Klebsiella phage KP34 (NCBI:txid674081) |
| 236 | AAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCAT | Klebsiella phage KP34 (NCBI:txid674081) |
| 237 | TGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTG | Klebsiella phage KP34 (NCBI:txid674081) |
| 238 | TGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTG | Klebsiella phage KP34 (NCBI:txid674081) |
| 239 | AGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCA | Klebsiella phage KP34 (NCBI:txid674081) |
| 240 | GCACCGATCTTCAGTTTCCGGATGATCTTGTTTTTCTTTTTTGATGCACCTTCCCCCACATAGAGATATGGATACCATTTCATATTTTTTCCTTTCTCCTGCCATCGAATTCTTGTTCTATTTTACCAGACACAGAAACATAATGCAACG | Klebsiella phage KP34 (NCBI:txid674081) |
| 241 | CTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTG | Klebsiella phage KP34 (NCBI:txid674081) |
| 242 | GCTGTCTTCGCATGGTTATCCACCAACAAGAGCAGGGGCAGAACAGGCATATCAGGATTACATGGATGGAAAGATTGATGTTCCAGAA | Klebsiella phage KP34 (NCBI:txid674081) |
| 243 | TTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAAC | Klebsiella phage KP34 (NCBI:txid674081) |
| 244 | GGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCTGTATC | Klebsiella phage KP34 (NCBI:txid674081) |
| 245 | GTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAG | Klebsiella phage KP34 (NCBI:txid674081) |
| 246 | GATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAACATCAA | Klebsiella phage KP34 (NCBI:txid674081) |
| 247 | AGAAGCTGTTAAGATGATGGCAAGAATCGCTGAACGTACAGAGAAGGAAATCAACTACCGCAAACGTTTCAATGATATGGCTAAATACACTGACCCAGGTATCACAGACGCTATCTGCCATGCTACCTGCAGCACAGCTTACGACCTCGA | Klebsiella phage KP34 (NCBI:txid674081) |
| 248 | TGATACCGATGAACGGGTTCGCAATTTTGCTGAATGCAATGTAAGCAGTTGTACCTTCTTCAAGAGTTTTGATAACAGATACAGCACCTACAGACAGCAGAGTTGTCAGCATCAGCCAAGAAGCCAGAGCTGCAAGTCCACCTGTACCGT | Klebsiella phage KP34 (NCBI:txid674081) |
| 249 | CTTCTTCAAGAGTTTTGATAACAGATACAGCACCTACAGACAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGT | Klebsiella phage KP34 (NCBI:txid674081) |
| 250 | CAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAGATCGGAAGAGCACACGTCTGAACTC | Klebsiella phage KP34 (NCBI:txid674081) |
| 251 | GCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTA | Klebsiella phage KP34 (NCBI:txid674081) |
| 252 | CCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAG | Klebsiella phage KP34 (NCBI:txid674081) |
| 253 | CAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAATGCAAGCAACAGGTAACATCAAAGC | Klebsiella phage KP34 (NCBI:txid674081) |
| 254 | TACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTG | Klebsiella phage KP34 (NCBI:txid674081) |
| 255 | GCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACA | Klebsiella phage KP34 (NCBI:txid674081) |
| 256 | AAATGCGCTATGATCACGAAATTACAGATGAGGAGACGGCTTATCTGGCCGTACATATCCGCAGAATCCAGCCAGAAAAAGAGCTGGAGGAGGATTCTCATTAAGTAAGAGAAGGAGGAAAAAAGTAATTATGATGAAATTCTTACAGAA | Klebsiella phage KP34 (NCBI:txid674081) |
| 257 | CTTCCGATAAACGAATATATTTGATTATCCGCCAAAAATTTGTTGAATTCTCGGCGGATTTGCGTTATTATTACATAGGTAACCGCTTGAATATCAAAACGGAAATTTTACCACGGTAAGGCTGTGAAATTGCGGCCGTATGCTTTAGGA | Klebsiella phage KP34 (NCBI:txid674081) |
| 258 | CGGGTCATGATTCTGGACGAGGCGACCTCCTCCGTCGATACCCGAACCGAAATCCAGATTCAAAAGGCAATGGATCACCTCATGGAGGGCAGAACCAGCTTCATCATTGCCCATCGGCTTTCGACCATCCGAAATGCAGATTTGATTCTG | Klebsiella phage KP34 (NCBI:txid674081) |
| 259 | CAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACC | Klebsiella phage KP34 (NCBI:txid674081) |
| 260 | CAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACC | Klebsiella phage KP34 (NCBI:txid674081) |
| 261 | TCAGAATATGGATCTGTCTCAATGTCCTTTGGAATGTATGATACCTATTATAATCCCTATACGTATGCATACAGCTACCCAGGAAGTGCAGGCGTGAACCATGCAATAACTCTGATTGGCTGGGATGACAATTATTCCAGGGAGAATTTT | Klebsiella phage KP34 (NCBI:txid674081) |
| 262 | CTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGAC | Klebsiella phage KP34 (NCBI:txid674081) |
| 263 | GGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTG | Klebsiella phage KP34 (NCBI:txid674081) |
| 264 | TGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCT | Klebsiella phage KP34 (NCBI:txid674081) |
| 265 | ATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATA | Klebsiella phage KP34 (NCBI:txid674081) |
| 266 | TGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTAT | Klebsiella phage KP34 (NCBI:txid674081) |
| 267 | TTAAACCCATACAAGGCCACCGCATACAGGAATGCCACAATCGTAAAAATCACCGTATGAGGGAATATCTTCCCTATGAGGCTCACCGTAAGCGAATTTCCACCCATCTGCAACCATTCGCGGGCCGTCGAGTATTTAATCTCGGAACCG | Klebsiella phage KP34 (NCBI:txid674081) |
| 268 | CCGAAAAAAGAGAAAAATACAAAGAAACTGCTTTTTGTGTTATAATACCAGTACATTATGAGCAGGTAATGAACTGTGAAGTATTTTTTCAGTTATGATAAATATACAGTTTTGTTT | Klebsiella phage KP34 (NCBI:txid674081) |
| 269 | CACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGA | Klebsiella phage KP34 (NCBI:txid674081) |
| 270 | CAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCA | Klebsiella phage KP34 (NCBI:txid674081) |
| 271 | TACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGG | Klebsiella phage KP34 (NCBI:txid674081) |
| 272 | TGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTG | Klebsiella phage KP34 (NCBI:txid674081) |
| 273 | ATAGCCGAAAGTCCAGCATCTCCTATGGTATTGCCTACAAAATATCCATCCACTATCGCATATATTCCTGATAATGCAAATGCTATAATAGATGGAATAACATATTTAAAAAATAATGATTTATGATTCATTTTTCTTACAATCTCCTT | Klebsiella phage KP34 (NCBI:txid674081) |
| 274 | GCGCAATCTCAGGAATCAGGACTATTGTCTGTTTTCCCTGGGCGATCGTGCTCTCGATCAATTTCATATATACCTGGGTTTTCCCACTTCCTGTCACGCCCTCAATCAACATTGGACGGGGAAATGGCTTTTCCCACTCTTCCAGAATCT | Klebsiella phage KP34 (NCBI:txid674081) |
| 275 | GGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTG | Klebsiella phage KP34 (NCBI:txid674081) |
| 276 | CGTTGAGCGAGGAGATCTGCCCGCGCAGCGGGATGCTGACGAGAAAGTCACACTTTTCCCGCACCAGCCGGCTGATGCCGGTGCCCTCCGACCCGATGACGATGCAGGCCGGGCCGGTCAGATCCGTTTTCCACAGCTCGTTTGCGCCCT | Klebsiella phage KP34 (NCBI:txid674081) |
| 277 | CTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCC | Klebsiella phage KP34 (NCBI:txid674081) |
| 278 | GCGCTCGATGCGCTCTTCGCCGAATGCTTCGGATACTTCAACGTGACCGACCTGCCATACGTGTCGATGGACTACTACGTGAACAACGATTACGTGCAGTCCGACAACGAATGGATTCACCTGTACAACCACCAGTGGCGCAACCAGGTG | Klebsiella phage KP34 (NCBI:txid674081) |
| 279 | TGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCG | Klebsiella phage KP34 (NCBI:txid674081) |
| 280 | ATTCTCAGCGTCTGTGATATTCTCGAATCCTGTTACCTTTGTCTCTCCGTCCTTTGGTTTGATCAGAACGACTTTGTTCTCAAGAAGATCCTTAATGGTGTCTTCCTTTGTCAGTTTTTCCTCTGTGAGGGCATCCATCTGTGTATCTGC | Klebsiella phage KP34 (NCBI:txid674081) |
| 281 | CCTACAGACAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAA | Klebsiella phage KP34 (NCBI:txid674081) |
| 282 | AGGAGGAAAAAAGTAATTATGATGAAATTCTTACAGAAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGT | Klebsiella phage KP34 (NCBI:txid674081) |
| 283 | TATCCCGCACCTGCTGTACTACTGGCGCTCCAGCCCCACCAGCGTGGCAAGCAACATTTCGGCCAAGACCTACTGTCTGGAAGCTGCGGTCAAGGCCCTGTATGCCCACTACGAGCGTGTGGGAGATCGGAAGAGCGTCGTGTAGGGAAA | Klebsiella phage KP34 (NCBI:txid674081) |
| 284 | AGCGAGAAGATTGCGCATTATATAAAAATGCGCTATGATCACGAAATTACAGATGAGGAGACGGCTTATCTGGCCGTACATATCCGCAGAATCCAGCCAGAAAAAGAGCTGGAGGAGGATTCTCATTAAGTAAGAGAAGGAGGAAAAAAG | Klebsiella phage KP34 (NCBI:txid674081) |
| 285 | CTACAGACAGCAGAGTTGTCAGCATCAGCCAAGAAGCCAGAGCTGCAAGTCCACCTGTACCGTCGTTGTCTTCGGACATACCAACACCGATACCGATTGCAAACAGGATAGCCATGTTATCGATCAGAGCTCCACCTGCTTTCACTAAAT | Klebsiella phage KP34 (NCBI:txid674081) |
| 286 | GCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGC | Klebsiella phage KP34 (NCBI:txid674081) |
| 287 | ATTACCGCTATCAGACAGACATCATGATCATAACGATTGTAATTCTGGTAGTGCTTGTGCAGATCCTGCAGGGACTTGGCATGATGCTCTCCAGGAAGCTGGATAAGCGAAAAGCATAAAAAGATATTTGACAGAAGATTGTAATCACGA | Klebsiella phage KP34 (NCBI:txid674081) |
| 288 | GATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGTATACCACAGAT | Klebsiella phage KP34 (NCBI:txid674081) |
| 289 | TGCAAGTCCACCTGTACCGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTG | Klebsiella phage KP34 (NCBI:txid674081) |
| 290 | CACGATGTCCGAAATCATTCAGTGCGTCGCAGATATGCGCACGCAGGATGTGAAGGCAATCCGCGAGTTCGGCAAGTTCGTCCCCGCCTTTGCCGCCTTTGAGAAGAACCGCCTGAGCCTCTATCGTGAGGCGGCGAAGGAAGCCGAGGA | Klebsiella phage KP34 (NCBI:txid674081) |
| 291 | TCGATCTCCGGCTGCAGCGGCCCAAAGGGCAGATAGAGGGCTGCGTCTGCCTCGCTGCAGAGCATTTCGTGGGGGTTGCGTGACACGGGCCGAGTGTCCACCGCAAACTCATATCCCGGCAGCGCGGCGTGCAGCCGTGCCATCAGGATG | Klebsiella phage KP34 (NCBI:txid674081) |
| 292 | TGGAAACAGTATGATATCGGTAAATGATTTTGCCATGCTGCAGCCGGGAGATATTATCCGGCTGGATCGCAAGGTGGAGGACGAATTGGATATTTATGTTGGAAATATTCGGAAATTTACTGCACTTCCCGGATCATCGGGGGATAAATA | Klebsiella phage KP34 (NCBI:txid674081) |
| 293 | ACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGAGATCGGA | Klebsiella phage KP34 (NCBI:txid674081) |
| 294 | CTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGAC | Klebsiella phage KP34 (NCBI:txid674081) |
| 295 | CTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGAC | Klebsiella phage KP34 (NCBI:txid674081) |
| 296 | GGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATC | Klebsiella phage KP34 (NCBI:txid674081) |
| 297 | GATCTCTTTGAAGAAATTGCTCGTATCTATGGATATGACCGCTTGCCAACCAGCCTTCCAAAAGACGATGGTACAGCAGGTGAATTGACTGCGACACAAAAACTTCGCCGCCAAGTTCGTACCATTGCTGAAGGGGCAGGTTTGACAGAA | Klebsiella phage KP34 (NCBI:txid674081) |
| 298 | CCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAG | Klebsiella phage KP34 (NCBI:txid674081) |
| 299 | GCTCTGGAGAAGATCGTCAAGGGCATCTGCTTCGACAACGCAAAGCGCTACTTCAACCTGTAAGAGAAGTGCCTGAACCTGAATAAGGCATAACACAAAGCCCCCGGAACGGTGTGAAACGTTCCGGGGGCTTTTGCTGTAAGCAGATAA | Klebsiella phage KP34 (NCBI:txid674081) |
| 300 | AAGGGCGATGTGTTCGTGGTGGGCTGCTCGTCCAGCGAGATCGTGGGCGGGCACATCGGCAAGGATTCCAGTCTGGAAGCCGCGTAGGCCGTGTACGCGGGCATTGCCCCAGTGCTGGCTGAAAACGGCATCTGGCTGGCCGCGCAGTGC | Klebsiella phage KP34 (NCBI:txid674081) |
| 301 | CTCCCATGGATCTTCTTTTAAGAAACGAAGGGATGCATCGAATGTAGCACCACCCCAGCACTCAACAGCGTTGTATCCGACTTTATCCATTTTATCAACGATCGGAAGCATCTGTTCTGTTGTCATACGTGTTGCGATCAGGGACTGAT | Klebsiella phage KP34 (NCBI:txid674081) |
| 302 | GCAGGGTGGTGACGTAGTAGGTATTGTACAGCAGATTGGTTTCTATTTAGTGAAAGCAGGTGGAGCTCTGATCGATAACATGTCTATCCTGTTTGCAATCGGTATCGGTGTTGGTATGTCCGAAGACAACGACGGTACAGGTGGACTTGC | Klebsiella phage KP34 (NCBI:txid674081) |
| 303 | GCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGC | Klebsiella phage KP34 (NCBI:txid674081) |
| 304 | AGAGAAGATAAGAATCAGCACCAATGCCATCCAGAACTCGGGAATACAGCTGGATATCGTACCCAGTTTACATAAAATCTGGTCTGCCAGCCGGTTTTCGTACCAGGCACACAGGATTCCCAGAAGCAATGCTCCCGCGAACATAATGAC | Klebsiella phage KP34 (NCBI:txid674081) |
| 305 | CGTCGTTGTCTTCGGACATACCAACACCGATACCGATTGCAAACAGGATAGCCATGTTATCGATCAGAGCTCCACCTGCTTTCACTAAATAGAAACCAATCTGCTGTACAATACCTACTACGTCACCACCCTGCATGGTAGATGGGCAAA | Klebsiella phage KP34 (NCBI:txid674081) |
| 306 | ATGATCACGAAATTACAGATGAGGAGACGGCTTATCTGGCCGTACATATCCGCAGAATCCAGCCAGAAAAAGAGCTGGAGGAGGATTCTCATTAAGTAAGAGAAGGAGGACAAAAGTAATTATGATGAAATTCT | Klebsiella phage KP34 (NCBI:txid674081) |
| 307 | ATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGAT | Klebsiella phage KP34 (NCBI:txid674081) |
| 308 | ACTTCCTATCCTCATAATTTATTTGAGTATTTTGATTATATTTATTACGTTCATAAACCTTTACATTCCCTTTATATTGAAATTCATAACAGTAGTTATACTCTTTTTTATATTCTAAAAAACGTAAATCAGGATTACAATAATCTATTA | Klebsiella phage KP34 (NCBI:txid674081) |
| 309 | ATTCGGAGGACTGCGTGCCGTATCCGAATTTGATATGAATATCGGGAAAGGTCAGCTCTATGGCCTGATCGGACCGAACGGTGCCGGAAAAACAACCATATTTAACTTGCTGACAGGTGTTTACAAACCGGATGAAGGTATTGTAAAATT | Klebsiella phage KP34 (NCBI:txid674081) |
| 310 | CTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGA | Klebsiella phage KP34 (NCBI:txid674081) |
| 311 | GTTATCGATCAGAGCTCCACCTGCTTTCACTAAATAGAAACCAATCTGCTGTACAATACCTACTACGTCACCACCCTGCATGGTAGATGGGCAAAGAGCATATCCAAGACCCATCAGGATACCACAGATAGGAAGGCAAGCAACAGGTAA | Klebsiella phage KP34 (NCBI:txid674081) |
| 312 | CGATCACACCGGCGATGATACCGATGAACGGGTTCGCAATTTTGCTGAATGCAATGTAAGCAGTTGTACCTTCTTCAAGAGTTTTGATAACAGATACAGCACCTACAGACAGCAGAGTTGTCAGCATCAGCCAAGAAGCCAGAGCTGCAA | Klebsiella phage KP34 (NCBI:txid674081) |
| 313 | CCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGT | Klebsiella phage KP34 (NCBI:txid674081) |
| 314 | TACAGAAATTAGGTAAAGCTTTGATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGA | Klebsiella phage KP34 (NCBI:txid674081) |
| 315 | CTCGGCCAGCAGCGCCCACGCAGCCCATGCCGTGGGCACCGCCGCCGCAAGGGCCAGCCCCGGCAGCGCCAGTGCCGGGCATGTATGGAACGCACTGCCCGGCGGCAGGGCTGCCACCGCGCACAAGGCCTGTGCAGCCCAAAGCTCCCA | Klebsiella phage KP34 (NCBI:txid674081) |
| 316 | GAAGCAGATTGCAGATGGTGATTATTCTGTCAGGGTTCCAGTGCATACAGAAGATGAAATTGGAGAACTTTCAGTCAGTTTTAATTATATGACAGAACAATTGATAGAAAAGTTAATGAAATTGGATCAATTGTTAAAAAACCAGGAAGA | Klebsiella phage KP34 (NCBI:txid674081) |
| 317 | GTTCAATGCTTGTTACCTTCTCTACAACCGGTTCCGGCTTTGGCTGTGCATTGACAGGCTTCTTACGCATTGTGTTTCTATTGTTATTGTTATTGTTGTTCTTATTATTGTTCTTGCCGTTTCTGGATGTATTTCTTCCTTCAAAATCTC | Klebsiella phage KP34 (NCBI:txid674081) |
| 318 | TGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGACGGTACAGGTGGACTTGCAGCTCTGGC | Klebsiella phage KP34 (NCBI:txid674081) |
| 319 | CTTTGCCCATCTACCATGCAGGGTGGTGACGTAGTAGGTATTGTACAGCAGATTGGTTTCTATTTAGTGAAAGCAGGTGGAGCTCTGATCGATAACATGGCTATCCTGTTTGCAATCGGTATCGGTGTTGGTATGTCCGAAGACAACGAC | Klebsiella phage KP34 (NCBI:txid674081) |
| 320 | ATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGAT | Klebsiella phage KP34 (NCBI:txid674081) |
| 321 | CCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGAGATCGGAAGAGCGTCGTGGAGGGACA | Klebsiella phage KP34 (NCBI:txid674081) |
| 322 | ATGTTACCTGTTGCTTGCCTTCCTATCTGTGGTATCCTGATGGGTCTTGGATATGCTCTTTGCCCATCTACCATGCAGGGTGGTGACGTAGTAGGTATTGTACAGCAGATTGGTTTCTATTTAGTGAAAGCAGGTGGAGCTCTGATCGAT | Klebsiella phage KP34 (NCBI:txid674081) |
| 323 | CAGGCGAAGAATGTGCCCTGCTGGTTGCAGCCCGCGTAAAATACCTCGCTTTTCTGCCCGATGCCAACGCTGTGCAGCTGGCCTTCCAGCCAGCTTACGCTGCGGCCGGAGGCTTTCAGGTTGTCTTCCATCAGCTGCCCGTCCAGAATC | Klebsiella phage KP34 (NCBI:txid674081) |
| 324 | AGGAATTCTGACGGTAACGGTCGTTCCGACATCAAGCTCACTTTCGATTTTAATGCCGTAACGCTCGCCGAAGTAGATTTTCAAACGGTCGTCAACATTCTTGACACCGATACCCTTTGAGTCGCTCCTGCCCTTTTCAAGCACCTGCC | Klebsiella phage KP34 (NCBI:txid674081) |

List 2: Sequences that mapped to Klebsiella virus KP27 (NCBI:txid129147) which targets bacteria Klebsiella variicola in whole-community metagenomes in donor/pre-FMT samples

| SEQ ID NO | Sequence | Sequence mapped to |
|---|---|---|
| 325 | TTTGTGGCGGGGTGCAGGGGCGGCAGCGCACTGCTGGGGTCAAGGGGCAACGCCCATTGGCAGGTTAAGGGCGGCAGCCATTATCGGGTCAAGGACGAAGTGCCTTGGCAGGTTGAGAGATCGGAAGAGCGTCGTGTAGGGAAAGAGTGT | Klebsiella phage KP27 (NCBI:txid129147) |
| 326 | CCTTTGTGGCGGGGTGCAGGGGCGGCAGCGCACTGCTGGGGTCAAGGGGCAACGCCCATTGGCAGGTTAAGGGCGGCAGCCATTATCGGGTCAAGGACGAAGTGCCTTGGCAGGTTGAGAGATCGGAAGAGCGTCGTGTAGGGAAAGAGT | Klebsiella phage KP27 (NCBI:txid129147) |
| 327 | GGGTGTAGTTGGGGCCGTCCGGCTCAAAGGTGCGGGTGCGCAGGGCGTCGGCAAAACTCTTGCCCTGACTCATCAGGTCGTAGATGGTATCGGTCTGGTCGCCGTTGGTCACAATGTGGGTCTTGCCAAGGGTCAGCACCGGATTATAAA | Klebsiella phage KP27 (NCBI:txid1129147) |
| 328 | CTCAACCTGCCAAGGCACTTCGTCCTTGACCCGATAATGGCTGCCGCCCTTAACCTGCCAATGGGCGTTGCCCCTTGACCCCAGCAGTGCGCTGCCGCCCCTGCACCCCGCCACAAAGGAGATCGGAAGAGCACACGTCTGAACTCCAGT | Klebsiella phage KP27 (NCBI:txid129147) |
| 329 | AAGTACACCACCAACTATTCCAATACCGCTTCCTGCAATCCCACCTTTTATAGCACCACCCACAGTATCTACGAAAGTTTCTTTCCAATTTACCTTTCTATCCAAAATCACCTGTGTTACTATTGACGAAGTAAAAGAAACAACACCACC | Klebsiella phage KP27 (NCBI:txid129147) |
| 330 | GGAAATTTCCAACAATAATTGCAGGGGCAATCAGTGGTGGTGTTGTTTCTTTTACTTCGTCAATAGTAACACAGGTGATTTTGGATAGAAAGGTAAATTGGAAAGAAACTTTCGTAGATACTGTGGGTGGTGCTATAAAAGGTGGGATTG | Klebsiella phage KP27 (NCBI:txid129147) |
| 331 | TTACTGAGTATCCAAAAACACTTAACAGATATAATTATTGTTGGAATAATTCCTTAATATATGTGGATTATGATGGGAAATTTCCAACAATAATTGCAGGGGCAATCAGTGGTGGTGTTGTTTCTTTTACTTCGTCAATAGTAACACAGG | Klebsiella phage KP27 (NCBI:txid1129147) |
| 332 | GCCCCTGTAGCACCTGTAGCAAGTACACCACCAACTATTCCAATACCGCTTCCTGCAATCCCACCTTTTATAGCACCACCCACAGTATCTACGAAAGTTTCTTTCCAATTTACCTTTCTATCCAAAATCACCTGTGTTACTATTGACGAA | Klebsiella phage KP27 (NCBI:txid129147) |

List 3: Sequences mapped to Klebsiella phage vB_KpnP_ KpV289 (NCBI:txid1671396) which targets bacteria Klebsiella pneumoniae in bulk DNA metagenome sequences in donor/pre-FMT samples

| | | |
|---|---|---|
| SEQ ID NO | Sequence | Sequence mapped to |
| 333 | GACTTTATCTTTACCACCTTCTGCTGATTCAAGAGTTTTAGCATTTTGATCAGCTTTCAGGTCAATTGTTTTCTTTTTGCTTTCACCAAGAGCTTTCATTTCGATAATCATCTTAGCTTTTGTATCTTTAATTTCAATTGTCATTGTCAA | Klebsiella phage vB_KpnP_KpV289 (NCBI:txid1671396) |
| 334 | AGAGTTTTAGCATTTTGATCAGCTTTCAGGTCAATTGTTTTCTTTTTGCTTTCACCAAGAGCTTTCATTTCGATAATCATCTTAGCTTTTGTATCTTTAATTTCAATTGTCATTGTCAAATCAAATTGATCAATCACCTGCTCAAGTTGT | Klebsiella phage vB_KpnP_KpV289 (NCBI:txid1671396) |

List 4: Sequence mapped to Klebsiella phage KLPN1 (NCBI:txid1647408) which targets bacteria Klebsiella pneumoniae in bulk DNA metagenome sequences in donor/pre-FMT samples

| SEQ ID NO | Sequence | Sequence mapped to |
|---|---|---|
| 335 | AAAGGTTACCTCGCTGCCAGCTTTTGCATATGGATTGTTCAAGATTGCACTAAGGTCTACTTTACCTTTGTGCGGCTCATCAACTGCATCTGTAGCAACCAGGAGATCCATACGTCCTACCATCTCTTTCAGGCTTCTCACACCCAGTTT | Klebsiella phage KLPN1 (NCBI:txid1647408) |

List 5: Sequences mapped to Escherichia virus 186 (NCBI:txid29252) which targets bacteria Escherichia coli in bulk DNA metagenome sequences in donor/pre-FMT samples

| SEQ ID NO | Sequence | Sequence mapped to |
|---|---|---|
| 336 | AGCAGCGCATCCTGAGAGCGGCACGGGTGCCAGCGCTTGCCATCCTTCCCCATAATCCAGCCATGACCGCAGTGCATTGCAGGACTTTGCTTAACGAGCAGTGATGCAAAAGATGGTTCTTTAGTCAGCATAACCACCTCAGATCAGACC | Escherichia virus 186 (NCBI:txid29252) |
| 337 | GCGTCCACATGAAATTCAAATTTATCCTTGAAACGAATATTCTGGATGGCCAGATAGCTGGAAGCATGTTCCAGTTCTTTTTCCAGAGTGATCATATCTTTTCCCTGACTTAAGGAGATCCGGAAAAATTTAGCCAGAAGAGAGACCATC | Escherichia virus 186 (NCBI:txid29252) |
| 338 | GTCATCGTCGCGATCGATGCAAGTGAGAACATTTTATTCTTCCAGATATTCTTCAGTCCCTGCATTATCGTGTAGAAAAATGTACTAATCCTCATCGCTATAATCACCCTCGCTGTCCTCAACGACAACGC | Escherichia virus 186 (NCBI:txid29252) |
| 339 | TAGAAAGCATCCTGCACCACGCGGCGCTTGACGCTCTCCGTCCAGCTTTCGTCCCAGCGGTCAACGGAAAACGCCCACGCCAGATATGGCAGAAAGCTGACCGGACACGTTGCCGGGTTCCACAAGTCGCGTAGCGGCACCTGCAGATCG | Escherichia virus 186 (NCBI:txid29252) |
| 340 | GATCCAAACATCAATGGGGACAATGGTGTCGCTGCCGCCCCAGACGCTGTAGAGATCGTGGAAAATACCGCCGCCGACACCGCACGCGCCGTAGGAAACACAGATTTTATGATCGGGGGCAGACTCATACGCCCGAAGTGCAGGCATACG | Escherichia virus 186 (NCBI:txid29252) |
| 341 | TCTTCTTTCCACCCATATTGTGTCGGATGTGGAACAGATTGCAGATAAAATTCTGATGATGAAAGAGGGGCAGCTTATTTTTGAAGGGACCAGACAGGAAACAGGAAGTGATCTGGAAAAGCTTTATCTTGAAAAATTTGGGGAGGTGAA | Escherichia virus 186 (NCBI:txid29252) |
| 342 | TCGAAGAAACCAGCCAGGCATTTTCCGCCCTGAAAAACACCCTCGACAAAACCGAAAGTTTCAGCCAGCCGCGACGTACAAAAGCCAGTGGCGGTGGTGGCGACGAGCTGCTGACCGACTGCTGATAAACCGCAGACCGAAACCGGGCGG | Escherichia virus 186 (NCBI:txid29252) |
| 343 | TCCCTGATGTGGATCCGAAGCGGGTGCGCGAGCTGATGCGCCTTGAGCAGACGGTTTCCGATGCGCGCCTGCGCAACGCCATCAAGACCGGCATGGCGGAAACCAATGCAGAGCTTTACGACTACCGGCTGCGCCAGATTGCCGCCGGGT | Escherichia virus 186 (NCBI:txid29252) |
| 344 | ATCAGGCAGGCCACCGACAGACCATTCGACGGTGAGCGCGTCATCGTCGAGGCCGAGGTCAATCGCCGCCGCGCCATTCATGCCGCCGCCGCGATAGTTTTCGAGCTTGCGGGTCAGCTTCGGCAGCGTCACGGATTCAACAACGCCCAT | Escherichia virus 186 (NCBI:txid29252) |
| 345 | CCACCGCAACAGCCTACGCCACCGCGCGCGCACTCGGCCTGCGTGCCTATATCGACCAGACCGTCGGCTGGCACAAAACCCTGTCTAACGTCGGCGTGCAGGGTGTTACCGGCATCAGTGCGTCAGTGTTTTGGGATTTGCAGGCATCCG | Escherichia virus 186 (NCBI:txid29252) |
| 346 | CGAACTGAGAAGCGATAAAGCTAAAAAAGCACGCTTTCATGCCGAGGCCATCGACCCGGAAAAGGGCAGCGCTACCGGCTATGTCGCTAAATACATTTCCAAAAATATTGACGGTTATGCACTTGATGGCGAGCTCGACGACGAAAGCGG | Escherichia virus 186 (NCBI:txid29252) |
| 347 | TGGTCAGCACCATTGCCAAGCGCAATAAACTGACGGCCAGTGTCGCGGATTCACTGAAAAAAAAACCGGTACCGCATATAGACCCGTCGCAGGAGACC | Escherichia virus 186 (NCBI:txid29252) |
| 348 | TGGTCAGCACCATTGCAAAGCGCAATAAACTGACGGCCAGTGTCGCGGATTCACTGAAAAAAATACCGGTACCGCATATCGACCAGTCGCAGGAGTCCGACGCCGTATTTCTGACCCGGCTCGCTGACCGCAACGGGGCGGCGGTGTCAG | Escherichia virus 186 (NCBI:txid29252) |
| 349 | TGATGTCGGGCGTGCTGCTGCTGCCCGTGCGGACCGGAACGGGGGCTTTCTACCGCAAGCGGATCGGCCGCGTCCTCCTGGCGCTGGTCTTCTGGTCGCTGACGCTGCCCGTGCTCTACTACCTGTATATGCGCTACGTCGGGACCTC | Escherichia virus 186 (NCBI:txid29252) |
| 350 | TTCTACGCTCCCGGCGGAAAACCGGACGGCATCAAAATCGTGCCACTGAGTGAGGTCGCCACAAAGGATGACTTTTTCAACATCAAGAAAGCCAGCGCCGCCGACCTGATGGACGCGCACCGCGTACCGTTCCAGCTCATGGGCGGCAAG | Escherichia virus 186 (NCBI:txid29252) |
| 351 | CAGCGACGATGCGCGCCTGAATGATGTGCATGAGGCGGTGACCACCGTCAGCGAACATGTGCAGACCAACCTGACCAAGCAAGACGAGCGCCTTTCCGCTATGGAAACCGCGTTTGCCACCTTCAAACAGGAGCTGACCGGCAAGGTCGA | Escherichia virus 186 (NCBI:txid29252) |
| 352 | TGCATCGACTGATAAGGCAGGGTGCGACGTTCAAACACAAACATTCCAAGAGCAAGCATCATCATCAGCCTCCTTAATCGTGCATCATGCTAGCGCGGGCTTTGGCTCGCTTGTCGCGTTCATATTTTTCTAACGCATCCTGCAACTGGT | Escherichia virus 186 (NCBI:txid29252) |
| 353 | AGAAAGGTGCTTTCTCTCCAGCCTGAGTGCGTGCGTTTTGATGTTTATCGCACTGCTGCGGTACTGGAGCAAAATCAGGGCAGTCAACGAGCCAATGCCTTTTTTATCAGCTTTTGCAAAAATGCATTGCCCCGTCTTGAACTGGTCGCC | Escherichia virus 186 (NCBI:txid29252) |
| 354 | CGAAAGCGGTATCAAGCTCACCACCGATTGCCAGCAGCAGAACCTGCTCACCGACCGAGGGAGCCCACCACGTCCGCGAACGACCGGCGCGGGTGGTCAGCCAGTTCAGCCATGTTGTCTGGATCCCGCCGCTTTGTACGCGGCACAGCC | Escherichia virus 186 (NCBI:txid29252) |
| 355 | GCCCGACTTTTCCAGCTTGCTGGTGATAGGTTCCAGATGCTTGGGGATGACATTTTTGATGAGCACGTTGCCGCCTGTGATCGTGGCGGCGACCATGTAGCTGCCGGCCTCGATCTGATCCGGGATGATGGAATAGGTGCAGCCGTGCAT | Escherichia virus 186 (NCBI:txid29252) |
| 356 | CTTCTAAAAATTTCAAAATTCCCTGATAAATCTCTCCGGAATCAGAATATTTCTGCTCTGCCACTTCCTCTGACAGCTTGGATGTATCGATCCAATTCTTCGAAATGGCATAATTTAAATACTGGTTCAGATTGATCGTTTCCTCTTTTG | Escherichia virus 186 (NCBI:txid29252) |
| 357 | CGAAAGCGGTATCAAGCTCACCACCGATTGCCAGCAGCAGAACCTGCTCACCGACCGAGGGAGCCCACCACGTCCGCGAACGACCGGCGCGGGTGGTCAGCCAGTTCAGCCATGTTGTCTGGATCCCGCCGCTTTGTACGCGGCACAGCC | Escherichia virus 186 (NCBI:txid29252) |
| 358 | GGTGCTTCATATGGAGCATGGCGGTGGAAATAATTCTACGTTTACTACCCGTGTAGTAACTTCTGCAGGATCCGACACCTATTCAGTTATTGCTGCGGCACTTTCTTCTCTGAAAGGTCCGAAACATGGCGGTGCCAATATCAAAGTAGT | Escherichia virus 186 (NCBI:txid29252) |
| 359 | AAAACCAACCAACAAACTCAACGTATATCCACAGTACGGAGCCAGATAAAGTCCGAAAGGGAAATAAATAATAGCTCCGGCAATAGATTGGGTGATATAATTAGTCAGACTCATCCTACCATAATAACGAAGATTTGATACAAAATTCTT | Escherichia virus 186 (NCBI:txid29252) |
| 360 | GGTACGCATCCCGGTAATATCGACATACATTTCGCCAGCGTCGCCGGTCTCAAAGCTGATGGCGGTAAGCCGGATGCGCGGCTCCCACTTCTGGATAGCCGAATAGCACGCCACCATAATTTGCAGCCTGAGCGCCGGGTTTTGCGGCAT | Escherichia virus 186 (NCBI:txid29252) |
| 361 | ATACGCAGGCGGTTTGCCGACCCGGTCATTAGACGCCCAGCGGTAATTCTTCGAGTGCTGCATCGACTGATAAGGCAGGGTGCGACGTTCAAACACAAACATTCCAAGAGCAAGCATCATCATCAGCCTCCTTAATCGTGCATCATGCTA | Escherichia virus 186 (NCBI:txid29252) |
| 362 | AAAACAGGATGAGAATCTGAAAGATCCTAACGCAGATCCAGAAGTTATGGAATTATTCAACGAAGTTTATACCAAATGGATGGGAAGTACATTAGTCAAGAATCTATATTCCAAAGTGCTTATTACACTGGGAATGGAGTATCTGGACAC | Escherichia virus 186 (NCBI:txid29252) |
| 363 | GTTCTGCTACGCGGATCCCGAAAATACGAATCTCTTCACGGTGCAGCTTCGCGCGGATACGCGCCCATAAGCCAGTAAGATAGCTTTGTGTATCTGCCGGGCTGGCTCCGTTCCATTTGCTGTTACGGTAGCCCGCTTTGGTGGTGGCGT | Escherichia virus 186 (NCBI:txid29252) |
| 364 | CGTTCAGTTTGGCGAGCTGGGTCAGATAGGCATTGAACTTAAAACGGGTTTCCTGACGCATAGTATTTCCTGTTTGAATTAATCGGTTAGTCACAGCATCGGGCGGGGTTGCCGCCCGGTTTCGGTCTGCGGTTTATCAGCAGTCGGTCA | Escherichia virus 186 (NCBI:txid29252) |
| 365 | CGTTCAGTTTGGCGAGCTGGGTCAGATAGGCATTGAACTTAAAACGGGTTTCCTGACGCATAGTATTTCCTGTTTGAATTAATCGGTTAGTCACAGCATCGGGCGGGGTTGCCGCCCGGTTTCGGTCTGCGGTTTATCAGCAGTCGGTCA | Escherichia virus 186 (NCBI:txid29252) |
| 366 | ATCGCCGTCACGGCGCTCAGGTAGTGGAAAACACGCACATTCTCGCCGTCGATTTCCTCGGCGTCAGGCACGTCGGCCAGTGTCTTAAACCCGGCGGCAATCTGGCGCAGCCGGTAGTCGTAAAGCTCTGCATTGGTTTCCGCCATGCCG | Escherichia virus 186 (NCBI:txid29252) |
| 367 | TCGACTACGACTATACGCCGGTTCCACCACTGGAAAGCCTGACCCTGCGCCAGCGTATCACCGATAAATATCTGGTGAATCTGGCCGAATCGGTCAACAGCTAAGGAGCCTGAAACAACATGGCACTACCCCGCAAACTCAAATATATGA | Escherichia virus 186 (NCBI:txid29252) |
| 368 | TAATACCGTCGACAATGTCACGGATAAGCGATGCGGTGATGGGCTTGTCGACCGCCCACATGTGCGCCTCGGCCATTGTGTCGGCCAGCACCTGAGCGGTGCGGGTGTAGTTCTCAAACAGGAAAAGCGGGTCATCAGAACAGGTGCGGT | Escherichia virus 186 (NCBI:txid29252) |
| 369 | TAGACCGTTACCGGCGCGCCACCGATAAACTGAAACTGACGAATATGCCAGCGCGCGGCCCAGGCAGAAACGGCAGGTGCGGTCTCTTTCAACAGTTCGCCGCTTTCGTCGTCGAGCTCGCCATCAAGTGCATAACCGTCAATATTTTTG | Escherichia virus 186 (NCBI:txid29252) |
| 370 | GTGCCGTGAAAACCGAATTGTGAAAAAGTGTCCAGTGCTGCGCTAAGAATCGCTTTTTTCTTCGCGCTTACTGCGCGCGCACGTTTACCCGTTGTTTTCACTGCGCCTTGCGTCATGCGCTCTCCCCTCTTGTAGATTGGCAACAGGATA | Escherichia virus 186 (NCBI:txid29252) |
| 371 | CTCTGGAAATGCTTCAAATTTTCTGGGATAACGGAAAGAGCTTTAATGAAGCTGATTCTGTGAGATATCTGTTCAGAAACGGGAAAATTCAGACGGAGTTTGAACTTCCGGAGAATACCACAATGCTTCGCCTTGATCCGGGCGAAATGT | Escherichia virus 186 (NCBI:txid29252) |
| 372 | CTGATCAGCGGCATGATCAGATAAAGTCCTATCAGCATATAGAGATACCAGAGCGGCGTGGTGTCGTAGCAGAAGTTGAAGACGAAGGTCCCCATCTTGTGCAGCGTCGCTTCGCCGGTGAACAGCGCGGGGTCGATCGACGGACTGGAG | Escherichia virus 186 (NCBI:txid29252) |
| 373 | AGTCGTTTACCTCCCTGAACCTGTCCTGCAGGGGTGACAGCTCGTTACGCACAAATACTTTTGCCACCTTCTCAACGTCACCGAGCGAACCGATATTCTCGGGCTTGCCGCCCATGAGCTGGAACGGTACGCGGTGCGCGTCCATCAGGT | Escherichia virus 186 (NCBI:txid29252) |
| 374 | CCATTCCAACAAGGAAAGGAGCCCAATATGGAATTTATCAGAGAAGATTGTATTTACGCAAGACAGTCAGTAGACCGCAAGGACAGTATCAGCATTGAAAGTCAGATCGACTTTTGCAAGTATGAATTGAAAGGTGGGAGCTGCCGGGTA | Escherichia virus 186 (NCBI:txid29252) |
| 375 | AGCTCGCTTGGTGGGTATCAGGCTTATCAGCCTGTCACGGCTCCCGCCGGTCGTACCTATATTGACCAGAGCAGCCCGACCTATCAAATCAACCTGCCGGGTGGTGGCGCGCCGGGTGGTCAATTGGGTAACCAGTTGCAGGATGCGTT | Escherichia virus 186 (NCBI:txid29252) |
| 376 | CACGCTGGAAGTTGACGATCCGGCGATGGTAGCAAAGCAGATGGAACTGGTGCTGGAAGGCTGTGTAAGCCGAATGCGGGTGAATCGTCGCCAGGCGGATGTCGACACTGCACATCGGCTGGCGGAAGATATCCTGCGCTTCGCCCGCTG | Escherichia virus 186 (NCBI:txid29252) |
| 377 | CAAGAAGTAAAAAGATGAAAAGAAAAATATTAGTTACTGTTCACATACCACTATCCCGCGAGGTGTTTTGGCATGAATATGCCAAAATCCCGAGACATACAAGCCAAAATTCCATTGCCGCAGGCAATCTGGAATGAATTTTGGCAAAGT | Escherichia virus 186 (NCBI:txid29252) |
| 378 | CAGGAGCTCGCACGCGCGATTCGCAACCTCATCCGCTCAGGTGTGGTGACTGAGGTTGATACCGTGCAGGGGCTGTGCCGCGTACAAAGCGGCGGGATCCAGACAACATGGCTGAACTGGCTGACCACCCGCGCCGGTCGTTCGCGGACG | Escherichia virus 186 (NCBI:txid29252) |
| 379 | TAAACCATGACGAGCCCCGCACAGCGTCACATGATGCGGGTCTCGGCCTCTCAAGCCGCGCAGCGGGAACAAGCCCCGCTGCGCCATGCAACCGCCTACGAGCAGATGCTGGTAAAGCTGGCCGATGACCGTCGCACGTTAAAAACCATC | Escherichia virus 186 (NCBI:txid29252) |
| 380 | GTCAGAGCATGCGCGACATACTGCTGACGCCGGTCGGCTCGCGGGTGATGCGCCGTGAATATGGCTCGCTTCTGTCGGCGCTGATTGATATGCCGCAAAACCCGGCGCTCAGGCTGCAAATTATGGTGGCGTGCTATTCGGCTATCCAGA | Escherichia virus 186 (NCBI:txid29252) |
| 381 | GAGTTGATTATGGCTAGTGACAGTGAACTCATTCGCCAGACTGCCGGAACTGCTTTTGATATGTGGCAAAAGTTCGCTTTTACATTTGTACTGGTTGCTTCTTTTGTATTGCTTTATCAACGGGATAGGTTCAAGAATTTTGTATCAAAT | Escherichia virus 186 (NCBI:txid29252) |
| 382 | ATCTTCTGCAACGCACTTCAGGCATAAAATTGCGCTATATCACATCTACTGGAGATACTTTGCCGGTAGAATGGATAAAAGAGACAGAGAAAATGCTTACTGGAACCATAGTGGTGCCGATGTACGGAATTACCGAATGTAAGAGAGTGG | Escherichia virus 186 (NCBI:txid29252) |
| 383 | GCTAAACGGTCATGAATTTATCGCCGGGCGACAGGCTTGCCCGGTCAGACAACCCGATTTAACCAAACAGGAAAGACTATGCGTCAGGAAACCCGTTTTAAATTCAATGCCTACATGTCCCGCGTTGCTGAGTTGAACGGCATAGACCCG | Escherichia virus 186 (NCBI:txid29252) |
| 384 | GAGAATGCGCGGCTTAACGCCGGTGACCGCTTCGGCAGTCAACAGCGCCTTGATACCGGTGTATTTACCGTTCTCATCCGTGCCGCCGATGATATTGGAAATGGTCTGCGCTTCCGCGTCTTCTCCGGTACCTTCGGCAACACGCACGAC | Escherichia virus 186 (NCBI:txid29252) |

List 6 Sequence classified as Klebsiella phage vB_Kpn_IME260 (NCBI: taxid 1912318), Klebsiella phage vB_KpnM_KB57 (NCBI: taxid 1719140), Klebsiella phage vB_KpnM_KpV52 (NCBI: taxid 1912321), Klebsiella virus 0507KN21 (NCBI: taxid 2169687), Klebsiella phage F19 (NCBI: taxid 1416011), Klebsiella phage K5 (NCBI: taxid 1647374), Klebsiella phage KP34 (NCBI: taxid 674081), Klebsiella phage Matisse (NCBI: taxid 1675607), Klebsiella phage Sugarland (NCBI: taxid 2053603), and Klebsiella phage PKP126 (NCBI: taxid 1654927) in mice fecal metagenomic data

| SEQ ID NO | Klebsiella virus (target bacteria Klebsiella pneumoniae) sequences in whole-community metagenomes in post-FMT samples | Notes |
|---|---|---|
| 385 | GAGGGAAGGAGACACAGTGGTTATAACGGAACTGACCCGGCTAGGGCGTTCTGTAAAGGAACTGTTCACCATCATCGAGAGGGTGCATGATGCCGGAGCCTCGATAAAGTCGCTCCGTGAGACTTGGCTTGACACCACCACACCGCAGGG | Klebsiella_phage_vB_ Kpn_IME260 |
| 386 | GCCGTTGAGAAACTGAACGAGGGCGGCATATTGGCTTTCGTCACATCAAGAGGTATCGCTGACACACAAGGCAACCAATTCGTGCGTGACTATCTTGTACACCGTTGCAATCTGATTACGGCATTACGTTTGTCTGATAGCCTGTTCATG | Klebsiella_phage_vB_ Kpn_IME260 |
| 387 | GGTTCGCAAATCACGTTCAATATCGGACCGTATGCCGCTCGGAATTTGGAGCTTGGCAACGATTTCGTTAAATTTGACGCAACGTTTAACGGTGTCGCTGAAAGTATTCAGGTTCCCGTCGGCAACATAGAGGCGGTGTTTGTCAGAGAG | Klebsiella_phage_vB_ Kpn_IME260 |
| 388 | GCTTCTGCAATGGCTGTTTCTGAGCTTTCAGCACGTATTTGCAATGTTTGGGGCAACGATCCTGGTGCCTACCCTGACGGGCTTACCCGTTTCGGTGGCACTTTTTGCAAGCGGTGTGGGAACGCTGATTTATACGTTTGTGACAAAGCG | Klebsiella_phage_vB_ Kpn_IME260 |
| 389 | AATCAAGTCTCCTGATGAATATCAAAAAAAGGCGGCTGAAATTAAAGCAACACAGCTACTTGGCATAGAATTACTTTCCAGCGTTTATATGCTTGCTATTTTGAATATGATTCTTATGGGTGATGGCAGTTCAAATATTTTGAA | Klebsiella_phage_vB_ Kpn_IME260 |
| 390 | ATGACTCATATCTGACACCGCCGTTAAACCGCGTACGGCAGTCTCTACCGTGCGGGCAAAGTTGGGGGAATCCATGGTGTGGGCAATATTCAACGAACCTAAATTGCAGGAAATATCATGACCTGTACGGGCATAGTCGAGATTCTCGTC | Klebsiella_phage_vB_ Kpn_IME260 |
| 391 | ACCGTATTTACGACTCGCTGTGACACATTATTTGGTGCGACGTATTGTGTAATGGCTCCAGAACATCCTTATGTGGACGAGATTACAACTTCAGAACAGAAGGAAGCTGTTGATGCCTATAAAGCCATCTGCGCAACGAAATCCGATCTG | Klebsiella_phage_vB_ Kpn_IME260 |
| 392 | CGGCCTCTTAATTGGTGTAACTGCGAAAGGCCGAAGCGTTCTGCGTTTTCAATAAGCATTACCGAGGCGTTCGGCACATTCACTCCAACCTCGATGACAGTTGTGGCTACCATGATGTGTGCCTGTCCGGATGCAAATAATTGCATCTGA | Klebsiella_phage_vB_ Kpn_IME260 |
| 393 | AATCCCGATGTCAAGTCGATATTTGATTTCCGGTATGAAGACTTCTCACTTGAAGATTATGATCCTCATCCACACATTAAAGCCATTGTAGCCGTATGATTGCAGAACGACCTCAAGTAATATCGATAATTGTGGCCTGCGCCGGTGCAG | Klebsiella_phage_vB_ Kpn_IME260 |
| 394 | GGTATAATACACCTTCCAGTTCGTGTGCTGATCCAAAAGTGCCTTCAGCTCCAGTACAATAGCCAGGTCCAAATCCTTTTCCTGAACCCCCAGCTTAATTGCCCCCGGCTGTCCGCCCCCATGGCCGGCATCAATCACCAGTACCTTTTC | Klebsiella_phage_vB_ Kpn_IME260 |
| 395 | ACTCCATCAGGATCATCCAGGGTATCGCCCCTTACTACGAGAAGTACCACTTTGTCTCCATCTCCCCCGAGATGTGCCGCCTGGCCGTGACCATGAGCGAGCGGTACATCACCGACCGCTTCCTGCCCGACAAGGCCATCGACCTCATTG | Klebsiella_phage_vB_ Kpn_IME260 |
| 396 | ACCAGCTCTGAAAGCCGCATTAGTAATTTTCTCTTGTGGGAGCTTGCCTATAGTGAGCTCTACTTTACCTCCGTTTTATGGCCGGATTTTGGTAAAAGAGAATTTAAAGAAGCATTATTGTGGTACGCATCAAGAGAAAGGCGCTTTGGT | Klebsiella_phage_vB_ Kpn_IME260 |
| 397 | CATGGTTTTTACCCCAGAATAGAGAGAGAATCTATCTTGTCGGACGTCTTGCAAAAAAATGTACCGGAGACGTATTTCCTTTCCCGGCGCCGGGTGGAATGCATGGTTCGTTCGGGAAAGATGCGACTGCTGCCTCTGTATGTGGAACAA | Klebsiella_phage_vB_ Kpn_IME260 |
| 398 | CTTTTTGCAAGAAATCTTCAAATCGGAGACTCAGGAGACGTAGACCTTTTTATCCGAACCAGCTCTGAAAGCCGCATTAGTAATTTTCTCTTGTGGGAGCTTGCCTATAGTGAGCTCTACTTTACCTCCGTTTTATGGCCGGATTTTGGT | Klebsiella_phage_vB_ Kpn_IME260 |
| 399 | AAAATCGATTGCTTGCAAGATCTGGTACGTAAATTCAGTGTAAGAGATCCCTACTTCTAAACGGCTAGCAACAACTTCTTTATTCAACATCGTATTAACGTTGAACAATTTACCATAATCGCGCAAGAAATCTAATAAACTGATCTCTTT | Klebsiella_phage_vB_ Kpn_IME260 |
| 400 | TTTACCTACCCCCGGCGGCCCTACCAGGCAGAGGATCGGTCCCTTGATTTTGTTGACACGGCTTTGAACCGCAAGATACTCAAGGATACGATCTTTCACGCGCTCCAGACCATAATGGTCGGTATCAAGGATTTCCTGCGCCTGACGCAG | Klebsiella_phage_vB_ Kpn_IME260 |
| 401 | TAAAAAGGCCGTTACATTGCCACTTGGTTCTTCTAGAAATTTCAACAGACTGTTAGCGGCACTGATCGTCATCTTTTCAGCATTTTGAATGATAAAAAGCTTCCGATTGCTCTCGACCCCACTTTTGACGAATTCAGCTTTCAAATATCT | Klebsiella_phage_vB_ Kpn_IME260 |
| 402 | GGACCGTATGCCGCTCGGAATTTGGAGCTTGGCAACGATTTCGTTAAATTTGACGCAACGTTTAACGGTGTCGCTGAAAGTATTCAGGTTCCCGTCGGCAACATAGAGGCGGTGTTTGTCAGAGAGACCGGCTTTGGCATCCCGTTCGAG | Klebsiella_phage_vB_ Kpn_IME260 |
| 403 | CAGCGTCCGATATTTGATCGGCCACTTTGTCGGGGTGTCCTTCAGACACCGATTCGGATGTGAATAAATATCCCATTCTAAATTGAAAATTAAGAATTAAAAATTAAAAGTTAATTCGTTAACAACCTTGGGAAGTTATCAGAGTAGATA | Klebsiella_phage_vB_ Kpn_IME260 |
| 404 | AGCTTGGCAACGATTTCGTTAAATTTGACGCAACGTTTAACGGTGTCGCTGAAAGTATTCAGGTTCCCGTCGGCAACATAGAGGCGGTGTTTGTCAGAGAGACTGGCTTTGGCATCCCGTTCAAGGTCAAACTTAAACCTGACCAAGAAC | Klebsiella_phage_vB_ Kpn_IME260 |
| 405 | AAGGCCCGCGAGGCTGCCCGCAAAGCCCGCGAGATGACCCGCCGCAAGAACGTGCTTGACTCGTTTAGTATGCCCGGCAAGCTGGCCGACTGCGCCAGCAAGGATGCCTCCCAGTCCGAAATCTTCATTGTAGAGGGCGACTCGGCAGGT | Klebsiella_phage_vB_ Kpn_IME260 |
| 406 | GGGGAATATCGGAGGCCATGACCGGATCAAAGCGGTGGTGGATACGGCAAAAGAGCGGGGAATTCCGATCCGTGTGGGCGTAAACAGCGGCTCTCTGGAAAAAGAGCTGGTGGAAAAATACCATGGAGTTACAGCGGAAGGAATCGTAGA | Klebsiella_phage_vB_ Kpn_IME260 |
| 407 | AGCACGTGCTCTATGCAGCATGCTGCTGGCGCATCAAGCCTGTCGAAAAACAGCCCTGAGTGCTTGTCGAAGAATGATACAAAGCGATGGCCCATTTTGTTTATGGTGAGCACCTCCTGCTCATCCTCCGGCATCCGCTCGCTTACCGGA | Klebsiella phage_vB_KpnM_KB 57 |
| 408 | CGACTATTCAAAATCGCACAGCGCATCAAAGGGCCAACCTTCGATAAATACTCTCATTCGCCGTCGCAGGCTATCGACGTGCTGGAAGTTGCAATATTCGGCGAGGATGAATCTTGCCGCGCCGCCATGCTCGCAGCCGCCCCGCAGCTA | Klebsiella phage_vB_KpnM_KB 57 |
| 409 | AAACTGGCAAAAGTCATTCGGGAGTTCAACGAAGAAGACGAAAGGCCATTAGCAAAAATGGCGCGGATCATTCGTGATAATCCACACCCGACAAACGAATGCGACATGCCGAAAGCGCAGCCAGTGCCGGATGACTACCAGCACCTACGA | Klebsiella phage_vB_KpnM_KB 57 |
| 410 | ATCCTCCGGCATCCGCTCGCTTACCGGAATCCATTTACCCGGCGCGGTGGCGGGTTCACTGCCGGGTAGCTGCGGGGCGGCTGCGAGCATGGCGGCGCGGCAAGATTCATCCTCGCCGAATATTGCAACTTCCAGCACGTCGATAGCCTG | Klebsiella phage_vB_KpnM_KB 57 |
| 411 | CTCCTGCGGTGCGGCCGGCAGCGGCATCCAGTGAGTTACCAGCACGTGCTCTATGCAGCATGCTGCTGGCGCATCAAGCCTGTCGAAAAACAGCCCTGAGTGCTTGTCGAAGAATGATACAAAGCGATGGCCCATTTTGTTTATGGTGAG | Klebsiella phage_vB_KpnM_KB 57 |
| 412 | ACGGCAAAAACGGGAAATACAATGTCACGGAGCGTGTACGAGGATGGACAGATGAAGACGAAATTGGGTTATTCGTCCAGGTCGGCGCGATTCTGCGAGGTGAATCAGAAATCACCTGGGGGGAGCCACTTTATCTCTCTGGAGTCGTCA | Klebsiella phage_vB_KpnM_KB 57 |
| 413 | GAGTGCTTGTCGAAGAATGATACAAAGCGATGGCCCATTTTGTTTATGGTGAGCACCTCCTGCTCATCCTCCGGCATCCGCTCGCTTACCGGAATCCATTTACCCGGCGCGGTGGCGGGTTCACTGCCGGGTAGCTGCGGGGCGGCTGCG | Klebsiella phage_vB_KpnM_KB 57 |
| 414 | TGAGACGACAAATCCCTGGTTGATAATTGTGGTTAGCTTGGAAAGGTCGTCCGCACTTGTGCCGTACTGCCTTGTCGCTCTTTCCAGTCTCGCATACAGCGACGCTGTGGCATCTAAACTTCCTCTCGTTTGCTGTGTAATGTTGAATAC | Klebsiella phage_vB_KpnM_KB 57 |
| 415 | GCCACGCGCAGCAGCCGGTACTGCAGCCAGTGATGTTTATTGATGGTGACATTTCATCCGAGGACGCCGATAAACTGGCAAAAGTCATTCGGGAGTTCAACGAAGAAGACGAAAGGCCATTAGCAAAAATGGCGCGGATCATTCGTGATA | Klebsiella phage_vB_KpnM_KB 57 |
| 416 | AGACGAAAGGCCATTAGCAAAAATGGCGCGGATCATTCGTGATAATCCACACCCGACAAACGAATGCGACATGCCGAAAGCGCAGCCAGTGCCGGATGACTACCAGCACCTACGAGAGCTCTACCACGCGCAGGAAAAGCGACTATTCAA | Klebsiella phage_vB_KpnM_KB 57 |
| 417 | GAATGCGACATGCCGAAAGCGCAGCCAGTGCCGGATGACTACCAGCACCTACGAGAGCTCTACCACGCGCAGGAAAAGCGACTATTCAAAATCGCACAGCGCATCAAAGGGCCAACCTTCGATAAATACTCTCATTCGCCGTCGCAGGCT | Klebsiella phage_vB_KpnM_KB 57 |
| 418 | CAGCCAGTGATGTTTATTGATGGTGACATTTCATCCGAGGACGCCGATAAACTGGCAAAAGTCATTCGGGAGTTCAACGAAGAAGACGAAAGGCCATTAGCAAAAATGGCGCGGATCATTCGTGATAATCCACACCCGACAAACGAATGC | Klebsiella phage_vB_KpnM_KB 57 |
| 419 | GGCATCCAGTGAGTTACCAGCACGTGCTCTATGCAGCATGCTGCTGGCGCATCAAGCCTGTCGAAAAACAGCCCTGAGTGCTTGTCGAAGAATGATACAAAGCGATGGCCCATTTTGTTTATGGTGAGCACCTCCTGCTCATCCTCCGGC | Klebsiella phage_vB_KpnM_KB 57 |
| 420 | AATAGAGATCGCCCGTGCGTAAACCAGCCTCAACGTGACCAACAGGAATACGCTGATAAAACGCCGCCAGGCTGGTTGCCAGCGTCGTTGTTGTGTCGCCGTGAACCAGCACGACGTCTGGTTTGAACTCGGCAAGAATAGGTTTTAGCC | Klebsiella phage_vB_KpnM_KB 57 |
| 421 | CGCGGATCATTCGTGATAATCCACACCCGACAAACGAATGCGACATGCCGAAAGCGCAGCCAGTGCCGGATGACTACCAGCACCTACGAGAGCTCTACCACGCGCAGGAAAAGCGACTATTCAAAATCGCACAGCGCATCAAAGGGCCAA | Klebsiella phage_vB_KpnM_KB 57 |
| 422 | CTTCTGCTGCAGCAACCCTTCGCCGGCGGGCTGCATGCTTTCGACGATAATCTGGTAGTCGCCGCGCGGCTCATACAGCGTGATGTTGGCGCGAACCAGCACCTGCTGCCCGTGCTGCGGGCGGAACGTGACCCGGCGGTTGCTGTTGCG | Klebsiella phage_vB_KpnM_KB 57 |
| 423 | AGACCGATTATCTCGATCTCTATCAGGTACACTGGCCGCAGCGCCCAACCAACTGTTTCGGCAAACTGGGCTACACCTGGGCCGATTCCGCGCCGGTCGTCACTCTCCTGGATACGCTGGATGCTCTGGCTGAATTCCAGCGTGCCGGCA | Klebsiella phage_vB_KpnM_KB 57 |
| 424 | CCCTCATCAACATCATAAAAACGCGTAATCAGGCTGGCGATGGTAGATTTACCTGACCCTGAGCGCCCTACCAGCGCCACGGTTTTCCCTTCCGGAATATTGAGGTCAATATTGCGCAACGCAGCGACTTCACGCCCCGGATATCTGAAG | Klebsiella phage_vB_KpnM_KB 57 |
| 425 | CATCGTTACTCTTATCAAACGACAGCGTGTTAATGCCCATCAGTTCTCCCAGCGAGTTCACCAGCGCGCCGCCAGAGTTACCGTGGTTAATGGAAGCATCGGTTTGTAGGAAGTTTTGCCGCCCGGTCGGGTTCAGACCGATTCGCCCCG | Klebsiella phage_vB_KpnM_KB 57 |
| 426 | ATTTTCGATAAAGATGGTTTCGCCCGGTTGAACCAGTGACGCGGCAAACTCGGCGAGTTCACGCTTCAGCGTATAGTTGCTCATCATACGGGTTTCGACGTCATCACTATCAAGCGAAACTGCAAAGCCATGTGCACGGCGGAGGTAACT | Klebsiella phage_vB_KpnM_KB 57 |
| 427 | CAGCTCACCGATGAAGGAAAAAAAATCTATTCCAGTATTGAGCTGCATCCGCAGTTTGCCCTTAACGGTAAGGCGTATGTGGTCGGCCTGGCGATGACGGACACCCCGGCAAGTCTGGGGACTGAGCGCCTTAAATTTGCCGCGCAGCAG | Klebsiella phage_vB_KpnM_KB 57 |
| 428 | TTCCGGCGCACAGACGTATTCCGCTCTATCGCCACGCGCAGCAGCCGGTACTGCAGCCAGTGATGTTTATTGATGGTGACATTTCATCCGAGGACGCCGATAAACTGGCAAAAGTCATTCGGGAGTTCAACGAAGAAGACGAAAGGCCAT | Klebsiella phage_vB_KpnM_KB 57 |
| 429 | GCATCAAAGGGCCAACCTTCGATAAATACTCTCATTCGCCGTCGCAGGCTATCGACGTGCTGGAAGTTGCAATATTCGGCGAGGATGAATCTTGCCGCGCCGCCATGCTCGCAGCCGCCCCGCAGCTACCCGGCAGTGAACCCGCCACCG | Klebsiella phage_vB_KpnM_KB 57 |
| 430 | GAAAGCGTGAACTCAATTTTTTTCGCCTTACCATAGCGATCAAACTCCTGATGCGTTTCCTGCAAGCCCGTGATGACATACATTCCGTAAATGGATCCGACGCCATCTATGAGCGGCCAGGCCAGCCCGGTGTAGGCCATTGTCGAGACG | Klebsiella phage_vB_KpnM_KB 57 |
| 431 | CAGCAGGTTAGCTTGAACGGTAAGTTGATCGGCACCAGGTCTGGGTGGCAGATTTTCTTTCTGTCGGGCTTCATTACGGGACATCAGCCCGTTCTGAGTCATCGTTGAATAAAACGCAGCACGAGCGGCACTGTCCGCACGACGCAGCCC | Klebsiella phage_vB_KpnM_KB 57 |
| 432 | CCTGTCGAAAAACAGCCCTGAGTGCTTGTCGAAGAATGATACAAAGCGATGGCCCATTTTGTTTATGGTGAGCACCTCCTGCTCATCCTCCGGCATCCGCTCGCTTACCGGAATCCATTTACCCGGCGCGGTGGCGGGTTCACTGCCGGG | Klebsiella phage_vB_KpnM_KB 57 |
| 433 | TGGAGTAACTCCGGCTGGAGCACGCTGGCAGCGCAGAACCCGAAGTCCGGCAAATACATCATTCAGGCCACGAAGACATCCAGCGGCTCAACGGCCTGTGACCAGGTGGTGAAGCTGACGGGTGGCCAGACGTACCGCGTGGGTGCCTGG | Klebsiella phage_vB_KpnM_KB 57 |
| 434 | AAGATAGTTTTGGTTTAACTCCGGTTTCTCCGCTGAAACGGACCTCGATGGTAACTGGTCGCGCGAAACAGCGGCGTGCCTATACCTCGACACCAACCCAAACAGATATGGCATGGATTTTTAATGATGCGCAAGCTCAGGCCTTTGAAG | Klebsiella phage_vB_KpnM_KB 57 |
| 435 | ATAGGGGGTCAATTTGTTGAGAATATCCAGTATGGCCACGAACACCTCCACGGCGATGATCCCGCCGCTGTCACGGTCGACCAGCTCCAGACGGTCGCCTGCGTAATCGACATACATCTTGTCGCCGGCCTTGTGTTCCAGATGCGCTAT | Klebsiella phage_vB_KpnM_KB 57 |
| 436 | TACCAGCACCTACGAGAGCTCTACCACGCGCAGGAAAAGCGACTATTCAAAATCGCACAGCGCATCAAAGGGCCAACCTTCGATAAATACTCTCATTCGCCGTCGCAGGCTATCGACGTGCTGGAAGTTGCAATATTCGGCGAGGATGAA | Klebsiella phage_vB_KpnM_KB 57 |
| 437 | CGGACAGCGGAACGGGGATTTCCCGGGAAGAGCGGCAGAAGCTGTTTCAGCGGTTCTACCGGGGCGGCAATGCGGCGGAGGATAGCGTGGGCATCGGCCTGGCACTGGCCAAAGAGGTGGTGGAACAACAACATGGGCATATTCAGGTGG | Klebsiella phage_vB_KpnM_KB 57 |
| 438 | GGCGGAATATTCTTTGGAAGAAGTTTCACAGCCGATTGGTATTACAGGGTATGAATTAAGCAATGCGCTCAGCAAAGAATATCAACATAGTCTTCCTACAATAGAAACGCTGGAGTCTGAAGTCGCAAAAATGATGGATACAAAATAGTC | Klebsiella phage_vB_KpnM_KB 57 |
| 439 | GGTCAGCAGCATGACGCAGCGGTCGATGCCGATGCCCATGCCGCCCGTGGGGGGCAGGCCGTATTCCAGGGCGGTGAGGAAGTCCTCGTCCATCATGCCCGCCTCGTCGTCCCCCTTGGCCCGCAGCTCCACCTGCTTCTGGAACCGCTG | Klebsiella phage_vB_KpnM_KB 57 |
| 440 | GGCGTAATAGACCAGACCACTGCAATCAAATCCGGTGCGCGGAGAGGTCCCGCCCCAGCGATAAGGTTTGCCAATCTGATCCATCAACTTTGACATCGCGGTGCTTTGCGCTTTTTGTACCCGCGCTTTATGCGCATCGGCGATAGT CAG | Klebsiella phage_vB_KpnM_KB 57 |
| 441 | TATGGTGAGCACCTCCTGCTCATCCTCCGGCATCCGCTCGCTTACCGGAATCCATTTACCCGGCGCGGTGGCGGGTTCACTGCCGGGTAGCTGCGGGGCGGCTGCGAGCATGGCGGCGCGGCAAGATTCATCCTCGCCGAATATTGCAAC | Klebsiella phage_vB_KpnM_KB 57 |
| 442 | TCGGTTTAGCGTGTCGGTGAACATTTTTTCAACATTCTCATCCCTTATATCGTTCCAGGCTGAGAGTTTTTCAAGTGTTGAGTATTCACTAGTCCCGCGATAGTTCTCTAATAGCTGCCCCGTGGTCAGGCCCGGCTGCGCAACACAGAG | Klebsiella phage_vB_KpnM_KB 57 |
| 443 | AGGCGAAAACGTAGCCCCGCGACTGCAACAGCTCGCGCAGCGGCTCATGGTTCTCGATAATTCCGTTATGCACGACCACAATATGGTCAGAAACATGCGGATGCGCGTTGCCTTCAGAGGGTTCGCCGTGCGTCGCCCAGCGGGTATGGG | Klebsiella phage_vB_KpnM_KB 57 |
| 444 | CCAGTAACTGGTGGGTCTGTGCTTCATGCAGATACATTTCCAGCTTTGGAAAGGTCTGGTGCAGNATAGGGATAATATGCGGTAGCAGGTACGGTCCAACTGTGGGAATCAAACCAATGTGCAGCGGCCCGGACATCGTCTCGCCCTGCT | Klebsiella phage_vB_KpnM_KB 57 |
| 445 | GGGCGGCTTTATTATGTGCGGCGTGCTGTTCTGCCTGGTTTCTTTTATCGTGAAGAAAGCGGGGACCGGCTGGCTGGACGTGCTGTTTCCACCTGCGGCAATGGGCGCAATCGTTGCCGTCATCGGTCTGGAGCTGGCGGGCGTAGCTGC | Klebsiella phage_vB_KpnM_KB 57 |
| 446 | CTGTCGGAGAGAATGCTGTGGTCGGTGCCGGTAGCGTGGTTACCCGCGATGTTCCGCCCATGACCATTGTAGCTGGTAATCCGGCAAAGATAATCAGAAAAATAGACGAAAAATAAACCTATAGTGATGATTGAGAACAAGGAAATAAGC | Klebsiella phage_vB_KpnM_KB 57 |
| 447 | AGGATGAATCTTGCCGCGCCGCCATGCTCGCAGCCGCCCCGCAGCTACCCGGCAGTGAACCCGCCACCGCGCCGGGTAAATGGATTCCGGTAAGCGAGCGGATGCCGGAGGATGAGCAGGAGGTGCTCACCATAAACAAAATGGGCCATC | Klebsiella phage_vB_KpnM_KB 57 |
| 448 | AATGGACATTTCACCATACTTACAATGGTACTCCCCAAGGAGGTATCATCAGTCCTATACTGGCGAATATCTACCTTGATAAACTGGATAAGTATATTGTGGAATATATCTCGAAGTTCAACAAGGGTAAGGCGCGGAAACGCAATCCCG | Klebsiella phage_vB_KpnM_KB 57 |
| 449 | CGAAGGCTTACACCGGAACAGCGTCTTCTATCGTGGTCGTTCTGGATCCAGGGCACGGCGGAAAGGATAATGGCGCATCAGCGAATGGTCTGATAGAAAAAAATATCAACCTGAAGATTGCGCAGGCATGTAAGGCTGAATTAGAGAAGC | Klebsiella phage_vB_KpnM_KB 57 |
| 450 | TTCACATTTAAATAAGTATCTTGTTGTCCTGCAAAACTCGCATCAGGTAGATCTTCAGCTGTTGCAGAGCTGCGCACTGCCACATCAGCTTCTTTCATACCATATTCTCCACTCAAAATTTCATATGCTTGAAAAATCTCTTTGCGTAAA | Klebsiella phage_vB_KpnM_KB 57 |
| 451 | AAACTTTTAATAAAGCAGAAAAAAGCTTTAAATTATTCTGATAAATGGCAACTTGATGGCAGTTTAGCAAAAGGCGCAGCCATGAATAACGATAACATGAAGTTATACCTTAGAGCGTTTAACAGTGAATGTGATGTATTGATTTCAAAA | Klebsiella phage_vB_KpnM_KB 57 |
| 452 | CGCGCGAACGGACGGTTTCCGGACGCGCCTGGACGATAAACAGCTTACCGGTATGGCCATCTTTCGCCCATTCGATATCCATCGGGCGGTTGTAGTGCTTCTCGATTTGCACTGCCTGCTGGGCCAGCTCCTGCACTTCCGCGTCGGTCA | Klebsiella phage_vB_KpnM_KB 57 |
| 453 | TTCGATAAATACTCTCATTCGCCGTCGCAGGCTATCGACGTGCTGGAAGTTGCAATATTCGGCGAGGATGAATCTTGCCGCGCCGCCATGCTCGCAGCCGCCCCGCAGCTACCCGGCAGTGAACCCGCCACCGCGCCGGGTAAATGGATT | Klebsiella phage_vB_KpnM_KB 57 |
| 454 | CTGGAATCTAACGGCGATCTGTATAACTGCGATCACTTTGTTTATCCGGAGCATAAGCTGGGTAATATTCATCAGCAGAGCATCCGCGAGCTGAATAATAGCGAGCAGGCCATCGCCTTTGGTCAGACACAACAAGAGACGCTAACGGCG | Klebsiella phage_vB_KpnM_KB 57 |
| 455 | TCCCCTCTGCCGTGGTCACGCGCGTGGTCAGGCTACTGATGGCCCCGGCGTTGGCGGCAATATTCACTTCATCCGTGATATCCACAAAATAGAAGTCGTCAAAATACTGAGAGCCAGACTTCAGGGAAGAGTAGATAGCCACATCAACCA | Klebsiella phage_vB_KpnM_KB 57 |
| 456 | ACTGGAAGAGCCTTAATCTTAACGGCCCAATTGTCGATAAACACTCTACCGGCGGCGTAGGCGATGTGACTTCCCTGATGCTGGGGCCGATGGTCGCTGCCTGCGGCGGCTATGTACCGATGATCTCCGGGCGCGGACTCGGTCATACCG | Klebsiella phage_vB_KpnM_KB 57 |
| 457 | GTCGAAGCGAAAGACGCGACTCCGGCACAAAAAGAGATCAAAGAAATCAATGCCGGCATGTACTGTTTTAAAAATAAGGATCTTTTCGAAGGTCTGAAAAAATTAACCAATGACAATGCTCAAAAGGAGTATTACTTAACGGATCT AGCG | Klebsiella phage vB_KpnM_KpV52 |
| 458 | GGGTCGCGGATCGCGCCGCCAAGGCAGGTTGCGGCCCCGCCAAACGGCTCTATCTCCGTAGGATGGTTGTGCGTCTCATTCTTGAAATTCACCAGCCATTCCTCCGTCTGTCCATCCACTTCAACCGGAACGACAATCGAGCAGGCGTTG | Klebsiella phage vB_KpnM_KpV52 |
| 459 | GATGAAGATGTACTATGTGGGACCGCAATGTCGCCATGAACGCCCGCAAAAGGGCAGAATGCGTATCTTCAATCAGTTTGGTGTTGAGGTCATCGGTGCCAAAAGTCCGTTGCTGGATGTGGAAACGATTGCACTTGGATGGTCGTTTAT | Klebsiella virus 0507KN21 |
| 460 | AGCCCATTGGCGGAACTGGACTCCACGCGGGTTACGGATGCGGAATCCTACTGCAAGAATCATTTCGAGCGAGTAGAATTTCACGGAGTATTGTTTGCCATCGGAGGCAGTTGTAAAGAAATCCTTTACAACTGAATTGTATAACAATTC | Klebsiella virus 0507KN21 |
| 461 | AAACATAACACCCTGCTGATCAGTGCCGGTTCTACGTTCTACGGCTGCGCGGCACTGGTGAACATTCCGCCGTCGCTGTTTGCATCGTGCCCGCTTATCACCGCATTCGGCGCAACGTTCCAGAATACCGGCGTGGTGGAAATACCGGAA | Klebsiella phage K5 |
| 462 | GGTCCAGCTCCAGTTCTCCCCCAAGAAGCCGCAGCAAGGTGGTCTTCCCTGCCCCGTTGATCCCCACCACCCCAATCTTTTCTCTGCCTTTTATCTCAAAATCAATGTGGGACAGCACCTGAATCCCTCCCAGGGAGATCGGAAGAGCAC | Klebsiella phage K5 |
| 463 | CAGTTCTGCCGACCAGCGATAATCACCTTGCTTGTTCGCTTCTTGCGCCAGGTTGATGACGCGGGCAGAACCGCCCAGCGCCTGCACGTAACGTTTACCCATCTCCACCTGACCATACGGATGCAGGTTAGCCGGGTTACCGTCGTAATA | Klebsiella phage K5 |
| 464 | ATATACATCAAACGATATACCCAACTCCTCAAACGAGTCCTTGATTATCTTGTGGTAGCGGTCAACGATATCCTGTGGAGTCACACCCTCGCGGCGTGCCTTTATAGTGATAGGCACACCGTGCTCGTCGCTTCCGCCTACCATCACAAC | Klebsiella phage K5 |
| 465 | CGCATGATTGACGAACTTGCCAAGGAGAGCAGGAACAGAAGTATGAACGACAAGAAGCGGAAGGATGAATAGTAAACTAACACATGGCTCTCTGTTCAGCGGCATTGGTGGTCCAGAAATAGCTGCTGAAATGATGGGCTGGAAAAACGT | Klebsiella phage K5 |
| 466 | AGTATGAAGAGATAAGATGCAAGCCAGATTTAATCGAGCCTTTTCACCGCCGGAGAACTTATGATAGGAGCCACAGTCAATGCCGTCGCGCATCACCTGAACTGAAATCTTATCTCTCAGCTTACCAGTCTTGGTTACAGTGAATCCTTC | Klebsiella phage K5 |
| 467 | CAAGCCCCAAACAGGTCCGCCATGCTGCCCACAAAGATGCGCTGGGGCTTCTTCATCTGGGCAGGGTCCCCCAGGCGGTAGCGGTGCAAGGTGGGCTTGAAGCCGAAGGGGAAGGGGGCGACCACCTCCTTGCCATTCTTCCTGAGGACA | Klebsiella phage K5 |
| 468 | CCATCCGTAAGCGGTTCTTCATGGTGATGCGCTGCGATGGGAAGCCGATTGTATGGCCGGAAGCCACTCATGGAGATCCGAAATCACCGGCGGTGCTAGCTGGCAAACTGGCACCATGGCGCACAGCTGCGGAATGCATCGACTGGTCCA | Klebsiella phage K5 |
| 469 | CGCCTGGCCGATACGATCCAGACGACCAATACGCTGCTCCAGTAGATCCGGGTTGAATGGCAGGTCAAACATCACCATGTGGCTGGCGAACTGGAAGTTACGTCCTTCAGAACCGATTTCTGAGCACAGCAGTACCTGTGCGCCGGTGTC | Klebsiella phage K5 |
| 470 | CTCCTTCGATATAAATATTCTGCGTCTCATCCCAGTTTTTAGACTTTTCTTTATTTGGTTTTAAAGTCGCAGTGGTTGGTTGTTGAGCTAACTTCATTGCTTGTTTTTTACCGTTCCACGTAAAGTTATATCGCTCGGGAGAATCATCTA | Klebsiella phage K5 |
| 471 | AGCCCCATCTCTTTCCCGAGCTTACGGTGGTCGCGCTTCTTAGCTTCCTCAAGAAGAGTGAGGTATTCATCGAGCATCTTTTTCTTTGGGAAAGTGATGCCGTATACTCTGACGAGCTGTCCTCTCTTTTCGTCACCGCGCCAATAAGCG | Klebsiella phage K5 |
| 472 | ACCCACTCGCCTCCCCAGCCGACGACCGAGGAGATGCCCCGCTCGTTGAGCTGGTTCGCGGCCACCTGGTCGAACCCGCGGCTCTTGGAGCCCTCGGCGAAGCGCTGGCGCACGACGGGGACGGCGCGGTTGCTCCACCCCTCGAAGGGC | Klebsiella phage K5 |
| 473 | CACGCCTTTGCCTCGGCCTCGGTCTTACCGTAACAGTGCAGGGTGCGTTTTTCTCCGTCCGGATCGCCAATCTCCACCTTGATTTTCTTTTTGTTGTCGGGTTGTAGGCTAACCACTTTCAGCTTAATTTTCACGTCCTCGGCTTTCTGT | Klebsiella phage K5 |
| 474 | ATAGCCACTATGATTGTAGTAGGGCTATATTTATTACCGATATTGTTTTGAACAATTAAAACTGGTCTAACTCCTCCTTGTTCAGAACCTACCGTAGTTTCCAGATCAGCATAATAGATTTGACCTCTTAGTATATGATTGTACATATCG | Klebsiella phage KP34 |
| 475 | GTCTCAAAACGTTCGCCGTTGTTATTGTTTACAATCTGCACTTTCTCACCGGCAATCAAATTTGCAGCATCCATCAGATCTTCATCGATAGTAATGCTACCCATATAATTCAAATTAGCTTCCGTCACACGTACACAGTGCAACTTTGAC | Klebsiella phage Matisse |
| 476 | CGACCAGACAGCCTACCAGCAGCGACAGGCGCGCGCCGTACATCAGGCGCGACAGGATATCGCGGCCAACGTCATCGGTGCCGAGCAGGTGGGACATGCTGCCGCCCTCCTGCCAGAACGGCGGAGCCAGCAGCGCATCGCGGAACTGAT | Klebsiella phage PKP 126 |
| 477 | ACACCTTCCCAGGCAAGGCTTGAGTGGCCTCAAACTCCAACAGTCATCCCCACCCAGCACCCATTGAGACGCTGGGGTGCCTTGCCACATGGCTACGTGGAGATGTATTCAGGGAAACACTGAATGAGCCAGGATGGAAGCAGAGTCCAC | Klebsiella_phage F19 |
| 478 | TGTCCTGTGGACAGACAGATATGCACCTCCCACCGCAAGCGGCGGGCCCCGACCGGAGCCACTTTAGTTACAACACTCAAATACAACCACCAGAAAAACCCCGGTCCCGCGCAGAACTGAAACCACAAAGCCCCTCCTCATAACTGAAAA | Klebsiella_phageF19 |
| 479 | ATAATAATGACCACTCTGTTTTCAGGGTGGTTTTTTATTTAAGCCGATCAAGCTCAATTTTTAGGAAAAGGGCCTTAGATATGGTTAAATAAGAGTAAAAAGGAGATTTGCGATGAAAGATGAGGTAAAGTGGCGCCAGAAAAATTTATT | Klebsiella_phage F19 |
| 480 | TGCCCCTGCTTAGCTGTGCGGCGACGGCATTGTAGTCAAGCGAGGCGAAGCCGAGGAGAGACGTGGTCATTTCCCTGTCGAGCGGGAACGAGTTTCCGTACCCTGCGGCCGAGCCGAGAGGATTCTGGTCCGTTACATTATATGCCCCGT | Klebsiella_phageF19 |
| 481 | TTCTTTTTTCTCATACTGTTTATCCTTTCGACAATTGTCAATTTATATTTTCACTTTACATTTCCAAATAGACAATGTAACTTATACTCATTGCCCCCCACAATCATAATACCGTCAATATAATTGTGTAGTTTTAAGCAACCGATTGTG | Klebsiella_phageF19 |
| 482 | AGATAGGATACTAATCATTGCTGGAATAACAACTTTCCTTTGTTCCTCTAAACATCTGAATTTCTTATTTTTCTCCTGTTCAAATGATAAGCATACAAAATATAATTTATCTAAACATAAAAAATAAAAAGCAAAAATTCCCTTAGGTTT | Klebsiella_phageF19 |
| 483 | CATTGGCTTGAGGCAACTGCGCCCATTGATCGGGGCTGGAATACCCTGCTGAATCTGCGTTCATGGGCAATGGTCGGCAGCGGCCTCGTGGCAATATGGTCAGTTCGTCATCCGCGCTTTTTGATGCGCTGGGCAAAACGCAGCCTCGGT | Klebsiella phage Sugarland |
| 484 | ACTATTCTGCAAATTAAGGGTTCATTATTTGTTGGTCAGTTGTTGCTTTCCACTGCAAGGCTTTAAAAAATAAATAAAGGTATCTTCAGAATTCCAACATTTGGAATTTGAAGATACCTTTTTAGTTTCCTTATAAATTAGGGGGTGTAA | Klebsiella phage Sugarland |
| 485 | GGCAGCGGCGGACCACAGTTTCGCGGCGACGTGGCGGTGAAGGGCGATCGAATTGTGGATATGGCCCCGGCGCTGAACCTGGCGGCGGAGCAGGTGATTGACGGGCAGGGGCGCGTGCTGGCTCCCGGATTTATCGATGTGCATACCCAT | Klebsiella phage Sugarland |
| 486 | GGAAGGCGTGGCGCGGCCTGGGCTTGGTCGCATGGATGACGACGGGGTCGGGGTGGTCCGGCGATGGAGGCGGGGCAGAGGACGGGCGGGGCAGGGGAGCGCCGGCGAGCTGCGGTAGAAGATGGCCGGGTCCAGCGAGAGCTCCTGTAG | Klebsiella phage Sugarland |
| 487 | ACCCGGGATCAAACCTACAACCATATCCCACATGGTAGCGGGAGTACCGTTCACATTAATCAGTTCGCTTGCACCCGGAGCGGCTGTAGCAGCCTGTCCCAACATCGTAGCGCCGGTAAAACCGTCCACTACCTGTCCGCCACCGATACC | Klebsiella phage Sugarland |
| 488 | ATCTCATCTCTTAATAGTTCATTAATCAATGTCATTTCATTCATAGGCATGATCAGTGAAAAAAGACTGGAAATCATAAGAGATAATGACATTACAATAAATTCTTTTTTTTCATGAAATGATAGTTTTTGAGCTCGTTCACCTTGCAAC | Klebsiella phage Sugarland |
| 489 | ATGCACTCTTTTTTGCGCAAAAAGGGCTACGCGTGGAATTTATCGACCTTGATACCACCACAGTTACGACTGATAAGAAGTGGTTGATCTGTATTTTAGAGCAATTGATCTTCAATGCGATCAAATATACCAACACTGGAACGATCACGT | Klebsiella phage Sugarland |
| 490 | CATTGGCTTGAGGCAACTGCGCCCATTGATCGGGGCTGGAATACCCTGCTGAATCTGCGTTCATGGGCAATGGTCGGCAGCGGCCTCGTGGCAATATGGTCAGTTCGTCATCCGCGCTTTTTGATGCGCTGGGCAAAACGCAGCCTCGGT | Klebsiella phage Sugarland |

List 7 Sequence classified as Klebsiella_phage_vB_Kpn_IME260 (NCBI: taxid 1912318), Klebsiella_phage_K64-1 (NCBI: taxid 1439894), Klebsiella_virus_0507KN21 (NCBI: taxid 2169687), Klebsiella_phage_KpV71 (NCBI: taxid 1796998), and Klebsiella phage_Matisse (NCBI: taxid 1912318) in mice fecal metagenomics data

| SEQ ID NO: | Klebsiella virus (target bacteria Klebsiella pneumoniae) sequences in whole-community metagenomes in post-VMT samples | Notes |
|---|---|---|
| 491 | ATGCCGCTCGGAATTTGGAGCTTGGCAACGATTTCGTTAAATTTGACGCAACGTTTAACGGTGTCGCTGAAAGTATTCAGGTTCCCGTCGGCAACATAGAGGCGGTGTTTGTCAGAGAGACCGGCTTTGGCATCCCGTTCGAGGTCAAAC | Klebsiella_phage_vB_Kpn_IME260 |
| 492 | CTTCGGCAGTACAATATCAGGATTTCCCGGCAGCTTCCTTACCTGAACGCGGAAACGCAAACCTCGTGAGAAAAGATACTTCCGCACAATCAGTTCCGGTTTGGTATCCTTTCCCTTGATAGCCGCCATACAGCGGCTACGCTGCTCCGG | Klebsiella_phage_vB_Kpn_IME260 |
| 493 | TACAGGCACTGTTTTTGGCCGCGGTTCCCGATCAATTCCTTTGCAAGCTTCAGCCGTTGTCCTTCACCGCCGGATAAGGTAGTCAAAGGCTGGCCAAGCTGCAGATAGCCAAGTCCCACATCCTGCAATAGAGCCAAGATTCCCGTGATC | Klebsiella_phage_vB_Kpn_IME260 |
| 494 | TCTTCAAATCGGAGACTCAGGAGACGTAGACCTTTTTATCCGAACCAGCTCTGAAAGCCGCATTAGTAATTTTCTCTTGTGGGAGCTTGCCTATAGTGAGCTCTACTTTACCTCCGTTTTATGGCCGGATTTTGGTAAAAGAGAATTTA | Klebsiella_phage_vB_Kpn_IME260 |
| 495 | ACCATCAACTAAGAGATAACGGTAGCTAAAATCTTGCGCCATCCGGACCATCGATTCATAGATCGCCGAGTCCCCATGCGGGTGGTACTTACCCATAACATCCCCAACGATCCGGGCTGATTTCTTATATGGTTTATCCGGAGTCACACC | Klebsiella_phage_vB_Kpn_IME260 |
| 496 | TTCAATATCGGACCGTATGCCGCTCGGAATTTGGAGCTTGGCAACGATTTCGTTAAATTTGACGCAACGTTTAACGGTGTCGCTGAAAGTATTCAGGTTCCCGTCGGCAACATAGAGGCGGTGTTTGTCAGAGAGACCGGCTTTGGCATC | Klebsiella_phage_vB_Kpn_IME260 |
| 497 | CTACGCGGTACATGGGCACACTGCCGCCGAATTGATCGTAGACCGGGCCGACGCCCAGAAAGAGCACATGGGCTTGACAACGTGGGAGAACGCTCCAGATGGTAAAATCCTGAAACCGGATGTAAGCATCGCCAAGAACTATCTCAAAGA | Klebsiella_phage_vB_Kpn_IME260 |
| 498 | TACGCTTCCTTTATCGCTACGAGCAATCATACGGATTTGCTGGGCGATCCTTCCGGCAGCCGCCGTTTCATTTGTATCGAGGTGAAAGGGATGATAGACAATGCCCAGCCTATCGATTATCTCCAGCTCTATGCGCAAGCGGTTGCCGCC | Klebsiella_phage_vB_Kpn_IME260 |
| 499 | AGAACGGGCGAACAAAGCCAAAAGTGAATTCTTATCCAATATGAGTCATGATATCCGTACGCCTATGAATGCCATTGTCGGTATGACATCCATTGCTTCGGCCAATTTGGAGAATACAAAACAGGTGCAAAATTGTCTGAAGAAGATTAC | Klebsiella_phage_vB_Kpn_IME260 |
| 500 | AGCGTACGCAAACGGTTCGCAAATCACGTTCAATATCGGACCGTATGCCGCTCGGAATTTGGAGCTTGGCAACGATTTCGTTAAATTTGACGCAACGTTTAACGGTGTCGCTGAAAGTATTCAGGTTCCCGTCGGCAACATAGAGGCGGT | Klebsiella_phage_vB_Kpn_IME260 |
| 501 | AACGATTTCGTTAAATTTGACGCAACGTTTAACGGTGTCGCTGAAAGTATTCAGGTTCCCGTCGGCAACATAGAGGCGGTGTTTGTCAGAGAGACCGGCTTTGGCATCCCGTTCGAGGTCAAACTTAAACCTGACCAAGAACCCGCGCCC | Klebsiella_phage_vB_Kpn_IME260 |
| 502 | GCTCTGAAAGCCGCATTAGTAATTTTCTCTTGTGGGAGCTTGCCTATAGTGAGCTCTACTTTACCTCCGTTTTATGGCCGGATTTTGGTAAAAGAGAATTTAAAGAAGCATTATTGTGGTACGCATCAAGAGAAAGGCGCTTTGGTAAGA | Klebsiella_phage_vB_Kpn_IME260 |
| 503 | TGACTATATCATCGACGTACTCCTTGCACAACGCAAATGTATTATCGCCCGGTTCTTTTACCGATATGCCGTCGGCCACGGTCGACACACTCGGCAGGCACTCTATCTTACCGTGCTTCAGCGACTCTACCATGCTCGGCGCTCCGGCGG | Klebsiella_phage_vB_Kpn_IME260 |
| 504 | CGACGTTATAAACAATGTGTTCTGCACACTCTTCTAAAGTGAAAAAATCATAGAAATACTGCCGAAATTCTTCTAACCATTCCTCTGTGAAATATTCTGGATCAAGTTCAACTTTTACTTTGTCACGTCTTACTAATTCAATTTCAAATT | Klebsiella_phage_K64-1 |
| 505 | CATACATCAGTCGACTGCAGCCAAGATATATCATGTGAATCTGCCGGATGTCACCGAGGAGCAGCGTCGCCGTGCCAAGACCGCCAATTTCGGTATAATTTACGGTATATCGGCATTCGGTCTCGCTCAGAGACTGCGCATACCGCGATA | Klebsiella_phage_K64-1 |
| 506 | CGATTTTCGCCCGTAGCTCGGCAATCTCTTTGCGCAGCCGCTCCTCCTCCAGCAGCTGCGCCGGTTCGCTTTTGCTAATACTCTCCAGATCTTCTTCCAGAGCATGCAGCTCCCGGCGCTTACCGGCGGTCATCGCCGTGCGCCGCGCCA | Klebsiella_phage_K64-1 |
| 507 | AACTTCTTCCCCTTGCGTTTAAATTCAGGGAAGATAGCTCCGGTCATCGTACCGGATGAATCAAACTCCGTCGGGTAACAATTACCGGCCCGTACGATATCTACAGCCACCTTCTCCCCGGTAAGGTATGGCTTAAAGCAAATACCACCA | Klebsiella_phage_K64-1 |
| 508 | GCCCCCGGATTCGGACAGCGCGGCATCGAAGGCAAATTCTCAGCGCTCAAATGGCTTCGTGAGCACGACATACCCACGCTCGGAATATGCCTCGGTATGCAGTGTATGGTCATCGAATACGCCCGCAACGTACTCGGACTTCAGCAGGCC | Klebsiella_phage_K64-1 |
| 509 | GCAGGGATCGTGCTGCAAGGGACTGAGATCAAATCGATCCGCAACAGTCGGATCAATCTAAAAGACGGCTTTGTTCGTATCCGTAATGGAGAAGACTATTTGTATAATGTTCATATAAGTCCTTATGAACAGGGAAACATTTTTAATCAT | Klebsiella_virus_0507K21 |
| 510 | AATATGGGAATGGTCAGCGGGATCCTGCCCGTTGTCGGCGTGCCCCTTCCCTTTATGAGTTACGGCGGTACTGCGCTTCTGATTTTAGGCATCTGCTGCGGACTGCTGATGAAAATTTCCGCCGGACGCCGCATAAAAGAATCTTTGTAC | Klebsiella_virus_0507KN21 |
| 511 | CTATCTGCGCACCTTCCGCTTCACGGGCGACCTGTGGGCTGTGCCGGAGGGCACTCCCATCTTCCCCCGGGAGCCCTTCCTCACCGTCCGCGCCCCCGCCATTGAGGCCCAGTTCATCGAGACCTTCCTGCTGCTCACCCTCAACCACCA | Klebsiella_virus_0507KN21 |
| 512 | GTATATTCTTTATATGTCTTGAAATGGTCGACTTATTGCGTTGAAACAGCTCCGTCATCTGGTCGGCAGTAAGCCACACCGATTCATTGGCAAGCTGAACCTCAATCTTCGTCTCGCCACCCTGCGTCTGAAAAAGTATTATATTTCCGC | Klebsiella_virus_0507KN21 |
| 513 | ATTCATCGCATGCAGAATACGGCGATGAACAGGCTTCATTCCATCACGAACATCAGGCAGTGCACGATCTACGATTACAGACATTGAATAGTCCAGAAAGGAGGTTTTCATTTCTTTTGAAATATCTACCTGAATCAGTTTTTCCTTATT | Klebsiella_virus_0507KN21 |
| 514 | AGATGGGGCTGGAGGACAAGGAAAATGCCTACCCCTGCCAGCTTTCAGGGGGCCAGTGCCAGCGTGTGGCCATTGCAAGGGCATTGGCCCTGAATCCCCAGATCCTGTTCTTTGACGAGCCTACCTCGGCGCTGGATCCGGAACTGACAG | Klebsiella_phage_KpV71 |
| 515 | ATGCCGCTCGGAATTTGGAGCTTGGCAACGATTTCGTTAAATTTGACGCAACGTTTAACGGTGTCGCTGAAAGTATTCAGGTTCCCGTCGGCAACATAGAGGCGGTGTTTGTCAGAGAGACCGGCTTTGGCATCCCGTTCGAGGTCAAAC | Klebsiella_phage_Matisse |
| 516 | CTTCGGCAGTACAATATCAGGATTTCCCGGCAGCTTCCTTACCTGAACGCGGAAACGCAAACCTCGTGAGAAAAGATACTTCCGCACAATCAGTTCCGGTTTGGTATCCTTTCCCTTGATAGCCGCCATACAGCGGCTACGCTGCTCCGG | Klebsiella_phage_Matisse |
| 517 | TACAGGCACTGTTTTTGGCCGCGGTTCCCGATCAATTCCTTTGCAAGCTTCAGCCGTTGTCCTTCACCGCCGGATAAGGTAGTCAAAGGCTGGCCAAGCTGCAGATAGCCAAGTCCCACATCCTGCAATAGAGCCAAGATTCCCGTGATC | Klebsiella_phage_Matisse |
| 518 | TCTTCAAATCGGAGACTCAGGAGACGTAGACCTTTTTATCCGAACCAGCTCTGAAAGCCGCATTAGTAATTTTCTCTTGTGGGAGCTTGCCTATAGTGAGCTCTACTTTACCTCCGTTTTATGGCCGGATTTTGGTAAAAGAGAATTTA | Klebsiella_phage_Matisse |
| 519 | ACCATCAACTAAGAGATAACGGTAGCTAAAATCTTGCGCCATCCGGACCATCGATTCATAGATCGCCGAGTCCCCATGCGGGTGGTACTTACCCATAACATCCCCAACGATCCGGGCTGATTTCTTATATGGTTTATCCGGAGTCACACC | Klebsiella_phage_Matisse |
| 520 | TTCAATATCGGACCGTATGCCGCTCGGAATTTGGAGCTTGGCAACGATTTCGTTAAATTTGACGCAACGTTTAACGGTGTCGCTGAAAGTATTCAGGTTCCCGTCGGCAACATAGAGGCGGTGTTTGTCAGAGAGACCGGCTTTGGCATC | Klebsiella_phage_Matisse |
| 521 | CTACGCGGTACATGGGCACACTGCCGCCGAATTGATCGTAGACCGGGCCGACGCCCAGAAAGAGCACATGGGCTTGACAACGTGGGAGAACGCTCCAGATGGTAAAATCCTGAAACCGGATGTAAGCATCGCCAAGAACTATCTCAAAGA | Klebsiella_phage_Matisse |
| 522 | TACGCTTCCTTTATCGCTACGAGCAATCATACGGATTTGCTGGGCGATCCTTCCGGCAGCCGCCGTTTCATTTGTATCGAGGTGAAAGGGATGATAGACAATGCCCAGCCTATCGATTATCTCCAGCTCTATGCGCAAGCGGTTGCCGCC | Klebsiella_phage_Matisse |
| 523 | AGAACGGGCGAACAAAGCCAAAAGTGAATTCTTATCCAATATGAGTCATGATATCCGTACGCCTATGAATGCCATTGTCGGTATGACATCCATTGCTTCGGCCAATTTGGAGAATACAAAACAGGTGCAAAATTGTCTGAAGAAGATTAC | Klebsiella_phage_Matisse |
| 524 | AGCGTACGCAAACGGTTCGCAAATCACGTTCAATATCGGACCGTATGCCGCTCGGAATTTGGAGCTTGGCAACGATTTCGTTAAATTTGACGCAACGTTTAACGGTGTCGCTGAAAGTATTCAGGTTCCCGTCGGCAACATAGAGGCGGT | Klebsiella_phage_Matisse |
| 525 | AACGATTTCGTTAAATTTGACGCAACGTTTAACGGTGTCGCTGAAAGTATTCAGGTTCCCGTCGGCAACATAGAGGCGGTGTTTGTCAGAGAGACCGGCTTTGGCATCCCGTTCGAGGTCAAACTTAAACCTGACCAAGAACCCGCGCCC | Klebsiella_phage_Matisse |
| 526 | GCTCTGAAAGCCGCATTAGTAATTTTCTCTTGTGGGAGCTTGCCTATAGTGAGCTCTACTTTACCTCCGTTTTATGGCCGGATTTTGGTAAAAGAGAATTTAAAGAAGCATTATTGTGGTACGCATCAAGAGAAAGGCGCTTTGGTAAGA | Klebsiella_phage_Matisse |
| 527 | TGACTATATCATCGACGTACTCCTTGCACAACGCAAATGTATTATCGCCCGGTTCTTTTACCGATATGCCGTCGGCCACGGTCGACACACTCGGCAGGCACTCTATCTTACCGTGCTTCAGCGACTCTACCATGCTCGGCGCTCCGGCGG | Klebsiella_phage_Matisse |

### REFERENCES

1 Ferri, M., Ranucci, E., Romagnoli, P. & Giaccone, V. Antimicrobial resistance: a global emerging threat to public health systems. Critical reviews in food science and nutrition 57, 2857-2876 (2017).

2 Guh, A. Y. et al. Epidemiology of carbapenem-resistant Enterobacteriaceae in 7 US communities, 2012-2013. JAMA 314, 1479-1487 (2015).

3 Sievert, D. M. et al. Antimicrobial-resistant pathogens associated with healthcare-associated infections summary of data reported to the National Healthcare Safety Network at the Centers for Disease Control and Prevention, 2009-2010. Infection Control & Hospital Epidemiology 34, 1-14 (2013).

4 Satlin, M. J. et al. Multicenter clinical and molecular epidemiological analysis of bacteremia due to carbapenem-resistant Enterobacteriaceae (CRE) in the CRE epicenter of the United States. Antimicrobial agents and chemotherapy 61, e02349-02316 (2017).

5 Gupta, N., Kallen, A. J., Limbago, B. M. & Patel, J. B. Carbapenem-Resistant Enterobacteriaceae: Epidemiology and Prevention. Clinical Infectious Diseases 53, 60-67, doi:10.1093/cid/cir202 (2011).

6 Urban, C. et al. Carbapenem-resistant Escherichia coli harboring Klebsiella pneumoniae carbapenemase β-lactamases associated with long-term care facilities. Clinical Infectious Diseases 46, e127-e130 (2008).

7 Oostdijk, E. A., de Smet, A. M. G., Kesecioglu, J. & Bonten, M. J. J. J. o. A. C. Decontamination of cephalosporin-resistant Enterobacteriaceae during selective digestive tract decontamination in intensive care units. 67, 2250-2253 (2012).

8 Van Nood, E. et al. Duodenal infusion of donor feces for recurrent Clostridium difficile. 368, 407-415 (2013).

9 Manges, A. R., Steiner, T. S. & Wright, A. J. J. I. D. Fecal microbiota transplantation for the intestinal decolonization of extensively antimicrobial-resistant opportunistic pathogens: a review. 48, 587-592 (2016).

10 Manges, A. R., Steiner, T. S. & Wright, A. J. Fecal microbiota transplantation for the intestinal decolonization of extensively antimicrobial-resistant opportunistic pathogens: a review. Infectious Diseases 48, 587-592 (2016).

11 Davido, B. et al. Is faecal microbiota transplantation an option to eradicate highly drug-resistant enteric bacteria carriage? Journal of Hospital Infection 95, 433-437 (2017).

12 Gopalsamy, S. N., Sherman, A., Woodworth, M. H., Lutgring, J. D. & Kraft, C. S. Fecal Microbiota Transplant for Multidrug-Resistant Organism Decolonization Administered During Septic Shock. Infection Control & Hospital Epidemiology 39, 490-492 (2018).

13 Dinh, A. et al. Clearance of carbapenem-resistant Enterobacteriaceae vs vancomycin-resistant enterococci carriage after faecal microbiota transplant: a prospective comparative study. Journal of Hospital Infection 99, 481-486 (2018).

14 Davido, B. et al. Is faecal microbiota transplantation an option to eradicate highly drug-resistant enteric bacteria carriage? 95, 433-437 (2017).

15 Zuo, T. et al. Bacteriophage transfer during faecal microbiota transplantation in Clostridium difficile infection is associated with treatment outcome. 67, 634-643 (2018).

16 Bolger, A. M., Lohse, M. & Usadel, B. Trimmomatic: a flexible trimmer for Illumina sequence data. Bioinformatics 30, 2114-2120 (2014).

17 Wood, D. E. & Salzberg, S. L. Kraken: ultrafast metagenomic sequence classification using exact alignments. Genome biology 15, R46 (2014).

18 Croswell, A., Amir, E., Teggatz, P., Barman, M. & Salzman, N. H. Prolonged impact of antibiotics on intestinal microbial ecology and susceptibility to enteric Salmonella infection. Infection and immunity 77, 2741-2753 (2009).

19 Minot, S. et al. The human gut virome: inter-individual variation and dynamic response to diet. Genome research 21, 1616-1625 (2011).

20 Reyes, A. et al. Viruses in the faecal microbiota of monozygotic twins and their mothers. Nature 466, 334 (2010).

21 Minot, S. et al. Rapid evolution of the human gut virome. Proceedings of the National Academy of Sciences 110, 12450-12455 (2013).

It is understood that the examples and embodiments described herein are for illustrative purposes only and that various modifications or changes in light thereof will be suggested to persons skilled in the art and are to be included within the spirit and purview of this application and scope of the appended claims.

1. A method for identifying a donor subject for fecal microbiota transplantation (FMT), comprising:
(a) analyzing a fecal sample obtained from a candidate subject to detect the presence of one or more predetermined species of bacteriophages in the fecal sample; and
(b) determining the candidate subject as a donor subject when the presence of the one or more predetermined species of bacteriophages is detected in the fecal sample.

2. The method of claim 1, further comprising step (c) administering a fecal material obtained from the donor subject to a subject in need of FMT.

3. (canceled)

4. A method of treating or preventing a bacterial infection in a subject in need of FMT, comprising:
(a) analyzing a fecal sample to detect the presence of one or more predetermined species of bacteriophages in the fecal sample; and
(b) administering to the subject in need of FMT a processed fecal sample containing the predetermined species of bacteriophages.

5. The method of claim 4, wherein the bacterial infection is an antibiotic-resistant bacterial infection.

6. The method of claim 4, wherein the bacterial infection is caused by bacteria in the family Enterobacteriaceae.

7. The method of claim 4, wherein the bacterial infection is caused by bacteria in the genus Enterococcus, Klebsiella, or Escherichia.

8. The method of claim 4, wherein the bacterial infection is caused by carbapenem-resistant Enterobacteriaceae (CRE).

9. The method of claim 4, wherein the bacterial infection is caused by vancomycin-resistant Enterococci (VRE).

10. The method of claim 4, wherein the bacteria infection is caused by Klebsiella pneumonia, Klebsiella variicola, or Escherichia coli.

11. The method of claim 1, wherein the bacteriophage is selected from the group consisting of Klebsiella phage KP34 (NCBI:txid674081), genus KP32virus (NCBI: txid1985720), genus Kp36virus (NCBI: txid1920860), Klebsiella virus Kp15 (NCBI:txid1985328), Klebsiella phage KP27 (NCBI:txid1129147).

12. The method of claim 11, wherein the bacteriophage in the genus KP32virus (NCBI: txid1985720) is selected from the group consisting of Klebsiella phage K5 (NCBI:txid1647374), Klebsiella phage K11 (NCBI:txid532077), Klebsiella phage vB_Kp1 (NCBI:txid1701804), Klebsiella phage KP32 (NCBI:txid674082), and Klebsiella phage vB_KpnP_KpV289 (NCBI:txid1671396).

13. The method of claim 4, wherein the bacterial infection is caused by carbapenem-resistant Klebsiella pneumonia, and the bacteriophage is selected from the group consisting of Klebsiella phage KP34 (NCBI:txid674081), genus KP32virus (NCBI: txid1985720), and genus Kp36virus (NCBI: txid1920860).

14. The method of claim 13, wherein the bacteriophage comprises a genome comprising a nucleic acid sequence of any one of SEQ ID NOS:1-324 and 333-335.

15. The method of claim 4, wherein the bacterial infection is caused by carbapenem-resistant Klebsiella variicola, and the bacteriophage is selected from the group consisting of Klebsiella virus Kp15 (NCBI:txid1985328) and Klebsiella phage KP27 (NCBI:txid1129147).

16. The method of claim 15, wherein the bacteriophage comprises a genome comprising a nucleic acid sequence of any one of SEQ ID NOS:325-332.

17. The method of claim 4, wherein the bacterial infection is caused by carbapenem-resistant Escherichia coli, and the bacteriophage comprises a genome comprising a nucleic acid sequence of any one of SEQ ID NOS:336-384.

18-20. (canceled)

21. The method of claim 1, wherein the fecal sample comprises a bacteriophage selected from the group consisting of Klebsiella phage KP34 (NCBI:txid674081), genus KP32virus (NCBI: txid1985720), and genus Kp36virus (NCBI: txid1920860).

22. The method of claim 21, wherein the fecal sample comprises a bacteriophage comprising a sequence of any one of SEQ ID NOS: 1-324 and 333-335.

23. The method of claim 1, wherein the fecal sample comprises a bacteriophage selected from the group consisting of Klebsiella virus Kp15 (NCBI:txid1985328) and Klebsiella phage KP27 (NCBI:txid1129147).

24. The method of claim 23, wherein the fecal sample comprises a bacteriophage comprising a sequence of any one of SEQ ID NOS:325-332.

25-31. (canceled)
